(19) United States
(12) Patent Application Publication
(10) Pub. No.: US 2023/0270729 A1
(43) Pub. Date: Aug. 31, 2023

(54) USE OF N-PHENYLACETAMIDES HAVING P2X4 RECEPTOR ANTAGONISTIC ACTIVITY FOR TREATING CERTIAN OCULAR DISORDERS

(71) Applicants: Bayer Aktiengesellschaft, Leverkusen (DE); The Johns Hopkins University, Baltimore, MD (US)

(72) Inventors: Stefan BÄURLE, Berlin (DE); William SCHUBERT, Wuppertal (DE); Jens NAGEL, Daxweiler (DE); Carsten TERJUNG, Bochum (DE); David FINIS, Düsseldorf (DE); Samuel YIU, Baltimore, MD (US); Hui LIN, Baltimore, MD (US); Minjie CHEN, Baltimore, MD (US); Stefanie SEO, Baltimore, MD (US)

(73) Assignees: Bayer Aktiengesellschaft, Leverkusen (DE); The Johns Hopkins University, Baltimore, MD (US)

(21) Appl. No.: 18/012,446

(22) PCT Filed: Jun. 28, 2021
(86) PCT No.: PCT/EP2021/067714

§ 371 Date: Dec. 22, 2022

Related U.S. Application Data
(60) Provisional application No. 63/202,797, filed on Jun. 24, 2021.

(30) Foreign Application Priority Data
Jun. 30, 2020 (EP) 20183306.8
Jan. 15, 2021 (EP) 21151884.0

(51) Int. Cl. A61K 31/44

(52) U.S. Cl. CPC A61K 31/44

(57) ABSTRACT

The use of P2X4 antagonists for the treatment of dry eye syndrome, more in particular substituted N-phenylacetamide compounds of general formula (I), pharmaceutical compositions and combinations comprising said compounds for use in the treatment or prophylaxis of dry eye syndrome and in particular dry eye, ocular neuropathic pain, ocular trauma and post-operative ocular pain.

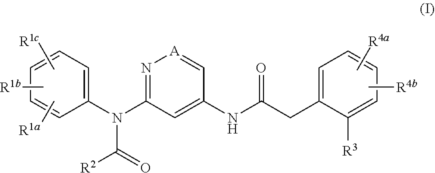

The present invention covers the use of P2X4 antagonists for the treatment of dry eye syndrome, more in particular substituted N-phenylacetamide compounds of general formula (I) as described and defined herein, for manufacturing pharmaceutical compositions for the treatment or prophylaxis of dry eye syndrome and in particular dry eye, ocular neuropathic pain, ocular trauma and post-operative ocular pain.

### BACKGROUND

Eye pain is an unpleasant sensory and emotional experience including sensory-discriminative, emotional, cognitive, and behavioral components and supported by distinct, interconnected peripheral and central nervous system elements. Normal or physiological pain results of the stimulation by noxious stimuli of sensory axons of trigeminal ganglion (TG) neurons innervating the eye. These are functionally heterogeneous. Mechano-nociceptors are only excited by noxious mechanical forces. Polymodal nociceptors also respond to heat, exogenous irritants, and endogenous inflammatory mediators, whereas cold thermoreceptors detect moderate temperature changes. Their distinct sensitivity to stimulating forces is determined by the expression of specific classes of ion channels: Piezo2 for mechanical forces, TRPV1 and TRPA1 for heat and chemical agents, and TRPM8 for cold. Pricking pain is evoked by mechano-nociceptors, while polymodal nociceptors are responsible of burning and stinging eye pain; sensations of dryness appear to be mainly evoked by cold thermoreceptors. Mediators released by local inflammation, increase the excitability of eye polymodal nociceptors causing their sensitization and the augmented pain sensations. During chronic inflammation, additional, longlasting changes in the expression and function of stimulus transducing and voltage-sensitive ion channels develop, thereby altering polymodal terminal's excitability and evoking chronic inflammatory pain. When trauma, infections, or metabolic processes directly damage eye nerve terminals, these display aberrant impulse firing due to an abnormal expression of transducing and excitability modulating ion channels. This malfunction evokes ‘neuropathic pain’ which may also result from abnormal function of higher brain structures where ocular TG neurons project. Eye diseases or ocular surface surgery cause different levels of inflammation and/or nerve injury, which in turn activate sensory fibers of the eye in a variable degree. When inflammation dominates (allergic or actinic kerato-conjunctivitis), polymodal nociceptors are primarily stimulated and sensitized, causing pain. In uncomplicated photorefractive surgery and moderate dry eye, cold thermoreceptors appear to be mainly affected, evoking predominant sensations of unpleasant dryness. (“What Causes Eye Pain?” Carlos Belmonte et al. Curr Ophthalmol Rep (2015) 3:111-121)

For the first time it has been identified that P2X4 plays an essential role in the treatment of pain in the eye.

The substituted N-phenylacetamides of general formula (I) as represented in the following are antagonists or negative allosteric modulators of P2X4. Adenosine triphosphate ATP is widely recognized as an important neurotransmitter implicated in various physiological and pathophysiological roles by acting through different subtypes of purinergic receptors (Burnstock 1993, Drug Dev Res 28:196-206; Burnstock 2011, Prog Neurobiol 95:229-274). To date, seven members of the P2X family have been cloned, comprising P2X1-7 (Burnstock 2013, Front Cell Neurosci 7:227). The P2X4 receptor is a ligand-gated ion channel that is expressed on a variety of cell types largely known to be involved in inflammatory/immune processes specifically including monocytes, macrophages, mast cells and microglia cells (Wang et al., 2004, BMC Immunol 5:16; Brone et al., 2007 Immunol Lett 113:83-89). Activation of P2X4 by extracellular ATP is known, amongst other things, to lead to release of pro-inflammatory cytokines and prostaglandins (PGE2) (Bo et al., 2003 Cell Tissue Res 313:159-165; Ulmann et al., 2010, EMBO Journal 29:2290-2300; de Ribero Vaccari et al., 2012, J Neurosci 32:3058-3066). Numerous lines of evidence in the literature using animal models implicate P2X4 receptor in nociception and pain. Mice lacking the P2X4 receptor do not develop pain hypersensitivity in response to numerous inflammatory challenges such as complete Freunds Adjuvant (CFA), carrageenan or formalin (Ulmann et al., 2010, EMBO Journal 29:2290-2300). In addition, mice lacking the P2X4R do not develop mechanical allodynia after peripheral nerve injury, indicating very prominent role of P2X4 in neuropathic pain conditions (Tsuda et al., 2009, Mol Pain 5:28; Ulmann et al., 2008, J Neurocsci 28:11263-11268). Moehring et al. (Elife. 2018 Jan. 16; 7 “Keratinocytes mediate innocuous and noxious touch via ATP-P2X4 signaling”) reported experiments identifying P2X4 signalling as a critical component of baseline mammalian tactile sensation. These experiments lay a vital foundation for subsequent studies into the dysfunctional signalling that occurs in cutaneous pain and itch disorders.

EP2597088A1 describes P2X4 receptor antagonists and a diazepine derivative of formula (III) or a pharmacologically acceptable salt thereof. Said document further disclosed the use of P2X4 receptor antagonist diazepine derivatives represented by the formula (I), (II), (III), or its pharmacologically acceptable salt, which shows P2X4 receptor antagonism, being effective as an agent for prevention or treatment of nociceptive, inflammatory, and neuropathic pain. In more detail, EP2597088A1 describes P2X4 receptor antagonists being effective as a preventive or therapeutic agent for pain caused by various cancers, diabetic neuritis, viral diseases such as herpes, and osteoarthritis. The preventive or therapeutic agent according to EP2597088A1 can also be used in combination with other agents such as opioid analgesic (e.g., morphine, fentanyl), sodium channel inhibitor (e.g., novocaine, lidocaine), or NSAIDs (e.g., aspirin, ibuprofen). The P2X4 receptor antagonist used for pain caused by cancers can be also used in combination with a carcinostatic such as a chemotherapic. Further P2X4 receptor antagonists and their use are disclosed in WO2013105608, WO2015005467 and WO2015005468, WO2016198374, WO2017191000, WO2018/104305, WO2018/104307.

“Discovery and characterization of novel, potent and selective P2X4 receptor antagonists for the treatment of pain” was presented at the Society for Neuroscience Annual Meeting 2014 (Carrie A Bowen et al.; poster N. 241.1) Said poster describes the methods to identify novel, potent and selective small-molecule antagonists that inhibit P2X4 across species, and how to evaluate selected compounds in experimental models of neuropathic and inflammatory pain. In particular a method for human, rat, mouse P2X4R FLIPR-based screening, a human P2X4R electrophysiology assay, a suitable mouse neuropathy model and a mouse inflammation model were described.

There is no reference in the state of the art about the use of P2X4 antagonists for the treatment of for the treatment or prophylaxis of dry eye syndrome and in particular dry eye, ocular neuropathic pain, ocular trauma and post-operative ocular pain, and particularly the use of substituted N-phenylacetamides of general formula (I) as described and defined herein for manufacturing a pharmaceutical composition for the treatment or prophylaxis of dry eye syndrome and in particular dry eye, ocular neuropathic pain, ocular trauma and post-operative ocular pain, as a sole agent or in combination with other active ingredients.

Therefore, the inhibitors of P2X4 in general and in in particular those disclosed herein represent valuable therapeutic options in the treatment or prophylaxis of dry eye syndrome and in particular dry eye, ocular neuropathic pain, ocular trauma and post-operative ocular pain either as single agents or in combination with other drugs.

### DESCRIPTION OF THE INVENTION

In accordance to the main aspect, the present invention covers compounds inhibiting P2X4 for use in the treatment or prophylaxis of dry eye syndrome and in particular dry eye, ocular neuropathic pain, ocular trauma, and post-operative ocular pain.

In accordance with a first aspect, the present invention covers compounds of general formula (I):

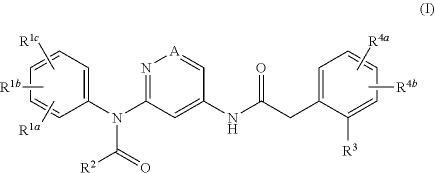

in which A is CH or N
R^{1a}, R^{1b}, and R^{1c} mean independently from each other a hydrogen atom, a halogen atom, cyano, (C₁-C₃)-alkyl, (C₁-C₃)-haloalkyl, (C₁-C₃)-alkoxy,
R² is (C₁-C₃)-alkyl;
R³ means a halogen atom, cyano, (C₁-C₃)-alkyl, (C₁-C₃)-haloalkyl, (C₁-C₃)-alkoxy,
R^{4a} and R^{4b} mean independently from each other a hydrogen atom, a halogen atom, cyano, (C₁-C₃)-alkyl, (C₁-C₃)-haloalkyl, (C₁-C₃)-alkoxy,
and stereoisomers, tautomers, N-oxides, hydrates, solvates, and salts thereof, and mixtures of same for use in the treatment or prophylaxis dry eye syndrome and in particular dry eye, ocular neuropathic pain, ocular trauma, and post-operative ocular pain.

In a second aspect, the present invention covers compounds of general formula (Ia):

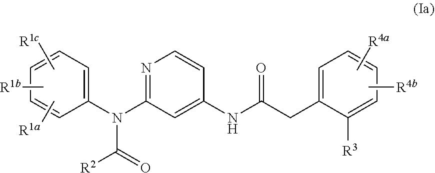

in which
R^{1a}, R^{1b}, and R^{1c} mean independently from each other a hydrogen atom, a halogen atom, cyano, (C₁-C₃)-alkyl, C₁-C₃-haloalkyl, (C₁-C₃)-alkoxy;
R² is (C₁-C₃)-alkyl;
R³ means a halogen atom, cyano, (C₁-C₃)-alkyl, (C₁-C₃)-haloalkyl, (C₁-C₃)-alkoxy;
R^{4a} and R^{4b} mean independently from each other a hydrogen atom, a halogen atom, cyano, (C₁-C₃)-alkyl, (C₁-C₃)-haloalkyl, (C₁-C₃)-alkoxy;
and stereoisomers, tautomers, N-oxides, hydrates, solvates, and salts thereof, and mixtures of same for use in the treatment or prophylaxis of dry eye syndrome and in particular dry eye, ocular neuropathic pain, ocular trauma, and post-operative ocular pain.

In a further aspect, the present invention covers compounds of general formula (Ib):

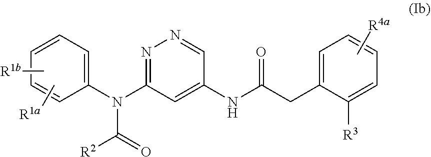

R^{1a}, R^{1b}, and R^{1c} mean independently from each other a hydrogen atom, a halogen atom, cyano, (C₁-C₃)-alkyl, (C₁-C₃)-haloalkyl, (C₁-C₃)-alkoxy,
R² is (C₁-C₃)-alkyl;
R³ means a halogen atom, cyano, (C₁-C₃)-alkyl, (C₁-C₃)-haloalkyl, (C₁-C₃)-alkoxy;
R^{4a} and R^{4b} mean independently from each other a hydrogen atom, a halogen atom, cyano, (C₁-C₃)-alkyl, (C₁-C₃)-haloalkyl, (C₁-C₃)-alkoxy;
and stereoisomers, tautomers, N-oxides, hydrates, solvates, and salts thereof, and mixtures of same for use in the treatment or prophylaxis of dry eye syndrome and in particular dry eye, ocular neuropathic pain, ocular trauma, and post-operative ocular pain.

A further aspect covers compounds of formula (I) according to WO2017191000

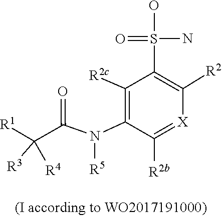

in which
X represents C—R^{2a} or N;
R¹ represents a group selected from:

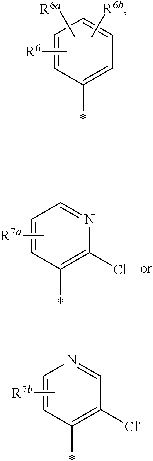

wherein * indicates the point of attachment of said group with the rest of the molecule;
R² represents phenyl or heteroaryl,
   wherein said phenyl or heteroaryl groups are optionally substituted one to three times with R¹¹, being, independently from each other, the same or different, or
   substituted one time with R^{11a} and optionally one to two times with R¹¹ being independently from each other, the same or different, or
   substituted with two adjacent substituents R¹¹ which together represent a methylendioxy group to form a 5-membered ring;
R^{2a} represents hydrogen, cyano, nitro, halogen, C₁-C₂-alkyl or C₁-C₂-haloalkyl;
R^{2b} represents hydrogen, halogen, C₁-C₂-alkyl or C₁-C₂-haloalkyl;
R^{2c} represents hydrogen, halogen, C₁-C₂-alkyl or C₁-C₂-haloalkyl, wherein not less than one of R^{2a}, R^{2b} and R^{2c} represents hydrogen;
R³ represents hydrogen or fluoro;
R⁴ represents hydrogen, fluoro, methyl or OH;
R⁵ represents hydrogen or C₁-C₃-alkyl;
R⁶ represents halogen, cyano, nitro, OH, C₁-C₄-alkyl, C₁-C₄-haloalkyl, C₁-C₄-alkoxy, C₁-C₄-haloalkoxy or F₃CS—;
R^{6a} and R^{6b} are the same or different and represent, independently from each other, respectively
R^{6a} hydrogen, halogen, hydroxy, nitro, cyano, C₁-C₄-alkyl, C₃-C₆-cycloalkyl, C₁-C₄-haloalkyl, C₁-C₄-alkoxy, C₁-C₄-haloalkoxy, HO—(C₂-C₄-alkoxy)-, (C₁-C₄-alkoxy)-(C₂-C₄-alkoxy)-, R⁹R¹⁰N—, R⁸—C(O)—NH—, R⁸—C(O)—, R⁸—O—C(O)—, R⁹R¹⁰N—C(O)— or (C₁-C₄-alkyl)-SO₂—;
R^{6b} hydrogen, halogen, hydroxy, nitro, cyano, C₁-C₄-alkyl, C₃-C₆-cycloalkyl, C₁-C₄-haloalkyl, C₁-C₄-haloalkoxy, HO—(C₂-C₄-alkoxy)-, (C₁-C₄-alkoxy)-(C₂-C₄-alkoxy)-, R⁹R¹⁰N—, R⁸—C(O)—NH—, R⁸—C(O)—, R⁸—O—C(O)—, R⁹R¹⁰N—C(O)— or (C₁-C₄-alkyl)-SO₂—; or
R^{6a} and R^{6b} adjacent to each other together represent a group selected from —O—CH₂—CH₂—, —O—CH₂—O— or —O—CH₂—CH₂—O—;
R^{7a} and R^{7b} are the same or different and represent, independently from each other, hydrogen, hydroxy, halogen, C₁-C₄-alkyl or C₁-C₄-haloalkyl;
R⁸ represents, independently from each respective occurrence, C₁-C₆-alkyl, C₁-C₄-alkoxy-C₁-C₄-alkyl, C₃-C₆-cycloalkyl or C₁-C₄-haloalkyl;
R⁹ and R¹⁰ are the same or different and represent, independently from each other, hydrogen, C₁-C₄-alkyl, C₃-C₆-cycloalkyl, C₁-C₄-haloalkyl or (CH₃)₂N—C₁-C₄-alkyl or
   together with the nitrogen atom to which they are attached form a 4- to 6-membered nitrogen containing heterocyclic ring, said ring optionally containing one additional heteroatom selected from O, S, NH, NR^{a} in which R^{a} represents a C₁-C₆-alkyl or C₁-C₆-haloalkyl group and being optionally substituted, one to three times, independently from each other, with halogen or C₁-C₄-alkyl;
R¹¹ represents, independently from each other, halogen, hydroxy, nitro, cyano,
   C₁-C₄-alkyl, C₂-C₄-alkenyl, C₁-C₄-haloalkyl, C₁-C₆-hydroxyalkyl, C₁-C₄-alkoxy, C₁-C₄-haloalkoxy, (C₁-C₄-alkoxy)-(C₁-C₄-alkyl)-, (C₁-C₄-haloalkoxy)-(C₁-C₄-alkyl)-, R⁹R¹⁰N—(C₁-C₄-alkyl)-, R⁹R¹⁰N—, R^{a}—C(O)—NH—, R^{a}—C(O)—, R^{a}—O—C(O)—, R⁹R¹⁰N—C(O)—, (C₁-C₄-alkyl)-S— or (C₁-C₄-alkyl)-SO₂—;
R^{11a} represents a group selected from C₃-C₆-cycloalkyl, morpholino,

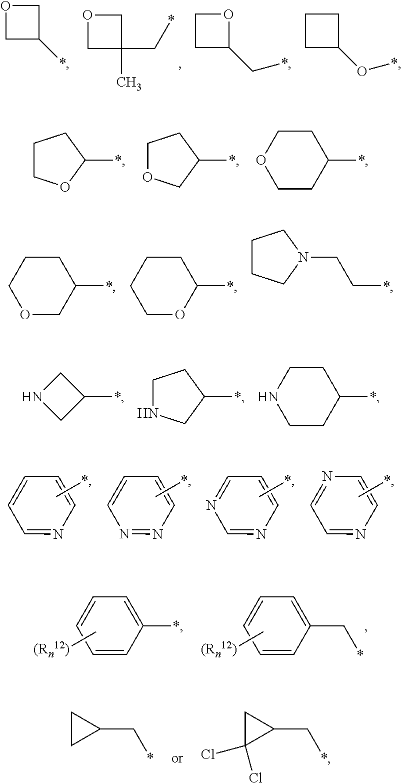

wherein * indicates the point of attachment of said group with the rest of the molecule;
R¹² represents, independently from each other, halogen, hydroxy, nitro, cyano,
   C₁-C₄-alkyl, C₂-C₄-alkenyl, C₁-C₄-haloalkyl, C₁-C₄-hydroxyalkyl, C₁-C₄-alkoxy, C₁-C₄-haloalkoxy, (C₁-C₄-alkoxy)-(C₂-C₄-alkyl)-, (C₁-C₄-haloalkoxy)-(C₂-C₄-alkyl)-, R⁹R¹⁰N—, R⁸—C(O)—NH—, R⁸—C(O)—, R⁸—O—C(O)—, R⁹R¹⁰N—C(O)— or (C₁-C₄-alkyl)-SO₂—;
n represents 0, 1, 2 or 3;
   or an N-oxide, a salt, a hydrate, a solvate, a tautomer or a stereoisomer of said compound, or a salt of said N-oxide, tautomer or stereoisomer for use in the treatment or prophylaxis of dry eye syndrome and in particular dry eye, ocular neuropathic pain, ocular trauma, and post-operative ocular pain.

The synthesis of the compounds of formula (I) according to WO2017191000 is described on pages 80 to 90 of WO2017191000 and is incorporated herein. Further General Experimental Procedure for the synthesis of said compounds and the synthesis of the relevant intermediates is described on pages 98 to 228 of WO2017191000 and is incorporated herein. specific examples of compounds of formula (I) according to WO2017191000 are

More in particular to the compound of formula 2-(2-Chlorophenyl)-N-[4-(4-cyano-1H-pyrazol-1-yl)-3-sulfamoylphenyl]acetamide or an N-oxide, a salt, a hydrate, a solvate, a tautomer or a stereoisomer of said compound, or a salt of said N-oxide, tautomer or stereoisomer, example 39 of WO2017191000 page 253 incorporated herein, for use in the treatment or prophylaxis of dry eye syndrome and in particular dry eye, ocular neuropathic pain, ocular trauma and post-operative ocular pain.
2-(Dimethylamino)-N-[4-(2-oxo-2,3-dihydro-1H-naphtho[1,2-e][1,4]diazepin-5-yl)phenyl]nicotinamide (US20180319752 Example 15×2 HCl, paragraphs 0183-0184 incorporated herein)
5-[4-(2-Iodobenzoylamino)phenyl]-1H-naphtho[1,2-b][1,4]diazepine-2,4(3H,5H)-dione (US2018201587 Example 48, paragraphs 0232-0233 incorporated herein)
5-[4-[(2-(Trifluoromethyl)benzoyl]aminophenyl]-1H-naphtho[1,2-b][1,4]diazepine-2,4(3H,5H)-dione (US2018201587 Example 2, paragraphs 0128-0129 incorporated herein)
5-[3-(1H-Tetrazol-5-yl)phenyl]-1H-naphtho[1,2-b][1,4]diazepine-2,4(3H,5H)-dione (US20130281441 Example 1, paragraphs 0093-0101 incorporated herein)
2-(2-chlorophenyl)-N-[2-(difluoromethyl)-4-sulfamoyl-2H-indazol-6-yl]acetamide (Example 1—WO2021104486 paragraph 0824-0827 incorporated herein)
2-(2-chlorophenyl)-N-(4-sulfamoyl-2H-indazol-6-yl)acetamide (Example 2—WO2021104486, paragraph 0828-0854 incorporated herein)
2-(2-chlorophenyl)-N-(2-methyl-4-sulfamoyl-2H-indazol-6-yl)acetamide (Example 3a —WO2021104486 paragraph 0855-0868 incorporated herein)
2-(2-chlorophenyl)-N-(1-methyl-4-sulfamoyl-1H-indazol-6-yl)acetamide (Example 3b—WO2021104486, paragraph 0855-0868 incorporated herein)

### Definitions

The term “comprising” when used in the specification includes “consisting of”.

If within the present text any item is referred to as “as mentioned herein”, it means that it may be mentioned anywhere in the present text.

The terms as mentioned in the present text have the following meanings:

The term “halogen atom” means a fluorine, chlorine, bromine or iodine atom, particularly a fluorine, chlorine or bromine atom, more particularly fluorine or chlorine atom.

In the context of the present invention, the substituents and residues have the following meanings, unless specified otherwise:

(C₁-C₃)-Alkyl in the context of the invention means a straight-chain or branched alkyl group having 1, 2, or 3 carbon atoms, such as: methyl, ethyl, n-propyl, isopropyl, and isobutyl, for example.

(C₁-C₃)-Alkoxy in the context of the invention means a straight-chain or branched alkoxy group having 1, 2, or 3 carbon atoms, such as: methoxy, ethoxy, n-propoxy, and isopropoxy, for example.

Where the plural form of the word compounds, salts, polymorphs, hydrates, solvates and the like, is used herein, this is taken to mean also a single compound, salt, polymorph, isomer, hydrate, solvate or the like.

By “stable compound” or “stable structure” is meant a compound that is sufficiently robust to survive isolation to a useful degree of purity from a reaction mixture, and formulation into an efficacious therapeutic agent.

The compounds of the present invention optionally contain one or more asymmetric centers, depending upon the location and nature of the various substituents desired. It is possible that one or more asymmetric carbon atoms are present in the (R) or (S) configuration, which can result in racemic mixtures In certain instances, it is possible that asymmetry also be present due to restricted rotation about a given bond, for example, the central bond adjoining two substituted aromatic rings of the specified compounds.

The purification and the separation of such materials can be accomplished by standard techniques known in the art.

The optically active compounds of the present invention can likewise be obtained by chiral syntheses utilizing optically active starting materials.

In order to distinguish different types of isomers from each other reference is made to IUPAC Rules Section E (Pure Appl Chem 45, 11-30, 1976).

The present invention also covers useful forms of the compounds of the present invention, such as metabolites, hydrates, solvates, prodrugs, salts, in particular pharmaceutically acceptable salts, and/or co-precipitates.

The compounds of the present invention can exist as a hydrate, or as a solvate, wherein the compounds of the present invention contain polar solvents, in particular water, methanol or ethanol for example, as structural element of the crystal lattice of the compounds. It is possible for the amount of polar solvents, in particular water, to exist in a stoichiometric or non-stoichiometric ratio. In the case of stoichiometric solvates, e.g. a hydrate, hemi-, (semi-), mono-, sesqui-, di-, tri-, tetra-, penta- etc. solvates or hydrates, respectively, are possible. The present invention includes all such hydrates or solvates.

Further, it is possible for the compounds of the present invention to exist in free form, e.g. as a free base, or as a free acid, or as a zwitterion, or to exist in the form of a salt. Said salt may be any salt, either an organic or inorganic addition salt, particularly any pharmaceutically acceptable organic or inorganic addition salt, which is customarily used in pharmacy, or which is used, for example, for isolating or purifying the compounds of the present invention.

The term “pharmaceutically acceptable salt” refers to an inorganic or organic acid addition salt of a compound of the present invention. For example, see S. M. Berge, et al. “Pharmaceutical Salts,” J. Pharm. Sci. 1977, 66, 1-19.

A suitable pharmaceutically acceptable salt of the compounds of the present invention may be, for example, an acid-addition salt of a compound of the present invention bearing a nitrogen atom, in a chain or in a ring, for example, which is sufficiently basic, such as an acid-addition salt with an inorganic acid, or “mineral acid”, such as hydrochloric, hydrobromic, hydroiodic, sulfuric, sulfamic, bisulfuric, phosphoric, or nitric acid, for example, or with an organic acid, such as formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-2-naphthoic, nicotinic, pamoic, pectinic, 3-phenylpropionic, pivalic, 2-hydroxyethanesulfonic, itaconic, trifluoromethanesulfonic, dodecylsulfuric, ethanesulfonic, benzenesulfonic, para-toluenesulfonic, methanesulfonic, 2-naphthalenesulfonic, naphthalinedisulfonic, camphorsulfonic acid, citric, tartaric, stearic, lactic, oxalic, malonic, succinic, malic, adipic, alginic, maleic, fumaric, D-gluconic, mandelic, ascorbic, glucoheptanoic, glycerophosphoric, aspartic, sulfosalicylic, or thiocyanic acid, for example.

Further, another suitably pharmaceutically acceptable salt of a compound of the present invention which is sufficiently acidic, is an alkali metal salt, for example a sodium or potassium salt, an alkaline earth metal salt, for example a calcium, magnesium or strontium salt, or an aluminium or a zinc salt, or an ammonium salt derived from ammonia or from an organic primary, secondary or tertiary amine having 1 to 20 carbon atoms, such as ethylamine, diethylamine, triethylamine, ethyldiisopropylamine, monoethanolamine, diethanolamine, triethanolamine, dicyclohexylamine, dimethylaminoethanol, diethylaminoethanol, tris(hydroxymethyl)aminomethane, procaine, dibenzylamine, N-methylmorpholine, arginine, lysine, 1,2-ethylenediamine, N-methylpiperidine, N-methyl-glucamine, N,N-dimethyl-glucamine, N-ethyl-glucamine, 1,6-hexanediamine, glucosamine, sarcosine, serinol, 2-amino-1,3-propanediol, 3-amino-1,2-propanediol, 4-amino-1,2,3-butanetriol, or a salt with a quaternary ammonium ion having 1 to 20 carbon atoms, such as tetramethylammonium, tetraethylammonium, tetra(n-propyl)ammonium, tetra(n-butyl)ammonium, N-benzyl-N,N,N-trimethylammonium, choline or benzalkonium.

Those skilled in the art will further recognise that it is possible for acid addition salts of the claimed compounds to be prepared by reaction of the compounds with the appropriate inorganic or organic acid via any of a number of known methods. Alternatively, alkali and alkaline earth metal salts of acidic compounds of the present invention are prepared by reacting the compounds of the present invention with the appropriate base via a variety of known methods.

The present invention includes all possible salts of the compounds of the present invention as single salts, or as any mixture of said salts, in any ratio.

In the present text, in particular in the Experimental Section, for the synthesis of intermediates and of examples of the present invention, when a compound is mentioned as a salt form with the corresponding base or acid, the exact stoichiometric composition of said salt form, as obtained by the respective preparation and/or purification process, is, in most cases, unknown.

Unless specified otherwise, suffixes to chemical names or structural formulae relating to salts, such as “hydrochloride”, “trifluoroacetate”, “sodium salt”, or “x HCl”, “x CF₃COOH”, “x Na⁺”, for example, mean a salt form, the stoichiometry of which salt form not being specified.

This applies analogously to cases in which synthesis intermediates or example compounds or salts thereof have been obtained, by the preparation and/or purification processes described, as solvates, such as hydrates, with (if defined) unknown stoichiometric composition.

Furthermore, the present invention includes all possible crystalline forms, or polymorphs, of the compounds of the present invention, either as single polymorph, or as a mixture of more than one polymorph, in any ratio.

Moreover, the present invention also includes prodrugs of the compounds according to the invention. The term “prodrugs” here designates compounds which themselves can be biologically active or inactive but are converted (for example metabolically or hydrolytically) into compounds according to the invention during their residence time in the body.

In a further embodiment of the first aspect, the present invention covers compounds of formula (I), supra, in which R^{1a}, and R^{1b} mean independently from each other a halogen atom, cyano, (C₁-C₃)-alkyl, (C₁-C₃)-haloalkyl, (C₁-C₃)-alkoxy; and R^{1c} is a hydrogen atom.

According to a further embodiment of the invention R^{1a} is in position 4 of the phenyl ring and means a halogen atom, cyano, (C₁-C₃)-alkyl, (C₁-C₃)-haloalkyl, (C₁-C₃)-alkoxy; R^{1b} means a hydrogen atom a halogen atom, cyano, (C₁-C₃)-alkyl, (C₁-C₃)-haloalkyl, (C₁-C₃)-alkoxy; and R^{1c} is a hydrogen atom.

Furthermore, in relation to a further form of the invention, R^{1a} is in position 4 of the phenyl ring and means a halogen atom, cyano, (C₁-C₃)-alkyl, (C₁-C₃)-haloalkyl, (C₁-C₃)-alkoxy; R^{1b} is in position 3 of the phenyl ring and a hydrogen atom a halogen atom, cyano, (C₁-C₃)-alkyl, (C₁-C₃)-haloalkyl, (C₁-C₃)-alkoxy; and R^{1c} is a hydrogen atom.

In a further specific embodiment of the invention, R^{1a} means a halogen atom, cyano, (C₁-C₃)-alkyl, (C₁-C₃)-haloalkyl, (C₁-C₃)-alkoxy; R^{1b} and R^{1c} are a hydrogen atom.

A specific embodiment of the invention is that in which R² means methyl, ethyl or n-propyl; more particularly R² means a methyl.

According to a further embodiment of the invention, R³ means a chlorine, fluorine, cyano, or a hydrogen atom

In a further specific embodiment of the invention, R^{4a} is a halogen atom, cyano, (C₁-C₃)-alkyl, (C₁-C₃)-haloalkyl, (C₁-C₃)-alkoxy; and R^{4b} is a hydrogen atom.

Furthermore, in relation to a further form of the invention, R^{4a} is a halogen atom, cyano, (C₁-C₃)-alkyl, (C₁-C₃)-haloalkyl, (C₁-C₃)— alkoxy; and R^{4b} is a halogen atom, cyano, (C₁-C₃)-alkyl, (C₁-C₃)-haloalkyl, (C₁-C₃)-alkoxy.

The invention further comprises particular embodiments in which R³ means a chlorine, fluorine, cyano, R^{4a} is a halogen atom, cyano, (C₁-C₃)-alkyl, (C₁-C₃)-haloalkyl, (C₁-C₃)-alkoxy in position 3 or 6 of the phenyl group; and R^{4b} is a hydrogen atom.

In a further specific embodiment of the invention R³ means a chlorine, fluorine, cyano, R^{4a} is a halogen atom, cyano, (C₁-C₃)-alkyl, (C₁-C₃)-haloalkyl, (C₁-C₃)-alkoxy in position 6 of the phenyl group; and R^{4b} is a halogen atom, cyano, (C₁-C₃)-alkyl, (C₁-C₃)-haloalkyl, (C₁-C₃)-alkoxy in position 4 of the phenyl group.

In a further embodiment of the first aspect, the present invention covers combinations of two or more of the above-mentioned embodiments under the heading “further embodiments of the first aspect of the present invention”.

The present invention covers any sub-combination within any embodiment or aspect of the present invention of compounds of general formula (I), supra.

The present invention covers any sub-combination within any embodiment or aspect of the present invention of intermediate compounds of general formula (VII), (VIII). (XIII). (XIV).

The present invention covers the compounds of general formula (I) for use in the treatment or prophylaxis of dry eye syndrome and in particular dry eye, ocular neuropathic pain, ocular trauma, and post-operative ocular pain which are disclosed in the Example Section of this text, infra, namely:
1. N-{4-[2-(2-chlorophenyl)acetamido]pyridin-2-yl}-N-(3,4-difluorophenyl)acetamide
2. N-{4-[2-(2-chloro-3-fluorophenyl)acetamido]pyridin-2-yl}-N-(4-fluorophenyl)acetamide
3. N-{4-[2-(2-chloro-4-fluorophenyl)acetamido]pyridin-2-yl}-N-[3-(difluoromethyl)phenyl]acetamide
4. N-{4-[2-(2-chloro-3-fluorophenyl)acetamido]pyridin-2-yl}-N-[3-(difluoromethyl)phenyl]acetamide
5. N-{4-[2-(2,6-dichlorophenyl)acetamido]pyridin-2-yl}-N-[3-(difluoromethyl)phenyl]acetamide
6. N-{4-[2-(2-chlorophenyl)acetamido]pyridin-2-yl}-N-[3-(trifluoromethyl)phenyl]acetamide
7. N-{4-[2-(2-chloro-4-fluorophenyl)acetamido]pyridin-2-yl}-N-[3-(trifluoromethyl)phenyl]acetamide
8. N-{4-[2-(2-chloro-3-fluorophenyl)acetamido]pyridin-2-yl}-N-[3-(trifluoromethyl)phenyl]acetamide
9. N-{4-[2-(2,6-dichlorophenyl)acetamido]pyridin-2-yl}-N-[3-(trifluoromethyl)phenyl]acetamide
10. N-{4-[2-(2-chlorophenyl)acetamido]pyridin-2-yl}-N-[4-cyano-3-(trifluoromethyl)phenyl]acetamide
11. N-{4-[2-(2-chloro-3-fluorophenyl)acetamido]pyridin-2-yl}-N-[4-cyano-3-(trifluoromethyl)phenyl]acetamide
12. N-[4-cyano-3-(trifluoromethyl)phenyl]-N-{4-[2-(2,6-dichlorophenyl)acetamido]pyridin-2-yl}acetamide
13. N-{4-[2-(2-chlorophenyl)acetamido]pyridin-2-yl}-N-(3-cyanophenyl)acetamide
14. N-(3-cyanophenyl)-N-{4-[2-(2,6-dichlorophenyl)acetamido]pyridin-2-yl}acetamide
15. N-{4-[2-(2-chloro-6-fluorophenyl)acetamido]pyridin-2-yl}-N-(3-cyanophenyl)acetamide
16. N-(4-chloro-3-cyanophenyl)-N-{4-[2-(2-chlorophenyl)acetamido]pyridin-2-yl}acetamide
17. N-(4-chloro-3-cyanophenyl)-N-{4-[2-(2-chloro-4-fluorophenyl)acetamido]pyridin-2-yl}acetamide
18. N-(4-chloro-3-cyanophenyl)-N-{4-[2-(2-chloro-3-fluorophenyl)acetamido]pyridin-2-yl}acetamide
19. N-(4-chloro-3-cyanophenyl)-N-{4-[2-(2,6-dichlorophenyl)acetamido]pyridin-2-yl}acetamide
20. N-(4-{2-[2-chloro-3-(trifluoromethyl)phenyl]acetamido}pyridin-2-yl)-N-(3-cyano-4-fluorophenyl)acetamide
21. N-{4-[2-(2-chlorophenyl)acetamido]pyridin-2-yl}-N-(3-cyano-4-fluorophenyl)acetamide
22. N-{4-[2-(2-chloro-4-fluorophenyl)acetamido]pyridin-2-yl}-N-(3-cyano-4-fluorophenyl)acetamide
23. N-{4-[2-(2-chloro-3-fluorophenyl)acetamido]pyridin-2-yl}-N-(3-cyano-4-fluorophenyl)acetamide
24. N-(3-cyano-4-fluorophenyl)-N-{4-[2-(2,6-dichlorophenyl)acetamido]pyridin-2-yl}acetamide
25. N-{4-[2-(2-chlorophenyl)acetamido]pyridin-2-yl}-N-phenylacetamide
26. N-{4-[2-(2-chloro-4-fluorophenyl)acetamido]pyridin-2-yl}-N-phenylacetamide
27. N-{4-[2-(2,6-dichlorophenyl)acetamido]pyridin-2-yl}-N-phenylacetamide
28. N-{4-[2-(2-chloro-6-fluorophenyl)acetamido]pyridin-2-yl}-N-phenylacetamide
29. N-{4-[2-(2,3-dimethylphenyl)acetamido]pyridin-2-yl}-N-(4-fluorophenyl)acetamide
30. N-(4-fluorophenyl)-N-(4-{2-[2-(trifluoromethyl)phenyl]acetamido}pyridin-2-yl)acetamide
31. N-(4-{2-[4-chloro-2-(trifluoromethyl)phenyl]acetamido}pyridin-2-yl)-N-(4-fluorophenyl)acetamide
32. N-(4-fluorophenyl)-N-(4-{2-[3-fluoro-2-(trifluoromethyl)phenyl]acetamido}pyridin-2-yl)acetamide
33. N-{4-[2-(2-chloro-6-cyanophenyl)acetamido]pyridin-2-yl}-N-(4-fluorophenyl)acetamide
34. N-{4-[2-(2,6-dimethylphenyl)acetamido]pyridin-2-yl}-N-(4-fluorophenyl)acetamide
35. N-(4-{2-[2-chloro-3-(trifluoromethyl)phenyl]acetamido}pyridin-2-yl)-N-(4-fluorophenyl)acetamide
36. N-{4-[2-(2-chlorophenyl)acetamido]pyridin-2-yl}-N-(4-fluorophenyl)acetamide
37. N-{4-[2-(2-chloro-4-fluorophenyl)acetamido]pyridin-2-yl}-N-(4-fluorophenyl)acetamide
38. N-{4-[2-(2,6-dichlorophenyl)acetamido]pyridin-2-yl}-N-(4-fluorophenyl)acetamide
39. N-{4-[2-(2,6-dichloro-4-fluorophenyl)acetamido]pyridin-2-yl}-N-(4-fluorophenyl)acetamide
40. N-{4-[2-(2-chloro-6-fluorophenyl)acetamido]pyridin-2-yl}-N-(4-fluorophenyl)acetamide
41. N-{4-[2-(2-chloro-4,6-difluorophenyl)acetamido]pyridin-2-yl}-N-(4-fluorophenyl)acetamide
42. N-(4-fluorophenyl)-N-(4-{2-[2-(trifluoromethoxy)phenyl]acetamido}pyridin-2-yl)acetamide
43. N-{4-[2-(2-chlorophenyl)acetamido]pyridin-2-yl}-N-(4-methylphenyl)acetamide
44. N-{4-[2-(2-chloro-4-fluorophenyl)acetamido]pyridin-2-yl}-N-(4-methylphenyl)acetamide
45. N-{4-[2-(2-chloro-3-fluorophenyl)acetamido]pyridin-2-yl}-N-(4-methylphenyl)acetamide
46. N-{4-[2-(2,6-dichlorophenyl)acetamido]pyridin-2-yl}-N-(4-methylphenyl)acetamide
47. N-{4-[2-(2-chlorophenyl)acetamido]pyridin-2-yl}-N-[4-(difluoromethoxy)phenyl]acetamide
48. N-{4-[2-(2-chloro-3-fluorophenyl)acetamido]pyridin-2-yl}-N-[4-(difluoromethoxy)phenyl]acetamide
49. N-{4-[2-(2,6-dichlorophenyl)acetamido]pyridin-2-yl}-N-[4-(difluoromethoxy)phenyl]acetamide
50. N-(3-chloro-4-fluorophenyl)-N-{4-[2-(2-chlorophenyl)acetamido]pyridin-2-yl}acetamide
51. N-(3-chloro-4-fluorophenyl)-N-{4-[2-(2-chloro-4-fluorophenyl)acetamido]pyridin-2-yl}acetamide
52. N-(3-chloro-4-fluorophenyl)-N-{4-[2-(2-chloro-3-fluorophenyl)acetamido]pyridin-2-yl}acetamide
53. N-(3-chloro-4-fluorophenyl)-N-{4-[2-(2,6-dichlorophenyl)acetamido]pyridin-2-yl}acetamide
54. N-{4-[2-(2-chlorophenyl)acetamido]pyridin-2-yl}-N-(3-fluorophenyl)acetamide
55. N-{4-[2-(2-chloro-4-fluorophenyl)acetamido]pyridin-2-yl}-N-(3-fluorophenyl)acetamide
56. N-{4-[2-(2,6-dichlorophenyl)acetamido]pyridin-2-yl}-N-(3-fluorophenyl)acetamide
57. N-{4-[2-(2,6-dichloro-4-fluorophenyl)acetamido]pyridin-2-yl}-N-(3-fluorophenyl)acetamide
58. N-(4-chloro-3-fluorophenyl)-N-{4-[2-(2-chlorophenyl)acetamido]pyridin-2-yl}acetamide
59. N-(4-chloro-3-fluorophenyl)-N-{4-[2-(2-chloro-4-fluorophenyl)acetamido]pyridin-2-yl}acetamide
60. N-(4-chloro-3-fluorophenyl)-N-{4-[2-(2-chloro-3-fluorophenyl)acetamido]pyridin-2-yl}acetamide
61. N-(4-chloro-3-fluorophenyl)-N-{4-[2-(2,6-dichlorophenyl)acetamido]pyridin-2-yl}acetamide
62. N-(3,4-difluorophenyl)-N-{4-[2-(2,3-dimethylphenyl)acetamido]pyridin-2-yl}acetamide
63. N-(3,4-difluorophenyl)-N-(4-{2-[2-(trifluoromethyl)phenyl]acetamido}pyridin-2-yl)acetamide
64. N-{4-[2-(2,4-dichloro-6-methylphenyl)acetamido]pyridin-2-yl}-N-(3,4-difluorophenyl)acetamide
65. N-{4-[2-(2-chloro-4,6-dimethylphenyl)acetamido]pyridin-2-yl}-N-(3,4-difluorophenyl)acetamide
66. N-(3,4-difluorophenyl)-N-{4-[2-(2,6-dimethylphenyl)acetamido]pyridin-2-yl}acetamide
67. N-{4-[2-(2,4-dichlorophenyl)acetamido]pyridin-2-yl}-N-(3,4-difluorophenyl)acetamide
68. N-{4-[2-(2-chloro-4-fluorophenyl)acetamido]pyridin-2-yl}-N-(3,4-difluorophenyl)acetamide
69. N-{4-[2-(2-chloro-4-nitrophenyl)acetamido]pyridin-2-yl}-N-(3,4-difluorophenyl)acetamide
70. N-{4-[2-(2-chloro-4-methoxyphenyl)acetamido]pyridin-2-yl}-N-(3,4-difluorophenyl)acetamide
71. N-{4-[2-(2-chloro-3-fluorophenyl)acetamido]pyridin-2-yl}-N-(3,4-difluorophenyl)acetamide
72. N-{4-[2-(2,6-dichlorophenyl)acetamido]pyridin-2-yl}-N-(3,4-difluorophenyl)acetamide
73. N-{4-[2-(2,6-dichloro-4-fluorophenyl)acetamido]pyridin-2-yl}-N-(3,4-difluorophenyl)acetamide
74. N-{4-[2-(2,6-dichloro-4-methylphenyl)acetamido]pyridin-2-yl}-N-(3,4-difluorophenyl)acetamide
75. N-{4-[2-(2,6-dichloro-4-ethylphenyl)acetamido]pyridin-2-yl}-N-(3,4-difluorophenyl)acetamide
76. N-(3,4-difluorophenyl)-N-(4-{2-[2-(trifluoromethoxy)phenyl]acetamido}pyridin-2-yl)acetamide
77. N-{4-[2-(2-chlorophenyl)acetamido]pyridin-2-yl}-N-(3-fluoro-4-methoxyphenyl)acetamide
78. N-{4-[2-(2-chloro-4-fluorophenyl)acetamido]pyridin-2-yl}-N-(3-fluoro-4-methoxyphenyl)acetamide
79. N-{4-[2-(2-chloro-3-fluorophenyl)acetamido]pyridin-2-yl}-N-(3-fluoro-4-methoxyphenyl)acetamide
80. N-{4-[2-(2,6-dichlorophenyl)acetamido]pyridin-2-yl}-N-(3-fluoro-4-methoxyphenyl)acetamide
81. N-{4-[2-(2-chloro-3-fluorophenyl)acetamido]pyridin-2-yl}-N-[3-fluoro-4-(methanesulfonyl)phenyl]acetamide
82. N-{4-[2-(2,6-dichlorophenyl)acetamido]pyridin-2-yl}-N-[3-fluoro-4-(methanesulfonyl)phenyl]acetamide
83. N-(3,5-difluorophenyl)-N-{4-[2-(2,3-dimethylphenyl)acetamido]pyridin-2-yl}acetamide
84. N-(3,5-difluorophenyl)-N-(4-{2-[2-(trifluoromethyl)phenyl]acetamido}pyridin-2-yl)acetamide
85. N-(3,5-difluorophenyl)-N-{4-[2-(2,6-dimethylphenyl)acetamido]pyridin-2-yl}acetamide
86. N-{4-[2-(2-chlorophenyl)acetamido]pyridin-2-yl}-N-(3,5-difluorophenyl)acetamide
87. N-{4-[2-(2-chloro-4-fluorophenyl)acetamido]pyridin-2-yl}-N-(3,5-difluorophenyl)acetamide
88. N-{4-[2-(2-chloro-3-fluorophenyl)acetamido]pyridin-2-yl}-N-(3,5-difluorophenyl)acetamide
89. N-{4-[2-(2,6-dichlorophenyl)acetamido]pyridin-2-yl}-N-(3,5-difluorophenyl)acetamide
90. N-{4-[2-(2,6-dichloro-4-fluorophenyl)acetamido]pyridin-2-yl}-N-(3,5-difluorophenyl)acetamide
91. N-(3,5-difluorophenyl)-N-(4-{2-[2-(trifluoromethoxy)phenyl]acetamido}pyridin-2-yl)acetamide
92. N-{4-[2-(2-chlorophenyl)acetamido]pyridin-2-yl}-N-(3,5-difluoro-4-methylphenyl)acetamide
93. N-{4-[2-(2-chloro-4-fluorophenyl)acetamido]pyridin-2-yl}-N-(3,5-difluoro-4-methylphenyl)acetamide
94. N-{4-[2-(2-chloro-3-fluorophenyl)acetamido]pyridin-2-yl}-N-(3,5-difluoro-4-methylphenyl)acetamide
95. N-{4-[2-(2,6-dichlorophenyl)acetamido]pyridin-2-yl}-N-(3,5-difluoro-4-methylphenyl)acetamide
96. N-{4-[2-(2-chlorophenyl)acetamido]pyridin-2-yl}-N-(3,5-difluoro-4-methoxyphenyl)acetamide
97. N-{4-[2-(2-chloro-4-fluorophenyl)acetamido]pyridin-2-yl}-N-(3,5-difluoro-4-methoxyphenyl)acetamide
98. N-{4-[2-(2-chloro-3-fluorophenyl)acetamido]pyridin-2-yl}-N-(3,5-difluoro-4-methoxyphenyl)acetamide
99. N-{4-[2-(2,6-dichlorophenyl)acetamido]pyridin-2-yl}-N-(3,5-difluoro-4-methoxyphenyl)acetamide
100. N-{4-[2-(2-chlorophenyl)acetamido]pyridin-2-yl}-N-(3-methoxyphenyl)acetamide
101. N-{4-[2-(2-chloro-3-fluorophenyl)acetamido]pyridin-2-yl}-N-(3-methoxyphenyl)acetamide
102. N-{4-[2-(2-chlorophenyl)acetamido]pyridin-2-yl}-N-[3-(trifluoromethoxy)phenyl]acetamide
103. N-{4-[2-(2-chloro-3-fluorophenyl)acetamido]pyridin-2-yl}-N-(4-fluoro-3-methoxyphenyl)acetamide
104. N-{4-[2-(2,6-dichlorophenyl)acetamido]pyridin-2-yl}-N-(4-fluoro-3-methoxyphenyl)acetamide
105. N-(2-chlorophenyl)-N-(4-{2-[2-chloro-3-(trifluoromethyl)phenyl]acetamido}pyridin-2-yl)acetamide
106. N-(2-chlorophenyl)-N-{4-[2-(2-chlorophenyl)acetamido]pyridin-2-yl}acetamide
107. N-{4-[2-(2-chloro-4-fluorophenyl)acetamido]pyridin-2-yl}-N-(2-chlorophenyl)acetamide
108. N-{4-[2-(2-chloro-3-fluorophenyl)acetamido]pyridin-2-yl}-N-(2-chlorophenyl)acetamide
109. N-(2-chlorophenyl)-N-{4-[2-(2,6-dichlorophenyl)acetamido]pyridin-2-yl}acetamide
110. N-(2-chloro-5-fluorophenyl)-N-(4-{2-[2-chloro-3-(trifluoromethyl)phenyl]acetamido}pyridin-2-yl)acetamide
111. N-(2-chloro-5-fluorophenyl)-N-{4-[2-(2-chlorophenyl)acetamido]pyridin-2-yl}acetamide
112. N-(2-chloro-5-fluorophenyl)-N-{4-[2-(2-chloro-4-fluorophenyl)acetamido]pyridin-2-yl}acetamide
113. N-(2-chloro-5-fluorophenyl)-N-{4-[2-(2-chloro-3-fluorophenyl)acetamido]pyridin-2-yl}acetamide
114. N-(2-chloro-5-fluorophenyl)-N-{4-[2-(2,6-dichlorophenyl)acetamido]pyridin-2-yl}acetamide
115. N-{4-[2-(2-chlorophenyl)acetamido]pyridin-2-yl}-N-(2-fluorophenyl)acetamide
116. N-{4-[2-(2-chloro-4-fluorophenyl)acetamido]pyridin-2-yl}-N-(2-fluorophenyl)acetamide
117. N-{4-[2-(2-chloro-3-fluorophenyl)acetamido]pyridin-2-yl}-N-(2-fluorophenyl)acetamide
118. N-{4-[2-(2,6-dichlorophenyl)acetamido]pyridin-2-yl}-N-(2-fluorophenyl)acetamide
119. N-{4-[2-(2-chloro-3-fluorophenyl)acetamido]pyridin-2-yl}-N-[2-fluoro-4-(trifluoromethyl)phenyl]acetamide
120. N-{4-[2-(2,6-dichlorophenyl)acetamido]pyridin-2-yl}-N-[2-fluoro-4-(trifluoromethyl)phenyl]acetamide
121. N-(4-{2-[2-chloro-3-(trifluoromethyl)phenyl]acetamido}pyridin-2-yl)-N-(2,3-difluorophenyl)acetamide.
122. N-{4-[2-(2-chlorophenyl)acetamido]pyridin-2-yl}-N-(2,3-difluorophenyl)acetamide
123. N-{4-[2-(2-chloro-4-fluorophenyl)acetamido]pyridin-2-yl}-N-(2,3-difluorophenyl)acetamide
124. N-{4-[2-(2-chloro-3-fluorophenyl)acetamido]pyridin-2-yl}-N-(2,3-difluorophenyl)acetamide
125. N-{4-[2-(2,6-dichlorophenyl)acetamido]pyridin-2-yl}-N-(2,3-difluorophenyl)acetamide
126. N-{4-[2-(2-chlorophenyl)acetamido]pyridin-2-yl}-N-(2,4-difluorophenyl)acetamide
127. N-{4-[2-(2-chloro-4-fluorophenyl)acetamido]pyridin-2-yl}-N-(2,4-difluorophenyl)acetamide
128. N-{4-[2-(2-chloro-3-fluorophenyl)acetamido]pyridin-2-yl}-N-(2,4-difluorophenyl)acetamide
129. N-{4-[2-(2,6-dichlorophenyl)acetamido]pyridin-2-yl}-N-(2,4-difluorophenyl)acetamide
130. N-{4-[2-(2-chloro-6-fluorophenyl)acetamido]pyridin-2-yl}-N-(2,4-difluorophenyl)acetamide
131. N-(3-chlorophenyl)-N-{4-[2-(2-chlorophenyl)acetamido]pyridin-2-yl}acetamide
132. N-{4-[2-(2-chloro-4-fluorophenyl)acetamido]pyridin-2-yl}-N-(3-chlorophenyl)acetamide
133. N-{4-[2-(2-chloro-3-fluorophenyl)acetamido]pyridin-2-yl}-N-(3-chlorophenyl)acetamide
134. N-(3-chlorophenyl)-N-{4-[2-(2,6-dichlorophenyl)acetamido]pyridin-2-yl}acetamide
135. N-{4-[2-(2-chloro-6-fluorophenyl)acetamido]pyridin-2-yl}-N-(3-chlorophenyl)acetamide
136. N-(3-chloro-5-fluorophenyl)-N-{4-[2-(2-chlorophenyl)acetamido]pyridin-2-yl}acetamide
137. N-(3-chloro-5-fluorophenyl)-N-{4-[2-(2-chloro-4-fluorophenyl)acetamido]pyridin-2-yl}acetamide
138. N-(3-chloro-5-fluorophenyl)-N-{4-[2-(2-chloro-3-fluorophenyl)acetamido]pyridin-2-yl}acetamide
139. N-(3-chloro-5-fluorophenyl)-N-{4-[2-(2,6-dichlorophenyl)acetamido]pyridin-2-yl}acetamide
140. N-(3-chloro-5-fluorophenyl)-N-{4-[2-(2-chloro-6-fluorophenyl)acetamido]pyridin-2-yl}acetamide
141. N-{4-[2-(2-chloro-4-fluorophenyl)acetamido]pyridin-2-yl}-N-(4-fluorophenyl)butanamide
142. N-{4-[2-(2-chloro-6-fluorophenyl)acetamido]pyridin-2-yl}-N-(4-fluorophenyl)butanamide
143. N-{4-[2-(2-chlorophenyl)acetamido]pyridin-2-yl}-N-(4-fluorophenyl)butanamide
144. N-{4-[2-(2,6-dichlorophenyl)acetamido]pyridin-2-yl}-N-(4-fluorophenyl)butanamide
145. N-{4-[2-(2-Chlorphenyl)acetamido]pyridin-2-yl}-N-(3-cyan-5-fluorphenyl)acetamide
146. N-(3-Cyan-5-fluorphenyl)-N-{4-[2-(2,6-dichlorphenyl)acetamido]pyridin-2-yl}acetamide
147. N-{4-[2-(2-Chlor-3-fluorphenyl)acetamido]pyridin-2-yl}-N-(3-cyan-5-fluorphenyl)acetamide
148. N-{4-[2-(2-Chlor-6-fluorphenyl)acetamido]pyridin-2-yl}-N-(3-cyan-5-fluorphenyl)acetamide
149. N-{4-[2-(2-Chlor-4-fluorphenyl)acetamido]pyridin-2-yl}-N-(3-cyan-5-fluorphenyl)acetamide
150. N-(2-Chlor-4-fluorphenyl)-N-{4-[2-(2-chlorphenyl)acetamido]pyridin-2-yl}acetamide
151. N-(2-Chlor-4-fluorphenyl)-N-{4-[2-(2-chlor-4-fluorphenyl)acetamido]pyridin-2-yl}acetamide
152. N-(2-Chlor-4-fluorphenyl)-N-{4-[2-(2,6-dichlorphenyl)acetamido]pyridin-2-yl}acetamide
153. N-(2-Chlor-4-fluorphenyl)-N-{4-[2-(2-chlor-6-fluorphenyl)acetamido]pyridin-2-yl}acetamide
154. N-(2-Chlor-4-fluorphenyl)-N-{4-[2-(2-chlor-3-fluorphenyl)acetamido]pyridin-2-yl}acetamide
155. N-[3-Chlor-4-(methylsulfonyl)phenyl]-N-{4-[2-(2,6-dichlorphenyl)acetamido]pyridin-2-yl}acetamide
156. N-{4-[2-(2-Chlor-3-fluorphenyl)acetamido]pyridin-2-yl}-N-[3-chlor-4-(methylsulfonyl)phenyl]acetamide
157. N-[3-Chlor-4-(methylsulfonyl)phenyl]-N-{4-[2-(2-chlorphenyl)acetamido]pyridin-2-yl}acetamide
158. N-{4-[2-(2-Chlor-6-fluorphenyl)acetamido]pyridin-2-yl}-N-[3-chlor-4-(methylsulfonyl)phenyl]acetamide
159. N-{4-[2-(2-Chlor-4-fluorphenyl)acetamido]pyridin-2-yl}-N-[3-chlor-4-(methylsulfonyl)phenyl]acetamide
160. N-{4-[2-(2-Chlor-3-fluorphenyl)acetamido]pyridin-2-yl}-N-(3-fluor-5-methoxyphenyl)acetamide
161. N-{4-[2-(2-Chlorphenyl)acetamido]pyridin-2-yl}-N-(3-fluor-5-methoxyphenyl)acetamide
162. N-{4-[2-(2,6-Dichlorphenyl)acetamido]pyridin-2-yl}-N-(3-fluor-5-methoxyphenyl)acetamide
163. N-{4-[2-(2-Chlor-4-fluorphenyl)acetamido]pyridin-2-yl}-N-(3-fluor-5-methoxyphenyl)acetamide
164. N-{4-[2-(2-Chlor-6-fluorphenyl)acetamido]pyridin-2-yl}-N-(3-fluor-5-methoxyphenyl)acetamide
165. N-{4-[2-(2,6-Dichlorphenyl)acetamido]pyridin-2-yl}-N-[2-(difluormethyl)phenyl]acetamide
166. N-{4-[2-(2-Chlorphenyl)acetamido]pyridin-2-yl}-N-[2-(difluormethyl)phenyl]acetamide
167. N-{4-[2-(2-Chlor-3-fluorphenyl)acetamido]pyridin-2-yl}-N-[2-(difluormethyl)phenyl]acetamide
168. N-{4-[2-(2-Chlor-6-fluorphenyl)acetamido]pyridin-2-yl}-N-[2-(difluormethyl)phenyl]acetamide
169. N-{4-[2-(2-Chlor-4-fluorphenyl)acetamido]pyridin-2-yl}-N-[2-(difluormethyl)phenyl]acetamide
170. N-{4-[2-(2-Chlorphenyl)acetamido]pyridin-2-yl}-N-(2,4-dimethylphenyl)acetamide
171. N-{4-[2-(2-Chlor-3-fluorphenyl)acetamido]pyridin-2-yl}-N-(2,4-dimethylphenyl)acetamide
172. N-{4-[2-(2-Chlor-6-fluorphenyl)acetamido]pyridin-2-yl}-N-(2,4-dimethylphenyl)acetamide
173. N-{4-[2-(2,6-Dichlorphenyl)acetamido]pyridin-2-yl}-N-(2,4-dimethylphenyl)acetamide
174. N-{4-[2-(2-Chlor-4-fluorphenyl)acetamido]pyridin-2-yl}-N-(2,4-dimethylphenyl)acetamide
175. N-{4-[2-(2-Chlorphenyl)acetamido]pyridin-2-yl}-N-(3-cyan-5-methylphenyl)acetamide
176. N-(3-Cyan-5-methylphenyl)-N-{4-[2-(2,6-dichlorphenyl)acetamido]pyridin-2-yl}acetamide
177. N-{4-[2-(2-Chlor-3-fluorphenyl)acetamido]pyridin-2-yl}-N-(3-cyan-5-methylphenyl)acetamide
178. N-{4-[2-(2-Chlor-4-fluorphenyl)acetamido]pyridin-2-yl}-N-(3-cyan-5-methylphenyl)acetamide
179. N-{4-[2-(2-Chlor-6-fluorphenyl)acetamido]pyridin-2-yl}-N-(3-cyan-5-methylphenyl)acetamide
180. N-(3-Chlor-4-methylphenyl)-N-{4-[2-(2-chlorphenyl)acetamido]pyridin-2-yl}acetamide
181. N-{4-[2-(2-Chlor-3-fluorphenyl)acetamido]pyridin-2-yl}-N-(3-chlor-4-methylphenyl)acetamide
182. N-(3-Chlor-4-methylphenyl)-N-{4-[2-(2,6-dichlorphenyl)acetamido]pyridin-2-yl}acetamide
183. N-{4-[2-(2-Chlor-6-fluorphenyl)acetamido]pyridin-2-yl}-N-(3-chlor-4-methylphenyl)acetamide
184. N-{4-[2-(2-Chlor-6-fluorphenyl)acetamido]pyridin-2-yl}-N-(4-fluor-2,3-dimethylphenyl)acetamide
185. N-{5-[2-(2,6-dichlorophenyl)acetamido]pyridazin-3-yl}-N-phenylacetamide
186. N-{5-[2-(2-chloro-3-fluorophenyl)acetamido]pyridazin-3-yl}-N-phenylacetamide
187. N-{5-[2-(2-chlorophenyl)acetamido]pyridazin-3-yl}-N-phenylacetamide
188. N-{5-[2-(2-chloro-4-fluorophenyl)acetamido]pyridazin-3-yl}-N-phenylacetamide
189. N-{5-[2-(2-chloro-6-fluorophenyl)acetamido]pyridazin-3-yl}-N-(4-fluorophenyl)acetamide
190. N-{5-[2-(2-chloro-4-fluorophenyl)acetamido]pyridazin-3-yl}-N-(4-fluorophenyl)acetamide
191. N-{5-[2-(2-chloro-3-fluorophenyl)acetamido]pyridazin-3-yl}-N-(4-fluorophenyl)acetamide
192. N-{5-[2-(2-chlorophenyl)acetamido]pyridazin-3-yl}-N-(4-fluorophenyl)acetamide
193. N-{5-[2-(2,6-dichlorophenyl)acetamido]pyridazin-3-yl}-N-(4-fluorophenyl)acetamide
194. N-{5-[2-(2,6-dichlorophenyl)acetamido]pyridazin-3-yl}-N-(3-fluorophenyl)acetamide
195. N-{5-[2-(2-chloro-3-fluorophenyl)acetamido]pyridazin-3-yl}-N-(3-fluorophenyl)acetamide
196. N-{5-[2-(2-chlorophenyl)acetamido]pyridazin-3-yl}-N-(3-fluorophenyl)acetamide
197. N-{5-[2-(2-chloro-6-fluorophenyl)acetamido]pyridazin-3-yl}-N-(3-fluorophenyl)acetamide
198. N-{5-[2-(2-chloro-4-fluorophenyl)acetamido]pyridazin-3-yl}-N-(3-fluorophenyl)acetamide
199. N-{5-[2-(2-chlorophenyl)acetamido]pyridazin-3-yl}-N-(3,4-difluorophenyl)acetamide
200. N-{5-[2-(2,6-dichlorophenyl)acetamido]pyridazin-3-yl}-N-(3,4-difluorophenyl)acetamide
201. N-{5-[2-(2-chloro-3-fluorophenyl)acetamido]pyridazin-3-yl}-N-(3,4-difluorophenyl)acetamide
202. N-{5-[2-(2-chloro-4-fluorophenyl)acetamido]pyridazin-3-yl}-N-(3,4-difluorophenyl)acetamide
203. N-{5-[2-(2-chloro-6-fluorophenyl)acetamido]pyridazin-3-yl}-N-(3,4-difluorophenyl)acetamide
204. N-{5-[2-(2,6-dichlorophenyl)acetamido]pyridazin-3-yl}-N-(3,5-difluorophenyl)acetamide
205. N-{5-[2-(2-chlorophenyl)acetamido]pyridazin-3-yl}-N-(3,5-difluorophenyl)acetamide
206. N-{5-[2-(2-chloro-3-fluorophenyl)acetamido]pyridazin-3-yl}-N-(3,5-difluorophenyl)acetamide
207. N-{5-[2-(2-chloro-4-fluorophenyl)acetamido]pyridazin-3-yl}-N-(3,5-difluorophenyl)acetamide
208. N-{5-[2-(2-chloro-6-fluorophenyl)acetamido]pyridazin-3-yl}-N-(3,5-difluorophenyl)acetamide

The compounds of general formula (I) can be prepared according to the following schemes 1, 2 and 3. The schemes and procedures described below illustrate synthetic routes to the compounds of general formula (I) and are not intended to be limiting. It is clear to the person skilled in the art that the order of transformations as exemplified in schemes 1, 2 and 3 can be modified in various ways. The order of transformations exemplified in these schemes is therefore not intended to be limiting. In addition, interconversion of any of the substituents, R^{1a}, R^{1b}, R^{1c}, R², R³, R^{4a}, or R^{4b} can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example T. W. Greene and P. G. M. Wuts in Protective Groups in Organic Synthesis, 3^{rd} edition, Wiley 1999). Specific examples are described in the subsequent paragraphs.

Scheme 1 depicts the synthesis starting from aromatic amines of the formula (II), and synthons of formula (III), wherein Hal stands for Cl, Br, I or a triflate, Br being preferred; and wherein A stands for CH. The two starting materials can be cross-coupled by Pd-mediated reactions (Buchwald-Hartwig-coupling) known to those skilled in the art. A suitable solvent like for example N,N-dimethylformamide,1,4-dioxane or toluene is used and a base such as potassium carbonate, potassium phosphate, caesium carbonate or potassium tert-butanolate is added. Appropriate palladium catalysts in combination with suitable phosphine ligands are utilized as catalyst catalyst-ligand system, for example bis(dibenzylidenaceton) palladium(0) and 4,5-bis-(diphenylphosphino)-9,9-dimethyl xanthene (Xantphos). The reaction is performed at temperatures between 80° C. and 120° C., preferred at 100° C. until complete conversion, typically for 18 h. Aromatic amines of general formula (IV) may react according to standard procedures with carboxylic acid anhydrides (V) or the corresponding acetyl chlorides (VI) to yield amides of general formula (VII). In case of the use of anhydrides (V) like e.g. acetanhyride, it may also serve as solvent. N,N-dimethylaminopyridine may be used as catalyst (0.1 eq). The reaction usually takes place between 100 and 130° C. until complete conversion (2-18 h). In case of the use of carboxylic acid chloride, e.g. acetyl chloride, dichloromethane may be used as solvent and a base, e.g. triethyl amine, is added. The nitro group in compounds of the general formula (VII) are reduced to the corresponding amino group of compounds of general formula (VIII) via procedures known to those skilled in the art, e.g. via hydrogenation in presence of a suitable catalyst like palladium or platinum, e.g. 10% Pd on activated charcoal. Preferably, atmospheric hydrogen pressure is utilized. Suitable solvents like ethanol, methanol or ethyl acetate (which is preferred) are used. Alternatively, other reduction methods are used, most notably the reduction with iron powder (5 eq.) in acetic acid. The mixture is stirred vigorously until complete conversion (2-18 h). Aromatic amines of general formula (VIII) may react with carboxylic acids of general formula (IX) by methods known to those skilled in the art to give the amide compounds of general formula (I). The reaction is mediated by activating a carboxylic acid of general formula (IX) with reagents such as dicyclohexylcarbodiimide (DCC), 1-(3-dimethylaminopropyl)-3-ethylcarbodiimide (EDCI), N-hydroxybenzotriazole (HOBT), N-[(dimethylamino)-(3H-[1,2,3]triazolo[4,5-b]pyridin-3-yloxy)methyliden]-N-methylmethanaminium hexafluorophosphate (HATU) or propylphosphonic anhydride (T3P). For example, the reaction with HATU or T3P takes place in an inert solvent, such as N,N-dimethylformamide, dichloromethane or dimethyl sulfoxide in the presence of the appropriate aromatic amine of general formula (VIII) and a tertiary amine (such as triethylamine or diisopropylethylamine) at temperatures between −30° C. and +80° C.

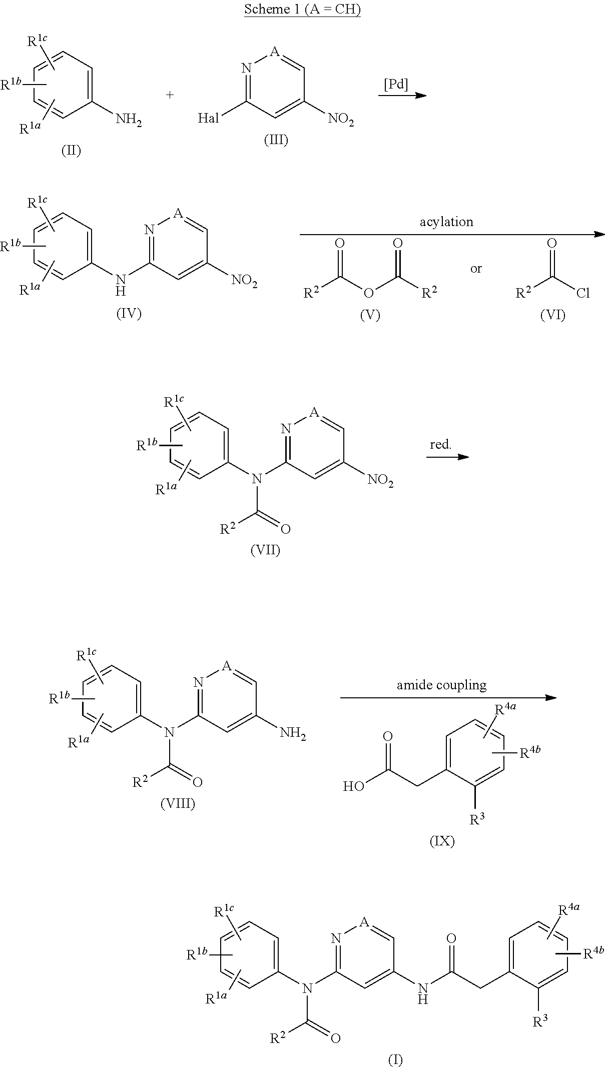

in which A is CH and R^{1a}, R^{1b}, R^{1c}, R² and R³, R^{4a}, R^{4b} are as defined for the compound of general formula (I) supra.

As an alternative, the first step described in scheme 1 may also be performed using an aromatic halide of general formula (X) and a synthon of general formula (XI) (scheme 2).

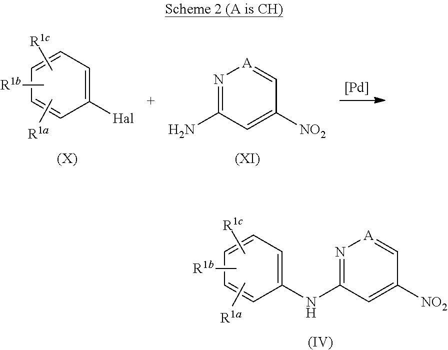

in which A is CH and R^{1a}, R^{1b}, and R^{1c} are as defined for the compound of general formula (I) supra.

The sequence of the synthesis steps may be changed as appropriate.

For example, in case A=N, the steps were performed as outlined in scheme 3.

Compound (XII) was used as starting materials. First, the amide coupling using carboxylic acids of type (IX) were carried out, followed by Pd-catalyzed cross coupling with aromatic amines of general formula (II) and acylation with acid chlorides of type (VI).

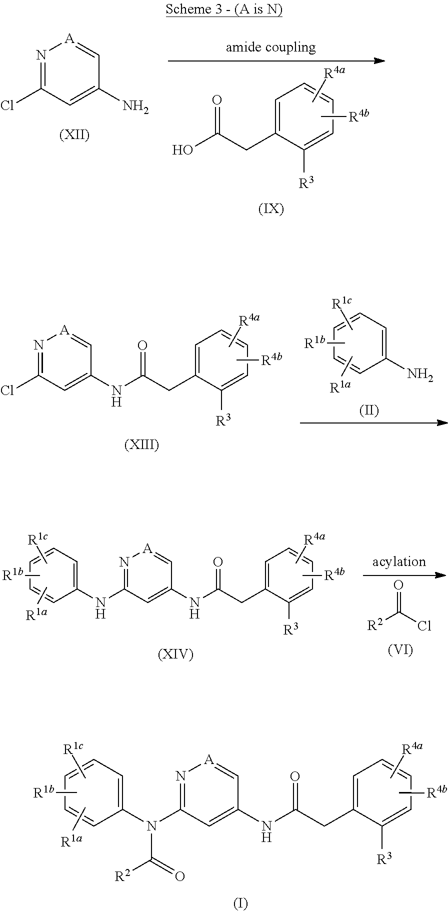

in which A is N and R^{1a}, R^{1b}, R^{1c}, R² and R³, R^{4a}, R^{4b} are as defined for the compound of general formula (I) supra.

Compounds (II), (III), (V), (VI), (IX), (X) and (XI) are either commercially available or can be prepared according to procedures available from the public domain, as understandable to the person skilled in the art. Specific examples are described in the Experimental Section.

An alternative approach to synthesize compounds of general formula (I) is depicted in scheme 3A.

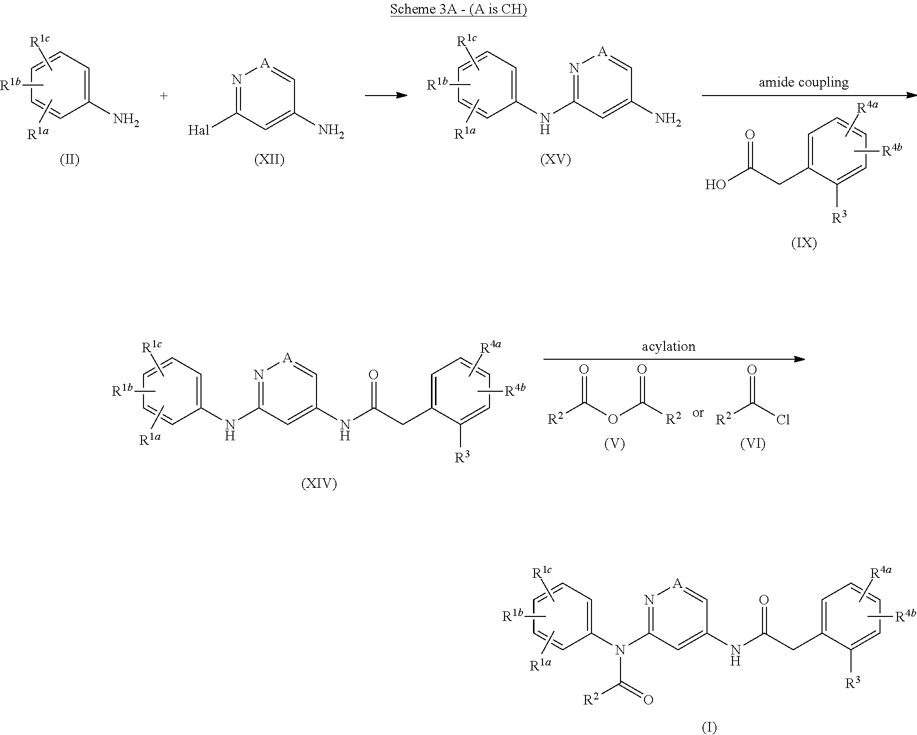

This synthesis starts from aromatic amines of the formula (II), and synthons of formula (XII), wherein Hal stands for Cl, Br, I or a triflate, Cl being preferred; and wherein A stands for CH. The two starting materials can be coupled by heating in higher boiling solvents, preferably in sulfolan (60°-130° C., 10-20 h, typically 130° C., for 18 h) in the presence of hydrochloric acid (1 eq). Alternatively, a cross-coupling by Pd-mediated reactions (Buchwald-Hartwig-coupling) known to those skilled in the art is also possible.

Aromatic amines of general formula (XV) may react with carboxylic acids of general formula (IX) by methods known to those skilled in the art to give the amide compounds of general formula (XIV). In particular, the coupling can be performed by activation with 1,1′-carbonyldiimidazole (1.0-1.5 eq.) in preferably N,N-dimethylacetamide as solvent. The reaction mixture is typically stirred at temperatures between r.t. and 80° C. (typically 40° C.) for 10 h to 24 h (typically 18 h).

Aromatic amines of general formula (XIV) may react according to standard procedures with carboxylic acid anhydrides (V) or the corresponding acyl chlorides (VI) to yield amides of general formula (I). In case of the use of anhydrides (V) like e.g. acetanhydride, it may also serve as solvent. N,N-dimethylaminopyridine may be used as catalyst (0.1 eq). The reaction usually takes place between 100 and 130° C. until complete conversion (2-18 h). In case of the use of carboxylic acid chloride, e.g. acetyl chloride, dichloromethane or, more preferred, rac-2-methyltetrahydrofuran, may be used as solvent. A base, e.g. triethyl amine or N,N-diisopropylethylamine (1-2 eq., typically 1.4 eq.), is added. Conversion takes place typically at room temperature in 1 to 24 h, typically in 18 h).

The compounds of general formula (I) can be converted to any salt, more particularly pharmaceutically acceptable salts, as described herein, by any method which is known to the person skilled in the art. Similarly, any salt of a compound of general formula (I) of the present invention can be converted into the free compound, by any method which is known to the person skilled in the art.

According to the invention a P2X4 inhibitors is used for the manufacture of a medicament for use in the treatment or prophylaxis of dry eye syndrome and in particular dry eye, ocular neuropathic pain, ocular trauma, and post-operative ocular pain.

A compound according to the invention is used for the manufacture of a medicament for use in the treatment or prophylaxis of dry eye syndrome and in particular dry eye, ocular neuropathic pain, ocular trauma, and post-operative ocular pain.

Compounds of general formula (I) of the present invention demonstrate a valuable pharmacological spectrum of action which could not have been predicted. Compounds of the present invention have surprisingly been found to effectively inhibit P2X4, as antagonists or negative allosteric modulators, and it is possible therefore that said compounds be used for the treatment or prophylaxis of diseases, in particular for use in the treatment or prophylaxis of dry eye syndrome and in particular dry eye, ocular neuropathic pain, ocular trauma and post-operative ocular pain.

Compounds of the present invention can be utilized to inhibit, antagonize, negative allosteric modulate, etc., the P2X4 receptor for use in the treatment or prophylaxis of dry eye syndrome and in particular dry eye, ocular neuropathic pain, ocular trauma and post-operative ocular pain. This method comprises administering to a mammal in need thereof, including a human, an amount of a compound of this invention, or a pharmaceutically acceptable salt, isomer, polymorph, metabolite, hydrate, solvate or ester thereof; which is effective to treat the disorder.

These disorders have been well characterized in humans, but also exist with a similar etiology in other mammals and can be treated by administering pharmaceutical compositions of the present invention.

The term “treating” or “treatment” as used in the present text is used conventionally, e.g., the management or care of a subject for the purpose of combating, alleviating, reducing, relieving, improving the condition of a disease or disorder, such as those reported above.

The compounds of the present invention can be used in therapy and prevention, i.e. prophylaxis, of the following syndromes, diseases or disorders:
ophthalmology indications, dry eye syndrome and in particular dry eye, ocular neuropathic pain, ocular trauma and post-operative ocular pain.

In accordance with a further aspect, the present invention covers compounds of general formula (I), as described supra, or stereoisomers, tautomers, N-oxides, hydrates, solvates, and salts thereof, particularly pharmaceutically acceptable salts thereof, or mixtures of same, for use in the treatment or prophylaxis of diseases, particularly of the diseases reported above.

The pharmaceutical activity of the compounds according to the invention can be explained by their activity as inhibitors, antagonizing and/or negative allosteric modulating, the P2X4 receptor in the eye.

In accordance with a further aspect, the present invention covers the use of compounds of general formula (I), as described supra, or stereoisomers, tautomers, N-oxides, hydrates, solvates, and salts thereof, particularly pharmaceutically acceptable salts thereof, or mixtures of same, for the treatment or prophylaxis of diseases, in particular of the diseases reported above.

In accordance with a further aspect, the present invention covers the use of compounds of general formula (I), as described supra, or stereoisomers, tautomers, N-oxides, hydrates, solvates, and salts thereof, particularly pharmaceutically acceptable salts thereof, or mixtures of same, in a method of treatment or prophylaxis of diseases, in particular of the diseases reported above.

In accordance with a further aspect, the present invention covers use of a compound of general formula (I), as described supra, or stereoisomers, tautomers, N-oxides, hydrates, solvates, and salts thereof, particularly pharmaceutically acceptable salts thereof, or mixtures of same, for the preparation of a pharmaceutical composition, preferably a medicament, for the prophylaxis or treatment of diseases, in particular of the diseases reported above.

In accordance with a further aspect, the present invention covers a method of treatment or prophylaxis of diseases, in particular of the diseases reported above, using an effective amount of a compound of general formula (I), as described supra, or stereoisomers, tautomers, N-oxides, hydrates, solvates, and salts thereof, particularly pharmaceutically acceptable salts thereof, or mixtures of same.

In accordance with a further aspect, the present invention covers pharmaceutical compositions, in particular a medicament, comprising a compound of general formula (I), as described supra, or a stereoisomer, a tautomer, an N-oxide, a hydrate, a solvate, a salt thereof, particularly a pharmaceutically acceptable salt, or a mixture of same, and one or more excipients), in particular one or more pharmaceutically acceptable excipient(s) for use in the treatment or prophylaxis of dry eye syndrome and in particular dry eye, ocular neuropathic pain, ocular trauma and post-operative ocular pain. Conventional procedures for preparing such pharmaceutical compositions in appropriate dosage forms can be utilized.

The present invention furthermore covers pharmaceutical compositions, in particular medicaments, which comprise at least one compound according to the invention, conventionally together with one or more pharmaceutically suitable excipients, and to their use for the above mentioned purposes.

It is possible for the compounds according to the invention to have systemic and/or local activity. For this purpose, they can be administered in a suitable manner, such as, for example, ocular route, conjunctival, or as an implant or ocular device.

For these administration routes, it is possible for the compounds according to the invention to be administered in suitable administration forms.

Examples which are suitable for ocular administration routes are pharmaceutical forms like eye drops, eye ointments, eye baths, ocular inserts or extended release formulations (such as, for example, injectable micro- or nanospheres), solutions, aqueous suspensions (lotions, mixturae agitandae), lipophilic suspensions, emulsions, ointments, creams.

The compounds according to the invention can be incorporated into the stated administration forms. This can be affected in a manner known per se by mixing with pharmaceutically suitable excipients.

The present invention furthermore relates to a pharmaceutical composition which comprise at least one compound according to the invention, conventionally together with one or more pharmaceutically suitable excipient(s), and to their use according to the present invention.

The compounds of the present invention can be administered as the sole pharmaceutical agent or in combination with one or more other pharmaceutically active ingredients where the combination causes no unacceptable adverse effects. The present invention also covers such pharmaceutical combinations.

Based upon standard laboratory techniques known to evaluate compounds useful for the treatment and prevention, i.e. prophylaxis, of the syndromes, diseases or disorders reported above, by standard toxicity tests and by standard pharmacological assays for the determination of treatment of the conditions identified above in mammals, and by comparison of these results with the results of known active ingredients or medicaments that are used to treat these conditions, the effective dosage of the compounds of the present invention can readily be determined for treatment of each desired indication. The amount of the active ingredient to be administered in the treatment of one of these conditions can vary widely according to such considerations as the particular compound and dosage unit employed, the mode of administration, the period of treatment, the age and sex of the patient treated, and the nature and extent of the condition treated.

The average daily dosage for administration by injection like intraocular injection and use of infusion techniques will preferably be from 0.01 to 200 mg/kg of total body weight. The average daily topical dosage regimen will preferably be from 0.1 to 200 mg administered between one to four times daily. The transcorneal or transcleral concentration will preferably be that required to maintain a daily dose of from 0.01 to 200 mg/kg.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

### EXPERIMENTAL SECTION

NMR peak forms are stated as they appear in the spectra, possible higher order effects have not been considered. Chemical shifts are given in ppm; all spectra were calibrated to solvent residual peak. Integrals are given in integers.

Alternatively, the ¹H-NMR data of selected compounds are listed in the form of ¹H-NMR peaklists. Therein, for each signal peak the δ value in ppm is given, followed by the signal intensity, reported in round brackets. The δ value-signal intensity pairs from different peaks are separated by commas. Therefore, a peaklist is described by the general form: δ₁ (intensity₁), δ₂ (intensity₂), . . . , δᵢ (intensityᵢ), . . . , δₙ (intensityₙ).

The intensity of a sharp signal correlates with the height (in cm) of the signal in a printed NMR spectrum. When compared with other signals, this data can be correlated to the real ratios of the signal intensities. In the case of broad signals, more than one peak, or the center of the signal along with their relative intensity, compared to the most intense signal displayed in the spectrum, are shown. A ¹H-NMR peaklist is similar to a classical ¹H-NMR readout, and thus usually contains all the peaks listed in a classical NMR interpretation. Moreover, similar to classical ¹H-NMR printouts, peaklists can show solvent signals, signals derived from stereoisomers of the particular target compound, peaks of impurities, ¹³C satellite peaks, and/or spinning sidebands. The peaks of stereoisomers, and/or peaks of impurities are typically displayed with a lower intensity compared to the peaks of the target compound (e.g., with a purity of >90%). Such stereoisomers and/or impurities may be typical for the particular manufacturing process, and therefore their peaks may help to identify a reproduction of the manufacturing process on the basis of “by-product fingerprints”. An expert who calculates the peaks of the target compound by known methods (MestReC, ACD simulation, or by use of empirically evaluated expectation values), can isolate the peaks of the target compound as required, optionally using additional intensity filters. Such an operation would be similar to peak-picking in classical ¹H-NMR interpretation. A detailed description of the reporting of NMR data in the form of peaklists can be found in the publication “Citation of NMR Peaklist Data within Patent Applications” (cf. http://www.researchdisclosure.com/searching-disclosures, Research Disclosure Database Number 605005, 2014, 1 Aug. 2014). In the peak picking routine, as described in the Research Disclosure Database Number 605005, the parameter “MinimumHeight” can be adjusted between 1% and 4%. However, depending on the chemical structure and/or depending on the concentration of the measured compound it may be reasonable to set the parameter “MinimumHeight”<1%.

Chemical names were generated using the ACD/Name software from ACD/Labs. In some cases generally accepted names of commercially available reagents were used in place of ACD/Name generated names.

The following table 1 lists the abbreviations used in this paragraph and in the Examples section as far as they are not explained within the text body. Other abbreviations have their meanings customary per se to the skilled person.

TABLE 1
Abbreviations
The following table lists the abbreviations used herein.

| Abbreviation | Meaning |
|---|---|
| ACN | acetonitrile |
| Ac₂O | acetic anhydride |
| ACOH | acetic acid (ethanoic acid) |
| aq. | aqueous |
| Boc | tert-butoxycarbonyl |
| Br | broad (¹H-NMR signal) |
| cat. | catalytic |
| conc. | concentrated |
| CI | chemical ionisation |
| d | doublet |
| DAD | diode array detector |
| DCM | dichloromethane |
| dd | double-doublet |
| DIPEA | diisopropylethylamine |
| DMA | N,N-dimethylacetamide |
| DMF | N,N-dimethylformamide |
| DMSO | dimethylsulfoxide |
| dt | double-triplet |
| EDC | 1-ethyl-3-(3-dimethylaminopropyl)carbodiimide |
| ELSD | Evaporative Light Scattering Detector |
| EtOAc | ethyl acetate |
| EtOH | ethanol |
| eq. | equivalent |
| ESI | electrospray (ES) ionisation |
| h or hrs | hour(s) |
| HATU | 1-[bis(dimethylamino)methylene]-1H-1,2,3-triazolo[4,5- |
| | b]pyridinium 3-oxid hexafluorophosphate |
| HCl | hydrochloric acid |
| HPLC | high performance liquid chromatography |
| LC-MS | liquid chromatography mass spectrometry |
| m | multiplet |
| min | minute(s) |
| MeCN | acetonitrile |
| MeOH | methanol |
| MS | mass spectrometry |
| NMR | nuclear magnetic resonance spectroscopy: chemical |
| | shifts (δ) are given in ppm. The chemical shifts were |
| | corrected by setting the DMSO signal to 2.50 ppm unless |
| | otherwise stated. |
| Pd/C | palladium on activated charcoal |
| PdCl₂(dppf) | [1,1′- |
| | bis(diphenylphosphino)ferrocene]dichloropalladium(II) |
| Pd(dba)₂ | bis(dibenzylideneacetone)palladium |
| q | quartet |
| r.t. or rt or RT | room temperature |
| rac | racemic |
| Rt | retention time (as measured either with HPLC or UPLC) |
| | in minutes |
| s | singlet |
| sat. | saturated |
| SM | starting material |
| SQD | Single-Quadrupole-Detector |
| t | triplet |
| T3P | propylphosphonic anhydride |
| td | triple-doublet |
| TEA | triethylamine |
| TFA | trifluoroacetic acid |
| THF | tetrahydrofuran |
| UPLC | ultra performance liquid chromatography |

Other abbreviations have their meanings customary per se to the skilled person.

The various aspects of the invention described in this application are illustrated by the following examples which are not meant to limit the invention in any way.

The example testing experiments described herein serve to illustrate the present invention and the invention is not limited to the examples given.

All reagents, for which the synthesis is not described in the experimental part, are either commercially available, or are known compounds or may be formed from known compounds by known methods by a person skilled in the art.

The compounds and intermediates produced according to the methods of the invention may require purification. Purification of organic compounds is well known to the person skilled in the art and there may be several ways of purifying the same compound. In some cases, no purification may be necessary. In some cases, the compounds may be purified by crystallization. In some cases, impurities may be stirred out using a suitable solvent. In some cases, the compounds may be purified by chromatography, particularly flash column chromatography, using for example prepacked silica gel cartridges, e.g. Biotage SNAP cartidges KP-Sil® or KP-NH® in combination with a Biotage autopurifier system (SP4® or Isolera Four®) and eluents such as gradients of hexane/ethyl acetate or DCM/methanol. In some cases, the compounds may be purified by preparative HPLC using for example a Waters autopurifier equipped with a diode array detector and/or on-line electrospray ionization mass spectrometer in combination with a suitable prepacked reverse phase column and eluents such as gradients of water and acetonitrile which may contain additives such as trifluoroacetic acid, formic acid or aqueous ammonia.

In some cases, purification methods as described above can provide those compounds of the present invention which possess a sufficiently basic or acidic functionality in the form of a salt, such as, in the case of a compound of the present invention which is sufficiently basic, a trifluoroacetate or formate salt for example, or, in the case of a compound of the present invention which is sufficiently acidic, an ammonium salt for example. A salt of this type can either be transformed into its free base or free acid form, respectively, by various methods known to the person skilled in the art, or be used as salts in subsequent biological assays. It is to be understood that the specific form (e.g. salt, free base etc.) of a compound of the present invention as isolated and as described herein is not necessarily the only form in which said compound can be applied to a biological assay in order to quantify the specific biological activity.

UPLC-MS Standard Procedures

Analytical UPLC-MS was performed as described below. The masses (m/z) are reported from the positive mode electrospray ionisation unless the negative mode is indicated (ESI−). In most of the cases method 1 is used. If not, it is indicated.

Method 1:

Instrument: Waters Acquity UPLCMS SingleQuad; Column: Acquity UPLC BEH C18 1.7 μm, 50×2.1 mm; eluent A: water+0.2 vol % aqueous ammonia (32%), eluent B: acetonitrile; gradient: 0-1.6 min 1-99% B, 1.6-2.0 min 99% B; flow 0.8 ml/min; temperature: 60° C.; DAD scan: 210-400 nm.

Method 2:

Instrument: Waters Acquity UPLCMS SingleQuad; Column: Acquity UPLC BEH C18 1.7 μm, 50×2.1 mm; eluent A: water+0.1 vol % formic acid (99%), eluent B: acetonitrile; gradient: 0-1.6 min 1-99% B, 1.6-2.0 min 99% B; flow 0.8 ml/min; temperature: 60° C.; DAD scan: 210-400 nm.

Method 3:

Instrument: Waters Acquity Platform ZQ4000; column: Waters BEHC 18, 50 mm×2.1 mm, 1.7μ; eluent A: water/0.05% formic acid, eluent B: acetonitrile/0.05% formic acid; gradient: 0.0 min 98% A→0.2 min: 98% A→1.7 min: 10% A→1.9 min: 10% A→2 min: 98% A→2.5 min: 98% A; flow: 1.3 ml/min; column temperature: 60° C.; UV-detection: 200-400 nm.

### Experimental Section—General Procedures

General Procedure A:

Formation of bisarylamines from aromatic amide and 2-bromo-4-nitropyridine (GP A): Aromatic amine (1.0 eq.) and 2-bromo-4-nitropyridine (1.4 eq) were dissolved in toluene or 1,4-dioxane or DMF (ca. 70 eq.). Under inert atmosphere (Argon), bis(dibenzylidenaceton) palladium(0) (CAS [32005-36-0], 0.03 eq.), 4,5-bis-(diphenylphosphino)-9,9-dimethyl xanthene (Xantphos, CAS [161265-03-8], 0.07 eq.), and caesium carbonate (1.6 eq.) were added and the mixture stirred at 100° C. for ca. 18 h. After cooling to rt, the catalyst was filtered off via celite and rinsed with ethyl acetate. The filtrate was partitioned between water and ethyl acetate and extracted with ethyl acetate. The combined arganic layers were washed with brine, dried with sodium sulfate and the solvents removed in vacuo. The crude product was purified via chromatography.

General Procedure B:

Formation of bisarylamines from arylhalogenide and 2-amino-4-nitro-pyridine (GP B):

Aromatic bromide (1.0-1.4 eq., alternatively, the corresponding iodide may be used), 4-nitropyridine-2-amine (1.0 eq.) and caesium carbonate (1.6 eq.) were dissolved in 1,4-dioxane or toluene. The mixture was degassed, and under argon atmosphere, bis(dibenzylidenaceton) palladium(0) (CAS [32005-36-0], 0.03 eq.) and 4,5-bis-(diphenylphosphino)-9,9-dimethyl xanthene (Xantphos, CAS [161265-03-8], 0.07 eq.) were added. The mixture was stirred at 100° C. for 18 h. After cooling to rt, the solids were filtered off and rinsed with ethyl acetate. The filtrate was partitioned between water and ethyl acetate and extracted with ethyl acetate. The combined arganic layers were washed with brine, dried with sodium sulfate and the solvents removed in vacuo. The crude product was purified via chromatography.

General Procedure C:

Acylation of Bisarylamines (GP C):

The bisarylamines were dissolved in acetic anhydride (or the respective corresponding homologue) as reagent and solvent (ca. 50 eq.), 4-N,N-dimethylaminopyridine (0.1 eq.) was added and the mixture stirred at 110-130° C. until complete conversion (2-18 h). After cooling to rt, the mixture was either concentrated to dryness in vacuo and directly purified via chromatography or an aqueous workup was done. In this case, the mixture was partitioned between ethyl acetate and water, extracted with ethyl acetate, washed with brine, dried with sodium sulfate and the solvents removed in vacuo. The crude product was purified by chromatography.

General Procedure D:

Reduction of Nitro Compounds by Catalytic Hydrogenation (GP D):

The nitro compound was dissolved in ethyl acetate and the palladium catalyst (10% Pd on activated carbon, 0.1 eq. Pd) was added. The mixture was degassed and charged with hydrogen and hydrogenated at 1 atm hydrogen pressure until complete conversion. Then the catalyst was filtered off and the filtrated concentrated to dryness. The product could be obtained without further purification.

General Procedure E:

Reduction of Nitro Compounds with Iron (GP E):

The nitro compound was dissolved in acetic acid and iron powder (5 eq.) was added. The mixture was vigorously stirred for 2-18 h, until complete conversion. Solids were filtered off via a celite pad and rinsed with ethyl acetate. The organic phase evaporated to dryness. Optionally, the residue was either codestilled several times with toluene until all acetic acid was removed or it was partitioned between ethyl acetate and water and sat. aqueous sodium bicarbonate solution added until pH>7. The phases were separated, the aqueous layer extracted with ethyl acetate and the combined organic layers were washed with sat. aqueous sodium bicarbonate solution and brine and dried with sodium sulfate. The solvents were removed in vacuo and the product was taken to the next step without further purification.

General Procedure F:

Acylation of amino-pyridazines (GP F):

6-Chloro-4-pyridazinamine and carboxylic acid (1-2 eq.) were dissolved in DMF and T3P (1-propanephosphinic anhydride, 50% in DMF, CAS [68957-94-8], 4.8 eq.) and N,N-Diisopropylethylamin (6 eq.) were added and the mixture stirred at 80° C. until complete conversion. Then the mixture was evaporated to a small volume, poured into water and filtered off. Then the solid was used in the following step as it was or it was purified by HPLC if necessary.

General Procedure G:

Aromatic Nucleophilic Substitution of Chloropyridazine (GP G):

The N-acylated (6-chloropyridazin-4-yl)acetamide was dissolved in ethanol and an aniline derivative (1 eq.) was added. Optionally, 4-methylbenzenesulfonic acid hydrate (1 eq.) could be added to enhance the turnover. Then the mixture was stirred at 80° C. for 48 hrs and evaporated. The residue was purified by HPLC.

General Procedure H:

Amide Formation with HATU (GP H):

Amine and carboxylic acid (1.2 eq.) were dissolved in DMF and HATU (2-(7-aza-1H-benzotriazole-1-yl)-1,1,3,3-tetramethyluronium hexafluorophosphate, CAS [148893-10-1], 1.2 eq.) and triethylamine (5 eq.) were added and the mixture stirred at rt until complete conversion. Then the mixture was poured into water, extracted with ethyl acetate, the combined organic layers washed with brine, dried with sodium sulfate and the solvents evaporated. The crude product was purified by chromatography.

General Procedure I:

Amide Formation with T3P (GP I):

Amine and carboxylic acid (1-2 eq.) were dissolved in DMF and T3P (1-propanephosphinic anhydride, 50% in DMF, CAS [68957-94-8], 3 eq.) and triethylamine (6 eq.) were added and the mixture stirred at rt until complete conversion. Then the mixture was poured into water, extracted with ethyl acetate, the combined organic layers washed with brine, dried with sodium sulfate and the solvents evaporated. The crude product was purified by chromatography.

General Procedure J:

Acetylation amino-pyrazines (GP J):

The aminopyrazines were dissolved in dichloromethane and acetal chloride (1.5 eq.) and triethylamine (1.8 eq.) were added and the mixture stirred at rt for 18 h. The mixture was concentrated in vacuo and directly purified via chromatography.

### Experimental Section—Intermediates

### Intermediate 1

### N-(3,4-difluorophenyl)-4-nitropyridin-2-amine

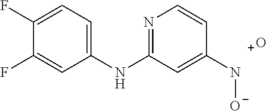

According to GP A, 3,4-difluoroaniline (454 mg, 3.52 mmol) and 2-bromo-4-nitropyridine (1.00 g, 4.93 mmol, 1.4 eq.) in toluene (25 mL) were converted to 658 mg of the title compound (74% of theory) as a yellow solid.

¹H NMR (400 MHz, DMSO-d6) δ[ppm] 7.29-7.34 (m, 1H), 7.35-7.42 (m, 1H), 6.92 (dd, 1H), 7.43-7.45 (m, 1H), 7.52 (dd, 1H), 8.00 (ddd, 1H), 8.49 (d, 1H), 9.92 (s, 1H).

LCMS (Method 1): Rt=1.24 min, MS (ESIpos) m/z=252 [M+H]⁺

### Intermediate 2

### N-(3-fluorophenyl)-4-nitropyridin-2-amine

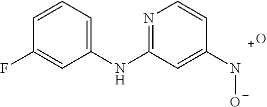

According to GP B, 4-nitropyridine-2-amine (2.00 g, 14.4 mmol) and 1-bromo-3-fluorobenzene (3.52 g, 20.1 mmol, 1.4 eq.) in toluene (75 mL) were converted to 810 mg of the title compound (20% of theory) as a reddish solid.

¹H NMR (400 MHz, DMSO-d6) δ[ppm] 6.77-6.81 (m, 1H), 7.32-7.35 (m, 2H), 7.45 (dd, 1H), 7.56 (d, 1H), 7.80-7.85 (m, 1H), 8.51 (d, 1H), 9.92 (s, 1H).

LCMS (Method 3): Rt=1.13 min, MS (ESIpos) m/z=234 [M+H]⁺

TABLE 1
Intermediates 3-38: the following intermediates were synthesized accordingly

| | | LC-MS | |
|---|---|---|---|
| | | method | |
| | | Retention | |
| | Structure | time | Synthesis |
| Intermediate | IUPAC-Name | Mass found | Method |
| Int. 3 | 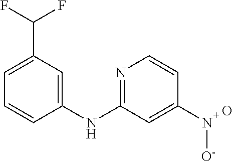 [SMILES: [O-]Nc1ccnc(Nc2cccc(C(F)F)c2)c1 ; O] | Method 3 Rₜ = 1.13 min m/z = 266 [M + H]⁺ | GP A |
| | N-[3-(difluoromethyl)phenyl]-4- | | |
| | nitropyridin-2-amine | | |
| Int. 4 | 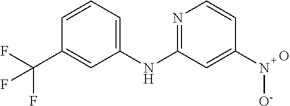 [SMILES: [O-]Nc1ccnc(Nc2cccc(C(F)(F)F)c2)c1 ; O] | Method 3 Rₜ = 1.27 min m/z = 284 [M + H]⁺ | GP A |
| | 4-nitro-N-[3- | | |
| | (trifluoromethyl)phenyl]pyridin-2- | | |
| | amine | | |
| Int. 5 | 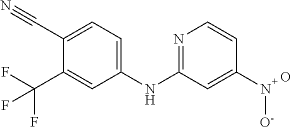 [SMILES: N#Cc1ccc(Nc2cc(N[O-])ccn2)cc1C(F)(F)F ; O] | Method 3 Rₜ = 1.19 min m/z = 309 [M + H]⁺ | GP A |
| | 4-[(4-nitropyridin-2-yl)amino]-2- | | |
| | (trifluoromethyl)benzonitrile | | |
| Int. 6 | 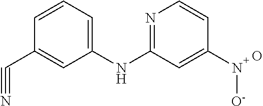 [SMILES: N#Cc1cccc(Nc2cc(N[O-])ccn2)c1 ; O] | Method 1 Rₜ = 1.13 min m/z = 239 [M − H]⁻ | GP B |
| | 3-[(4-nitropyridin-2- | | |
| | yl)amino]benzonitrile | | |
| Int. 7 | 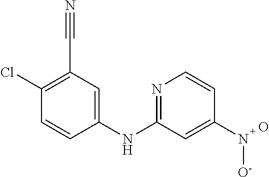 [SMILES: N#Cc1cc(Nc2cc(N[O-])ccn2)ccc1Cl ; O] | Method 3 Rₜ = 1.16 min m/z = 275 [M + H]⁺ | GP A |
| | 2-chloro-5-[(4-nitropyridin-2- | | |
| | yl)amino]benzonitrile | | |
| Int. 8 | 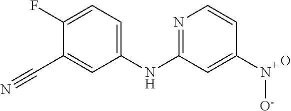 [SMILES: N#Cc1cc(Nc2cc(N[O-])ccn2)ccc1F ; O] | Method 3 Rₜ = 1.07 min m/z = 259 [M + H]⁺ | GP A |
| | 2-fluoro-5-[(4-nitropyridin-2- | | |
| | yl)amino]benzonitrile | | |
| Int. 9 | 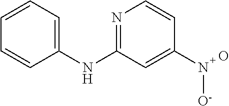 [SMILES: [O-]Nc1ccnc(Nc2ccccc2)c1 ; O] | Method 1 Rₜ = 1.15 min m/z = 216 [M + H]⁺ | GP B |
| | 4-nitro-N-phenylpyridin-2-amine | | |
| Int. 10 | 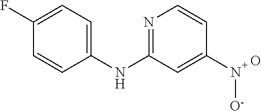 [SMILES: [O-]Nc1ccnc(Nc2ccc(F)cc2)c1 ; O] | Method 1 Rₜ = 1.16 min m/z = 234 [M + H]⁺ | GP A |
| | N-(4-fluorophenyl)-4-nitropyridin-2- | | |
| | amine | | |
| Int. 11 | 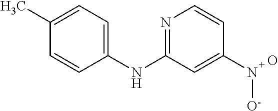 | Method 3 Rₜ = 1.15 min m/z = 230 [M + H]⁺ | GP A |
| | N-(4-methylphenyl)-4-nitropyridin-2- | | |
| | amine | | |
| Int. 12 | 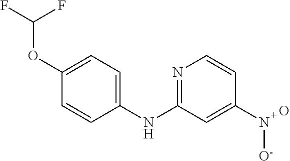 [SMILES: [O-]Nc1ccnc(Nc2ccc(OC(F)F)cc2)c1 ; O] | Method 3 Rₜ = 1.14 min m/z = 282 [M + H]⁺ | GP A |
| | N-[4-(difluoromethoxy)phenyl]-4- | | |
| | nitropyridin-2-amine | | |
| Int. 13 | 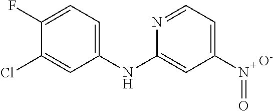 | Method 1 Rₜ = 1.30 min m/z = 268 [M + H]⁺ | GP A |
| | N-(3-chloro-4-fluorophenyl)-4- | | |
| | nitropyridin-2-amine | | |
| Int. 14 | 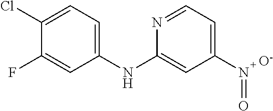 | Method 1 Rₜ = 1.29 min m/z = 266 [M − H]⁻ | GP A |
| | N-(4-chloro-3-fluorophenyl)-4- | | |
| | nitropyridin-2-amine | | |
| Int. 15 | 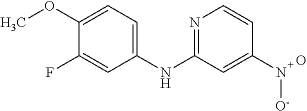 | Method 3 Rₜ = 1.07 min m/z = 264 [M + H]⁺ | GP A |
| | N-(3-fluoro-4-methoxyphenyl)-4- | | |
| | nitropyridin-2-amine | | |
| Int. 16 | 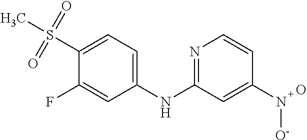 | Method 3 Rₜ = 0.91 min m/z = 312 [M + H]⁺ | GP A |
| | N-[3-fluoro-4-(methanesulfonyl)phenyl]- | | |
| | 4-nitropyridin-2-amine | | |
| Int. 17 | 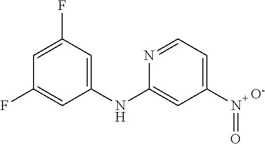 | Method 3 Rₜ = 1.22 min m/z = 252 [M + H]⁺ | GP B |
| | N-(3,5-difluorophenyl)-4-nitropyridin-2- | | |
| | amine | | |
| Int. 18 | 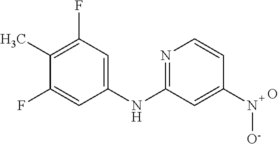 | Method 3 Rₜ = 1.29 min m/z = 266 [M + H]⁺ | GP A |
| | N-(3,5-difluoro-4-methylphenyl)-4- | | |
| | nitropyridin-2-amine | | |
| Int. 19 | 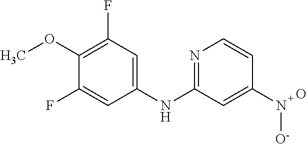 | Method 3 Rₜ = 1.18 min m/z = 282 [M + H]⁺ | GP A |
| | N-(3,5-difluoro-4-methoxyphenyl)-4- | | |
| | nitropyridin-2-amine | | |
| Int. 20 | 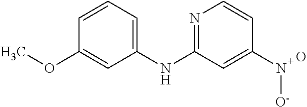 | Method 3 Rₜ = 1.06 min m/z = 246 [M + H]⁺ | GP A |
| | N-(3-methoxyphenyl)-4-nitropyridin-2- | | |
| | amine | | |
| Int. 21 | 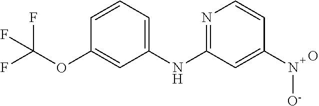 [SMILES: [O-]Nc1ccnc(Nc2cccc(OC(F)(F)F)c2)c1 ; O] | Method 3 Rₜ = 1.30 min m/z = 300 [M + H]⁺ | GP A |
| | 4-nitro-N-[3- | | |
| | (trifluoromethoxy)phenyl]pyridin-2- | | |
| | amine | | |
| Int. 22 | 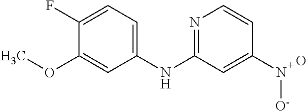 | Method 3 Rₜ = 1.07 min m/z = 264 [M + H]⁺ | GP A |
| | N-(4-fluoro-3-methoxyphenyl)-4- | | |
| | nitropyridin-2-amine | | |
| Int. 23 | 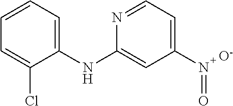 | Method 1 Rₜ = 1.21 min m/z = 250 [M + H]⁺ | GP A |
| | N-(2-chlorophenyl)-4-nitropyridin-2- | | |
| | amine | | |
| Int. 24 | 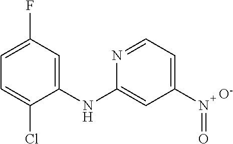 | Method 1 Rₜ = 1.26 min m/z = 266 [M − H]⁻ | GP A |
| | N-(2-chloro-5-fluorophenyl)-4- | | |
| | nitropyridin-2-amine | | |
| Int. 25 | 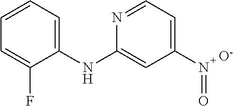 | Method 1 Rₜ = 1.13 min m/z = 234 [M + H]⁺ | GP A |
| | N-(2-fluorophenyl)-4-nitropyridin-2- | | |
| | amine | | |
| Int. 26 | 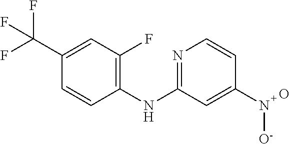 [SMILES: [O-]Nc1ccnc(Nc2ccc(C(F)(F)F)cc2F)c1 ; O] | Method 3 Rₜ = 1.29 min m/z = 302 [M + H]⁺ | GP A |
| | N-[2-fluoro-4-(trifluoromethyl)phenyl]-4- | | |
| | nitropyridin-2-amine | | |
| Int. 27 | 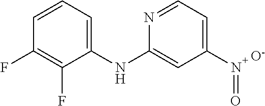 | Method 1 Rₜ = 1.16 min m/z = 250 [M − H]⁻ | GP A |
| | N-(2,3-difluorophenyl)-4-nitropyridin-2- | | |
| | amine | | |
| Int. 28 | 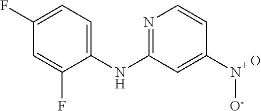 [SMILES: [O-]Nc1ccnc(Nc2ccc(F)cc2F)c1 ; O] | Method 1 Rₜ = 1.18 min m/z = 252 [M]⁺ | GP A |
| | N-(2,4-difluorophenyl)-4-nitropyridin-2- | | |
| | amine | | |
| Int. 29 | 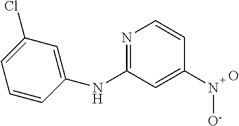 [SMILES: [O-]Nc1ccnc(Nc2cccc(Cl)c2)c1 ; O] | Method 1 Rₜ = 1.29 min m/z = 250 [M + H]⁺ | GP A |
| | N-(3-chlorophenyl)-4-nitropyridin-2- | | |
| | amine | | |
| Int. 30 | 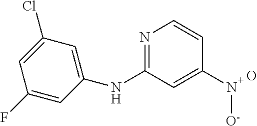 [SMILES: [O-]Nc1ccnc(Nc2cc(F)cc(Cl)c2)c1 ; O] | Method 1 Rₜ = 1.35 min m/z = 268 [M + H]⁺ | GP A |
| | N-(3-chloro-5-fluorophenyl)-4- | | |
| | nitropyridin-2-amine | | |
| Int. 31 | 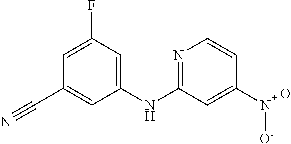 [SMILES: N#Cc1cc(F)cc(Nc2cc(N[O-])ccn2)c1 ; O] | Method 1 Rₜ = 1.20 min m/z = 257 [M − H]⁻ | GP A |
| | 3-Fluor-5-[(4-nitropyridin-2-yl)amino] | | |
| | benzonitril | | |
| Int. 32 | 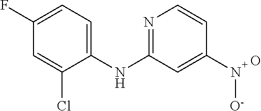 [SMILES: [O-]Nc1ccnc(Nc2ccc(F)cc2Cl)c1 ; O] | Method 1 Rₜ = 1.25 min m/z = 268 [M + H]⁺ | GP A |
| | N-(2-Chlor-4-fluorphenyl)-4-nitropyridin- | | |
| | 2-amin | | |
| Int. 33 | 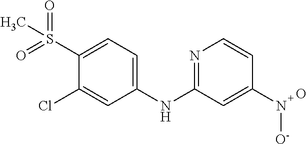 | Method 1 Rₜ = 1.07 min m/z = 326 [M − H]⁻ | GP A |
| | N-[3-Chlor-4-(methylsulfonyl)phenyl]-4- | | |
| | nitropyridin-2-amin | | |
| Int. 34 | 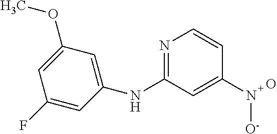 | Method 1 Rₜ = 1.25 min m/z = 264 [M + H]⁺ | GP A |
| | N-(3-Fluor-5-methoxyphenyl)-4- | | |
| | nitropyridin-2-amin | | |
| Int. 35 | 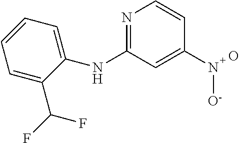 [SMILES: [O-]Nc1ccnc(Nc2ccccc2C(F)F)c1 ; O] | Method 1 Rₜ = 1.17 min m/z = 266 [M + H]⁺ | GP A |
| | N-[2-(Difluormethyl)phenyl]-4- | | |
| | nitropyridin-2-amin | | |
| Int. 36 | 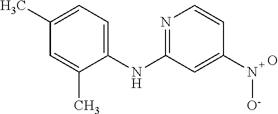 | Method 1 Rₜ = 1.27 min m/z = 244 [M + H]⁺ | GP A |
| | N-(2,4-Dimethylphenyl)-4-nitropyridin-2- | | |
| | amin | | |
| Int. 37 | 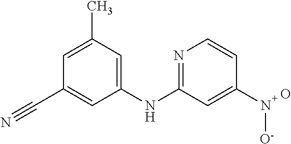 | Method 1 Rₜ = 1.21 min m/z = 255 [M + H]⁺ | GP A |
| | 3-Methyl-5-[(4-nitropyridin-2-yl)amino]- | | |
| | benzonitril | | |
| Int. 38 | 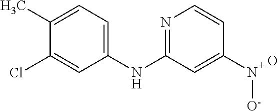 | Method 1 Rₜ = 1.37 min m/z = 264 [M + H]⁺ | GP A |
| | N-(3-Chlor-4-methylphenyl)- | | |
| | nitropyridin-2-amin | | |

### Intermediate 39

### N-(3,4-difluorophenyl)-N-(4-nitropyridin-2-yl)acetamide

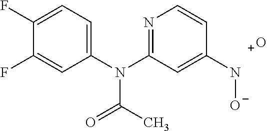

According to GP C, 655 mg (2.61 mmol) N-(3,4-difluorophenyl)-4-nitropyridin-2-amine (Int. 1) was dissolved in 13 mL acetic anhydride, DMAP (0.1 eq., 32 mg, 0.26 mmol) was added and the mixture stirred at 100° C. for 18 h. After cooling to rt, the reaction mixture was partitioned between ethyl acetate and water, extracted with ethyl acetate, washed with brine, dried with sodium sulfate and the solvents removed in vacuo. The crude product was purified by chromatography to yield 765 mg (93% of theory) of the title compound.

¹H NMR (400 MHz, DMSO-d6) δ[ppm] 2.03 (s, 3H), 7.30-7.34 (m, 1H), 7.52-7.58 (m, 1H), 7.66-7.72 (m, 1H), 7.95 (dd, 1H), 8.57 (d, 1H), 8.66 (d, 1H).

LCMS (method 1): Rt=1.08 min, MS (ESIpos) m/z=294 [M+H]⁺

TABLE 2
Intermediates 40-77: the following intermediates were synthesized accordingly

| | | LC-MS method |
|---|---|---|
| | Structure | Retention time |
| Intermediate | IUPAC-Name | Mass found |
| Int. 40 | 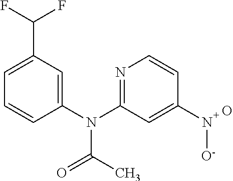 | Method 3 Rₜ = 0.96 min m/z = 308 [M + H]⁺ |
| | N-[3-(difluoromethyl)phenyl]-N-(4- | |
| | nitropyridin-2-yl)acetamide | |
| Int. 41 | 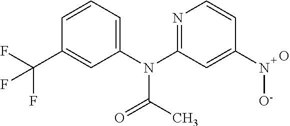 | Method 3 Rₜ = 1.09 min m/z = 326 [M + H]⁺ |
| | N-(4-nitropyridin-2-yl)-N-[3- | |
| | (trifluoromethyl)phenyl]acetamide | |
| Int. 42 | 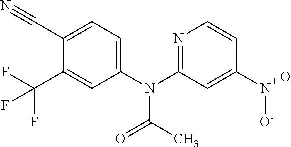 | Method 3 Rₜ = 1.04 min m/z = 351 [M + H]⁺ |
| | N-[4-cyano-3-(trifluoromethyl)phenyl]-N-(4- | |
| | nitropyridin-2-yl)acetamide | |
| Int. 43 | 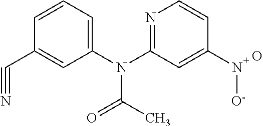 | Method 1 Rₜ = 0.95 min m/z = 283 [M + H]⁺ |
| | N-(3-cyanophenyl)-N-(4-nitropyridin-2- | |
| | yl)acetamide | |
| Int. 44 | 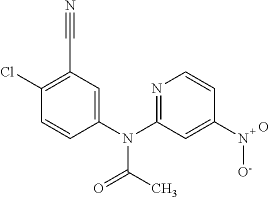 | Method 3 Rₜ = 0.97 min m/z = 317 [M + H]⁺ |
| | N-(4-chloro-3-cyanophenyl)-N-(4- | |
| | nitropyridin-2-yl)acetamide | |
| Int. 45 | 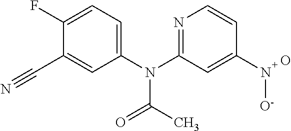 [SMILES: CC(=O)N(c1ccc(F)c(C#N)c1)c1cc(N[O-])ccn1 ; O] | Method 1 Rₜ = 0.98 min m/z = 300 [M]⁺ |
| | N-(3-cyano-4-fluorophenyl)-N-(4- | |
| | nitropyridin-2-yl)acetamide | |
| Int. 46 | 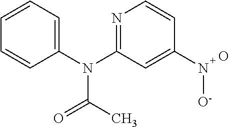 | Method 2 Rₜ = 1.00 min m/z = 258 [M + H]⁺ |
| | N-(4-nitropyridin-2-yl)-N-phenylacetamide | |
| Int. 47 | 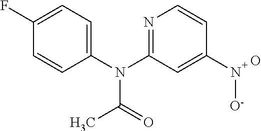 | Method 2 Rₜ = 1.03 min m/z = 276 [M + H]⁺ |
| | N-(4-fluorophenyl)-N-(4-nitropyridin-2- | |
| | yl)acetamide | |
| Int. 48 | 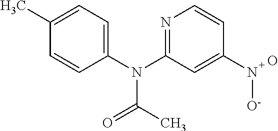 | Method 3 Rₜ = 1.00 min m/z = 272 [M + H]⁺ |
| | N-(4-methylphenyl)-N-(4-nitropyridin-2- | |
| | yl)acetamide | |
| Int. 49 | 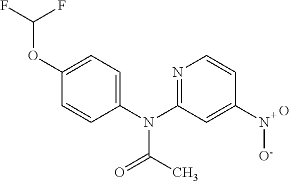 | Method 3 Rₜ = 0.99 min m/z = 324 [M + H]⁺ |
| | N-[4-(difluoromethoxy)phenyl]-N-(4- | |
| | nitropyridin-2-yl)acetamide | |
| Int. 50 | 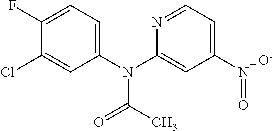 | Method 1 Rₜ = 1.13 min m/z = 310 [M + H]⁺ |
| | N-(3-chloro-4-fluorophenyl)-N-(4- | |
| | nitropyridin-2-yl)acetamide | |
| Int. 51 | 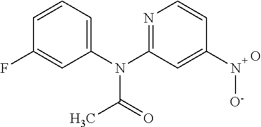 | Method 3 Rₜ = 0.94 min m/z = 276 [M + H]⁺ |
| | N-(3-fluorophenyl)-N-(4-nitropyridin-2- | |
| | yl)acetamide | |
| Int. 52 | 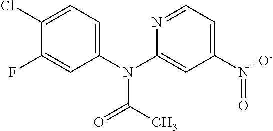 | Method 1 Rₜ = 1.14 min m/z = 310 [M + H]⁺ |
| | N-(4-chloro-3-fluorophenyl)-N-(4- | |
| | nitropyridin-2-yl)acetamide | |
| Int. 53 | 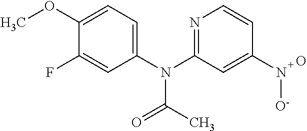 | Method 3 Rₜ = 0.93 min m/z = 306 [M + H]⁺ |
| | N-(3-fluoro-4-methoxyphenyl)-N-(4- | |
| | nitropyridin-2-yl)acetamide | |
| Int. 54 | 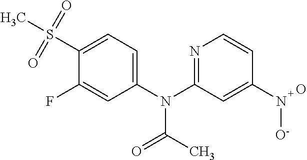 | Method 3 Rₜ = 0.74 min m/z = 354 [M + H]⁺ |
| | N-[3-fluoro-4-(methanesulfonyl)phenyl]-N- | |
| | (4-nitropyridin-2-yl)acetamide | |
| Int. 55 | 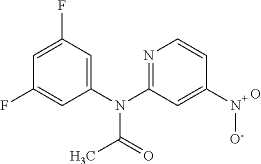 | Method 1 Rₜ = 1.09 min m/z = 294 [M + H]⁺ |
| | N-(3,5-difluorophenyl)-N-(4-nitropyridin-2- | |
| | yl)acetamide | |
| Int. 56 | 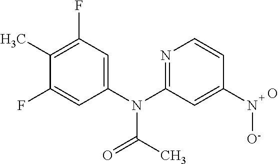 | Method 3 Rₜ = 1.10 min m/z = 308 [M + H]⁺ |
| | N-(3,5-difluoro-4-methylphenyl)-N-(4- | |
| | nitropyridin-2-yl)acetamide | |
| Int. 57 | 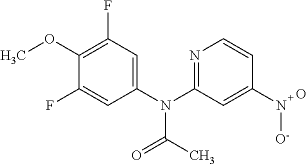 | Method 3 Rₜ = 1.01 min m/z = 324 [M + H]⁺ |
| | N-(3,5-difluoro-4-methoxyphenyl)-N-(4- | |
| | nitropyridin-2-yl)acetamide | |
| Int. 58 | 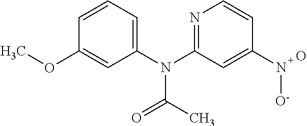 | Method 1 Rₜ = 1.01 min m/z = 288 [M + H]⁺ |
| | N-(3-methoxyphenyl)-N-(4-nitropyridin-2- | |
| | yl)acetamide | |
| Int. 59 | 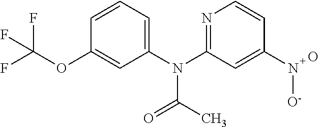 | Method 3 Rₜ = 1.14 min m/z = 342 [M + H]⁺ |
| | N-(4-nitropyridin-2-yl)-N-[3- | |
| | (trifluoromethoxy)phenyl]acetamide | |
| Int. 60 | 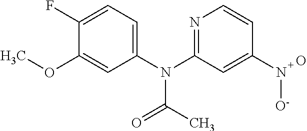 | Method 3 Rₜ = 0.94 min m/z = 306 [M + H]⁺ |
| | N-(4-fluoro-3-methoxyphenyl)-N-(4- | |
| | nitropyridin-2-yl)acetamide | |
| Int. 61 | 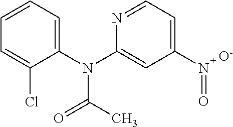 | Method 1 Rₜ = 1.11 min m/z = 292 [M + H]⁺ |
| | N-(2-chlorophenyl)-N-(4-nitropyridin-2- | |
| | yl)acetamide | |
| Int. 62 | 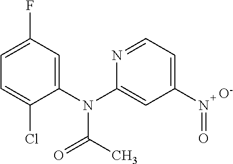 | Method 1 Rₜ = 1.13 min m/z = 310 [M + H]⁺ |
| | N-(2-chloro-5-fluorophenyl)-N-(4- | |
| | nitropyridin-2-yl)acetamide | |
| Int. 63 | 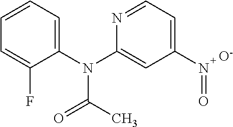 | Method 1 Rₜ = 1.03 min m/z = 276 [M + H]⁺ |
| | N-(2-fluorophenyl)-N-(4-nitropyridin-2- | |
| | yl)acetamide | |
| Int. 64 | 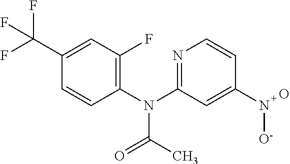 | Method 3 Rₜ = 1.16 min m/z = 344 [M + H]⁺ |
| | N-[2-fluoro-4-(trifluoromethyl)phenyl]-N- | |
| | (4-nitropyridin-2-yl)acetamide | |
| Int. 65 | 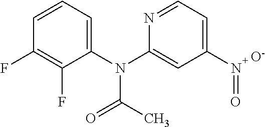 | Method 1 Rₜ = 1.07 min m/z = 294 [M + H]⁺ |
| | N-(2,3-difluorophenyl)-N-(4-nitropyridin-2- | |
| | yl)acetamide | |
| Int. 66 | 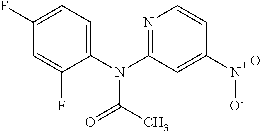 | Method 1 Rₜ = 1.10 min m/z = 294 [M + H]⁺ |
| | N-(2,4-difluorophenyl)-N-(4-nitropyridin-2- | |
| | yl)acetamide | |
| Int. 67 | 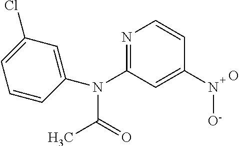 | Method 1 Rₜ = 1.13 min m/z = 292 [M + H]⁺ |
| | N-(3-chlorophenyl)-N-(4-nitropyridin-2- | |
| | yl)acetamide | |
| Int. 68 | 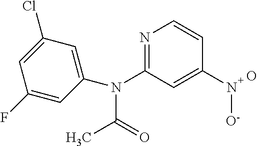 | Method 1 Rₜ = 1.17 min m/z = 310 [M + H]⁺ |
| | N-(3-chloro-5-fluorophenyl)-N-(4- | |
| | nitropyridin-2-yl)acetamide | |
| Int. 69 | 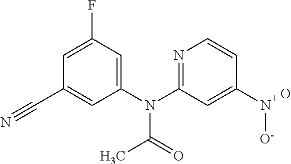 | Method 1 Rₜ = 1.03 min m/z = 301 [M + H]⁺ |
| | N-(3-Cyan-5-fluorphenyl)-N-(4- | |
| | nitropyridin-2-yl)acetamide | |
| Int. 70 | 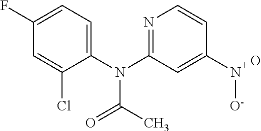 | Method 1 Rₜ = 1.18 min m/z = 310 [M + H]⁺ |
| | N-(2-Chlor-4-fluorphenyl)-N-(4- | |
| | nitropyridin-2-yl)acetamide | |
| Int. 71 | 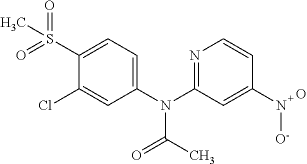 | Method 1 Rₜ = 0.93 min m/z = 370 [M + H]⁺ |
| | N-[3-Chlor-4-(methylsulfonyl)phenyl]-N-(4- | |
| | nitropyridin-2-yl)acetamide | |
| Int. 72 | 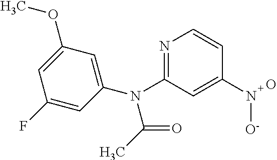 | Method 1 Rₜ = 1.09 min m/z = 306 [M + H]⁺ |
| | N-(3-Fluor-5-methoxyphenyl)-N-(4- | |
| | nitropyridin-2-yl)acetamide | |
| Int. 73 | 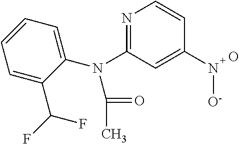 | Method 1 Rₜ = 1.13 min m/z = 308 [M + H]⁺ |
| | N-[2-(Difluormethyl)phenyl]-N-(4- | |
| | nitropyridin-2-yl)acetamide | |
| Int. 74 | 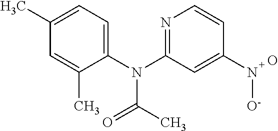 | Method 1 Rₜ = 1.22 min m/z = 286 [M + H]⁺ |
| | N-(2,4-Dimethylphenyl)-N-(4-nitropyridin- | |
| | 2-yl)acetamide | |
| Int. 75 | 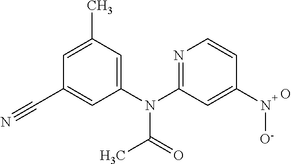 | Method 1 Rₜ = 1.05 min m/z = 297 [M + H]⁺ |
| | N-(3-Cyan-5-methylphenyl)-N-(4- | |
| | nitropyridin-2-yl)acetamide | |
| Int. 76 | 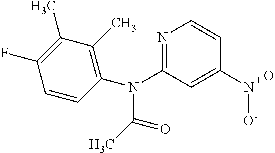 | Method 1 Rₜ = 1.22 min m/z = 304 [M + H]⁺ |
| | N-(4-Fluor-2,3-dimethylphenyl)-N-(4- | |
| | nitropyridin-2-yl)acetamide | |
| Int. 77 | 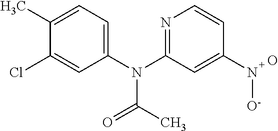 | Method 1 Rₜ = 1.23 min m/z = 306 [M + H]⁺ |
| | N-(3-Chlor-4-methylphenyl)-N-(4- | |
| | nitropyridin-2-yl)acetamide | |

TABLE 3
Intermediate 78: the following intermediate was synthesized according GP
C, using butanoic anhydride

| | | LC-MS method |
|---|---|---|
| | Structure | Retention time |
| Intermediate | IUPAC-Name | Mass found |
| Int. 78 | 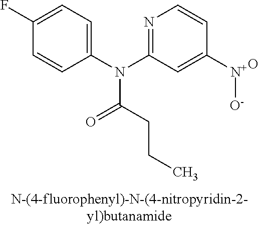 | Method 1 Rₜ = 1.24 min m/z = 304 [M + H]⁺ |

### Intermediate 79

### N-(4-aminopyridin-2-yl)-N-(3,4-difluorophenyl)acetamide

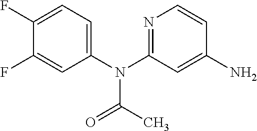 [SMILES: CC(=O)N(c1ccc(F)c(F)c1)c1cc(N)ccn1]

According to GP D, N-(3,4-difluorophenyl)-N-(4-nitropyridin-2-yl)acetamide (Int. 39, 745 mg, 2.54 mmol) were dissolved in ethyl acetate (15 mL), the palladium catalyst was added (10% Pd on activated charcoal, 270 mg, 0.1 eq.) and the mixture hydrogenated (1 atm hydrogen) for 3 h at rt. The catalyst was filtered off and the solvent evaporated to dryness, to yield 669 mg (94% of theory) of the title compound.

¹H NMR (400 MHz, DMSO-d6) δ[ppm] 1.97 (s, 3H), 6.27 (s, br, 2H), 6.43 (dd, 1H), 6.46 (s, br, 1H), 7.02-7.06 (m, 1H), 7.39-7.46 (m, 2H), 7.89 (d, 1H).

LCMS (method 1): Rt=0.78 min, MS (ESIpos) m/z=264 [M+H]⁺

### Intermediate 80

### N-(4-aminopyridin-2-yl)-N-(4-fluorophenyl)acetamide

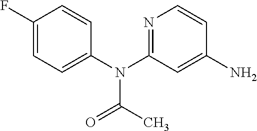 [SMILES: CC(=O)N(c1ccc(F)cc1)c1cc(N)ccn1]

According to GP E, N-(4-fluorophenyl)-N-(4-nitropyridin-2-yl)acetamide (Int. 47, 1.70 g, 6.18 mmol) were dissolved in acetic acid (70 mL) and iron powder (5 eq., 1.72 g, 30.9 mmol) was added portion wise. The mixture was vigorously stirred for 2 h at rt. Then the solids were filtered off via a pad of celite, rinsed with ethyl acetate, and the filtrate was concentrated in vacuo. The product was taken to the next step without further purification.

¹H NMR (400 MHz, DMSO-d6) δ[ppm] 1.96 (s, 3H), 6.23 (s, br, 2H), 6.41 (dd, 1H), 6.43 (s, br, 1H), 7.17-7.22 (m, 2H), 7.25-7.30 (m, 2H), 7.87 (d, 1H).

LCMS (Method 2): Rt=0.58 min, MS (ESIpos) m/z=246 [M+H]⁺

TABLE 4
Intermediates 81-116: the following aromatic amines were generated by
reduction of the corresponding nitro compounds, using an appropriate general
procedure (GP D, GP E)

| | | LC-MS method |
|---|---|---|
| | Structure | Retention time |
| Intermediate | IUPAC-Name | Mass found |
| Int. 81 | 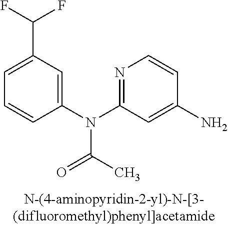 [SMILES: CC(=O)N(c1cccc(C(F)F)c1)c1cc(N)ccn1] | Method 1 Rₜ = 0.75 min m/z = 278 [M + H]⁺ |
| Int. 82 | 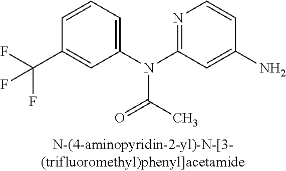 [SMILES: CC(=O)N(c1cccc(C(F)(F)F)c1)c1cc(N)ccn1] | Method 1 Rₜ = 0.86 min m/z = 296 [M + H]⁺ |
| Int. 83 | 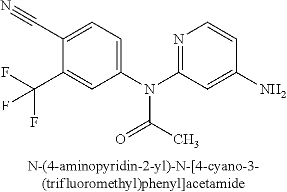 [SMILES: CC(=O)N(c1ccc(C#N)c(C(F)(F)F)c1)c1cc(N)ccn1] | Method 1 Rₜ = 0.87 min m/z = 321 [M + H]⁺ |
| Int. 84 | 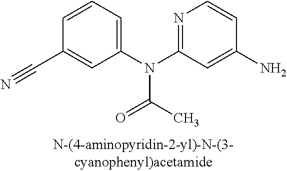 [SMILES: CC(=O)N(c1cccc(C#N)c1)c1cc(N)ccn1] | Method 1 Rₜ = 0.67 min m/z = 253 [M + H]⁺ |
| Int. 85 | 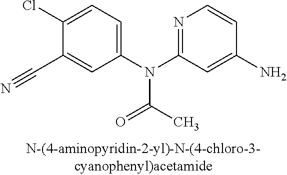 [SMILES: CC(=O)N(c1ccc(Cl)c(C#N)c1)c1cc(N)ccn1] | Method 1 Rₜ = 0.79 min m/z = 287 [M + H]⁺ |
| Int. 86 | 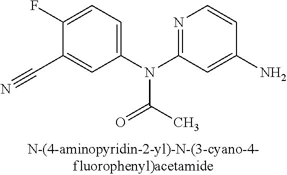 [SMILES: CC(=O)N(c1ccc(F)c(C#N)c1)c1cc(N)ccn1] | Method 1 Rₜ = 0.71 min m/z = 271 [M + H]⁺ |
| Int. 87 | 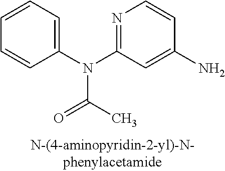 [SMILES: CC(=O)N(c1ccccc1)c1cc(N)ccn1] | Method 2 Rₜ = 0.57 min m/z = 227 [M]⁺ |
| Int. 88 | 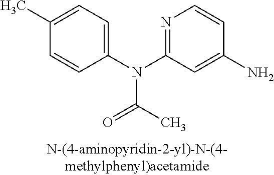 [SMILES: CC(=O)N(c1ccc(C)cc1)c1cc(N)ccn1] | Method 1 Rₜ = 0.75 min m/z = 242 [M + H]⁺ |
| Int. 89 | 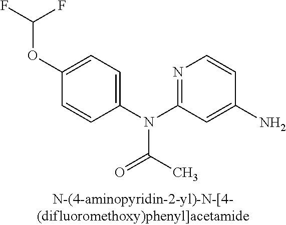 [SMILES: CC(=O)N(c1ccc(OC(F)F)cc1)c1cc(N)ccn1] | Method 1 Rₜ = 0.79 min m/z = 294 [M + H]⁺ |
| Int. 90 | 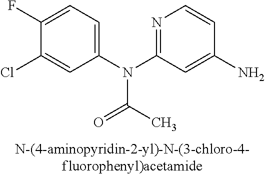 [SMILES: CC(=O)N(c1ccc(F)c(Cl)c1)c1cc(N)ccn1] | Method 1 Rₜ = 0.81 min m/z = 280 [M + H]⁺ |
| Int. 91 | 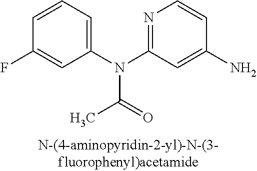 [SMILES: CC(=O)N(c1cccc(F)c1)c1cc(N)ccn1] | Method 1 Rₜ = 0.72 min m/z = 246 [M + H]⁺ |
| Int. 92 | 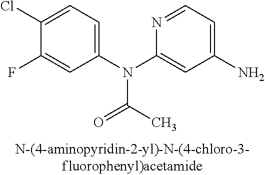 [SMILES: CC(=O)N(c1ccc(Cl)c(F)c1)c1cc(N)ccn1] | Method 1 Rₜ = 0.83 min m/z = 280 [M + H]⁺ |
| Int. 93 | 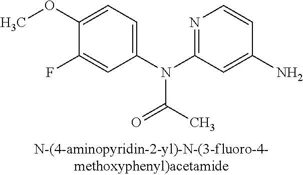 [SMILES: COc1ccc(N(C(C)=O)c2cc(N)ccn2)cc1F] | Method 1 Rₜ = 0.70 min m/z = 276 [M + H]⁺ |
| Int. 94 | 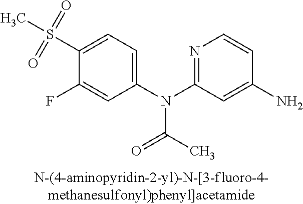 [SMILES: CC(=O)N(c1ccc(S(C)(=O)=O)c(F)c1)c1cc(N)ccn1] | Method 1 Rₜ = 0.63 min m/z = 324 [M + H]⁺ |
| Int. 95 | 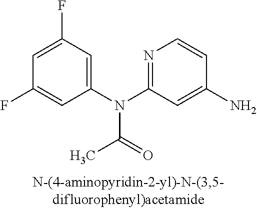 [SMILES: CC(=O)N(c1cc(F)cc(F)c1)c1cc(N)ccn1] | Method 1 Rₜ = 0.78 min m/z = 264 [M + H]⁺ |
| Int. 96 | 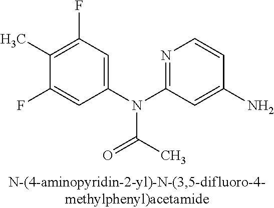 [SMILES: CC(=O)N(c1cc(F)c(C)c(F)c1)c1cc(N)ccn1] | Method 1 Rₜ = 0.87 min m/z = 278 [M + H]⁺ |
| Int. 97 | 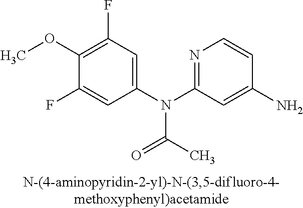 [SMILES: COc1c(F)cc(N(C(C)=O)c2cc(N)ccn2)cc1F] | Method 1 Rₜ = 0.78 min m/z = 294 [M + H]⁺ |
| Int. 98 | 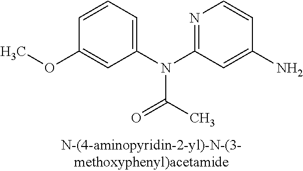 [SMILES: COc1cccc(N(C(C)=O)c2cc(N)ccn2)c1] | Method 1 Rₜ = 0.69 min m/z = 258 [M + H]⁺ |
| Int. 99 | 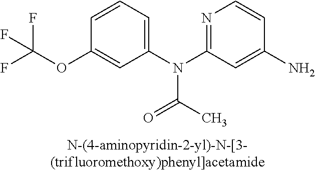 [SMILES: CC(=O)N(c1cccc(OC(F)(F)F)c1)c1cc(N)ccn1] | Method 1 Rₜ = 0.90 min m/z = 312 [M + H]⁺ |
| Int. 100 | 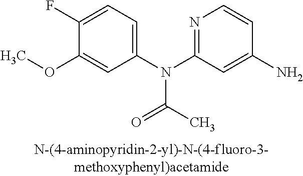 [SMILES: COc1cc(N(C(C)=O)c2cc(N)ccn2)ccc1F] | Method 1 Rₜ = 0.72 min m/z = 276 [M + H]⁺ |
| Int. 101 | 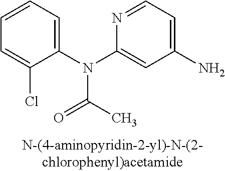 [SMILES: CC(=O)N(c1cc(N)ccn1)c1ccccc1Cl] | Method 1 Rₜ = 0.76 min m/z = 262 [M + H]⁺ |
| Int. 102 | 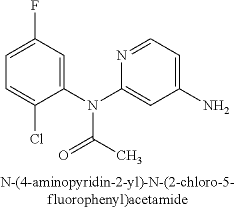 [SMILES: CC(=O)N(c1cc(N)ccn1)c1cc(F)ccc1Cl] | Method 1 Rₜ = 0.78 min m/z = 280 [M + H]⁺ |
| Int. 103 | 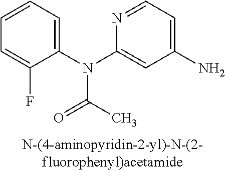 [SMILES: CC(=O)N(c1cc(N)ccn1)c1ccccc1F] | Method 1 Rₜ = 0.69 min m/z = 246 [M + H]⁺ |
| Int. 104 | 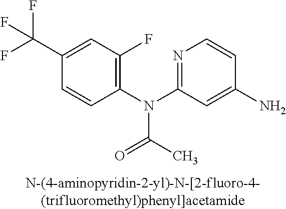 [SMILES: CC(=O)N(c1cc(N)ccn1)c1ccc(C(F)(F)F)cc1F] | Method 1 Rₜ = 0.90 min m/z = 314 [M + H]⁺ |
| Int. 105 | 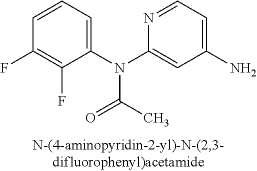 [SMILES: CC(=O)N(c1cc(N)ccn1)c1cccc(F)c1F] | Method 1 Rₜ = 0.73 min m/z = 264 [M + H]⁺ |
| Int. 106 | 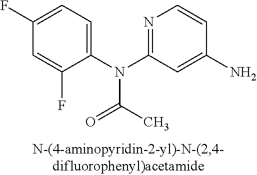 [SMILES: CC(=O)N(c1cc(N)ccn1)c1ccc(F)cc1F] | Method 1 R^{t} = 0.74 min m/z = 264 [M + H]⁺ |
| Int. 107 | 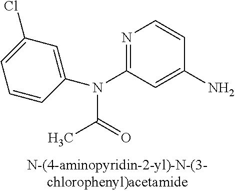 [SMILES: CC(=O)N(c1cccc(Cl)c1)c1cc(N)ccn1] | Method 1 Rₜ = 0.79 min m/z = 262 [M + H]⁺ |
| Int. 108 | 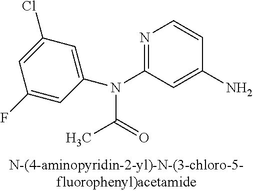 [SMILES: CC(=O)N(c1cc(F)cc(Cl)c1)c1cc(N)ccn1] | Method 1 Rₜ = 0.85 min m/z = 280 [M + H]⁺ |
| Int. 109 | 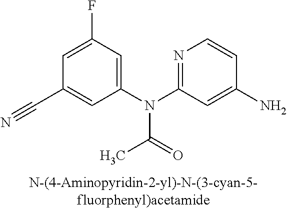 [SMILES: CC(=O)N(c1cc(F)cc(C#N)c1)c1cc(N)ccn1] | Method 1 Rₜ = 0.73 min m/z = 271 [M + H]⁺ |
| Int. 110 | 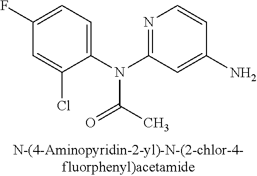 [SMILES: CC(=O)N(c1cc(N)ccn1)c1ccc(F)cc1Cl] | Method 1 Rₜ = 0.81 min m/z = 280 [M + H]⁺ |
| Int. 111 | 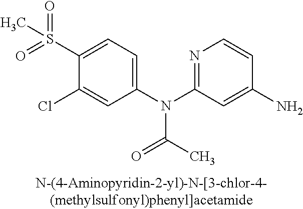 [SMILES: CC(=O)N(c1ccc(S(C)(=O)=O)c(Cl)c1)c1cc(N)ccn1] | Method 1 Rₜ = 0.69 min m/z = 340 [M + H]⁺ |
| Int. 112 | 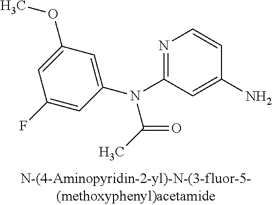 [SMILES: COc1cc(F)cc(N(C(C)=O)c2cc(N)ccn2)c1] | Method 1 Rₜ = 0.77 min m/z = 276 [M + H]⁺ |
| Int. 113 | 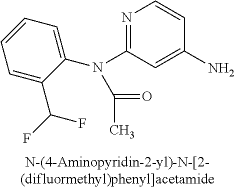 [SMILES: CC(=O)N(c1cc(N)ccn1)c1ccccc1C(F)F] | Method 1 Rₜ = 0.82 min m/z = 278 [M + H]⁺ |
| Int. 114 | 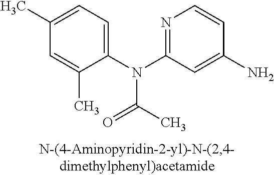 [SMILES: CC(=O)N(c1cc(N)ccn1)c1ccc(C)cc1C] | Method 1 Rₜ = 0.85 min m/z = 256 [M + H]⁺ |
| Int. 115 | 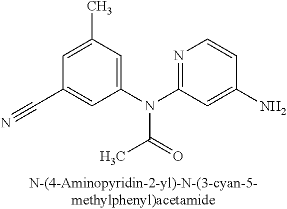 [SMILES: CC(=O)N(c1cc(C)cc(C#N)c1)c1cc(N)ccn1] | Method 1 Rₜ = 0.75 min m/z = 267 [M + H]⁺ |
| Int. 116 | 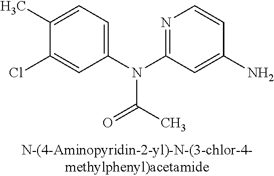 [SMILES: CC(=O)N(c1ccc(C)c(Cl)c1)c1cc(N)ccn1] | Method 1 Rₜ = 0.88 min m/z = 276 [M + H]⁺ |

TABLE 5
Intermediate 117: The following aromatic amine was generated by reduction
of the corresponding nitro compound Int. 78, using an appropriate GP E

| | | LC-MS method |
|---|---|---|
| | Structure | Retention time |
| Intermediate | IUPAC-Name | Mass found |
| Int. 117 | 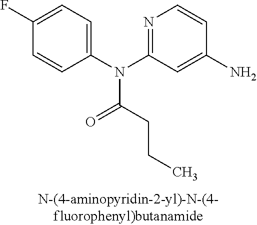 [SMILES: CCCC(=O)N(c1ccc(F)cc1)c1cc(N)ccn1] | Method 1 Rₜ = 0.88 min m/z = 272 [M − H] |

### Intermediate 118—Intermediates with Pyridazine Core

### N-(6-chloropyridazin-4-yl)-2-(2,6-dichlorophenyl)acetamide

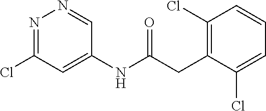 [SMILES: O=C(Cc1c(Cl)cccc1Cl)Nc1cnnc(Cl)c1]

According to GP F, 6-Chloro-4-pyridazinamine (500 mg, 3.86 mmol) and 2,6-dichlorophenylacetic acid (1.8 g, 1.5 eq.) were dissolved in DMF (10 mL) and T3P (11 ml, 18.5 mmol, 4.8 eq.) and diisopropylethylamin (4 ml, 23 mmol, 6 eq.) were added. The mixture was stirred at 80° C. for 18 h, then it was evaporated to a small volume, poured into water and filtered off to give the title compound as a solid.

¹H NMR (400 MHz, DMSO-d6) δ[ppm]: 2.518 (1.54), 2.523 (1.07), 2.888 (0.43), 4.155 (16.00), 5.758 (0.48), 7.356 (2.72), 7.375 (3.84), 7.378 (3.97), 7.397 (4.93), 7.507 (12.48), 7.527 (7.63), 8.050 (7.79), 8.056 (7.25), 9.184 (8.25), 9.190 (8.33), 11.326 (3.56).

LCMS (Method 1): Rt=1.01 min, MS (ESIpos) m/z=314 [M−H]⁻

TABLE 6
Intermediates 119-122: the following aminopyridazine amides were
generated using GP F

| | | LC-MS |
|---|---|---|
| | | method |
| Inter- | Structure | Retention time |
| mediate | IUPAC-Name | Mass found |
| Int. 119 | 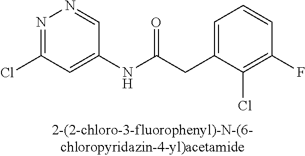 [SMILES: O=C(Cc1cccc(F)c1Cl)Nc1cnnc(Cl)c1] | Method 1 Rₜ = 0.94 min m/z = 300 [M + H]⁺ |
| Int. 120 | 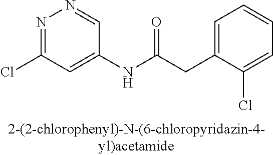 [SMILES: O=C(Cc1ccccc1Cl)Nc1cnnc(Cl)c1] | Method 1 Rₜ = 1.00 min m/z = 282 [M + H]⁺ |
| Int. 121 | 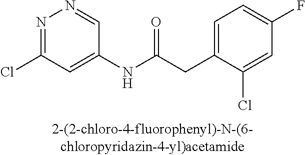 [SMILES: O=C(Cc1ccc(F)cc1Cl)Nc1cnnc(Cl)c1] | Method 1 Rₜ = 0.96 min m/z = 300 [M + H]⁺ |
| Int. 122 | 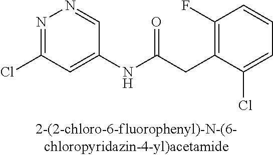 [SMILES: O=C(Cc1c(F)cccc1Cl)Nc1cnnc(Cl)c1] | Method 1 Rₜ = 1.00 min m/z = 300 [M + H]⁺ |

### Intermediate 123

### N-(6-anilinopyridazin-4-yl)-2-(2,6-dichlorophenyl)acetamide

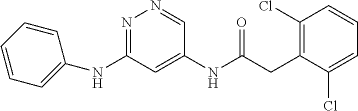 [SMILES: O=C(Cc1c(Cl)cccc1Cl)Nc1cnnc(Nc2ccccc2)c1]

According to GP G, N-(6-chloropyridazin-4-yl)-2-(2,6-dichlorophenyl)acetamide (100 mg, 0.31 mmol) was dissolved in 3 ml ethanol, aniline (29 μl, 0.31 mmol) was added and the mixture was stirred at 80° C. for 48 hrs. Then the mixture was evaporated and purified by HPLC. Yield 75 mg (63%) of the title compound.

LCMS (Method 1): Rt=1.10 min, MS (ESIpos) m/z=373 [M+H]⁺

TABLE 7
Intermediates 124-146: the following intermediates were generated
accordingly, using GP G

| | | LC-MS method |
|---|---|---|
| | Structure | Retention time |
| Intermediate | IUPAC-Name | Mass found |
| Int. 124 | 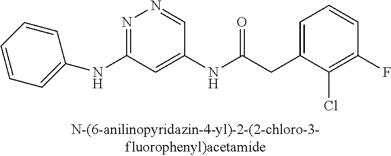 [SMILES: O=C(Cc1cccc(F)c1Cl)Nc1cnnc(Nc2ccccc2)c1] | Method 1 Rₜ = 1.10 min m/z = 357 [M + H]⁺ |
| Int. 125 | 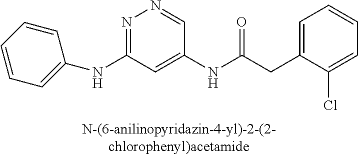 [SMILES: O=C(Cc1ccccc1Cl)Nc1cnnc(Nc2ccccc2)c1] | Method 1 Rₜ = 1.05 min m/z = 337 [M − H]⁻ |
| Int. 126 | 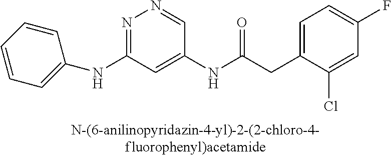 [SMILES: O=C(Cc1ccc(F)cc1Cl)Nc1cnnc(Nc2ccccc2)c1] | Method 1 Rₜ = 1.08 min m/z = 357 [M + H]⁺ |
| Int. 127 | 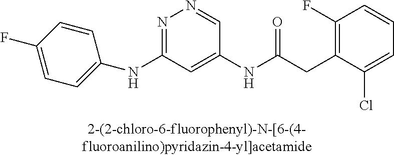 [SMILES: O=C(Cc1c(F)cccc1Cl)Nc1cnnc(Nc2ccc(F)cc2)c1] | Method 1 Rₜ = 1.13 min m/z = 375 [M + H]⁺ |
| Int. 128 | 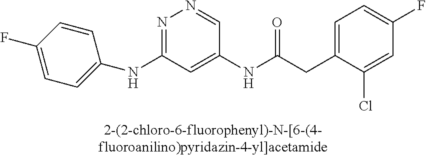 [SMILES: O=C(Cc1ccc(F)cc1Cl)Nc1cnnc(Nc2ccc(F)cc2)c1] | Method 1 Rₜ = 1.10 min m/z = 375 [M + H]⁺ |
| Int. 129 | 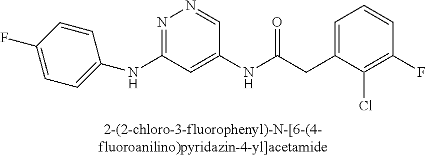 [SMILES: O=C(Cc1cccc(F)c1Cl)Nc1cnnc(Nc2ccc(F)cc2)c1] | Method 1 Rₜ = 1.13 min m/z = 375 [M + H]⁺ |
| Int. 130 | 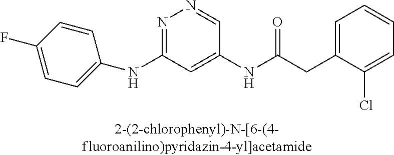 [SMILES: O=C(Cc1ccccc1Cl)Nc1cnnc(Nc2ccc(F)cc2)c1] | Method 1 Rₜ = 1.12 min m/z = 357 [M + H]⁺ |
| Int. 131 | 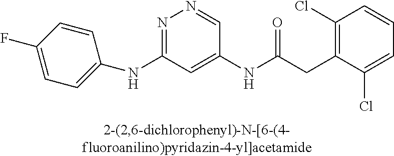 [SMILES: O=C(Cc1c(Cl)cccc1Cl)Nc1cnnc(Nc2ccc(F)cc2)c1] | Method 1 Rₜ = 1.11 min m/z = 391 [M + H]⁺ |
| Int. 132 | 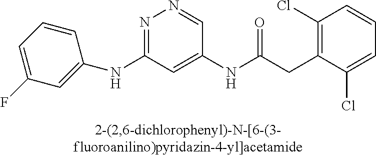 [SMILES: O=C(Cc1c(Cl)cccc1Cl)Nc1cnnc(Nc2cccc(F)c2)c1] | Method 1 Rₜ = 1.14 min m/z = 391 [M + H]⁺ |
| Int. 133 | 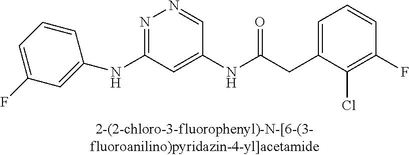 [SMILES: O=C(Cc1cccc(F)c1Cl)Nc1cnnc(Nc2cccc(F)c2)c1] | Method 1 Rₜ = 1.15 min m/z = 375 [M + H]⁺! |
| Int. 134 | 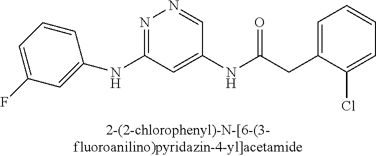 [SMILES: O=C(Cc1ccccc1Cl)Nc1cnnc(Nc2cccc(F)c2)c1] | Method 1 Rₜ = 1.08 min m/z = 357 [M + H]⁺ |
| Int. 135 | 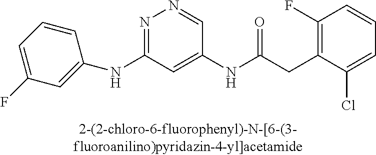 [SMILES: O=C(Cc1c(F)cccc1Cl)Nc1cnnc(Nc2cccc(F)c2)c1] | Method 1 Rₜ = 1.15 min m/z = 375 [M + H]⁺ |
| Int. 136 | 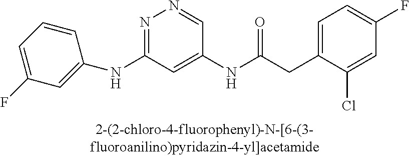 [SMILES: O=C(Cc1ccc(F)cc1Cl)Nc1cnnc(Nc2cccc(F)c2)c1] | Method 1 Rₜ = 1.12 min m/z = 375 [M + H]⁺ |
| Int. 137 | 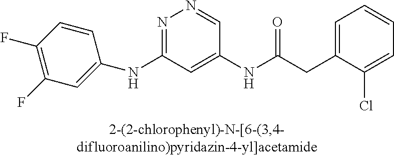 [SMILES: O=C(Cc1ccccc1Cl)Nc1cnnc(Nc2ccc(F)c(F)c2)c1] | Method 1 Rₜ = 1.16 min m/z = 375 [M + H]⁺ |
| Int. 138 | 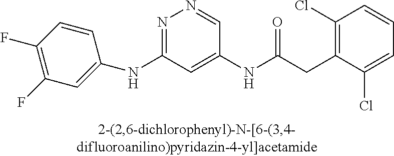 [SMILES: O=C(Cc1c(Cl)cccc1Cl)Nc1cnnc(Nc2ccc(F)c(F)c2)c1] | Method 1 Rₜ = 1.18 min m/z = 407 [M − H]⁻ |
| Int. 139 | 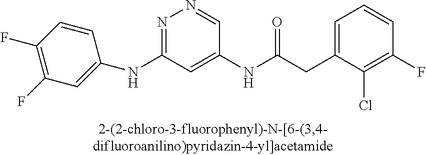 [SMILES: O=C(Cc1cccc(F)c1Cl)Nc1cnnc(Nc2ccc(F)c(F)c2)c1] | Method 1 Rₜ = 1.17 min m/z = 393 [M + H]⁺ |
| Int. 140 | 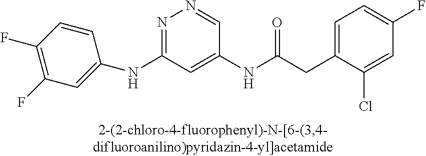 [SMILES: O=C(Cc1ccc(F)cc1Cl)Nc1cnnc(Nc2ccc(F)c(F)c2)c1] | Method 1 Rₜ = 1.14 min m/z = 393 [M + H]⁺ |
| Int. 141 | 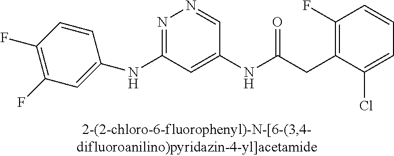 [SMILES: O=C(Cc1c(F)cccc1Cl)Nc1cnnc(Nc2ccc(F)c(F)c2)c1] | Method 1 Rₜ = 1.13 min m/z = 391 [M − H]⁻ |
| Int. 142 | 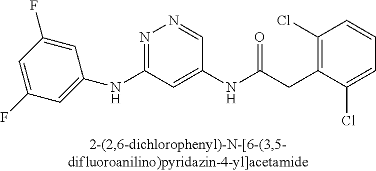 [SMILES: O=C(Cc1c(Cl)cccc1Cl)Nc1cnnc(Nc2cc(F)cc(F)c2)c1] | Method 1 Rₜ = 1.14 min m/z = 393 [M + H]⁺ |
| Int. 143 | 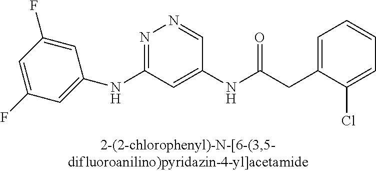 [SMILES: O=C(Cc1ccccc1Cl)Nc1cnnc(Nc2cc(F)cc(F)c2)c1] | Method 1 Rₜ = 1.19 min m/z = 375 [M + H]⁺ |
| Int. 144 | 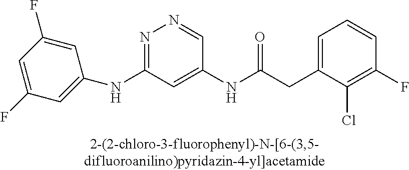 [SMILES: O=C(Cc1cccc(F)c1Cl)Nc1cnnc(Nc2cc(F)cc(F)c2)c1] | Method 1 Rₜ = 1.20 min m/z = 393 [M + H]⁺ |
| Int. 145 | 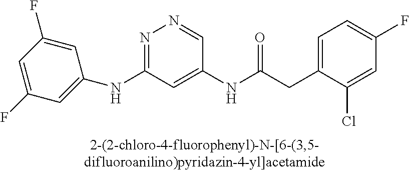 [SMILES: O=C(Cc1ccc(F)cc1Cl)Nc1cnnc(Nc2cc(F)cc(F)c2)c1] | Method 1 Rₜ = 1.17 min m/z = 393 [M + H]⁺ |
| Int. 146 | 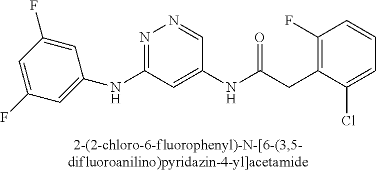 [SMILES: O=C(Cc1c(F)cccc1Cl)Nc1cnnc(Nc2cc(F)cc(F)c2)c1] | Method 1 Rₜ = 1.20 min m/z = 391 [M − H]⁻ |

### Intermediate 147

### N²-(4-fluorophenyl)pyridine-2,4-diamine

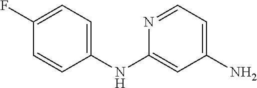 [SMILES: Nc1ccnc(Nc2ccc(F)cc2)c1]

110 mL (1.5 eq., 1.2 mol) of 4-fluoroaniline were dissolved in 500 mL of sulfolane. 24 mL (1.0 eq., 780 mmol) of aqueous conc. HCl were added and the suspension was heated up to 60° C. 100 g (1.0 eq., 778 mmol) of 2-chloropyridin-4-amine (1.0 eq., 778 mmol) were added in portions. The reaction solution was stirred at 130° C. for 18 h. The still warm reaction mixture was diluted with water and the pH value was adjusted to pH=10-11 using semi-concentrated aqueous NaOH solution. The mixture was poured into 4000 mL of water and stirred vigorously for 2 h. The precipitate was filtered off and it was washed intensively with water. The solid material was dried at 50° C. under vacuum. 159 g of the title compound (63% of theory) were obtained as a lilac solid.

¹H NMR (400 MHz, DMSO-d6) δ[ppm] 5.76 (s, 2H), 5.90 (d, J=1.52 Hz, 1H), 6.00 (dd, J=5.70, 1.90 Hz, 1H), 6.79-7.17 (m, 2H), 7.42-7.78 (m, 3H), 8.47 (s, 1H).

LCMS (Method 1): Rₜ=0.86 min, MS (ESIpos) m/z=204 [M+H]⁺

### Intermediate 148

### 2-(2-chloro-3-fluorophenyl)-N-[2-(4-fluoroanilino)pyridin-4-yl]acetamide

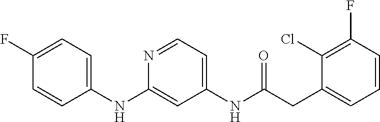 [SMILES: O=C(Cc1cccc(F)c1Cl)Nc1ccnc(Nc2ccc(F)cc2)c1]

115 g (1.15 eq., 610 mmol) of 2-(2-chloro-3-fluorophenyl)acetic acid were dissolved in 700 mL of N,N-dimethylacetamide and 103 g (1.2 eq., 636 mmol) of 1,1′-carbonyldiimidazole were added in portions at room temperature. The reaction mixture was heated up to 40° C. for 4 h. 108 g (1.0 eq., 530 mmol) of N²-(4-fluorophenyl)pyridine-2,4-diamine (Int. 147) were added in portions and the mixture was stirred at 40° C. for 18 h. The mixture was diluted with 5000 mL of water and extracted with ethyl acetate. The combined organic phases were washed with water. After drying over magnesium sulfate and evaporation of the organic phase the remaining residue was triturated with dichloromethane to colorless and finally triturated with n-hexane. 154 g of the title compound (68% of theory) were obtained as a white solid after drying at 50° C.

¹H NMR (400 MHz, DMSO-d6) δ[ppm] 3.93 (s, 2H), 6.71-6.86 (m, 1H), 6.99-7.13 (m, 2H), 7.21-7.45 (m, 4H), 7.57-7.73 (m, 2H), 7.99 (d, J=5.58 Hz, 1H), 8.99 (s, 1H), 10.38-10.53 (m, 1H).

LCMS (Method 1): Rₜ=1.25 min, MS (ESIpos) m/z=374 [M+H]⁺

### Experimental Section—Examples

### Example 1

### N-{4-[2-(2-chlorophenyl)acetamido]pyridin-2-yl}-N-(3,4-difluorophenyl)acetamide

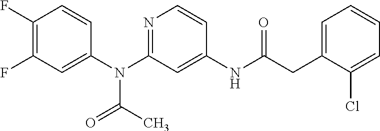 [SMILES: CC(=O)N(c1ccc(F)c(F)c1)c1cc(NC(=O)Cc2ccccc2Cl)ccn1]

According to GP H, N-(4-aminopyridin-2-yl)-N-(3,4-difluorophenyl)acetamide (Int. 79, 70 mg, 0.27 mmol) and 2-chlorophenylacetic acid (54 mg, 1.2 eq.) were dissolved in DMF (2 mL) and HATU (121 mg, 0.32 mmol, 1.2 eq.) and triethylamine (135 mg, 1.33 mmol, 5 eq.) were added. The mixture was stirred at rt for 2 h, then it was poured into water, extracted with ethyl acetate, the combined organic layers washed with brine, dried with sodium sulfate and the solvents evaporated. The crude product was purified by flash chromatography to yield 30 mg (24% of theory) of the title compound as pale yellow foam.

¹H NMR (400 MHz, DMSO-d6) δ[ppm] 2.00 (s, 3H), 3.88 (s, 2H), 7.10-7.14 (m, 1H), 7.29-7.34 (m, 2H), 7.39-7.55 (m, 5H), 7.71 (s, br, 1H), 8.28 (d, 1H), 10.8 (s, 1H).

LC-MS (method 1): Rₜ=1.13 min; MS (ESIpos): m/z=416 [M+H]⁺

### Example 2

### N-{4-[2-(2-chloro-3-fluorophenyl)acetamido]pyridin-2-yl}-N-(4-fluorophenyl)acetamide

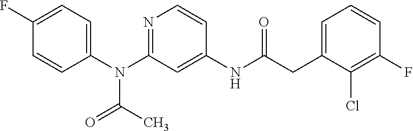 [SMILES: CC(=O)N(c1ccc(F)cc1)c1cc(NC(=O)Cc2cccc(F)c2Cl)ccn1]

According to GP I, N-(4-aminopyridin-2-yl)-N-(4-fluorophenyl)acetamide (Int. 80, 200 mg, 0.82 mmol) and 2-(2-chloro-3-fluorophenyl)acetic acid (154 mg, 1 eq.) were dissolved in DMF (10 mL) and T3P (778 mg, 2.45 mmol, 3 eq.) and triethylamine (495 mg, 4.89 mmol, 6 eq.) were added. The mixture was stirred at rt for 18 h, then it was poured into water, extracted with ethyl acetate, the combined organic layers washed with brine, dried with sodium sulfate and the solvents evaporated. The crude product was purified by preparative HPLC to yield 191 mg (56% of theory) of the title compound as a pale yellow solid. In an alternative procedure, 250 g (1.0 eq., 669 mmol) of 2-(2-chloro-3-fluorophenyl)-N-[2-(4-fluoroanilino)pyridin-4-yl]acetamide (Int. 148) and 160 mL (1.4 eq., 940 mmol) of N,N-diisopropylethylamine were dissolved in 2000 mL of rac-2-methyltetrahydrofuran. At room temperature 71 mL (1.5 eq., 1.0 mol) of acetyl chloride were added dropwise and the reaction mixture was stirred at room temperature for 18 h. The reaction mixture was diluted with ethyl acetate and quenched by adding water. The organic phase was washed with saturated NaHCO₃-solution and water once each. After drying over magnesium sulfate the filtrate was concentrated under vacuum and the remaining residue was purified via column chromatography (Biotage autopurifier system (Isolera LS®), 375 g Biotage SNAP cartridge KP-NH®, hexane/dichloromethane (50%) to hexane/dichloromethane (75%) to dichloromethane (100%) to dichloromethane/ethyl acetate (80%)) followed by a second chromatography (Biotage autopurifier system (Isolera LS®), 1500 g Biotage SNAP cartridge KP-Sil®, hexane (100%) to hexane/ethyl acetate (30%) to ethyl acetate (100%)). The material was triturated with 2-methoxy-2-methylpropane and finally filtered. After drying at 50° C. 219 g of the title compound (79% theoretical yield) were obtained as a white solid.

¹H NMR (400 MHz, DMSO-d6) δ[ppm] 1.99 (s, 3H), 3.94 (s, 2H), 7.21-7.28 (m, 3H), 7.31-7.37 (m, 4H), 7.46 (dd, 1H), 7.68 (s, 1H), 8.27 (d, 1H), 10.8 (s, 1H).

LC-MS (method 1): Rₜ=1.09 min; MS (ESIpos): m/z=416 [M+H]⁺

TABLE 8
Examples 3-184: The following examples were generated by amide coupling
of amines with the corresponding carboxylic acids, using an appropriate general
procedure (GP H, GP I)

| | Structure |
|---|---|
| | IUPAC-Name |
| | LC-MS (method): Retention time; Mass found |
| Example | ¹H-NMR |
| 3 | 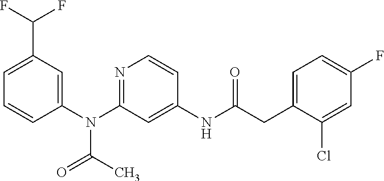 [SMILES: CC(=O)N(c1cccc(C(F)F)c1)c1cc(NC(=O)Cc2ccc(F)cc2Cl)ccn1] |
| | N-{4-[2-(2-chloro-4-fluorophenyl)acetamido]pyridin-2-yl}-N-[3- |
| | (difluoromethyl)phenyl]acetamide |
| | LC-MS (Method 2): Rₜ = 1.14 min; MS (ESIpos): m/z = 448 [M + H]⁺ |
| | ¹H-NMR (400 MHz, DMSO-d6) δ [ppm]: 2.005 (16.00), 2.518 (0.46), |
| | 3.337 (6.30), 6.899 (1.33), 7.038 (2.69), 7.178 (1.25), 7.183 (1.01), |
| | 7.190 (0.97), 7.204 (1.61), 7.211 (1.88), 7.226 (0.97), 7.233 (0.99), |
| | 7.421 (0.93), 7.428 (2.11), 7.434 (2.42), 7.442 (2.57), 7.450 (2.01), |
| | 7.457 (2.81), 7.464 (1.45), 7.480 (3.22), 7.488 (2.61), 7.493 (2.60), |
| | 7.502 (1.78), 7.507 (2.50), 7.512 (1.76), 7.537 (1.62), 7.556 (1.74), |
| | 7.575 (0.56), 7.657 (1.68), 8.297 (2.20), 8.312 (2.08), 10.771 (2.53). |
| 4 | 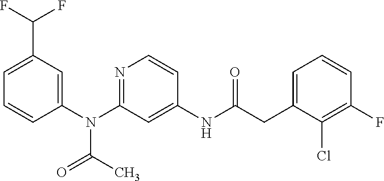 [SMILES: CC(=O)N(c1cccc(C(F)F)c1)c1cc(NC(=O)Cc2cccc(F)c2Cl)ccn1] |
| | N-{4-[2-(2-chloro-3-fluorophenyl)acetamido]pyridin-2-yl}-N-[3- |
| | (difluoromethyl)phenyl]acetamide |
| | LC-MS (Method 2): Rₜ = 1.13 min; MS (ESIneg): m/z = 446 [M − H]⁻ |
| | ¹H-NMR (400 MHz, DMSO-d6) δ [ppm]: 2.005 (16.00), 2.518 (0.99), |
| | 2.523 (0.67), 3.938 (7.22), 6.899 (1.27), 7.038 (2.57), 7.178 (1.16), |
| | 7.258 (0.81), 7.265 (1.21), 7.271 (0.85), 7.275 (0.93), 7.281 (1.25), |
| | 7.321 (0.48), 7.336 (1.99), 7.340 (2.32), 7.357 (4.47), 7.364 (1.43), |
| | 7.371 (1.30), 7.423 (0.79), 7.442 (1.09), 7.480 (1.93), 7.488 (2.57), |
| | 7.492 (2.45), 7.502 (1.85), 7.507 (2.34), 7.513 (1.64), 7.538 (1.55), |
| | 7.557 (1.67), 7.576 (0.53), 7.657 (1.59), 8.300 (2.13), 8.314 (2.01), |
| | 10.809 (2.42). |
| 5 | 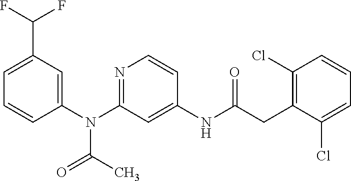 [SMILES: CC(=O)N(c1cccc(C(F)F)c1)c1cc(NC(=O)Cc2c(Cl)cccc2Cl)ccn1] |
| | N-{4-[2-(2,6-dichlorophenyl)acetamido]pyridin-2-yl}-N-[3- |
| | (difluoromethyl)phenyl]acetamide |
| | LC-MS (Method 2): Rₜ = 1.16 min; MS (ESIneg): m/z = 462 [M − H]⁻ |
| | ¹H-NMR (400 MHz, DMSO-d6) δ [ppm]: 2.003 (16.00), 2.518 (0.80), |
| | 2.523 (0.54), 4.077 (7.90), 6.899 (1.36), 7.038 (2.77), 7.178 (1.24), |
| | 7.333 (1.67), 7.351 (2.11), 7.354 (2.35), 7.373 (2.59), 7.426 (0.85), |
| | 7.445 (1.18), 7.465 (1.81), 7.470 (1.86), 7.480 (3.29), 7.484 (11.02), |
| | 7.504 (4.62), 7.513 (1.72), 7.537 (1.61), 7.557 (1.72), 7.576 (0.54), |
| | 7.657 (1.60), 8.300 (2.20), 8.315 (2.08), 10.886 (2.53). |
| 6 | 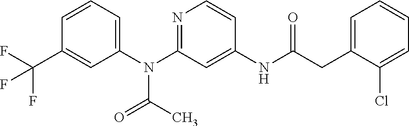 [SMILES: CC(=O)N(c1cccc(C(F)(F)F)c1)c1cc(NC(=O)Cc2ccccc2Cl)ccn1] |
| | N-{4-[2-(2-chlorophenyl)acetamido]pyridin-2-yl}-N-[3- |
| | (trifluoromethyl)phenyl]acetamide |
| | LC-MS (Method 2): Rₜ = 1.20 min; MS (ESIneg): m/z = 446 [M − H]⁻ |
| | ¹H-NMR (400 MHz, DMSO-d6) δ [ppm]: 2.013 (16.00), 2.518 (1.32), |
| | 2.523 (0.87), 3.877 (7.80), 7.300 (3.34), 7.309 (2.69), 7.314 (2.76), |
| | 7.323 (4.65), 7.333 (0.65), 7.394 (1.68), 7.402 (0.84), 7.404 (1.06), |
| | 7.409 (1.24), 7.418 (1.06), 7.423 (0.42), 7.433 (1.98), 7.442 (1.62), |
| | 7.447 (1.25), 7.457 (1.36), 7.514 (2.15), 7.519 (2.47), 7.528 (1.91), |
| | 7.533 (2.38), 7.538 (1.16), 7.615 (0.69), 7.635 (1.73), 7.654 (1.50), |
| | 7.663 (1.94), 7.683 (2.06), 7.703 (1.96), 8.304 (1.96), 8.318 (1.84), |
| | 10.782 (2.45). |
| 7 | 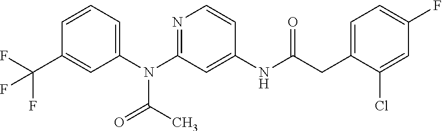 [SMILES: CC(=O)N(c1cccc(C(F)(F)F)c1)c1cc(NC(=O)Cc2ccc(F)cc2Cl)ccn1] |
| | N-{4-[2-(2-chloro-4-fluorophenyl)acetamido]pyridin-2-yl}-N-[3- |
| | (trifluoromethyl)phenyl]acetamide |
| | LC-MS (Method 2): Rₜ = 1.23 min; MS (ESIneg): m/z = 464 [M − H]⁻ |
| | ¹H-NMR (400 MHz, DMSO-d6) δ [ppm]: 2.012 (16.00), 2.518 (2.28), |
| | 2.522 (1.41), 3.819 (0.41), 3.868 (7.17), 7.185 (0.83), 7.192 (0.94), |
| | 7.206 (1.69), 7.212 (1.93), 7.227 (0.95), 7.234 (1.02), 7.429 (1.78), |
| | 7.436 (1.98), 7.443 (1.76), 7.451 (1.95), 7.458 (3.20), 7.464 (1.58), |
| | 7.480 (1.36), 7.505 (1.89), 7.510 (2.00), 7.519 (2.71), 7.524 (2.57), |
| | 7.538 (1.28), 7.616 (0.77), 7.635 (1.93), 7.655 (1.68), 7.664 (2.30), |
| | 7.680 (2.15), 7.702 (2.29), 8.304 (2.15), 8.317 (2.05), 10.779 (2.79). |
| 8 | 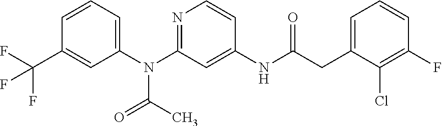 [SMILES: CC(=O)N(c1cccc(C(F)(F)F)c1)c1cc(NC(=O)Cc2cccc(F)c2Cl)ccn1] |
| | N-{4-[2-(2-chloro-3-fluorophenyl)acetamido]pyridin-2-yl}-N-[3- |
| | (trifluoromethyl)phenyl]acetamide |
| | LC-MS (Method 2): Rₜ = 1.22 min; MS (ESIneg): m/z = 464 [M − H]⁻ |
| | ¹H-NMR (400 MHz, DMSO-d6) δ [ppm]: 2.012 (16.00), 2.518 (1.22), |
| | 2.522 (0.79), 3.941 (7.50), 7.258 (0.80), 7.266 (1.23), 7.273 (0.88), |
| | 7.276 (0.91), 7.283 (1.27), 7.322 (0.56), 7.336 (2.10), 7.341 (2.45), |
| | 7.357 (4.71), 7.365 (1.48), 7.372 (1.34), 7.506 (1.73), 7.511 (1.89), |
| | 7.521 (2.55), 7.525 (2.43), 7.540 (1.10), 7.617 (0.71), 7.636 (1.75), |
| | 7.655 (1.49), 7.665 (1.95), 7.684 (2.03), 7.705 (1.99), 8.307 (2.01), |
| | 8.321 (1.91), 10.820 (2.51). |
| 9 | 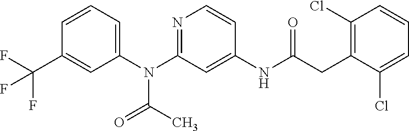 [SMILES: CC(=O)N(c1cccc(C(F)(F)F)c1)c1cc(NC(=O)Cc2c(Cl)cccc2Cl)ccn1] |
| | N-{4-[2-(2,6-dichlorophenyl)acetamido]pyridin-2-yl}-N-[3- |
| | (trifluoromethyl)phenyl]acetamide |
| | LC-MS (Method 2): Rₜ = 1.25 min; MS (ESIneg): m/z = 480 [M − H]⁻ |
| | ¹H-NMR (400 MHz, DMSO-d6) δ [ppm]: 2.010 (16.00), 2.332 (0.46), |
| | 2.518 (2.20), 2.523 (1.39), 2.673 (0.46), 4.030 (0.44), 4.079 (7.73), |
| | 7.334 (1.65), 7.352 (2.17), 7.355 (2.36), 7.374 (2.64), 7.469 (0.51), |
| | 7.485 (8.52), 7.489 (2.37), 7.499 (1.77), 7.504 (5.31), 7.525 (0.81), |
| | 7.544 (1.08), 7.616 (0.70), 7.636 (1.77), 7.655 (1.50), 7.664 (1.98), |
| | 7.683 (1.75), 7.709 (1.97), 8.307 (1.93), 8.321 (1.84), 10.896 (2.49). |
| 10 | 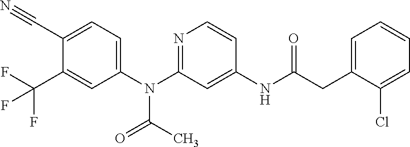 [SMILES: CC(=O)N(c1ccc(C#N)c(C(F)(F)F)c1)c1cc(NC(=O)Cc2ccccc2Cl)ccn1] |
| | N-{4-[2-(2-chlorophenyl)acetamido]pyridin-2-yl}-N-[4-cyano-3- |
| | (trifluoromethyl)phenyl]acetamide |
| | LC-MS (Method 2): Rₜ = 1.20 min; MS (ESIpos): m/z = 473 [M + H]⁺ |
| | ¹H-NMR (400 MHz, DMSO-d6) δ [ppm]: 2.036 (16.00), 2.518 (1.94), |
| | 2.522 (1.26), 3.887 (6.84), 7.300 (3.07), 7.309 (2.40), 7.315 (2.50), |
| | 7.324 (4.15), 7.334 (0.56), 7.395 (1.47), 7.402 (0.74), 7.405 (0.95), |
| | 7.409 (1.13), 7.418 (0.95), 7.424 (0.41), 7.433 (1.72), 7.443 (1.35), |
| | 7.447 (1.14), 7.457 (1.22), 7.563 (1.52), 7.568 (1.79), 7.578 (2.06), |
| | 7.582 (1.80), 7.594 (0.73), 7.710 (1.51), 8.012 (1.91), 8.017 (1.87), |
| | 8.143 (1.64), 8.164 (1.52), 8.376 (1.60), 8.390 (1.51), 10.846 (2.14). |
| 11 | 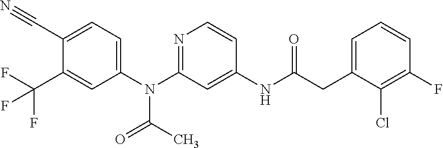 [SMILES: CC(=O)N(c1ccc(C#N)c(C(F)(F)F)c1)c1cc(NC(=O)Cc2cccc(F)c2Cl)ccn1] |
| | N-{4-[2-(2-chloro-3-fluorophenyl)acetamido]pyridin-2-yl}-N-[4- |
| | cyano-3-(trifluoromethyl)phenyl]acetamide |
| | LC-MS (Method 2): Rₜ = 1.21 min; MS (ESIpos): m/z = 491 [M + H]⁺ |
| | ¹H-NMR (400 MHz, DMSO-d6) δ [ppm]: 2.035 (16.00), 2.518 (3.85), |
| | 2.522 (2.47), 3.950 (6.51), 7.260 (0.68), 7.267 (1.08), 7.273 (0.76), |
| | 7.278 (0.82), 7.282 (1.12), 7.338 (1.74), 7.342 (2.04), 7.345 (1.44), |
| | 7.359 (4.32), 7.366 (1.24), 7.373 (1.20), 7.556 (1.49), 7.561 (1.53), |
| | 7.570 (1.68), 7.575 (2.20), 7.599 (0.74), 7.709 (1.54), 8.013 (1.95), |
| | 8.019 (1.92), 8.146 (1.70), 8.167 (1.57), 8.378 (1.60), 8.392 (1.52), |
| | 10.883 (2.23). |
| 12 | 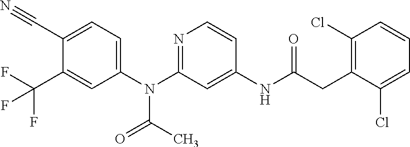 [SMILES: CC(=O)N(c1ccc(C#N)c(C(F)(F)F)c1)c1cc(NC(=O)Cc2c(Cl)cccc2Cl)ccn1] |
| | N-[4-cyano-3-(trifluoromethyl)phenyl]-N-{4-[2-(2,6- |
| | dichlorophenyl)acetamido]pyridin-2-yl}acetamide |
| | LC-MS (Method 2): Rₜ = 1.24 min; MS (ESIneg): m/z = 505 [M − H]⁻ |
| | ¹H-NMR (400 MHz, DMSO-d6) δ [ppm]: 2.034 (16.00), 2.518 (2.88), |
| | 2.522 (1.94), 4.088 (7.11), 7.334 (1.47), 7.352 (1.93), 7.355 (2.11), |
| | 7.374 (2.31), 7.485 (6.51), 7.504 (4.00), 7.535 (1.44), 7.540 (1.52), |
| | 7.549 (1.50), 7.554 (1.52), 7.579 (0.74), 7.597 (0.78), 7.707 (1.57), |
| | 8.019 (2.10), 8.024 (2.06), 8.143 (1.77), 8.163 (1.66), 8.379 (1.68), |
| | 8.393 (1.59), 10.963 (2.35). |
| 13 | 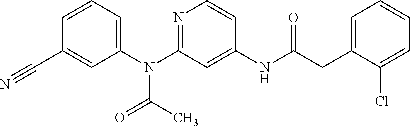 [SMILES: CC(=O)N(c1cccc(C#N)c1)c1cc(NC(=O)Cc2ccccc2Cl)ccn1] |
| | N-{4-[2-(2-chlorophenyl)acetamido]pyridin-2-yl}-N-(3- |
| | cyanophenyl)acetamide |
| | LC-MS (Method 1): Rₜ = 1.07 min; MS (ESIpos): m/z = 405 [M + H]⁺ |
| | 1H-NMR (400 MHz, DMSO-d6) δ [ppm]: 0.967 (0.17), 1.108 (12.87), |
| | 1.987 (0.18), 2.014 (16.00), 2.331 (0.30), 2.518 (1.94), 2.523 (1.21), |
| | 2.673 (0.30), 3.880 (7.97), 4.190 (1.25), 7.291 (0.32), 7.302 (3.17), |
| | 7.311 (2.32), 7.315 (2.39), 7.325 (4.39), 7.336 (0.56), 7.388 (0.28), |
| | 7.396 (1.73), 7.404 (0.91), 7.407 (1.09), 7.411 (1.17), 7.420 (1.10), |
| | 7.425 (0.45), 7.435 (1.97), 7.444 (1.67), 7.448 (1.19), 7.458 (1.35), |
| | 7.467 (0.19), 7.496 (1.78), 7.501 (1.71), 7.510 (1.74), 7.515 (1.83), |
| | 7.578 (0.30), 7.583 (0.32), 7.594 (3.00), 7.596 (3.54), 7.603 (1.51), |
| | 7.609 (2.28), 7.629 (0.51), 7.702 (1.55), 7.765 (1.09), 7.769 (1.30), |
| | 7.776 (0.93), 7.781 (0.94), 7.784 (1.04), 7.787 (0.92), 7.791 (0.89), |
| | 7.844 (2.46), 8.285 (1.95), 8.299 (1.86), 10.783 (2.55). |
| 14 | 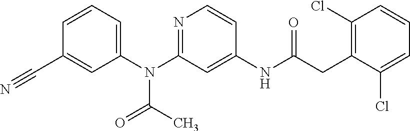 [SMILES: CC(=O)N(c1cccc(C#N)c1)c1cc(NC(=O)Cc2c(Cl)cccc2Cl)ccn1] |
| | N-(3-cyanophenyl)-N-{4-[2-(2,6-dichlorophenyl)acetamido]pyridin- |
| | 2-yl}acetamide |
| | LC-MS (Method 1): Rₜ = 1.12 min; MS (ESIpos): m/z = 439 [M + H]⁺ |
| | 1H-NMR (400 MHz, DMSO-d6) δ [ppm]: 0.967 (0.66), 1.054 (0.18), |
| | 1.071 (0.18), 1.107 (13.00), 1.144 (0.43), 1.208 (0.29), 1.232 (0.69), |
| | 1.983 (1.11), 2.011 (16.00), 2.332 (0.55), 2.336 (0.26), 2.518 (5.29), |
| | 2.522 (3.62), 2.673 (0.57), 4.081 (8.40), 4.189 (1.24), 6.461 (0.23), |
| | 7.335 (1.56), 7.354 (2.17), 7.356 (2.29), 7.375 (2.68), 7.464 (1.76), |
| | 7.469 (1.83), 7.479 (1.93), 7.486 (7.91), 7.506 (4.66), 7.583 (0.29), |
| | 7.596 (3.34), 7.599 (3.62), 7.603 (1.95), 7.610 (2.74), 7.612 (2.56), |
| | 7.630 (0.44), 7.701 (1.63), 7.758 (0.21), 7.765 (1.12), 7.769 (1.26), |
| | 7.773 (0.98), 7.780 (1.25), 7.782 (1.14), 7.787 (0.95), 7.791 (0.98), |
| | 7.848 (2.56), 8.288 (2.08), 8.302 (2.02), 10.899 (2.80). |
| 15 | 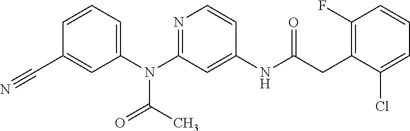 [SMILES: CC(=O)N(c1cccc(C#N)c1)c1cc(NC(=O)Cc2c(F)cccc2Cl)ccn1] |
| | N-{4-[2-(2-chloro-6-fluorophenyl)acetamido]pyridin-2-yl}-N-(3- |
| | cyanophenyl)acetamide |
| | LC-MS (Method 1): Rₜ = 1.08 min; MS (ESIpos): m/z = 423 [M + H]⁺ |
| | 1H-NMR (400 MHz, DMSO-d6) δ [ppm]: 0.899 (0.23), 0.916 (0.56), |
| | 0.936 (0.38), 0.939 (0.29), 0.967 (0.26), 1.107 (9.47), 1.220 (0.25), |
| | 1.239 (1.25), 1.254 (1.28), 1.269 (0.70), 1.482 (0.39), 1.985 (0.18), |
| | 2.011 (16.00), 2.332 (0.54), 2.336 (0.23), 2.518 (2.96), 2.522 (1.88), |
| | 2.673 (0.54), 2.678 (0.23), 3.880 (0.47), 3.925 (4.44), 3.929 (4.35), |
| | 7.226 (0.63), 7.231 (0.65), 7.244 (0.90), 7.249 (1.51), 7.252 (0.82), |
| | 7.269 (0.90), 7.273 (0.93), 7.302 (0.21), 7.325 (0.27), 7.337 (0.72), |
| | 7.341 (0.86), 7.357 (3.56), 7.363 (2.98), 7.377 (1.29), 7.382 (1.27), |
| | 7.397 (1.47), 7.402 (0.47), 7.417 (0.48), 7.469 (1.63), 7.473 (1.67), |
| | 7.483 (1.66), 7.488 (1.67), 7.495 (0.16), 7.580 (0.25), 7.585 (0.29), |
| | 7.594 (3.15), 7.597 (3.42), 7.605 (1.59), 7.610 (2.34), 7.630 (0.44), |
| | 7.704 (1.51), 7.765 (1.04), 7.769 (1.25), 7.772 (0.97), 7.777 (0.93), |
| | 7.780 (0.97), 7.783 (1.03), 7.787 (0.90), 7.791 (0.90), 7.846 (2.43), |
| | 8.289 (1.92), 8.303 (1.84), 10.889 (2.41). |
| 16 | 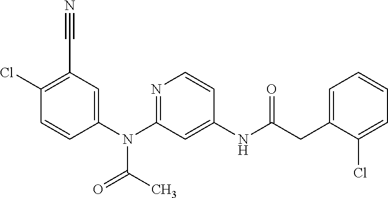 [SMILES: CC(=O)N(c1ccc(Cl)c(C#N)c1)c1cc(NC(=O)Cc2ccccc2Cl)ccn1] |
| | N-(4-chloro-3-cyanophenyl)-N-{4-[2-(2- |
| | chlorophenyl)acetamido]pyridin-2-yl}acetamide |
| | LC-MS (Method 2): Rₜ = 1.14 min; MS (ESIpos): m/z = 439 [M + H]⁺ |
| | ¹H-NMR (400 MHz, DMSO-d6) δ [ppm]: 2.022 (16.00), 2.518 (1.54), |
| | 2.522 (1.01), 3.884 (7.46), 7.303 (3.52), 7.311 (1.87), 7.313 (2.11), |
| | 7.317 (2.05), 7.319 (2.08), 7.327 (4.33), 7.336 (0.43), 7.338 (0.50), |
| | 7.399 (1.60), 7.406 (0.78), 7.410 (0.99), 7.414 (1.03), 7.423 (1.02), |
| | 7.437 (1.89), 7.445 (1.57), 7.450 (1.07), 7.452 (0.74), 7.460 (1.31), |
| | 7.502 (1.70), 7.508 (1.63), 7.516 (1.64), 7.521 (1.77), 7.605 (1.01), |
| | 7.611 (1.04), 7.627 (1.26), 7.633 (1.30), 7.728 (1.29), 7.760 (3.69), |
| | 7.782 (2.75), 8.009 (2.25), 8.015 (2.19), 8.284 (1.69), 8.298 (1.59), |
| | 10.794 (2.36). |
| 17 | 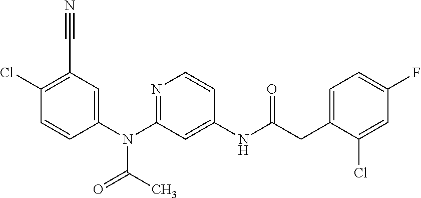 [SMILES: CC(=O)N(c1ccc(Cl)c(C#N)c1)c1cc(NC(=O)Cc2ccc(F)cc2Cl)ccn1] |
| | N-(4-chloro-3-cyanophenyl)-N-{4-[2-(2-chloro-4- |
| | fluorophenyl)acetamido]pyridin-2-yl}acetamide |
| | LC-MS (Method 2): Rₜ = 1.17 min; MS (ESIpos): m/z = 457 [M + H]⁺ |
| | ¹H-NMR (400 MHz, DMSO-d6) δ [ppm]: 2.021 (16.00), 2.518 (3.28), |
| | 2.523 (2.32), 3.875 (6.40), 7.189 (0.75), 7.196 (0.84), 7.210 (1.58), |
| | 7.217 (1.88), 7.232 (0.92), 7.239 (0.99), 7.433 (1.64), 7.439 (1.66), |
| | 7.448 (1.51), 7.455 (1.84), 7.462 (2.14), 7.464 (1.88), 7.470 (1.48), |
| | 7.485 (1.28), 7.494 (1.78), 7.498 (1.80), 7.508 (1.78), 7.513 (1.86), |
| | 7.605 (1.02), 7.612 (1.08), 7.627 (1.35), 7.634 (1.45), 7.726 (1.39), |
| | 7.762 (3.79), 7.783 (2.90), 8.009 (2.26), 8.015 (2.33), 8.283 (1.74), |
| | 8.297 (1.72), 10.793 (2.56). |
| 18 | 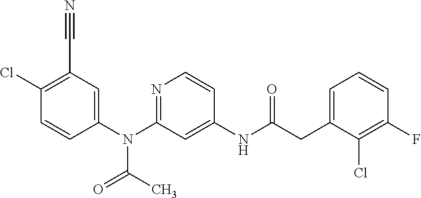 [SMILES: CC(=O)N(c1ccc(Cl)c(C#N)c1)c1cc(NC(=O)Cc2cccc(F)c2Cl)ccn1] |
| | N-(4-chloro-3-cyanophenyl)-N-{4-[2-(2-chloro-3- |
| | fluorophenyl)acetamido]pyridin-2-yl}acetamide |
| | LC-MS (Method 2): Rₜ = 1.16 min; MS (ESIpos): m/z = 457 [M + H]⁺ |
| | ¹H-NMR (400 MHz, DMSO-d6) δ [ppm]: 2.021 (16.00), 2.669 (0.41), |
| | 3.947 (8.83), 7.263 (1.04), 7.270 (1.34), 7.278 (1.27), 7.286 (1.79), |
| | 7.325 (0.43), 7.340 (2.31), 7.345 (2.90), 7.361 (4.48), 7.368 (1.65), |
| | 7.376 (1.38), 7.382 (0.50), 7.495 (2.03), 7.500 (1.78), 7.510 (2.16), |
| | 7.514 (1.87), 7.608 (1.26), 7.614 (1.25), 7.629 (1.68), 7.636 (1.63), |
| | 7.728 (2.10), 7.762 (3.76), 7.784 (2.91), 8.011 (2.83), 8.016 (2.65), |
| | 8.285 (2.19), 8.299 (2.14), 10.833 (3.45). |
| 19 | 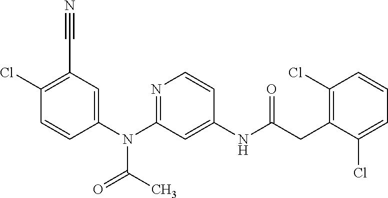 [SMILES: CC(=O)N(c1ccc(Cl)c(C#N)c1)c1cc(NC(=O)Cc2c(Cl)cccc2Cl)ccn1] |
| | N-(4-chloro-3-cyanophenyl)-N-{4-[2-(2,6- |
| | dichlorophenyl)acetamido]pyridin-2-yl}acetamide |
| | LC-MS (Method 2): Rₜ = 1.20 min; MS (ESIpos): m/z = 474 [M + H]⁺ |
| | ¹H-NMR (400 MHz, DMSO-d6) δ [ppm]: 2.019 (16.00), 2.518 (4.03), |
| | 2.523 (2.87), 4.086 (7.51), 7.336 (1.56), 7.355 (2.01), 7.358 (2.31), |
| | 7.376 (2.48), 7.471 (1.57), 7.476 (1.72), 7.488 (7.72), 7.507 (4.33), |
| | 7.610 (0.99), 7.617 (1.07), 7.632 (1.35), 7.638 (1.45), 7.728 (1.33), |
| | 7.760 (3.81), 7.782 (2.86), 8.011 (2.26), 8.018 (2.32), 8.288 (1.72), |
| | 8.302 (1.69), 10.911 (2.55). |
| 20 | 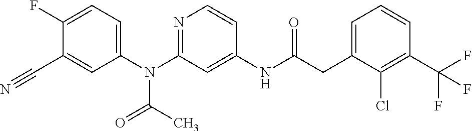 [SMILES: CC(=O)N(c1ccc(F)c(C#N)c1)c1cc(NC(=O)Cc2cccc(C(F)(F)F)c2Cl)ccn1] |
| | N-(4-{2-[2-chloro-3-(trifluoromethyl)phenyl]acetamido}pyridin-2- |
| | yl)-N-(3-cyano-4-fluorophenyl)acetamide |
| | LC-MS (Method 2): Rₜ = 1.20 min; MS (ESIpos): m/z = 491 [M + H]⁺ |
| | ¹H-NMR (400 MHz, DMSO-d6) δ [ppm]: 2.006 (16.00), 2.331 (0.41), |
| | 2.518 (1.86), 2.522 (1.19), 4.033 (7.17), 7.469 (1.75), 7.474 (1.84), |
| | 7.484 (1.79), 7.488 (1.86), 7.529 (0.85), 7.549 (3.17), 7.571 (3.50), |
| | 7.593 (1.72), 7.694 (0.67), 7.700 (0.77), 7.706 (0.79), 7.713 (0.82), |
| | 7.723 (0.66), 7.728 (0.62), 7.744 (1.69), 7.764 (2.56), 7.797 (1.65), |
| | 7.800 (1.50), 7.816 (1.42), 7.820 (1.24), 7.974 (1.13), 7.980 (1.16), |
| | 7.988 (1.18), 7.995 (1.08), 8.270 (1.90), 8.283 (1.82), 10.870 (2.75). |
| 21 | 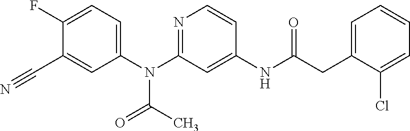 [SMILES: CC(=O)N(c1ccc(F)c(C#N)c1)c1cc(NC(=O)Cc2ccccc2Cl)ccn1] |
| | N-{4-[2-(2-chlorophenyl)acetamido]pyridin-2-yl}-N-(3-cyano-4- |
| | fluorophenyl)acetamide |
| | LC-MS (Method 2): Rₜ = 1.09 min; MS (ESIpos): m/z = 423 [M + H]⁺ |
| | ¹H-NMR (400 MHz, DMSO-d6) δ [ppm]: 2.010 (16.00), 2.518 (2.76), |
| | 2.522 (1.74), 3.885 (8.30), 7.305 (3.60), 7.312 (2.01), 7.314 (2.21), |
| | 7.318 (2.22), 7.320 (2.31), 7.328 (4.65), 7.339 (0.55), 7.400 (1.79), |
| | 7.407 (0.85), 7.411 (1.10), 7.415 (1.17), 7.424 (1.15), 7.438 (2.04), |
| | 7.447 (1.63), 7.451 (1.14), 7.461 (1.46), 7.486 (1.80), 7.491 (1.87), |
| | 7.500 (1.84), 7.505 (1.94), 7.548 (1.27), 7.570 (3.03), 7.593 (1.71), |
| | 7.691 (0.68), 7.698 (0.77), 7.704 (0.80), 7.710 (0.81), 7.721 (0.66), |
| | 7.726 (0.61), 7.733 (0.64), 7.751 (1.42), 7.973 (1.12), 7.980 (1.15), |
| | 7.987 (1.19), 7.994 (1.08), 8.266 (1.92), 8.280 (1.81), 10.787 (2.65). |
| 22 | 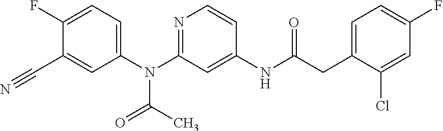 [SMILES: CC(=O)N(c1ccc(F)c(C#N)c1)c1cc(NC(=O)Cc2ccc(F)cc2Cl)ccn1] |
| | N-{4-[2-(2-chloro-4-fluorophenyl)acetamido]pyridin-2-yl}-N-(3- |
| | cyano-4-fluorophenyl)acetamide |
| | LC-MS (Method 2): Rₜ = 1.12 min; MS (ESIpos): m/z = 441 [M + H]⁺ |
| | ¹H-NMR (400 MHz, DMSO-d6) δ [ppm]: 2.009 (16.00), 2.518 (0.89), |
| | 2.522 (0.56), 3.876 (7.01), 7.190 (0.82), 7.197 (0.92), 7.212 (1.68), |
| | 7.218 (1.86), 7.233 (0.92), 7.240 (1.01), 7.433 (1.80), 7.440 (1.81), |
| | 7.449 (1.61), 7.455 (1.94), 7.462 (2.17), 7.465 (1.84), 7.471 (1.51), |
| | 7.478 (1.93), 7.483 (2.03), 7.486 (1.57), 7.492 (1.84), 7.497 (1.93), |
| | 7.548 (1.29), 7.571 (3.06), 7.593 (1.69), 7.691 (0.70), 7.698 (0.80), |
| | 7.704 (0.82), 7.710 (0.84), 7.721 (0.68), 7.726 (0.63), 7.733 (0.69), |
| | 7.749 (1.43), 7.973 (1.17), 7.979 (1.19), 7.987 (1.20), 7.993 (1.09), |
| | 8.265 (1.96), 8.279 (1.85), 10.785 (2.75). |
| 23 | 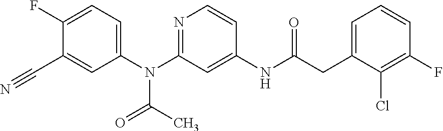 [SMILES: CC(=O)N(c1ccc(F)c(C#N)c1)c1cc(NC(=O)Cc2cccc(F)c2Cl)ccn1] |
| | N-{4-[2-(2-chloro-3-fluorophenyl)acetamido]pyridin-2-yl}-N-(3- |
| | cyano-4-fluorophenyl)acetamide |
| | LC-MS (Method 2): Rₜ = 1.11 min; MS (ESIpos): m/z = 441 [M + H]⁺ |
| | ¹H-NMR (400 MHz, DMSO-d6) δ [ppm]: 2.009 (16.00), 2.518 (2.32), |
| | 2.522 (1.51), 3.948 (7.79), 7.265 (0.85), 7.272 (1.25), 7.279 (0.93), |
| | 7.283 (0.99), 7.289 (1.36), 7.327 (0.45), 7.341 (2.02), 7.346 (2.71), |
| | 7.362 (4.42), 7.370 (1.46), 7.377 (1.30), 7.479 (1.79), 7.483 (1.86), |
| | 7.493 (1.84), 7.497 (1.87), 7.549 (1.29), 7.572 (3.03), 7.594 (1.70), |
| | 7.693 (0.68), 7.700 (0.81), 7.705 (0.79), 7.712 (0.85), 7.723 (0.67), |
| | 7.728 (0.65), 7.735 (0.67), 7.751 (1.41), 7.974 (1.14), 7.981 (1.17), |
| | 7.988 (1.19), 7.995 (1.08), 8.268 (1.93), 8.282 (1.82), 10.825 (2.68). |
| 24 | 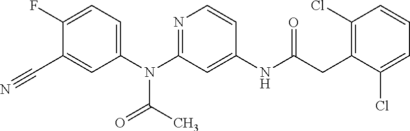 [SMILES: CC(=O)N(c1ccc(F)c(C#N)c1)c1cc(NC(=O)Cc2c(Cl)cccc2Cl)ccn1] |
| | N-(3-cyano-4-fluorophenyl)-N-{4-[2-(2,6- |
| | dichlorophenyl)acetamido]pyridin-2-yl}acetamide |
| | LC-MS (Method 2): Rₜ = 1.15 min; MS (ESIneg): m/z = 455 [M − H]⁻ |
| | ¹H-NMR (400 MHz, DMSO-d6) δ [ppm]: 2.007 (16.00), 2.074 (1.24), |
| | 2.518 (1.47), 2.523 (0.97), 4.087 (8.24), 7.337 (1.62), 7.356 (2.19), |
| | 7.358 (2.28), 7.377 (2.69), 7.453 (1.80), 7.457 (1.77), 7.467 (1.77), |
| | 7.472 (1.81), 7.489 (7.48), 7.509 (4.64), 7.547 (1.33), 7.569 (3.01), |
| | 7.592 (1.75), 7.619 (0.48), 7.697 (0.70), 7.703 (0.80), 7.709 (0.81), |
| | 7.716 (0.86), 7.726 (0.70), 7.731 (0.67), 7.738 (0.75), 7.752 (1.41), |
| | 7.975 (1.15), 7.981 (1.19), 7.989 (1.21), 7.996 (1.09), 8.270 (1.94), |
| | 8.284 (1.85), 10.902 (2.77). |
| 25 | 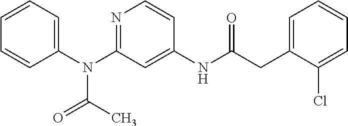 [SMILES: CC(=O)N(c1ccccc1)c1cc(NC(=O)Cc2ccccc2Cl)ccn1] |
| | N-{4-[2-(2-chlorophenyl)acetamido]pyridin-2-yl}-N- |
| | phenylacetamide |
| | ¹H NMR (250 MHz, DMSO-d6) δ [ppm]: 1.98 (s, 3H), 3.86 (s, 2H), 7.17- |
| | 7.54 (m, 10H), 7.65 (d, J = 1.6, 1H), 8.27 (d, J = 5.6, 1H), 10.72 (s, |
| | 1H). |
| 26 | 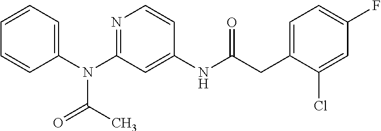 [SMILES: CC(=O)N(c1ccccc1)c1cc(NC(=O)Cc2ccc(F)cc2Cl)ccn1] |
| | N-{4-[2-(2-chloro-4-fluorophenyl)acetamido]pyridin-2-yl}-N- |
| | phenylacetamide |
| | LC-MS (Method 2): Rₜ = 1.09 min; MS (ESIpos): m/z = 308 [M + H]⁺ |
| | ¹H-NMR (400 MHz, DMSO-d6) δ [ppm]: 1.986 (16.00), 2.518 (3.27), |
| | 2.523 (2.08), 3.860 (6.30), 5.759 (2.71), 7.184 (0.69), 7.190 (0.79), |
| | 7.205 (1.51), 7.212 (1.74), 7.226 (0.84), 7.233 (0.92), 7.254 (2.07), |
| | 7.257 (2.45), 7.269 (0.85), 7.275 (3.42), 7.278 (2.92), 7.283 (1.03), |
| | 7.297 (0.69), 7.302 (1.82), 7.317 (0.94), 7.320 (1.34), 7.323 (0.68), |
| | 7.380 (0.48), 7.386 (2.75), 7.390 (1.13), 7.405 (3.43), 7.419 (0.69), |
| | 7.424 (1.50), 7.426 (1.20), 7.428 (1.83), 7.435 (1.73), 7.440 (1.60), |
| | 7.445 (2.02), 7.450 (2.95), 7.457 (3.23), 7.463 (2.56), 7.478 (1.19), |
| | 7.649 (1.82), 7.653 (1.83), 8.266 (2.29), 8.279 (2.22), 10.737 (2.33). |
| 27 | 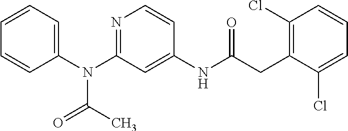 [SMILES: CC(=O)N(c1ccccc1)c1cc(NC(=O)Cc2c(Cl)cccc2Cl)ccn1] |
| | N-{4-[2-(2,6-dichlorophenyl)acetamido]pyridin-2-yl}-N- |
| | phenylacetamide |
| | LC-MS (Method 2): Rₜ = 1.12 min; MS (ESIpos): m/z = 415 [M + H]⁺ |
| | ¹H-NMR (400 MHz, DMSO-d6) δ [ppm]: 1.959 (2.02), 1.985 (16.00), |
| | 2.518 (2.49), 2.522 (1.59), 2.532 (1.38), 2.686 (8.92), 4.072 (7.21), |
| | 5.758 (5.72), 7.261 (2.47), 7.274 (0.85), 7.279 (3.39), 7.283 (3.08), |
| | 7.297 (0.72), 7.302 (1.83), 7.308 (0.66), 7.317 (1.14), 7.321 (1.44), |
| | 7.324 (0.83), 7.333 (1.91), 7.351 (1.94), 7.354 (2.14), 7.373 (2.35), |
| | 7.381 (0.56), 7.386 (2.67), 7.390 (1.16), 7.405 (3.46), 7.420 (2.58), |
| | 7.425 (3.05), 7.434 (1.85), 7.439 (1.91), 7.484 (6.29), 7.503 (3.82), |
| | 7.520 (0.73), 7.541 (0.43), 7.653 (1.74), 7.656 (1.72), 8.270 (2.27), |
| | 8.284 (2.16), 10.854 (2.26). |
| 28 | 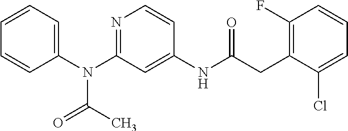 [SMILES: CC(=O)N(c1ccccc1)c1cc(NC(=O)Cc2c(F)cccc2Cl)ccn1] |
| | N-{4-[2-(2-chloro-6-fluorophenyl)acetamido]pyridin-2-yl}-N- |
| | phenylacetamide |
| | LC-MS (Method 1): Rₜ = 1.07 min; MS (ESIpos): m/z = 398 [M + H]⁺ |
| | H-NMR (400 MHz, DMSO-d6) δ [ppm]: 1.232 (0.55), 1.959 (1.27), |
| | 1.969 (0.64), 1.985 (16.00), 2.331 (0.78), 2.518 (3.95), 2.523 (2.60), |
| | 2.673 (0.78), 2.686 (0.67), 3.159 (4.24), 3.172 (3.97), 3.915 (4.24), |
| | 3.918 (4.08), 4.096 (0.91), 4.109 (0.95), 7.224 (0.66), 7.229 (0.69), |
| | 7.243 (0.96), 7.247 (1.57), 7.250 (1.09), 7.259 (2.58), 7.267 (1.20), |
| | 7.271 (1.62), 7.277 (3.44), 7.280 (3.06), 7.283 (1.67), 7.297 (0.66), |
| | 7.302 (1.82), 7.306 (0.75), 7.317 (1.06), 7.320 (1.37), 7.324 (0.84), |
| | 7.329 (0.53), 7.335 (0.84), 7.339 (0.89), 7.355 (3.44), 7.361 (2.68), |
| | 7.375 (1.42), 7.380 (1.73), 7.386 (2.77), 7.390 (1.22), 7.395 (1.80), |
| | 7.405 (3.48), 7.409 (1.07), 7.415 (0.91), 7.419 (0.96), 7.426 (2.64), |
| | 7.430 (1.89), 7.440 (1.84), 7.445 (1.73), 7.652 (1.75), 8.270 (2.26), |
| | 8.283 (2.11), 10.843 (2.18). |
| 29 | 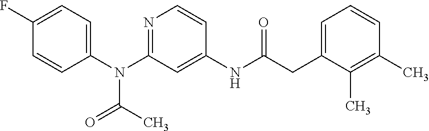 [SMILES: CC(=O)N(c1ccc(F)cc1)c1cc(NC(=O)Cc2cccc(C)c2C)ccn1] |
| | N-{4-[2-(2,3-dimethylphenyl)acetamido]pyridin-2-yl}-N-(4- |
| | fluorophenyl)acetamide |
| | L LC-MS (Method 1): Rₜ = 1.18 min; MS (ESIpos): m/z = 392 [M + H]⁺ |
| | ¹H-NMR (400 MHz, DMSO-d6) δ [ppm]: 1.011 (0.32), 1.029 (0.70), |
| | 1.047 (0.32), 1.090 (0.28), 1.108 (0.63), 1.126 (0.27), 1.981 (16.00), |
| | 2.044 (1.01), 2.077 (0.23), 2.139 (11.41), 2.212 (0.23), 2.234 (11.07), |
| | 2.308 (0.46), 2.332 (0.48), 2.336 (0.21), 2.518 (2.41), 2.523 (1.66), |
| | 2.530 (0.66), 2.673 (0.49), 2.678 (0.21), 3.271 (0.26), 3.288 (0.27), |
| | 3.349 (0.34), 3.639 (0.65), 3.730 (6.84), 6.980 (0.21), 6.993 (0.40), |
| | 6.998 (0.18), 7.012 (0.76), 7.017 (0.60), 7.028 (2.54), 7.043 (3.80), |
| | 7.057 (0.94), 7.062 (0.69), 7.067 (0.69), 7.209 (1.74), 7.214 (0.62), |
| | 7.226 (0.79), 7.231 (3.92), 7.236 (0.77), 7.240 (0.42), 7.247 (0.72), |
| | 7.253 (2.85), 7.261 (0.32), 7.299 (0.28), 7.307 (2.27), 7.312 (0.86), |
| | 7.319 (2.39), 7.324 (1.00), 7.329 (1.61), 7.336 (0.60), 7.342 (1.41), |
| | 7.458 (1.64), 7.463 (1.77), 7.472 (1.69), 7.476 (1.71), 7.678 (1.56), |
| | 7.681 (1.55), 8.250 (2.15), 8.264 (2.02), 10.293 (0.23), 10.639 (2.18). |
| 30 | 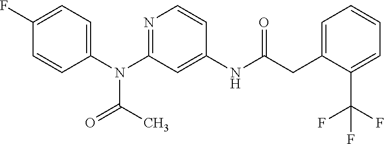 [SMILES: CC(=O)N(c1ccc(F)cc1)c1cc(NC(=O)Cc2ccccc2C(F)(F)F)ccn1] |
| | N-(4-fluorophenyl)-N-(4-{2-[2- |
| | (trifluoromethyl)phenyl]acetamido}pyridin-2-yl)acetamide |
| | LC-MS (Method 1): Rₜ = 1.16 min; MS (ESIpos): m/z = 432 [M + H]⁺ |
| | ¹H-NMR (400 MHz, DMSO-d6) δ [ppm]: 0.994 (0.27), 1.012 (0.62), |
| | 1.030 (0.27), 1.130 (0.23), 1.148 (0.55), 1.166 (0.24), 1.983 (16.00), |
| | 2.331 (0.17), 2.518 (0.95), 2.523 (0.60), 2.673 (0.17), 3.261 (0.22), |
| | 3.278 (0.22), 3.355 (0.23), 3.373 (0.21), 3.839 (0.27), 3.960 (3.41), |
| | 7.211 (1.83), 7.217 (0.66), 7.228 (0.81), 7.234 (3.91), 7.238 (0.80), |
| | 7.242 (0.44), 7.249 (0.76), 7.255 (2.87), 7.264 (0.31), 7.303 (0.28), |
| | 7.312 (2.24), 7.317 (0.88), 7.324 (2.40), 7.330 (0.99), 7.334 (1.68), |
| | 7.341 (0.62), 7.346 (1.43), 7.431 (1.74), 7.436 (1.82), 7.445 (1.74), |
| | 7.450 (1.91), 7.479 (0.55), 7.498 (2.48), 7.518 (2.44), 7.633 (0.86), |
| | 7.650 (1.27), 7.675 (1.73), 7.702 (1.44), 7.721 (1.25), 8.256 (2.20), |
| | 8.270 (2.10), 10.731 (2.26). |
| 31 | 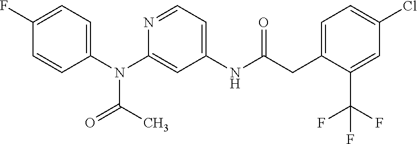 [SMILES: CC(=O)N(c1ccc(F)cc1)c1cc(NC(=O)Cc2ccc(Cl)cc2C(F)(F)F)ccn1] |
| | N-(4-{2-[4-chloro-2-(trifluoromethyl)phenyl]acetamido}pyridin-2- |
| | yl)-N-(4-fluorophenyl)acetamide |
| | LC-MS (Method 2): Rₜ = 1.23 min; MS (ESIpos): m/z = 466 [M + H]⁺ |
| | ¹H-NMR (400 MHz, DMSO-d6) δ [ppm]: 1.981 (16.00), 2.518 (2.06), |
| | 2.522 (1.43), 3.967 (3.64), 7.212 (1.87), 7.218 (0.71), 7.229 (0.92), |
| | 7.235 (4.24), 7.239 (0.93), 7.243 (0.54), 7.250 (0.83), 7.256 (3.04), |
| | 7.310 (2.44), 7.316 (1.03), 7.323 (2.64), 7.328 (1.17), 7.333 (1.87), |
| | 7.340 (0.75), 7.346 (1.61), 7.419 (1.61), 7.424 (1.63), 7.433 (1.63), |
| | 7.438 (1.64), 7.547 (1.56), 7.567 (1.88), 7.667 (1.79), 7.747 (1.09), |
| | 7.752 (1.44), 7.773 (1.62), 7.779 (3.06), 7.784 (1.83), 8.255 (2.34), |
| | 8.269 (2.24), 10.744 (2.47). |
| 32 | 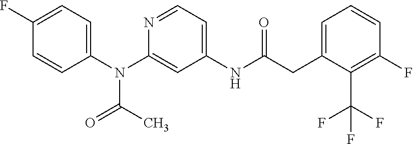 [SMILES: CC(=O)N(c1ccc(F)cc1)c1cc(NC(=O)Cc2cccc(F)c2C(F)(F)F)ccn1] |
| | N-(4-fluorophenyl)-N-(4-{2-[3-fluoro-2- |
| | (trifluoromethyl)phenyl]acetamido}pyridin-2-yl)acetamide |
| | LC-MS (Method 1): Rₜ = 1.15 min; MS (ESIpos): m/z = 450 [M + H]⁺ |
| | ¹H-NMR (400 MHz, DMSO-d6) δ [ppm]: 1.984 (16.00), 2.518 (0.89), |
| | 2.522 (0.58), 4.033 (2.59), 4.039 (2.53), 7.212 (1.84), 7.218 (0.69), |
| | 7.229 (0.88), 7.235 (4.17), 7.240 (0.85), 7.243 (0.47), 7.251 (0.78), |
| | 7.256 (2.93), 7.307 (1.48), 7.314 (2.60), 7.320 (1.22), 7.327 (3.86), |
| | 7.337 (1.80), 7.344 (0.68), 7.350 (1.51), 7.398 (0.68), 7.419 (0.94), |
| | 7.426 (2.08), 7.431 (1.80), 7.440 (1.51), 7.445 (1.91), 7.657 (2.16), |
| | 7.670 (0.76), 7.676 (0.94), 7.691 (0.88), 8.254 (2.26), 8.268 (2.12), |
| | 10.726 (2.22). |
| 33 | 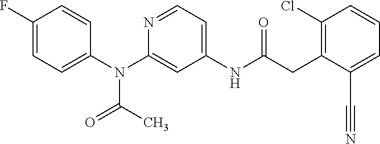 [SMILES: CC(=O)N(c1ccc(F)cc1)c1cc(NC(=O)Cc2c(Cl)cccc2C#N)ccn1] |
| | N-{4-[2-(2-chloro-6-cyanophenyl)acetamido]pyridin-2-yl}-N-(4- |
| | fluorophenyl)acetamide |
| | ¹H NMR (500 MHz, DMSO-d6) δ [ppm]: 1.99 (s, 3H), 4.13 (s, 2H), 7.21- |
| | 7.26 (m, 2H), 7.32 - 7.38 (m, 2H), 7.45 (dd, J = 5.7, 1.9, 1H), 7.50 - |
| | 7.57 (m, 1H), 7.69 (s, 1H), 7.85 (dd, J = 8.2, 1.1, 1H), 7.88 (dd, J = 7.7, |
| | 1.1, 1H), 8.29 (d, J = 5.6, 1H), 10.99 (s, 1H). |
| 34 | 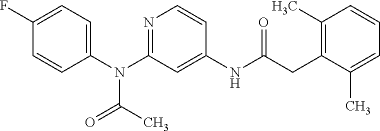 [SMILES: CC(=O)N(c1ccc(F)cc1)c1cc(NC(=O)Cc2c(C)cccc2C)ccn1] |
| | N-{4-[2-(2,6-dimethylphenyl)acetamido]pyridin-2-yl}-N-(4- |
| | fluorophenyl)acetamide |
| | LC-MS (Method 1): Rₜ = 1.18 min; MS (ESIpos): m/z = 392 [M + H]⁺ |
| | ¹H-NMR (400 MHz, DMSO-d6) δ [ppm]: 1.107 (10.95), 1.980 (10.74), |
| | 2.235 (16.00), 2.332 (0.31), 2.518 (1.57), 2.523 (1.02), 2.539 (1.50), |
| | 2.673 (0.32), 3.772 (4.37), 4.190 (0.89), 6.989 (0.57), 6.997 (0.70), |
| | 7.012 (3.14), 7.026 (2.23), 7.038 (0.78), 7.048 (0.52), 7.061 (0.31), |
| | 7.209 (1.19), 7.214 (0.44), 7.225 (0.54), 7.231 (2.59), 7.236 (0.51), |
| | 7.240 (0.28), 7.247 (0.50), 7.253 (1.83), 7.261 (0.20), 7.300 (0.19), |
| | 7.309 (1.47), 7.314 (0.57), 7.321 (1.58), 7.327 (0.63), 7.331 (1.05), |
| | 7.338 (0.39), 7.344 (0.93), 7.447 (1.13), 7.451 (1.09), 7.461 (1.12), |
| | 7.466 (1.13), 7.687 (1.04), 7.691 (1.00), 8.254 (1.44), 8.268 (1.36), |
| | 10.715 (1.44). |
| 35 | 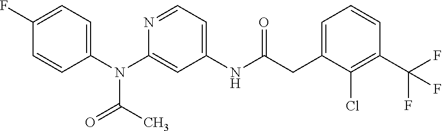 [SMILES: CC(=O)N(c1ccc(F)cc1)c1cc(NC(=O)Cc2cccc(C(F)(F)F)c2Cl)ccn1] |
| | N-(4-{2-[2-chloro-3-(trifluoromethyl)phenyl]acetamido}pyridin-2- |
| | yl)-N-(4-fluorophenyl)acetamide |
| | LC-MS (Method 2): Rₜ = 1.19 min; MS (ESIpos): m/z = 466 [M + H]⁺ |
| | ¹H-NMR (400 MHz, DMSO-d6) δ [ppm]: 1.984 (16.00), 2.014 (2.14), |
| | 2.518 (0.82), 2.522 (0.54), 4.022 (6.96), 7.212 (1.84), 7.218 (0.69), |
| | 7.229 (0.89), 7.234 (4.38), 7.239 (0.86), 7.243 (0.47), 7.250 (0.81), |
| | 7.256 (2.92), 7.264 (0.46), 7.286 (0.43), 7.307 (0.52), 7.314 (2.65), |
| | 7.319 (1.08), 7.326 (2.59), 7.332 (1.21), 7.336 (1.99), 7.343 (0.70), |
| | 7.349 (1.59), 7.437 (1.79), 7.442 (1.88), 7.452 (1.77), 7.456 (1.88), |
| | 7.524 (0.83), 7.543 (1.84), 7.563 (1.07), 7.694 (1.83), 7.698 (1.88), |
| | 7.738 (1.51), 7.757 (1.18), 7.793 (1.55), 7.796 (1.44), 7.813 (1.35), |
| | 7.816 (1.20), 8.263 (2.44), 8.277 (2.31), 10.834 (2.61). |
| 36 | 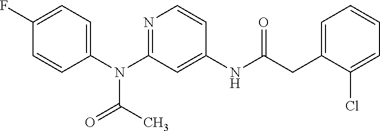 [SMILES: CC(=O)N(c1ccc(F)cc1)c1cc(NC(=O)Cc2ccccc2Cl)ccn1] |
| | N-{4-[2-(2-chlorophenyl)acetamido]pyridin-2-yl}-N-(4- |
| | fluorophenyl)acetamide |
| | LC-MS (Method 1): Rₜ = 0.81 min; MS (ESIpos): m/z = 398 [M + H]⁺ |
| | ¹H-NMR (400 MHz, DMSO-d6) δ [ppm]: 1.986 (16.00), 2.518 (1.96), |
| | 2.522 (1.23), 3.873 (7.55), 5.759 (1.67), 7.212 (1.74), 7.218 (0.70), |
| | 7.229 (0.93), 7.234 (4.08), 7.240 (0.86), 7.243 (0.49), 7.250 (0.85), |
| | 7.256 (2.86), 7.264 (0.42), 7.291 (0.45), 7.301 (3.19), 7.311 (3.99), |
| | 7.316 (3.25), 7.324 (6.59), 7.334 (2.12), 7.341 (0.68), 7.347 (1.43), |
| | 7.386 (0.45), 7.395 (1.65), 7.405 (1.17), 7.409 (1.21), 7.417 (1.02), |
| | 7.424 (0.46), 7.434 (1.83), 7.443 (1.63), 7.448 (1.29), 7.452 (2.10), |
| | 7.457 (2.57), 7.466 (1.81), 7.471 (1.81), 7.681 (1.71), 8.260 (2.25), |
| | 8.273 (2.12), 10.749 (2.32). |
| 37 | 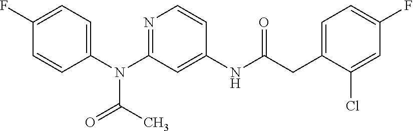 [SMILES: CC(=O)N(c1ccc(F)cc1)c1cc(NC(=O)Cc2ccc(F)cc2Cl)ccn1] |
| | N-{4-[2-(2-chloro-4-fluorophenyl)acetamido]pyridin-2-yl}-N-(4- |
| | fluorophenyl)acetamide |
| | LC-MS (Method 1): Rₜ = 1.12 min; MS (ESIpos): m/z = 416 [M + H]⁺ |
| | ¹H-NMR (400 MHz, DMSO-d6) δ [ppm]: 1.154 (0.49), 1.172 (0.96), |
| | 1.189 (0.46), 1.985 (16.00), 2.518 (1.16), 2.523 (0.69), 2.727 (8.49), |
| | 2.729 (8.55), 2.888 (10.69), 3.865 (6.13), 4.017 (0.40), 5.758 (0.59), |
| | 7.186 (0.75), 7.193 (0.82), 7.207 (1.61), 7.214 (2.83), 7.218 (0.83), |
| | 7.229 (1.55), 7.235 (4.83), 7.240 (0.90), 7.243 (0.50), 7.250 (0.79), |
| | 7.257 (2.86), 7.311 (2.29), 7.317 (0.94), 7.324 (2.47), 7.329 (1.03), |
| | 7.333 (1.64), 7.341 (0.64), 7.346 (1.42), 7.429 (1.57), 7.436 (1.73), |
| | 7.443 (3.13), 7.448 (2.10), 7.452 (1.92), 7.459 (4.02), 7.462 (2.48), |
| | 7.481 (1.13), 7.678 (1.65), 7.950 (1.30), 8.259 (2.22), 8.273 (2.11), |
| | 10.752 (2.30). |
| 38 | 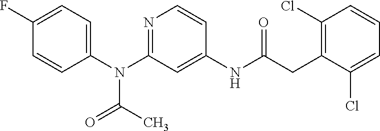 [SMILES: CC(=O)N(c1ccc(F)cc1)c1cc(NC(=O)Cc2c(Cl)cccc2Cl)ccn1] |
| | N-{4-[2-(2,6-dichlorophenyl)acetamido]pyridin-2-yl}-N-(4- |
| | fluorophenyl)acetamide |
| | LC-MS (Method 2): Rₜ = 1.15 min; MS (ESIpos): m/z = 432 [M + H]⁺ |
| | ¹H-NMR (400 MHz, DMSO-d6) δ [ppm]: 1.172 (0.62), 1.984 (16.00), |
| | 2.518 (3.36), 2.523 (2.16), 4.076 (7.64), 5.759 (5.64), 7.212 (1.76), |
| | 7.218 (0.68), 7.229 (0.90), 7.235 (4.17), 7.250 (0.81), 7.256 (2.90), |
| | 7.316 (2.33), 7.321 (1.03), 7.329 (2.52), 7.335 (2.56), 7.338 (1.89), |
| | 7.353 (2.63), 7.356 (2.29), 7.374 (2.38), 7.418 (1.74), 7.423 (1.78), |
| | 7.432 (1.77), 7.437 (1.79), 7.486 (6.72), 7.506 (4.18), 7.682 (1.71), |
| | 8.264 (2.28), 8.278 (2.19), 10.862 (2.43). |
| 39 | 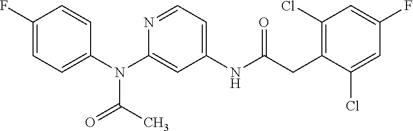 [SMILES: CC(=O)N(c1ccc(F)cc1)c1cc(NC(=O)Cc2c(Cl)cc(F)cc2Cl)ccn1] |
| | N-{4-[2-(2,6-dichloro-4-fluorophenyl)acetamido]pyridin-2-yl}-N-(4- |
| | fluorophenyl)acetamide |
| | ¹H NMR (500 MHz, DMSO-d6) δ [ppm]: 1.98 (s, 3H), 4.04 (s, 2H), 7.20- |
| | 7.28 (m, 2H), 7.29 - 7.38 (m, 2H), 7.43 (dd, J = 5.6, 1.9, 1H), 7.56 (d, |
| | J = 8.5, 2H), 7.68 (s, 1H), 8.27 (d, J = 5.6, 1H), 10.84 (s, 1H). |
| 40 | 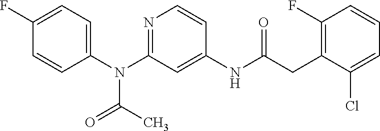 [SMILES: CC(=O)N(c1ccc(F)cc1)c1cc(NC(=O)Cc2c(F)cccc2Cl)ccn1] |
| | N-{4-[2-(2-chloro-6-fluorophenyl)acetamido]pyridin-2-yl}-N-(4- |
| | fluorophenyl)acetamide |
| | LC-MS (Method 1): Rₜ = 0.85 min; MS (ESIpos): m/z = 416 [M + H]⁺ |
| | ¹H-NMR (400 MHz, DMSO-d6) δ [ppm]: 1.984 (16.00), 2.518 (2.98), |
| | 2.522 (1.92), 3.919 (4.37), 3.923 (4.16), 5.759 (5.50), 7.212 (1.82), |
| | 7.217 (0.71), 7.226 (0.94), 7.234 (4.15), 7.239 (0.96), 7.244 (1.19), |
| | 7.250 (2.12), 7.256 (3.02), 7.264 (0.45), 7.269 (0.95), 7.272 (0.94), |
| | 7.314 (2.24), 7.319 (0.95), 7.326 (2.41), 7.332 (1.10), 7.336 (2.35), |
| | 7.341 (1.18), 7.349 (1.62), 7.357 (3.49), 7.362 (3.01), 7.376 (1.33), |
| | 7.381 (1.21), 7.396 (1.33), 7.402 (0.45), 7.417 (0.51), 7.424 (1.79), |
| | 7.428 (1.71), 7.438 (1.74), 7.443 (1.78), 7.682 (1.66), 8.263 (2.24), |
| | 8.277 (2.14), 10.851 (2.27). |
| 41 | 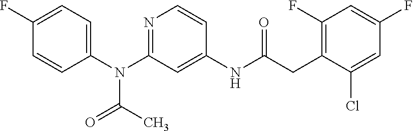 [SMILES: CC(=O)N(c1ccc(F)cc1)c1cc(NC(=O)Cc2c(F)cc(F)cc2Cl)ccn1] |
| | N-{4-[2-(2-chloro-4,6-difluorophenyl)acetamido]pyridin-2-yl}-N-(4- |
| | fluorophenyl)acetamide |
| | LC-MS (Method 1): Rₜ = 1.16 min; MS (ESIpos): m/z = 434 [M]⁺ |
| | ¹H-NMR (400 MHz, DMSO-d6) δ [ppm]: 1.983 (16.00), 2.518 (1.49), |
| | 2.523 (0.95), 3.887 (3.83), 3.890 (3.78), 7.212 (1.79), 7.218 (0.65), |
| | 7.230 (0.83), 7.235 (4.09), 7.240 (0.83), 7.244 (0.45), 7.251 (0.77), |
| | 7.257 (3.08), 7.314 (2.25), 7.320 (0.97), 7.327 (2.41), 7.331 (1.03), |
| | 7.336 (1.75), 7.344 (1.16), 7.350 (2.02), 7.367 (0.90), 7.374 (1.17), |
| | 7.391 (0.58), 7.397 (0.97), 7.404 (1.10), 7.408 (1.01), 7.414 (0.84), |
| | 7.419 (1.94), 7.425 (2.41), 7.433 (2.22), 7.438 (2.09), 7.681 (1.63), |
| | 8.263 (2.25), 8.277 (2.14), 10.863 (2.14). |
| 42 | 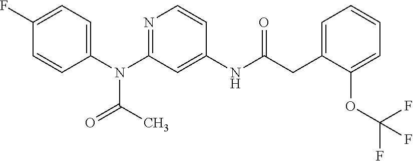 [SMILES: CC(=O)N(c1ccc(F)cc1)c1cc(NC(=O)Cc2ccccc2OC(F)(F)F)ccn1] |
| | N-(4-fluorophenyl)-N-(4-{2-[2- |
| | (trifluoromethoxy)phenyl]acetamido}pyridin-2-yl)acetamide |
| | LC-MS (Method 1): Rₜ = 1.18 min; MS (ESIpos): m/z = 448 [M + H]⁺ |
| | ¹H-NMR (400 MHz, DMSO-d6) δ [ppm]: 1.012 (0.26), 1.127 (0.25), |
| | 1.984 (16.00), 2.518 (1.36), 2.522 (0.83), 3.710 (0.28), 3.819 (6.90), |
| | 7.212 (1.85), 7.218 (0.68), 7.229 (0.83), 7.235 (4.12), 7.240 (0.83), |
| | 7.244 (0.45), 7.250 (0.79), 7.257 (2.88), 7.265 (0.32), 7.304 (0.30), |
| | 7.312 (2.38), 7.317 (0.99), 7.324 (2.57), 7.330 (1.20), 7.335 (2.29), |
| | 7.342 (1.56), 7.347 (2.26), 7.354 (0.99), 7.358 (1.46), 7.362 (2.80), |
| | 7.380 (1.83), 7.383 (1.10), 7.400 (1.30), 7.405 (1.72), 7.419 (0.99), |
| | 7.425 (1.50), 7.439 (0.56), 7.445 (2.15), 7.449 (1.88), 7.459 (2.80), |
| | 7.463 (2.64), 7.476 (1.15), 7.480 (0.83), 7.675 (1.64), 8.257 (2.28), |
| | 8.271 (2.15), 10.736 (2.30). |
| 43 | 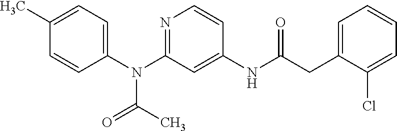 [SMILES: CC(=O)N(c1ccc(C)cc1)c1cc(NC(=O)Cc2ccccc2Cl)ccn1] |
| | N-{4-[2-(2-chlorophenyl)acetamido]pyridin-2-yl}-N-(4- |
| | methylphenyl)acetamide |
| | LC-MS (Method 2): Rₜ = 1.10 min; MS (ESIneg): m/z = 392 [M − H]⁻ |
| | ¹H-NMR (400 MHz, DMSO-d6) δ [ppm]: 1.975 (16.00), 2.301 (10.88), |
| | 2.518 (3.41), 2.523 (2.15), 3.866 (7.50), 7.131 (2.45), 7.147 (1.44), |
| | 7.151 (5.05), 7.191 (3.97), 7.211 (2.00), 7.299 (3.32), 7.308 (2.52), |
| | 7.314 (2.57), 7.323 (4.22), 7.333 (0.58), 7.391 (1.66), 7.401 (1.06), |
| | 7.405 (1.21), 7.414 (1.03), 7.422 (0.49), 7.433 (2.20), 7.435 (2.12), |
| | 7.440 (2.51), 7.449 (2.21), 7.454 (2.32), 7.641 (1.87), 7.644 (1.83), |
| | 8.244 (2.42), 8.257 (2.32), 10.724 (2.31). |
| 44 | 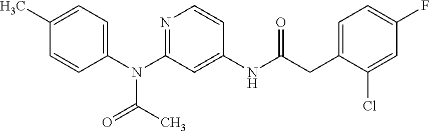 [SMILES: CC(=O)N(c1ccc(C)cc1)c1cc(NC(=O)Cc2ccc(F)cc2Cl)ccn1] |
| | N-{4-[2-(2-chloro-4-fluorophenyl)acetamido]pyridin-2-yl}-N-(4- |
| | methylphenyl)acetamide |
| | LC-MS (Method 2): Rₜ = 1.13 min; MS (ESIpos): m/z = 412 [M + H]⁺ |
| | ¹H-NMR (400 MHz, DMSO-d6) δ [ppm]: 1.974 (16.00), 2.301 (10.75), |
| | 2.518 (1.66), 2.522 (1.07), 3.806 (0.58), 3.858 (6.18), 7.131 (2.47), |
| | 7.135 (1.00), 7.146 (1.51), 7.151 (5.04), 7.184 (1.37), 7.191 (4.79), |
| | 7.205 (2.16), 7.212 (3.59), 7.227 (0.95), 7.233 (0.95), 7.428 (2.80), |
| | 7.433 (2.27), 7.436 (2.04), 7.441 (2.55), 7.447 (1.99), 7.451 (1.92), |
| | 7.457 (2.25), 7.461 (1.37), 7.477 (1.13), 7.637 (1.89), 7.641 (1.84), |
| | 8.243 (2.39), 8.257 (2.26), 10.721 (2.36). |
| 45 | 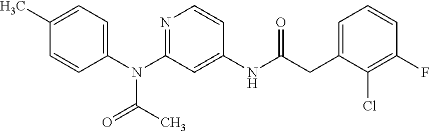 [SMILES: CC(=O)N(c1ccc(C)cc1)c1cc(NC(=O)Cc2cccc(F)c2Cl)ccn1] |
| | N-{4-[2-(2-chloro-3-fluorophenyl)acetamido]pyridin-2-yl}-N-(4- |
| | methylphenyl)acetamide |
| | LC-MS (Method 2): Rₜ = 1.12 min; MS (ESIpos): m/z = 412 [M + H]⁺ |
| | ¹H-NMR (400 MHz, DMSO-d6) δ [ppm]: 1.975 (16.00), 2.301 (10.84), |
| | 2.518 (1.14), 2.523 (0.74), 3.930 (6.96), 7.132 (2.49), 7.137 (0.97), |
| | 7.148 (1.38), 7.153 (5.14), 7.192 (3.99), 7.212 (2.04), 7.256 (0.77), |
| | 7.263 (1.12), 7.269 (0.80), 7.274 (0.88), 7.279 (1.18), 7.322 (0.48), |
| | 7.336 (1.88), 7.340 (2.29), 7.357 (4.52), 7.364 (1.35), 7.372 (1.24), |
| | 7.429 (1.75), 7.434 (1.75), 7.443 (1.64), 7.448 (1.81), 7.640 (1.88), |
| | 7.644 (1.81), 8.246 (2.44), 8.260 (2.28), 10.762 (2.33). |
| 46 | 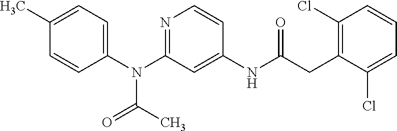 [SMILES: CC(=O)N(c1ccc(C)cc1)c1cc(NC(=O)Cc2c(Cl)cccc2Cl)ccn1] |
| | N-{4-[2-(2,6-dichlorophenyl)acetamido]pyridin-2-yl}-N-(4- |
| | methylphenyl)acetamide |
| | LC-MS (Method 2): Rₜ = 1.16 min; MS (ESIpos): m/z = 429 [M + H]⁺ |
| | ¹H-NMR (400 MHz, DMSO-d6) δ [ppm]: 1.973 (16.00), 2.301 (10.94), |
| | 2.518 (2.42), 2.522 (1.55), 4.016 (0.53), 4.069 (7.37), 7.134 (2.50), |
| | 7.139 (0.94), 7.150 (1.33), 7.155 (5.20), 7.191 (4.08), 7.212 (2.05), |
| | 7.333 (1.48), 7.351 (1.97), 7.355 (2.10), 7.373 (2.43), 7.400 (1.69), |
| | 7.405 (1.74), 7.414 (1.72), 7.419 (1.70), 7.466 (0.58), 7.485 (6.85), |
| | 7.504 (4.04), 7.641 (1.82), 7.645 (1.77), 8.247 (2.41), 8.261 (2.28), |
| | 10.837 (2.35). |
| 47 | 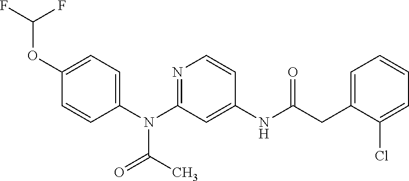 [SMILES: CC(=O)N(c1ccc(OC(F)F)cc1)c1cc(NC(=O)Cc2ccccc2Cl)ccn1] |
| | N-{4-[2-(2-chlorophenyl)acetamido]pyridin-2-yl}-N-[4- |
| | (difluoromethoxy)phenyl]acetamide |
| | LC-MS (Method 2): Rₜ = 1.12 min; MS (ESIpos): m/z = 446 [M + H]⁺ |
| | ¹H-NMR (500 MHz, DMSO-d6) δ [ppm]: 1.990 (16.00), 2.514 (2.04), |
| | 2.518 (2.06), 2.522 (1.64), 3.874 (8.51), 7.114 (1.80), 7.190 (3.62), |
| | 7.194 (1.11), 7.203 (1.31), 7.207 (4.61), 7.214 (0.43), 7.262 (3.86), |
| | 7.303 (3.53), 7.311 (2.32), 7.316 (2.84), 7.322 (9.41), 7.327 (1.44), |
| | 7.336 (1.20), 7.340 (3.35), 7.396 (1.71), 7.402 (0.85), 7.404 (1.10), |
| | 7.408 (1.40), 7.410 (1.90), 7.414 (1.20), 7.436 (1.98), 7.443 (1.68), |
| | 7.447 (1.06), 7.455 (1.56), 7.458 (1.98), 7.462 (2.04), 7.470 (1.88), |
| | 7.474 (1.96), 7.678 (1.53), 8.268 (2.20), 8.280 (2.13), 10.754 (2.65). |
| 48 | 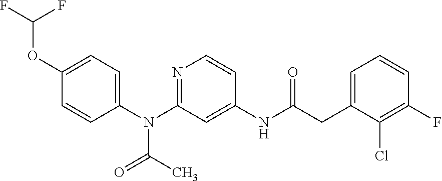 [SMILES: CC(=O)N(c1ccc(OC(F)F)cc1)c1cc(NC(=O)Cc2cccc(F)c2Cl)ccn1] |
| | N-{4-[2-(2-chloro-3-fluorophenyl)acetamido]pyridin-2-yl}-N-[4- |
| | (difluoromethoxy)phenyl]acetamide |
| | LC-MS (Method 2): Rₜ = 1.13 min; MS (ESIpos): m/z = 464 [M + H]⁺ |
| | ¹H-NMR (500 MHz, DMSO-d6) δ [ppm]: 1.990 (16.00), 2.514 (1.80), |
| | 2.518 (1.80), 2.522 (1.46), 3.937 (8.12), 7.115 (1.75), 7.191 (3.70), |
| | 7.195 (1.13), 7.204 (1.35), 7.209 (4.71), 7.215 (0.44), 7.263 (4.47), |
| | 7.267 (1.37), 7.273 (0.93), 7.276 (1.01), 7.280 (1.31), 7.317 (0.52), |
| | 7.323 (4.80), 7.328 (1.82), 7.341 (4.23), 7.345 (3.44), 7.358 (3.25), |
| | 7.362 (1.64), 7.371 (1.26), 7.375 (0.45), 7.411 (1.66), 7.452 (1.83), |
| | 7.456 (1.86), 7.463 (1.78), 7.467 (1.86), 7.678 (1.57), 8.271 (2.27), |
| | 8.282 (2.15), 10.791 (2.74). |
| 49 | 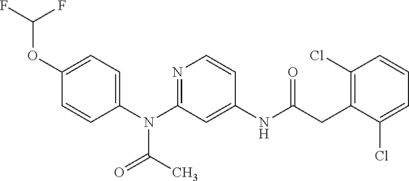 [SMILES: CC(=O)N(c1ccc(OC(F)F)cc1)c1cc(NC(=O)Cc2c(Cl)cccc2Cl)ccn1] |
| | N-{4-[2-(2,6-dichlorophenyl)acetamido]pyridin-2-yl}-N-[4- |
| | (difluoromethoxy)phenyl]acetamide |
| | LC-MS (Method 2): Rₜ = 1.17 min; MS (ESIpos): m/z = 481 [M + H]⁺ |
| | ¹H-NMR (400 MHz, DMSO-d6) δ [ppm]: 1.988 (16.00), 2.518 (9.92), |
| | 2.523 (7.01), 4.076 (7.76), 7.079 (1.99), 7.188 (3.36), 7.193 (1.27), |
| | 7.204 (1.35), 7.210 (4.67), 7.218 (0.70), 7.264 (3.83), 7.317 (0.53), |
| | 7.325 (4.93), 7.331 (1.67), 7.334 (2.13), 7.342 (1.39), 7.348 (3.73), |
| | 7.353 (2.57), 7.356 (2.78), 7.375 (2.77), 7.424 (1.82), 7.428 (1.85), |
| | 7.438 (1.92), 7.443 (1.98), 7.448 (2.06), 7.486 (7.11), 7.506 (4.32), |
| | 7.681 (1.79), 8.271 (2.35), 8.285 (2.29), 10.870 (2.60). |
| 50 | 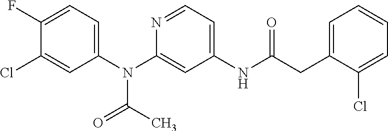 [SMILES: CC(=O)N(c1ccc(F)c(Cl)c1)c1cc(NC(=O)Cc2ccccc2Cl)ccn1] |
| | N-(3-chloro-4-fluorophenyl)-N-{4-[2-(2- |
| | chlorophenyl)acetamido]pyridin-2-yl}acetamide |
| | LC-MS (Method 2): Rₜ = 1.18 min; MS (ESIpos): m/z = 432 [M + H]⁺ |
| | ¹H-NMR (400 MHz, DMSO-d6) δ [ppm]: 2.005 (16.00), 2.518 (0.42), |
| | 3.337 (1.52), 7.275 (0.51), 7.281 (0.60), 7.285 (0.64), 7.291 (0.85), |
| | 7.297 (0.88), 7.302 (3.87), 7.309 (2.19), 7.311 (2.38), 7.315 (2.43), |
| | 7.317 (2.22), 7.325 (4.18), 7.334 (0.41), 7.336 (0.48), 7.398 (1.58), |
| | 7.405 (0.78), 7.409 (1.00), 7.413 (1.04), 7.421 (1.04), 7.430 (1.86), |
| | 7.435 (1.91), 7.444 (1.50), 7.448 (1.25), 7.452 (3.29), 7.458 (1.39), |
| | 7.475 (1.30), 7.485 (1.53), 7.489 (1.58), 7.499 (1.55), 7.503 (1.56), |
| | 7.622 (1.19), 7.629 (1.19), 7.639 (1.21), 7.646 (1.14), 7.706 (1.44), |
| | 8.272 (1.96), 8.286 (1.86), 10.771 (2.20). |
| 51 | 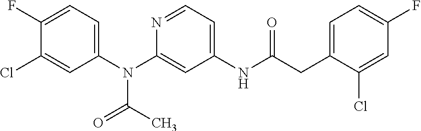 [SMILES: CC(=O)N(c1ccc(F)c(Cl)c1)c1cc(NC(=O)Cc2ccc(F)cc2Cl)ccn1] |
| | N-(3-chloro-4-fluorophenyl)-N-{4-[2-(2-chloro-4- |
| | fluorophenyl)acetamido]pyridin-2-yl}acetamide |
| | LC-MS (Method 2): Rₜ = 1.21 min; MS (ESIpos): m/z = 450 [M + H]⁺ |
| | ¹H-NMR (400 MHz, DMSO-d6) δ [ppm]: 2.004 (16.00), 2.518 (0.42), |
| | 3.339 (2.08), 7.186 (0.72), 7.193 (0.81), 7.208 (1.49), 7.214 (1.64), |
| | 7.229 (0.81), 7.236 (0.92), 7.275 (0.47), 7.281 (0.57), 7.285 (0.57), |
| | 7.291 (0.58), 7.296 (0.72), 7.303 (0.76), 7.307 (0.69), 7.314 (0.65), |
| | 7.430 (3.24), 7.436 (1.63), 7.447 (1.54), 7.452 (4.16), 7.458 (1.78), |
| | 7.463 (1.53), 7.468 (1.36), 7.475 (2.87), 7.480 (1.91), 7.484 (1.38), |
| | 7.490 (1.67), 7.494 (1.74), 7.622 (1.17), 7.628 (1.18), 7.639 (1.21), |
| | 7.645 (1.15), 7.703 (1.43), 8.271 (2.01), 8.285 (1.90), 10.768 (2.32). |
| 52 | 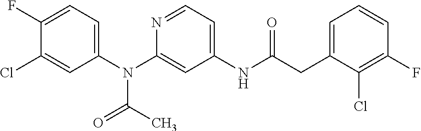 [SMILES: CC(=O)N(c1ccc(F)c(Cl)c1)c1cc(NC(=O)Cc2cccc(F)c2Cl)ccn1] |
| | N-(3-chloro-4-fluorophenyl)-N-{4-[2-(2-chloro-3- |
| | fluorophenyl)acetamido]pyridin-2-yl}acetamide |
| | LC-MS (Method 2): Rₜ = 1.20 min; MS (ESIpos): m/z = 450 [M + H]⁺ |
| | ¹H-NMR (400 MHz, DMSO-d6) δ [ppm]: 2.005 (16.00), 3.943 (7.72), |
| | 7.263 (0.91), 7.270 (1.34), 7.276 (1.51), 7.287 (2.07), 7.293 (0.87), |
| | 7.298 (1.03), 7.305 (1.03), 7.309 (0.95), 7.316 (0.92), 7.322 (0.56), |
| | 7.337 (1.91), 7.343 (2.79), 7.359 (4.27), 7.366 (1.60), 7.374 (1.33), |
| | 7.380 (0.47), 7.431 (1.82), 7.453 (3.02), 7.476 (2.78), 7.482 (1.89), |
| | 7.491 (1.77), 7.495 (1.88), 7.624 (1.34), 7.630 (1.38), 7.641 (1.38), |
| | 7.647 (1.31), 7.706 (1.80), 8.274 (2.29), 8.288 (2.20), 10.808 (2.70). |
| 53 | 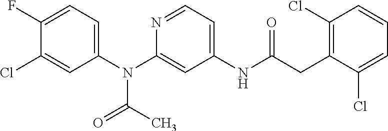 [SMILES: CC(=O)N(c1ccc(F)c(Cl)c1)c1cc(NC(=O)Cc2c(Cl)cccc2Cl)ccn1] |
| | N-(3-chloro-4-fluorophenyl)-N-{4-[2-(2,6- |
| | dichlorophenyl)acetamido]pyridin-2-yl}acetamide |
| | LC-MS (Method 2): Rₜ = 1.20 min; MS (ESIpos): m/z = 466 [M + H]⁺ |
| | ¹H-NMR (400 MHz, DMSO-d6) δ [ppm]: 2.002 (16.00), 4.083 (6.98), |
| | 7.280 (0.48), 7.287 (0.57), 7.291 (0.59), 7.297 (0.62), 7.302 (0.73), |
| | 7.309 (0.77), 7.312 (0.72), 7.319 (0.68), 7.333 (1.50), 7.352 (1.96), |
| | 7.355 (2.07), 7.374 (2.50), 7.429 (1.73), 7.451 (4.09), 7.456 (1.66), |
| | 7.466 (1.58), 7.471 (1.79), 7.473 (1.63), 7.485 (6.66), 7.505 (3.99), |
| | 7.627 (1.20), 7.633 (1.20), 7.644 (1.19), 7.650 (1.13), 7.708 (1.39), |
| | 8.276 (1.99), 8.290 (1.88), 10.885 (2.30). |
| 54 | 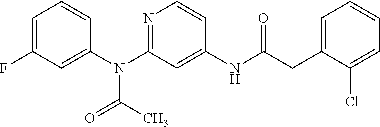 [SMILES: CC(=O)N(c1cccc(F)c1)c1cc(NC(=O)Cc2ccccc2Cl)ccn1] |
| | N-{4-[2-(2-chlorophenyl)acetamido]pyridin-2-yl}-N-(3- |
| | fluorophenyl)acetamide |
| | LC-MS (Method 1): Rₜ = 1.09 min; MS (ESIpos): m/z = 398 [M + H]⁺ |
| | ¹H-NMR (400 MHz, DMSO-d6) δ [ppm]: 1.232 (0.42), 1.988 (1.61), |
| | 2.007 (16.00), 2.518 (4.82), 2.523 (3.23), 3.875 (6.91), 5.759 (3.23), |
| | 7.058 (0.90), 7.081 (1.01), 7.140 (0.52), 7.155 (0.78), 7.161 (1.00), |
| | 7.176 (0.46), 7.183 (0.59), 7.202 (0.73), 7.208 (1.00), 7.214 (0.63), |
| | 7.228 (0.73), 7.233 (1.01), 7.239 (0.61), 7.301 (3.22), 7.310 (2.30), |
| | 7.316 (2.45), 7.324 (4.08), 7.335 (0.54), 7.386 (0.46), 7.395 (1.57), |
| | 7.402 (1.68), 7.409 (1.24), 7.419 (1.74), 7.423 (1.59), 7.434 (1.84), |
| | 7.439 (1.49), 7.443 (2.09), 7.448 (1.19), 7.457 (1.49), 7.482 (1.59), |
| | 7.487 (1.63), 7.496 (1.55), 7.501 (1.68), 7.670 (1.63), 8.288 (2.05), |
| | 8.302 (1.93), 10.765 (2.11). |
| 55 | 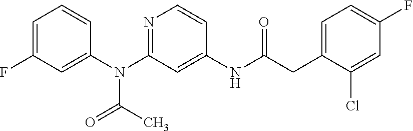 [SMILES: CC(=O)N(c1cccc(F)c1)c1cc(NC(=O)Cc2ccc(F)cc2Cl)ccn1] |
| | N-{4-[2-(2-chloro-4-fluorophenyl)acetamido]pyridin-2-yl}-N-(3- |
| | fluorophenyl)acetamide |
| | LC-MS (Method 1): Rₜ = 1.12 min; MS (ESIpos): m/z = 416 [M + H]⁺ |
| | ¹H-NMR (400 MHz, DMSO-d6) δ [ppm]: 1.232 (0.99), 1.986 (0.88), |
| | 2.005 (16.00), 2.518 (13.82), 2.523 (8.62), 3.866 (6.18), 5.760 (1.51), |
| | 7.058 (0.99), 7.078 (1.09), 7.141 (0.57), 7.164 (1.09), 7.186 (1.25), |
| | 7.193 (0.88), 7.207 (2.44), 7.214 (2.13), 7.228 (1.51), 7.235 (1.56), |
| | 7.403 (0.83), 7.423 (1.45), 7.431 (1.61), 7.444 (2.18), 7.453 (1.71), |
| | 7.459 (3.48), 7.464 (1.51), 7.473 (1.66), 7.478 (1.97), 7.487 (1.71), |
| | 7.492 (1.77), 7.667 (1.77), 8.288 (2.13), 8.302 (2.13), 10.763 (2.29). |
| 56 | 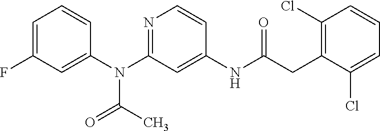 [SMILES: CC(=O)N(c1cccc(F)c1)c1cc(NC(=O)Cc2c(Cl)cccc2Cl)ccn1] |
| | N-{4-[2-(2,6-dichlorophenyl)acetamido]pyridin-2-yl}-N-(3- |
| | fluorophenyl)acetamide |
| | LC-MS (Method 1): Rₜ = 1.15 min; MS (ESIpos): m/z = 433 [M + H]⁺ |
| | ¹H-NMR (400 MHz, DMSO-d6) δ [ppm]: 1.232 (0.44), 1.985 (2.42), |
| | 2.004 (16.00), 2.518 (5.61), 2.523 (3.68), 4.078 (7.36), 5.759 (3.88), |
| | 7.067 (1.02), 7.086 (1.11), 7.136 (0.44), 7.141 (0.55), 7.158 (0.86), |
| | 7.162 (1.06), 7.177 (0.49), 7.184 (0.62), 7.207 (0.78), 7.212 (1.06), |
| | 7.218 (0.64), 7.233 (0.78), 7.238 (1.09), 7.244 (0.64), 7.279 (0.49), |
| | 7.335 (1.53), 7.354 (2.02), 7.356 (2.22), 7.375 (2.48), 7.387 (0.40), |
| | 7.403 (1.06), 7.420 (1.29), 7.424 (1.68), 7.440 (1.51), 7.450 (1.66), |
| | 7.454 (1.66), 7.464 (1.75), 7.469 (1.73), 7.486 (6.96), 7.506 (4.45), |
| | 7.668 (1.71), 8.292 (2.15), 8.306 (2.06), 10.880 (2.30). |
| 57 | 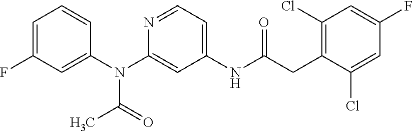 [SMILES: CC(=O)N(c1cccc(F)c1)c1cc(NC(=O)Cc2c(Cl)cc(F)cc2Cl)ccn1] |
| | N-{4-[2-(2,6-dichloro-4-fluorophenyl)acetamido]pyridin-2-yl}-N-(3- |
| | fluorophenyl)acetamide |
| | LC-MS (Method 1): Rₜ = 1.19 min; MS (ESIpos): m/z = 450 [M + H]⁺ |
| | ¹H-NMR (400 MHz, DMSO-d6) δ [ppm]: 1.172 (0.67), 1.232 (0.83), |
| | 1.988 (1.19), 2.003 (16.00), 2.331 (1.03), 2.336 (0.45), 2.518 (5.22), |
| | 2.523 (3.41), 2.673 (1.03), 2.678 (0.45), 4.041 (6.32), 5.759 (3.65), |
| | 7.064 (0.95), 7.067 (0.95), 7.087 (1.05), 7.138 (0.41), 7.142 (0.55), |
| | 7.144 (0.50), 7.159 (0.86), 7.163 (1.07), 7.179 (0.48), 7.185 (0.64), |
| | 7.207 (0.79), 7.213 (1.03), 7.218 (0.64), 7.233 (0.76), 7.238 (1.00), |
| | 7.244 (0.60), 7.403 (0.79), 7.421 (0.95), 7.424 (1.34), 7.441 (1.48), |
| | 7.445 (2.38), 7.450 (1.69), 7.459 (1.79), 7.464 (1.79), 7.565 (6.37), |
| | 7.587 (6.03), 7.664 (1.62), 8.291 (2.12), 8.305 (2.00), 10.879 (2.31). |
| 58 | 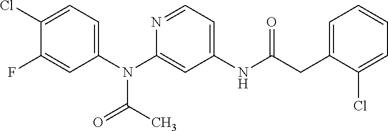 [SMILES: CC(=O)N(c1ccc(Cl)c(F)c1)c1cc(NC(=O)Cc2ccccc2Cl)ccn1] |
| | N-(4-chloro-3-fluorophenyl)-N-{4-[2-(2- |
| | chlorophenyl)acetamido]pyridin-2-yl}acetamide |
| | LC-MS (Method 2): Rₜ = 1.20 min; MS (ESIneg): m/z = 430 [M − H]⁻ |
| | ¹H-NMR (400 MHz, DMSO-d6) δ [ppm]: 2.017 (16.00), 2.518 (0.47), |
| | 3.340 (1.01), 7.091 (0.83), 7.094 (0.84), 7.113 (0.90), 7.116 (0.91), |
| | 7.301 (3.20), 7.310 (2.24), 7.316 (2.30), 7.324 (4.25), 7.334 (0.52), |
| | 7.396 (1.66), 7.403 (0.84), 7.407 (1.05), 7.411 (1.13), 7.420 (1.07), |
| | 7.424 (0.43), 7.434 (1.87), 7.443 (1.50), 7.448 (1.05), 7.457 (1.36), |
| | 7.469 (1.37), 7.474 (1.26), 7.492 (2.02), 7.497 (2.40), 7.500 (1.45), |
| | 7.506 (1.79), 7.511 (1.74), 7.586 (1.59), 7.607 (2.97), 7.629 (1.42), |
| | 7.693 (1.62), 8.289 (2.08), 8.302 (1.96), 10.781 (2.37). |
| 59 | 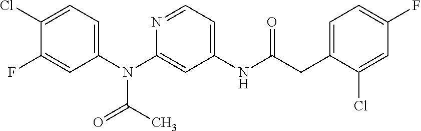 [SMILES: CC(=O)N(c1ccc(Cl)c(F)c1)c1cc(NC(=O)Cc2ccc(F)cc2Cl)ccn1] |
| | N-(4-chloro-3-fluorophenyl)-N-{4-[2-(2-chloro-4- |
| | fluorophenyl)acetamido]pyridin-2-yl}acetamide |
| | LC-MS (Method 2): Rₜ = 1.22 min; MS (ESIpos): m/z = 450 [M + H]⁺ |
| | ¹H-NMR (400 MHz, DMSO-d6) δ [ppm]: 2.016 (16.00), 2.518 (0.42), |
| | 3.341 (1.00), 7.091 (0.84), 7.094 (0.85), 7.112 (0.91), 7.116 (0.93), |
| | 7.186 (0.74), 7.193 (0.80), 7.207 (1.48), 7.214 (1.71), 7.228 (0.84), |
| | 7.235 (0.91), 7.429 (1.58), 7.436 (1.64), 7.445 (1.47), 7.451 (1.71), |
| | 7.458 (2.01), 7.467 (2.16), 7.474 (1.38), 7.483 (2.68), 7.488 (1.90), |
| | 7.494 (1.58), 7.497 (2.24), 7.502 (2.23), 7.586 (1.64), 7.607 (2.90), |
| | 7.629 (1.48), 7.690 (1.64), 8.287 (2.08), 8.302 (1.98), 10.778 (2.44). |
| 60 | 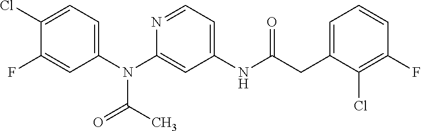 [SMILES: CC(=O)N(c1ccc(Cl)c(F)c1)c1cc(NC(=O)Cc2cccc(F)c2Cl)ccn1] |
| | N-(4-chloro-3-fluorophenyl)-N-{4-[2-(2-chloro-3- |
| | fluorophenyl)acetamido]pyridin-2-yl}acetamide |
| | LC-MS (Method 2): Rₜ = 1.21 min; MS (ESIpos): m/z = 450 [M + H]⁺ |
| | ¹H-NMR (400 MHz, DMSO-d6) δ [ppm]: 2.017 (16.00), 3.340 (0.76), |
| | 3.943 (6.21), 7.093 (0.75), 7.097 (0.78), 7.099 (0.71), 7.115 (0.83), |
| | 7.118 (0.83), 7.121 (0.75), 7.261 (0.68), 7.268 (1.03), 7.275 (0.76), |
| | 7.278 (0.81), 7.285 (1.12), 7.336 (1.65), 7.341 (2.19), 7.357 (3.72), |
| | 7.362 (0.68), 7.364 (1.28), 7.373 (1.16), 7.469 (1.19), 7.475 (1.18), |
| | 7.485 (1.51), 7.490 (1.66), 7.495 (1.39), 7.499 (2.00), 7.502 (1.77), |
| | 7.504 (1.96), 7.587 (1.52), 7.608 (2.77), 7.629 (1.32), 7.693 (1.45), |
| | 8.291 (1.87), 8.305 (1.76), 10.818 (2.12). |
| 61 | 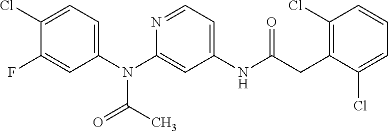 [SMILES: CC(=O)N(c1ccc(Cl)c(F)c1)c1cc(NC(=O)Cc2c(Cl)cccc2Cl)ccn1] |
| | N-(4-chloro-3-fluorophenyl)-N-{4-[2-(2,6- |
| | dichlorophenyl)acetamido]pyridin-2-yl}acetamide |
| | LC-MS (Method 2): Rₜ = 1.25 min; MS (ESIneg): m/z = 464 [M − H]⁻ |
| | ¹H-NMR (400 MHz, DMSO-d6) δ [ppm]: 2.014 (16.00), 4.082 (7.19), |
| | 7.096 (0.83), 7.100 (0.83), 7.118 (0.88), 7.122 (0.89), 7.332 (1.45), |
| | 7.351 (1.88), 7.354 (2.02), 7.373 (2.39), 7.461 (1.63), 7.466 (1.67), |
| | 7.472 (1.60), 7.476 (2.22), 7.480 (2.47), 7.484 (6.80), 7.498 (1.46), |
| | 7.504 (5.12), 7.585 (1.53), 7.606 (2.92), 7.627 (1.43), 7.693 (1.55), |
| | 8.292 (2.03), 8.306 (1.92), 10.896 (2.36). |
| 62 | 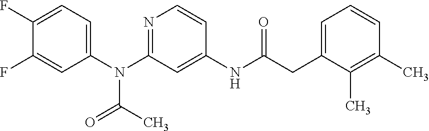 [SMILES: CC(=O)N(c1ccc(F)c(F)c1)c1cc(NC(=O)Cc2cccc(C)c2C)ccn1] |
| | N-(3,4-difluorophenyl)-N-{4-[2-(2,3- |
| | dimethylphenyl)acetamido]pyridin-2-yl}acetamide |
| | LC-MS (Method 1): Rₜ = 1.20 min; MS (ESIpos): m/z = 410 [M + H]⁺ |
| | ¹H-NMR (400 MHz, DMSO-d6) δ [ppm]: 1.998 (10.16), 2.168 (0.17), |
| | 2.230 (1.32), 2.239 (16.00), 2.331 (0.31), 2.336 (0.16), 2.518 (1.62), |
| | 2.523 (1.04), 2.673 (0.30), 3.455 (0.23), 3.602 (0.28), 3.778 (4.34), |
| | 6.976 (0.16), 6.983 (0.19), 6.991 (0.57), 6.999 (0.71), 7.013 (3.15), |
| | 7.027 (2.31), 7.039 (0.77), 7.049 (0.55), 7.062 (0.31), 7.105 (0.38), |
| | 7.116 (0.33), 7.123 (0.35), 7.127 (0.43), 7.426 (0.38), 7.448 (0.79), |
| | 7.452 (0.50), 7.469 (1.36), 7.474 (1.67), 7.483 (1.17), 7.488 (1.28), |
| | 7.497 (0.67), 7.509 (0.36), 7.515 (0.39), 7.519 (0.39), 7.525 (0.35), |
| | 7.538 (0.33), 7.543 (0.32), 7.712 (0.98), 8.266 (1.31), 8.280 (1.24), |
| | 10.734 (1.43). |
| 63 | 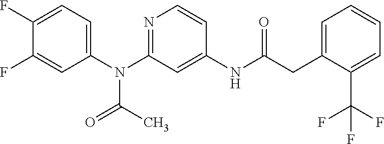 [SMILES: CC(=O)N(c1ccc(F)c(F)c1)c1cc(NC(=O)Cc2ccccc2C(F)(F)F)ccn1] |
| | N-(3,4-difluorophenyl)-N-(4-{2-[2- |
| | (trifluoromethyl)phenyl]acetamido}pyridin-2-yl)acetamide |
| | LC-MS (Method 1): Rₜ = 1.19 min; MS (ESIpos): m/z = 450 [M + H]⁺ |
| | ¹H-NMR (400 MHz, DMSO-d6) δ [ppm]: 2.000 (16.00), 2.331 (0.28), |
| | 2.518 (1.60), 2.523 (1.00), 2.673 (0.28), 3.615 (0.26), 3.965 (3.48), |
| | 7.108 (0.59), 7.120 (0.52), 7.127 (0.55), 7.131 (0.66), 7.429 (0.60), |
| | 7.453 (2.39), 7.458 (1.90), 7.467 (1.77), 7.472 (2.23), 7.478 (1.65), |
| | 7.500 (3.27), 7.521 (2.87), 7.533 (0.64), 7.545 (0.55), 7.551 (0.52), |
| | 7.635 (0.87), 7.652 (1.28), 7.672 (0.56), 7.704 (2.95), 7.723 (1.32), |
| | 8.266 (2.06), 8.280 (1.95), 10.749 (2.29). |
| 64 | 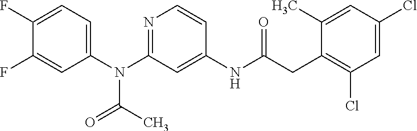 [SMILES: CC(=O)N(c1ccc(F)c(F)c1)c1cc(NC(=O)Cc2c(C)cc(Cl)cc2Cl)ccn1] |
| | N-{4-[2-(2,4-dichloro-6-methylphenyl)acetamido]pyridin-2-yl}-N- |
| | (3,4-difluorophenyl)acetamide |
| | LC-MS (Method 1): Rₜ = 1.30 min; MS (ESIpos): m/z = 464 [M + H]⁺ |
| | ¹H-NMR (400 MHz, DMSO-d6) δ [ppm]: 1.154 (3.97), 1.172 (7.59), |
| | 1.190 (3.96), 1.987 (16.00), 1.998 (11.38), 2.302 (8.22), 2.518 (1.58), |
| | 2.522 (0.99), 3.926 (4.51), 3.999 (1.14), 4.017 (3.45), 4.035 (3.43), |
| | 4.053 (1.11), 5.758 (4.90), 7.108 (0.45), 7.131 (0.50), 7.333 (1.48), |
| | 7.337 (1.55), 7.428 (0.45), 7.449 (3.18), 7.454 (2.85), 7.463 (1.34), |
| | 7.467 (1.32), 7.473 (0.62), 7.477 (0.95), 7.495 (0.43), 7.500 (0.73), |
| | 7.513 (0.41), 7.519 (0.46), 7.523 (0.45), 7.530 (0.40), 7.705 (1.13), |
| | 8.269 (1.52), 8.283 (1.45), 10.835 (1.62). |
| 65 | 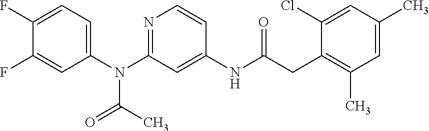 [SMILES: CC(=O)N(c1ccc(F)c(F)c1)c1cc(NC(=O)Cc2c(C)cc(C)cc2Cl)ccn1] |
| | N-{4-[2-(2-chloro-4,6-dimethylphenyl)acetamido]pyridin-2-yl}-N- |
| | (3,4-difluorophenyl)acetamide |
| | LC-MS (Method 1): Rₜ = 1.27 min; MS (ESIpos): m/z = 444 [M + H]⁺ |
| | ¹H-NMR (400 MHz, DMSO-d6) δ [ppm]: 2.000 (11.92), 2.250 (16.00), |
| | 2.518 (1.94), 2.523 (1.32), 2.540 (0.41), 3.882 (4.73), 7.003 (1.65), |
| | 7.112 (2.02), 7.128 (0.54), 7.426 (0.48), 7.449 (0.97), 7.453 (0.69), |
| | 7.459 (1.36), 7.463 (1.39), 7.473 (1.76), 7.477 (1.73), 7.493 (0.46), |
| | 7.498 (0.80), 7.511 (0.44), 7.518 (0.48), 7.521 (0.48), 7.528 (0.43), |
| | 7.540 (0.42), 7.546 (0.40), 7.698 (1.20), 8.266 (1.62), 8.279 (1.52), |
| | 10.774 (1.79). |
| 66 | 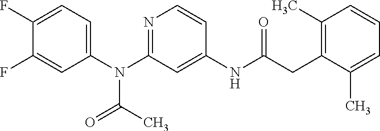 [SMILES: CC(=O)N(c1ccc(F)c(F)c1)c1cc(NC(=O)Cc2c(C)cccc2C)ccn1] |
| | N-(3,4-difluorophenyl)-N-{4-[2-(2,6- |
| | dimethylphenyl)acetamido]pyridin-2-yl}acetamide |
| | LC-MS (Method 1): Rₜ = 1.21 min; MS (ESIpos): m/z = 410 [M + H]⁺ |
| | ¹H-NMR (400 MHz, DMSO-d6) δ [ppm]: 1.998 (16.00), 2.091 (0.17), |
| | 2.142 (11.40), 2.235 (11.01), 2.518 (1.54), 2.522 (0.97), 3.599 (0.26), |
| | 3.736 (6.71), 6.994 (0.39), 7.007 (0.55), 7.017 (0.87), 7.030 (2.43), |
| | 7.046 (4.59), 7.060 (1.11), 7.068 (0.87), 7.102 (0.58), 7.113 (0.50), |
| | 7.120 (0.56), 7.124 (0.65), 7.426 (0.58), 7.448 (1.18), 7.452 (0.76), |
| | 7.471 (0.73), 7.474 (1.29), 7.480 (1.71), 7.485 (1.78), 7.494 (2.07), |
| | 7.498 (2.28), 7.509 (0.58), 7.516 (0.59), 7.520 (0.60), 7.526 (0.54), |
| | 7.538 (0.52), 7.545 (0.50), 7.704 (1.51), 8.262 (2.00), 8.275 (1.89), |
| | 10.658 (2.20). |
| 67 | 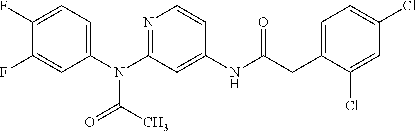 [SMILES: CC(=O)N(c1ccc(F)c(F)c1)c1cc(NC(=O)Cc2ccc(Cl)cc2Cl)ccn1] |
| | N-{4-[2-(2,4-dichlorophenyl)acetamido]pyridin-2-yl}-N-(3,4- |
| | difluorophenyl)acetamide |
| | LC-MS (Method 2): Rₜ = 1.24 min; MS (ESIpos): m/z = 451 [M + H]⁺ |
| | ¹H-NMR (400 MHz, DMSO-d6) δ [ppm]: 1.154 (0.61), 1.172 (1.18), |
| | 1.190 (0.63), 1.987 (2.51), 2.002 (16.00), 2.518 (1.65), 2.523 (1.08), |
| | 3.885 (6.08), 4.017 (0.51), 4.035 (0.52), 7.109 (0.54), 7.120 (0.47), |
| | 7.127 (0.50), 7.131 (0.62), 7.403 (0.89), 7.408 (0.83), 7.424 (2.78), |
| | 7.429 (3.36), 7.440 (3.67), 7.453 (1.23), 7.457 (0.91), 7.461 (1.34), |
| | 7.463 (1.79), 7.468 (1.76), 7.478 (2.25), 7.482 (1.88), 7.497 (0.54), |
| | 7.502 (0.92), 7.515 (0.53), 7.521 (0.57), 7.526 (0.57), 7.532 (0.51), |
| | 7.544 (0.50), 7.550 (0.48), 7.617 (2.53), 7.622 (2.52), 7.700 (1.40), |
| | 8.270 (1.90), 8.284 (1.79), 10.779 (2.14). |
| 68 | 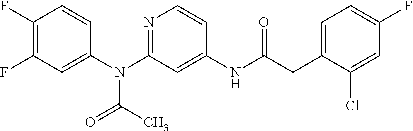 [SMILES: CC(=O)N(c1ccc(F)c(F)c1)c1cc(NC(=O)Cc2ccc(F)cc2Cl)ccn1] |
| | N-{4-[2-(2-chloro-4-fluorophenyl)acetamido]pyridin-2-yl}-N-(3,4- |
| | difluorophenyl)acetamide |
| | LC-MS (Method 1): Rₜ = 1.16 min; MS (ESIpos): m/z = 434 [M + H]⁺ |
| | ¹H-NMR (400 MHz, DMSO-d6) δ [ppm]: 1.154 (1.17), 1.171 (2.56), |
| | 1.189 (1.31), 1.986 (4.84), 2.002 (16.00), 2.518 (1.23), 2.522 (0.82), |
| | 3.870 (5.94), 3.998 (0.34), 4.016 (0.97), 4.034 (0.95), 4.052 (0.31), |
| | 7.108 (0.57), 7.119 (0.49), 7.126 (0.54), 7.130 (0.65), 7.187 (0.71), |
| | 7.194 (0.79), 7.209 (1.42), 7.215 (1.66), 7.230 (0.82), 7.237 (0.89), |
| | 7.430 (1.96), 7.437 (1.65), 7.445 (1.48), 7.452 (2.63), 7.459 (2.37), |
| | 7.464 (2.34), 7.467 (1.96), 7.469 (2.16), 7.474 (1.00), 7.478 (2.86), |
| | 7.483 (2.83), 7.495 (0.57), 7.501 (1.05), 7.513 (0.55), 7.520 (0.60), |
| | 7.524 (0.60), 7.530 (0.54), 7.542 (0.52), 7.549 (0.49), 7.704 (1.51), |
| | 8.269 (2.03), 8.283 (1.92), 10.765 (2.26). |
| 69 | 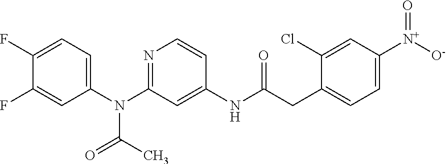 [SMILES: CC(=O)N(c1ccc(F)c(F)c1)c1cc(NC(=O)Cc2ccc([N+](=O)[O-])cc2Cl)ccn1] |
| | N-{4-[2-(2-chloro-4-nitrophenyl)acetamido]pyridin-2-yl}-N-(3,4- |
| | difluorophenyl)acetamide |
| | LC-MS (Method 1): Rₜ = 1.15 min; MS (ESIpos): m/z = 461 [M + H]⁺ |
| | ¹H-NMR (400 MHz, DMSO-d6) δ [ppm]: 1.987 (0.49), 2.004 (16.00), |
| | 2.030 (0.62), 2.518 (2.07), 2.522 (1.35), 4.057 (6.46), 5.758 (3.46), |
| | 7.114 (0.62), 7.125 (0.54), 7.136 (0.70), 7.433 (0.62), 7.455 (1.24), |
| | 7.459 (0.88), 7.465 (1.73), 7.470 (1.81), 7.480 (2.34), 7.484 (2.16), |
| | 7.504 (0.90), 7.520 (0.60), 7.526 (0.65), 7.530 (0.66), 7.536 (0.59), |
| | 7.548 (0.57), 7.554 (0.55), 7.707 (1.60), 7.725 (2.60), 7.746 (2.72), |
| | 8.188 (1.79), 8.194 (2.02), 8.209 (1.58), 8.215 (1.86), 8.276 (2.16), |
| | 8.290 (2.05), 8.301 (3.52), 8.307 (2.99), 10.873 (2.43). |
| 70 | 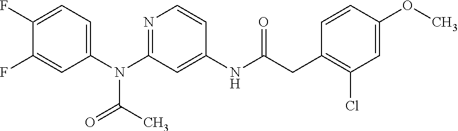 [SMILES: COc1ccc(CC(=O)Nc2ccnc(N(C(C)=O)c3ccc(F)c(F)c3)c2)c(Cl)c1] |
| | N-{4-[2-(2-chloro-4-methoxyphenyl)acetamido]pyridin-2-yl}-N-(3,4- |
| | difluorophenyl)acetamide |
| | LC-MS (Method 1): Rₜ = 1.14 min; MS (ESIpos): m/z = 446 [M + H]⁺ |
| | ¹H-NMR (400 MHz, DMSO-d6) δ [ppm]: 1.154 (1.77), 1.172 (2.94), |
| | 1.190 (1.49), 1.231 (0.40), 1.987 (6.03), 1.996 (14.46), 2.518 (2.13), |
| | 2.522 (1.33), 3.625 (6.07), 3.721 (0.77), 3.827 (16.00), 3.999 (0.42), |
| | 4.017 (1.24), 4.035 (1.23), 7.084 (2.16), 7.105 (3.35), 7.123 (0.72), |
| | 7.216 (1.47), 7.222 (1.49), 7.238 (1.07), 7.243 (1.12), 7.367 (2.71), |
| | 7.372 (2.52), 7.425 (0.57), 7.448 (1.17), 7.471 (2.26), 7.475 (2.32), |
| | 7.485 (1.67), 7.490 (2.13), 7.497 (1.18), 7.509 (0.59), 7.516 (0.65), |
| | 7.519 (0.65), 7.526 (0.55), 7.538 (0.54), 7.544 (0.52), 7.701 (1.65), |
| | 8.260 (1.99), 8.274 (1.90), 10.657 (2.15). |
| 71 | 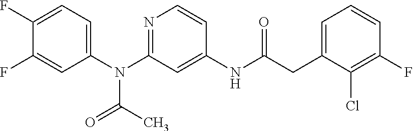 [SMILES: CC(=O)N(c1ccc(F)c(F)c1)c1cc(NC(=O)Cc2cccc(F)c2Cl)ccn1] |
| | N-{4-[2-(2-chloro-3-fluorophenyl)acetamido]pyridin-2-yl}-N-(3,4- |
| | difluorophenyl)acetamide |
| | LC-MS (Method 1): Rₜ = 1.16 min; MS (ESIpos): m/z = 434 [M + H]⁺ |
| | ¹H-NMR (400 MHz, DMSO-d6) δ [ppm]: 2.003 (16.00), 2.518 (1.09), |
| | 2.522 (0.69), 3.635 (0.31), 3.943 (6.79), 7.110 (0.58), 7.122 (0.52), |
| | 7.129 (0.55), 7.132 (0.67), 7.262 (0.73), 7.269 (1.11), 7.276 (0.82), |
| | 7.280 (0.89), 7.284 (1.17), 7.324 (0.40), 7.338 (1.81), 7.343 (2.36), |
| | 7.360 (3.90), 7.367 (1.34), 7.375 (1.22), 7.381 (0.37), 7.396 (0.19), |
| | 7.430 (0.58), 7.453 (1.21), 7.457 (0.80), 7.467 (1.74), 7.472 (1.93), |
| | 7.475 (1.01), 7.481 (2.17), 7.486 (1.86), 7.498 (0.64), 7.502 (0.86), |
| | 7.517 (0.56), 7.523 (0.60), 7.527 (0.61), 7.533 (0.55), 7.545 (0.53), |
| | 7.552 (0.50), 7.709 (1.53), 8.273 (2.04), 8.287 (1.95), 10.805 (2.26). |
| 72 | 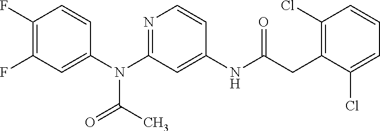 [SMILES: CC(=O)N(c1ccc(F)c(F)c1)c1cc(NC(=O)Cc2c(Cl)cccc2Cl)ccn1] |
| | N-{4-[2-(2,6-dichlorophenyl)acetamido]pyridin-2-yl}-N-(3,4- |
| | difluorophenyl)acetamide |
| | LC-MS (Method 1): Rₜ = 1.20 min; MS (ESIneg): m/z = 448 [M − H]⁻ |
| | ¹H-NMR (400 MHz, DMSO-d6) δ [ppm]: 2.001 (16.00), 2.518 (2.14), |
| | 2.523 (1.36), 3.159 (1.08), 3.172 (1.23), 4.082 (7.79), 4.095 (0.44), |
| | 7.114 (0.66), 7.125 (0.60), 7.136 (0.77), 7.336 (1.49), 7.355 (2.06), |
| | 7.357 (2.12), 7.376 (2.40), 7.429 (0.66), 7.441 (1.84), 7.445 (1.88), |
| | 7.455 (2.55), 7.459 (1.93), 7.474 (0.92), 7.478 (1.45), 7.487 (6.79), |
| | 7.500 (1.41), 7.507 (4.83), 7.519 (0.70), 7.525 (0.72), 7.529 (0.69), |
| | 7.535 (0.61), 7.548 (0.59), 7.554 (0.53), 7.709 (1.74), 8.274 (2.21), |
| | 8.288 (2.09), 10.885 (2.43). |
| 73 | 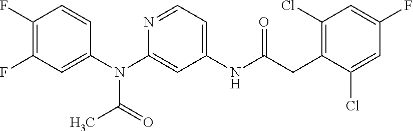 [SMILES: CC(=O)N(c1ccc(F)c(F)c1)c1cc(NC(=O)Cc2c(Cl)cc(F)cc2Cl)ccn1] |
| | N-{4-[2-(2,6-dichloro-4-fluorophenyl)acetamido]pyridin-2-yl}-N- |
| | (3,4-difluorophenyl)acetamide |
| | LC-MS (Method 1): Rₜ = 1.22 min; MS (ESIpos): m/z = 469 [M + H]⁺ |
| | ¹H-NMR (400 MHz, DMSO-d6) δ [ppm]: 1.232 (0.79), 1.987 (0.73), |
| | 2.000 (16.00), 2.332 (0.83), 2.518 (5.12), 2.522 (3.21), 2.673 (0.83), |
| | 4.045 (6.58), 5.758 (2.77), 7.115 (0.62), 7.126 (0.56), 7.136 (0.69), |
| | 7.431 (0.77), 7.435 (1.73), 7.440 (1.67), 7.449 (1.75), 7.454 (2.85), |
| | 7.475 (0.75), 7.480 (1.25), 7.502 (1.00), 7.507 (0.58), 7.519 (0.58), |
| | 7.526 (0.63), 7.530 (0.65), 7.536 (0.58), 7.548 (0.58), 7.554 (0.58), |
| | 7.567 (6.31), 7.589 (6.06), 7.707 (1.56), 8.273 (2.12), 8.288 (2.02), |
| | 10.880 (2.48). |
| 74 | 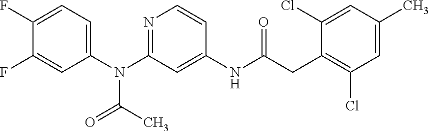 [SMILES: CC(=O)N(c1ccc(F)c(F)c1)c1cc(NC(=O)Cc2c(Cl)cc(C)cc2Cl)ccn1] |
| | N-{4-[2-(2,6-dichloro-4-methylphenyl)acetamido]pyridin-2-yl}-N- |
| | (3,4-difluorophenyl)acetamide |
| | LC-MS (Method 1): Rₜ = 1.27 min; MS (ESIpos): m/z = 464 [M + H]⁺ |
| | ¹H-NMR (400 MHz, DMSO-d6) δ [ppm]: 1.154 (1.72), 1.172 (3.12), |
| | 1.189 (1.52), 1.987 (5.73), 2.001 (16.00), 2.304 (10.37), 2.322 (0.42), |
| | 2.326 (0.48), 2.518 (1.85), 2.522 (1.14), 2.668 (0.42), 3.999 (0.49), |
| | 4.018 (7.26), 4.035 (1.30), 4.053 (0.41), 7.111 (0.64), 7.122 (0.59), |
| | 7.129 (0.62), 7.133 (0.73), 7.333 (7.34), 7.428 (0.61), 7.444 (1.81), |
| | 7.449 (2.54), 7.454 (1.15), 7.458 (1.85), 7.463 (1.83), 7.472 (0.80), |
| | 7.477 (1.26), 7.499 (1.01), 7.504 (0.59), 7.516 (0.60), 7.523 (0.66), |
| | 7.527 (0.65), 7.533 (0.58), 7.545 (0.57), 7.552 (0.53), 7.695 (1.66), |
| | 8.270 (2.14), 8.284 (2.05), 10.856 (2.09). |
| 75 | 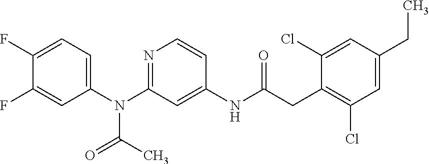 [SMILES: CCc1cc(Cl)c(CC(=O)Nc2ccnc(N(C(C)=O)c3ccc(F)c(F)c3)c2)c(Cl)c1] |
| | N-{4-[2-(2,6-dichloro-4-ethylphenyl)acetamido]pyridin-2-yl}-N-(3,4- |
| | difluorophenyl)acetamide |
| | LC-MS (Method 1): Rₜ = 1.36 min; MS (ESIpos): m/z = 478 [m + H]⁺ |
| | ¹H-NMR (400 MHz, DMSO-d6) δ [ppm]: 1.155 (4.47), 1.174 (9.99), |
| | 1.188 (1.72), 1.193 (4.62), 1.206 (0.42), 2.001 (16.00), 2.331 (0.56), |
| | 2.518 (3.03), 2.522 (1.85), 2.580 (1.12), 2.599 (3.12), 2.618 (2.98), |
| | 2.637 (0.95), 2.673 (0.63), 4.023 (6.98), 7.112 (0.76), 7.133 (0.83), |
| | 7.357 (9.79), 7.429 (0.72), 7.440 (1.95), 7.445 (1.96), 7.454 (2.67), |
| | 7.459 (2.02), 7.474 (1.01), 7.478 (1.34), 7.497 (1.35), 7.516 (0.71), |
| | 7.523 (0.80), 7.526 (0.76), 7.533 (0.65), 7.545 (0.65), 7.551 (0.56), |
| | 7.698 (1.98), 8.271 (2.26), 8.285 (2.13), 10.848 (2.78). |
| 76 | 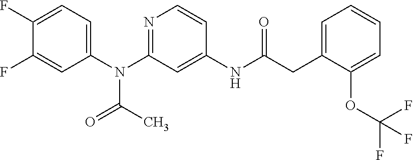 [SMILES: CC(=O)N(c1ccc(F)c(F)c1)c1cc(NC(=O)Cc2ccccc2OC(F)(F)F)ccn1] |
| | N-(3,4-difluorophenyl)-N-(4-{2-[2- |
| | (trifluoromethoxy)phenyl]acetamido}pyridin-2-yl)acetamide |
| | LC-MS (Method 1): Rₜ = 1.22 min; MS (ESIpos): m/z = 466 [M + H]⁺ |
| | ¹H-NMR (400 MHz, DMSO-d6) δ [ppm]: 1.012 (0.21), 1.127 (0.21), |
| | 2.002 (16.00), 2.518 (2.05), 2.522 (1.28), 2.673 (0.35), 2.678 (0.16), |
| | 3.709 (0.22), 3.824 (6.51), 7.109 (0.57), 7.120 (0.50), 7.127 (0.53), |
| | 7.131 (0.65), 7.335 (0.62), 7.339 (0.84), 7.344 (0.96), 7.348 (0.85), |
| | 7.355 (0.85), 7.359 (1.36), 7.363 (2.64), 7.381 (1.71), 7.385 (1.02), |
| | 7.402 (1.23), 7.407 (1.61), 7.421 (0.99), 7.427 (1.46), 7.430 (0.75), |
| | 7.441 (0.52), 7.446 (0.52), 7.453 (1.26), 7.459 (1.89), 7.465 (2.81), |
| | 7.470 (1.95), 7.479 (3.72), 7.484 (2.48), 7.496 (0.58), 7.502 (1.02), |
| | 7.514 (0.56), 7.521 (0.59), 7.525 (0.59), 7.531 (0.53), 7.543 (0.53), |
| | 7.550 (0.51), 7.701 (1.47), 8.269 (1.99), 8.283 (1.91), 10.754 (2.20). |
| 77 | 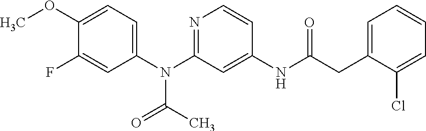 [SMILES: COc1ccc(N(C(C)=O)c2cc(NC(=O)Cc3ccccc3Cl)ccn2)cc1F] |
| | N-{4-[2-(2-chlorophenyl)acetamido]pyridin-2-yl}-N-(3-fluoro-4- |
| | methoxyphenyl)acetamide |
| | LC-MS (Method 2): Rₜ = 1.07 min; MS (ESIpos): m/z = 428 [M + H]⁺ |
| | ¹H-NMR (400 MHz, DMSO-d6) δ [ppm]: 1.987 (14.56), 2.518 (1.73), |
| | 2.523 (1.16), 3.841 (16.00), 3.874 (6.88), 7.031 (0.66), 7.035 (0.59), |
| | 7.053 (0.95), 7.057 (0.83), 7.145 (1.27), 7.167 (1.71), 7.190 (0.94), |
| | 7.244 (1.21), 7.250 (1.17), 7.275 (1.22), 7.280 (1.21), 7.302 (3.00), |
| | 7.311 (1.93), 7.316 (2.03), 7.317 (1.99), 7.325 (3.96), 7.336 (0.48), |
| | 7.396 (1.47), 7.403 (0.71), 7.407 (0.88), 7.411 (0.99), 7.419 (0.91), |
| | 7.435 (1.70), 7.444 (1.43), 7.449 (1.09), 7.452 (1.95), 7.457 (2.22), |
| | 7.466 (1.74), 7.471 (1.70), 7.677 (1.62), 7.681 (1.60), 8.247 (2.18), |
| | 8.260 (2.06), 10.737 (2.16). |
| 78 | 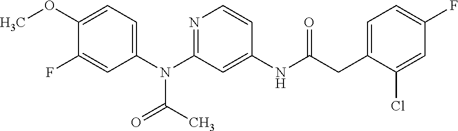 [SMILES: COc1ccc(N(C(C)=O)c2cc(NC(=O)Cc3ccc(F)cc3Cl)ccn2)cc1F] |
| | N-{4-[2-(2-chloro-4-fluorophenyl)acetamido]pyridin-2-yl}-N-(3- |
| | fluoro-4-methoxyphenyl)acetamide |
| | LC-MS (LC-ES+): Rₜ = 1.04 min; MS (ESIpos): m/z = 446 [M + H]⁺ |
| | ¹H-NMR (400 MHz, DMSO-d6) δ [ppm]: 1.986 (13.64), 2.518 (1.92), |
| | 2.523 (1.25), 3.842 (16.00), 3.865 (5.62), 7.031 (0.68), 7.053 (0.93), |
| | 7.145 (1.24), 7.168 (1.70), 7.187 (0.82), 7.190 (1.06), 7.193 (0.89), |
| | 7.208 (1.36), 7.215 (1.54), 7.230 (0.76), 7.237 (0.85), 7.244 (1.23), |
| | 7.250 (1.12), 7.274 (1.18), 7.280 (1.12), 7.431 (1.42), 7.438 (1.50), |
| | 7.444 (2.16), 7.447 (1.89), 7.453 (1.71), 7.460 (3.19), 7.466 (1.31), |
| | 7.482 (1.05), 7.674 (1.62), 7.677 (1.60), 8.246 (2.14), 8.260 (2.03), |
| | 10.733 (2.17). |
| 79 | 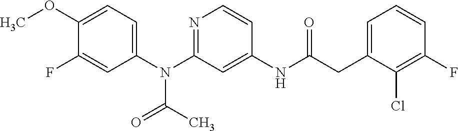 [SMILES: COc1ccc(N(C(C)=O)c2cc(NC(=O)Cc3cccc(F)c3Cl)ccn2)cc1F] |
| | N-{4-[2-(2-chloro-3-fluorophenyl)acetamido]pyridin-2-yl}-N-(3- |
| | fluoro-4-methoxyphenyl)acetamide |
| | LC-MS (Method 2): Rₜ = 1.09 min; MS (ESIneg): m/z = 444 [M − H]⁻ |
| | ¹H-NMR (400 MHz, DMSO-d6) δ [ppm]: 1.987 (14.51), 2.518 (0.89), |
| | 2.523 (0.56), 3.842 (16.00), 3.938 (6.32), 7.031 (0.64), 7.033 (0.67), |
| | 7.037 (0.60), 7.053 (0.88), 7.055 (0.94), 7.058 (0.83), 7.145 (1.30), |
| | 7.168 (1.70), 7.191 (0.93), 7.246 (1.24), 7.252 (1.18), 7.261 (0.74), |
| | 7.268 (1.09), 7.276 (1.69), 7.283 (2.16), 7.338 (1.69), 7.343 (2.14), |
| | 7.359 (3.72), 7.366 (1.23), 7.374 (1.07), 7.445 (1.55), 7.450 (1.59), |
| | 7.459 (1.48), 7.464 (1.63), 7.677 (1.60), 7.681 (1.57), 8.250 (2.16), |
| | 8.264 (2.04), 10.775 (2.17). |
| 80 | 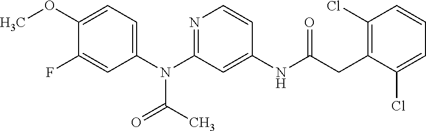 [SMILES: COc1ccc(N(C(C)=O)c2cc(NC(=O)Cc3c(Cl)cccc3Cl)ccn2)cc1F] |
| | N-{4-[2-(2,6-dichlorophenyl)acetamido]pyridin-2-yl}-N-(3-fluoro-4- |
| | methoxyphenyl)acetamide |
| | LC-MS (Method 2): Rₜ = 1.13 min; MS (ESIneg): m/z = 460 [M − H]⁻ |
| | ¹H-NMR (400 MHz, DMSO-d6) δ [ppm]: 1.985 (13.60), 2.332 (0.55), |
| | 2.518 (2.48), 2.523 (1.63), 2.673 (0.58), 3.842 (16.00), 4.076 (6.43), |
| | 7.036 (0.65), 7.058 (0.92), 7.062 (0.79), 7.145 (1.26), 7.168 (1.65), |
| | 7.190 (0.90), 7.248 (1.15), 7.254 (1.13), 7.278 (1.14), 7.284 (1.09), |
| | 7.335 (1.34), 7.354 (1.73), 7.357 (1.89), 7.375 (2.11), 7.416 (1.48), |
| | 7.421 (1.46), 7.430 (1.46), 7.435 (1.52), 7.487 (5.93), 7.506 (3.60), |
| | 7.679 (1.51), 8.250 (2.11), 8.264 (1.97), 10.849 (2.08). |
| 81 | 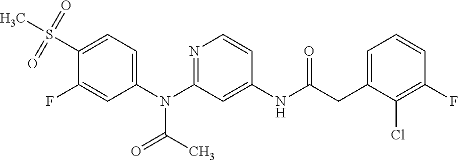 [SMILES: CC(=O)N(c1ccc(S(C)(=O)=O)c(F)c1)c1cc(NC(=O)Cc2cccc(F)c2Cl)ccn1] |
| | N-{4-[2-(2-chloro-3-fluorophenyl)acetamido]pyridin-2-yl}-N-[3- |
| | fluoro-4-(methanesulfonyl)phenyl]acetamide |
| | LC-MS (Method 2): Rₜ = 1.03 min; MS (ESIpos): m/z = 494 [M + H]⁺ |
| | ¹H-NMR (400 MHz, DMSO-d6) δ [ppm]: 2.038 (13.07), 2.518 (3.33), |
| | 2.522 (2.37), 3.334 (16.00), 3.340 (13.48), 3.949 (5.75), 7.193 (0.87), |
| | 7.198 (0.91), 7.215 (0.96), 7.219 (1.00), 7.261 (0.65), 7.268 (0.99), |
| | 7.275 (0.73), 7.279 (0.78), 7.285 (1.12), 7.338 (1.53), 7.343 (1.84), |
| | 7.359 (3.54), 7.367 (1.28), 7.374 (1.14), 7.537 (1.32), 7.542 (1.65), |
| | 7.551 (1.99), 7.556 (1.61), 7.574 (1.06), 7.578 (1.07), 7.682 (1.61), |
| | 7.685 (1.63), 7.823 (1.17), 7.844 (2.05), 7.864 (1.10), 8.367 (1.68), |
| | 8.381 (1.66), 10.872 (2.13). |
| 82 | 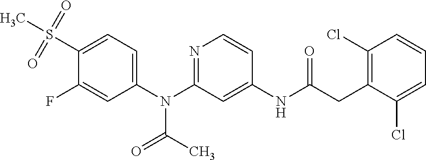 [SMILES: CC(=O)N(c1ccc(S(C)(=O)=O)c(F)c1)c1cc(NC(=O)Cc2c(Cl)cccc2Cl)ccn1] |
| | N-{4-[2-(2,6-dichlorophenyl)acetamido]pyridin-2-yl}-N-[3-fluoro-4- |
| | (methanesulfonyl)phenyl]acetamide |
| | LC-MS (Method 2): Rₜ = 1.07 min; MS (ESIpos): m/z = 510 [M + H]⁺ |
| | ¹H-NMR (400 MHz, DMSO-d6) δ [ppm]: 2.036 (5.17), 2.518 (2.11), |
| | 2.522 (1.49), 3.332 (16.00), 3.339 (5.42), 4.088 (2.45), 7.334 (0.50), |
| | 7.353 (0.63), 7.356 (0.74), 7.375 (0.80), 7.486 (2.26), 7.505 (1.42), |
| | 7.517 (0.59), 7.522 (0.58), 7.531 (0.54), 7.536 (0.61), 7.548 (0.45), |
| | 7.552 (0.45), 7.577 (0.41), 7.581 (0.43), 7.679 (0.64), 7.682 (0.64), |
| | 7.821 (0.48), 7.842 (0.84), 7.863 (0.45), 8.368 (0.68), 8.382 (0.67), |
| | 10.951 (0.87). |
| 83 | 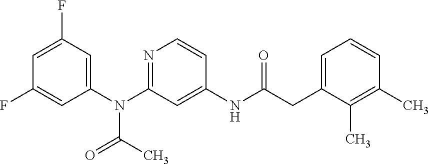 [SMILES: CC(=O)N(c1cc(F)cc(F)c1)c1cc(NC(=O)Cc2cccc(C)c2C)ccn1] |
| | N-(3,5-difluorophenyl)-N-{4-[2-(2,3- |
| | dimethylphenyl)acetamido]pyridin-2-yl}acetamide |
| | LC-MS (Method 1): Rₜ = 1.23 min; MS (ESIpos): m/z = 410 [M + H]⁺ |
| | ¹H-NMR (400 MHz, DMSO-d6) δ [ppm]: 1.108 (5.83), 2.014 (16.00), |
| | 2.142 (10.68), 2.235 (10.48), 2.327 (0.61), 2.331 (0.45), 2.336 (0.20), |
| | 2.518 (2.77), 2.523 (1.64), 2.539 (2.07), 2.669 (0.62), 2.673 (0.46), |
| | 2.678 (0.20), 3.740 (6.40), 4.191 (0.52), 6.994 (0.40), 7.008 (0.59), |
| | 7.017 (0.85), 7.030 (2.28), 7.047 (4.73), 7.061 (2.64), 7.068 (1.34), |
| | 7.077 (1.49), 7.082 (1.45), 7.093 (0.29), 7.188 (0.32), 7.194 (0.60), |
| | 7.200 (0.36), 7.212 (0.66), 7.217 (1.19), 7.223 (0.68), 7.235 (0.33), |
| | 7.241 (0.59), 7.247 (0.32), 7.511 (1.48), 7.515 (1.52), 7.524 (1.49), |
| | 7.530 (1.53), 7.695 (1.57), 7.698 (1.54), 8.296 (1.85), 8.310 (1.76), |
| | 10.681 (2.10). |
| 84 | 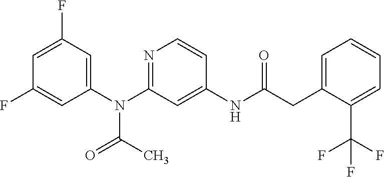 [SMILES: CC(=O)N(c1cc(F)cc(F)c1)c1cc(NC(=O)Cc2ccccc2C(F)(F)F)ccn1] |
| | N-(3,5-difluorophenyl)-N-(4-{2-[2- |
| | (trifluoromethyl)phenyl]acetamido}pyridin-2-yl)acetamide |
| | LC-MS (Method 1): Rₜ = 1.21 min; MS (ESIpos): m/z = 450 [M + H]⁺ |
| | ¹H-NMR (400 MHz, DMSO-d6) δ [ppm]: 0.967 (0.22), 1.108 (8.14), |
| | 2.016 (16.00), 2.337 (0.19), 2.518 (2.29), 2.523 (1.61), 2.540 (2.42), |
| | 2.659 (0.20), 2.678 (0.20), 3.969 (3.05), 4.191 (0.75), 7.053 (0.25), |
| | 7.063 (1.20), 7.069 (1.45), 7.084 (1.35), 7.089 (1.33), 7.100 (0.26), |
| | 7.193 (0.30), 7.198 (0.57), 7.205 (0.35), 7.216 (0.67), 7.222 (1.15), |
| | 7.228 (0.62), 7.240 (0.32), 7.245 (0.57), 7.251 (0.29), 7.483 (1.86), |
| | 7.488 (1.64), 7.497 (2.29), 7.502 (3.50), 7.522 (2.03), 7.635 (0.75), |
| | 7.653 (1.09), 7.673 (0.49), 7.691 (1.48), 7.695 (1.48), 7.704 (1.39), |
| | 7.724 (1.12), 8.301 (1.80), 8.315 (1.70), 10.774 (2.00). |
| 85 | 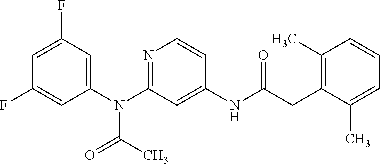 [SMILES: CC(=O)N(c1cc(F)cc(F)c1)c1cc(NC(=O)Cc2c(C)cccc2C)ccn1] |
| | N-(3,5-difluorophenyl)-N-{4-[2-(2,6- |
| | dimethylphenyl)acetamido]pyridin-2-yl}acetamide |
| | LC-MS (Method 1): Rₜ = 1.22 min; MS (ESIpos): m/z = 410 [M + H]⁺ |
| | ¹H-NMR (400 MHz, DMSO-d6) δ [ppm]: 0.967 (0.20), 1.108 (9.09), |
| | 2.014 (12.16), 2.240 (16.00), 2.327 (0.49), 2.331 (0.35), 2.518 (1.73), |
| | 2.523 (1.22), 2.540 (0.30), 2.669 (0.49), 2.673 (0.34), 3.782 (4.33), |
| | 4.191 (0.75), 6.991 (0.56), 6.999 (0.69), 7.013 (3.11), 7.027 (2.29), |
| | 7.040 (0.86), 7.050 (0.76), 7.056 (0.89), 7.062 (1.25), 7.076 (0.95), |
| | 7.082 (0.95), 7.093 (0.18), 7.186 (0.23), 7.192 (0.42), 7.198 (0.25), |
| | 7.210 (0.49), 7.215 (0.85), 7.221 (0.46), 7.233 (0.24), 7.239 (0.42), |
| | 7.245 (0.23), 7.502 (1.06), 7.507 (1.07), 7.516 (1.01), 7.521 (1.12), |
| | 7.702 (1.04), 7.705 (1.01), 8.301 (1.27), 8.315 (1.20), 10.758 (1.42). |
| 86 | 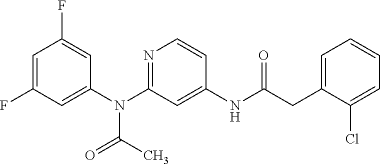 [SMILES: CC(=O)N(c1cc(F)cc(F)c1)c1cc(NC(=O)Cc2ccccc2Cl)ccn1] |
| | N-{4-[2-(2-chlorophenyl)acetamido]pyridin-2-yl}-N-(3,5- |
| | difluorophenyl)acetamide |
| | LC-MS (Method 1): Rₜ = 1.16 min; MS (ESIpos): m/z = 416 [M + H]⁺ |
| | ¹H-NMR (400 MHz, DMSO-d6) δ [ppm]: 1.987 (0.59), 2.020 (16.00), |
| | 2.518 (2.19), 2.522 (1.39), 3.884 (7.33), 7.062 (1.38), 7.068 (1.65), |
| | 7.083 (1.55), 7.088 (1.53), 7.198 (0.64), 7.215 (0.70), 7.221 (1.22), |
| | 7.227 (0.69), 7.245 (0.61), 7.303 (2.90), 7.312 (2.11), 7.318 (2.20), |
| | 7.326 (3.86), 7.336 (0.49), 7.399 (1.53), 7.406 (0.76), 7.409 (0.96), |
| | 7.414 (1.06), 7.422 (0.98), 7.437 (1.71), 7.445 (1.37), 7.450 (1.00), |
| | 7.459 (1.20), 7.505 (1.51), 7.510 (1.60), 7.520 (1.56), 7.524 (1.62), |
| | 7.698 (1.72), 8.305 (2.01), 8.319 (1.90), 10.791 (2.26). |
| 87 | 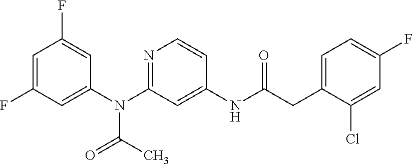 [SMILES: CC(=O)N(c1cc(F)cc(F)c1)c1cc(NC(=O)Cc2ccc(F)cc2Cl)ccn1] |
| | N-{4-[2-(2-chloro-4-fluorophenyl)acetamido]pyridin-2-yl}-N-(3,5- |
| | difluorophenyl)acetamide |
| | LC-MS (Method 1): Rₜ = 1.19 min; MS (ESIpos): m/z = 434 [M + H]⁺ |
| | ¹H-NMR (400 MHz, DMSO-d6) δ [ppm]: 2.019 (16.00), 2.518 (1.21), |
| | 2.522 (0.79), 3.874 (6.02), 7.062 (1.33), 7.067 (1.60), 7.083 (1.49), |
| | 7.088 (1.48), 7.188 (0.72), 7.194 (0.97), 7.199 (0.72), 7.205 (0.49), |
| | 7.209 (1.49), 7.216 (2.24), 7.222 (1.34), 7.228 (0.89), 7.230 (1.01), |
| | 7.237 (1.01), 7.245 (0.64), 7.431 (1.46), 7.438 (1.51), 7.447 (1.36), |
| | 7.454 (1.58), 7.461 (1.82), 7.469 (1.24), 7.485 (1.13), 7.497 (1.58), |
| | 7.502 (1.56), 7.511 (1.54), 7.516 (1.66), 7.694 (1.64), 7.698 (1.61), |
| | 8.304 (1.99), 8.319 (1.87), 10.788 (2.25). |
| 88 | 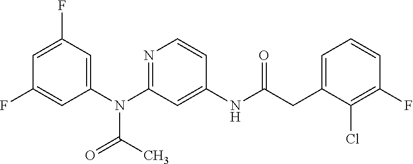 [SMILES: CC(=O)N(c1cc(F)cc(F)c1)c1cc(NC(=O)Cc2cccc(F)c2Cl)ccn1] |
| | N-{4-[2-(2-chloro-3-fluorophenyl)acetamido]pyridin-2-yl}-N-(3,5- |
| | difluorophenyl)acetamide |
| | LC-MS (Method 1): Rₜ = 1.18 min; MS (ESIpos): m/z = 434 [M + H]⁺ |
| | ¹H-NMR (400 MHz, DMSO-d6) δ [ppm]: 0.967 (0.20), 1.107 (11.67), |
| | 1.144 (0.18), 2.019 (16.00), 2.327 (0.57), 2.331 (0.40), 2.336 (0.18), |
| | 2.518 (2.29), 2.523 (1.45), 2.540 (0.16), 2.669 (0.58), 2.673 (0.42), |
| | 2.678 (0.19), 3.946 (6.42), 4.191 (1.19), 7.053 (0.27), 7.065 (1.29), |
| | 7.070 (1.55), 7.085 (1.45), 7.090 (1.44), 7.101 (0.28), 7.194 (0.32), |
| | 7.200 (0.61), 7.206 (0.35), 7.217 (0.68), 7.223 (1.20), 7.229 (0.66), |
| | 7.241 (0.34), 7.247 (0.62), 7.252 (0.34), 7.263 (0.68), 7.270 (1.04), |
| | 7.277 (0.75), 7.281 (0.81), 7.286 (1.12), 7.325 (0.38), 7.339 (1.67), |
| | 7.344 (2.05), 7.360 (3.83), 7.368 (1.23), 7.376 (1.12), 7.381 (0.33), |
| | 7.396 (0.18), 7.498 (1.52), 7.503 (1.48), 7.512 (1.49), 7.517 (1.58), |
| | 7.697 (1.57), 7.700 (1.54), 8.308 (1.91), 8.321 (1.81), 10.829 (2.14). |
| 89 | 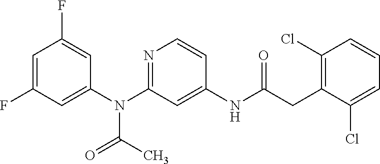 [SMILES: CC(=O)N(c1cc(F)cc(F)c1)c1cc(NC(=O)Cc2c(Cl)cccc2Cl)ccn1] |
| | N-{4-[2-(2,6-dichlorophenyl)acetamido]pyridin-2-yl}-N-(3,5- |
| | difluorophenyl)acetamide |
| | LC-MS (Method 1): Rₜ = 1.22 min; MS (ESIpos): m/z = 450 [M + H]⁺ |
| | ¹H-NMR (400 MHz, DMSO-d6) δ [ppm]: 2.017 (16.00), 2.518 (2.37), |
| | 2.522 (1.50), 4.086 (7.45), 7.068 (1.49), 7.074 (1.76), 7.088 (1.66), |
| | 7.093 (1.63), 7.198 (0.64), 7.216 (0.75), 7.221 (1.29), 7.227 (0.71), |
| | 7.245 (0.64), 7.336 (1.36), 7.355 (1.86), 7.357 (2.02), 7.376 (2.23), |
| | 7.475 (1.62), 7.480 (1.76), 7.488 (7.59), 7.494 (1.94), 7.508 (4.06), |
| | 7.695 (1.79), 8.308 (2.07), 8.323 (1.99), 10.906 (2.33). |
| 90 | 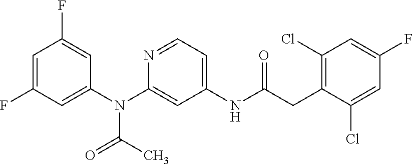 [SMILES: CC(=O)N(c1cc(F)cc(F)c1)c1cc(NC(=O)Cc2c(Cl)cc(F)cc2Cl)ccn1] |
| | N-{4-[2-(2,6-dichloro-4-fluorophenyl)acetamido]pyridin-2-yl}-N- |
| | (3,5-difluorophenyl)acetamide |
| | ¹H NMR (500 MHz, DMSO-d6) δ [ppm]: 2.02 (s, 3H), 4.05 (s, 2H), 7.04- |
| | 7.12 (m, 2H), 7.17 - 7.25 (m, 1H), 7.48 (dd, J = 5.6, 1.9, 1H), 7.57 (d, |
| | J = 8.5, 2H), 7.69 (s, 1H), 8.32 (d, J = 5.6, 1H), 10.89 (s, 1H). |
| 91 | 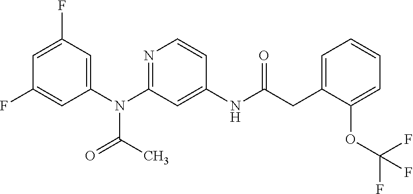 [SMILES: CC(=O)N(c1cc(F)cc(F)c1)c1cc(NC(=O)Cc2ccccc2OC(F)(F)F)ccn1] |
| | N-(3,5-difluorophenyl)-N-(4-{2-[2- |
| | (trifluoromethoxy)phenyl]acetamido}pyridin-2-yl)acetamide |
| | LC-MS (Method 1): Rₜ = 1.24 min; MS (ESIpos): m/z = 466 [M + H]⁺ |
| | ¹H-NMR (400 MHz, DMSO-d6) δ [ppm]: 1.108 (1.32), 2.019 (16.00), |
| | 2.331 (0.47), 2.336 (0.20), 2.518 (2.65), 2.523 (1.81), 2.540 (1.58), |
| | 2.673 (0.45), 2.678 (0.20), 3.828 (5.95), 7.049 (0.25), 7.060 (1.20), |
| | 7.066 (1.45), 7.081 (1.35), 7.086 (1.35), 7.098 (0.26), 7.193 (0.31), |
| | 7.199 (0.57), 7.205 (0.35), 7.216 (0.66), 7.222 (1.11), 7.228 (0.63), |
| | 7.240 (0.32), 7.245 (0.56), 7.252 (0.31), 7.336 (0.53), 7.339 (0.73), |
| | 7.344 (0.85), 7.348 (0.72), 7.355 (0.79), 7.360 (1.21), 7.363 (2.38), |
| | 7.381 (1.56), 7.385 (0.92), 7.403 (1.11), 7.408 (1.42), 7.422 (0.91), |
| | 7.427 (1.27), 7.441 (0.41), 7.446 (0.41), 7.461 (1.45), 7.466 (1.04), |
| | 7.480 (1.02), 7.484 (0.73), 7.496 (1.51), 7.500 (1.51), 7.510 (1.52), |
| | 7.515 (1.51), 7.691 (1.48), 7.694 (1.42), 8.304 (1.80), 8.318 (1.71), |
| | 10.780 (1.99). |
| 92 | 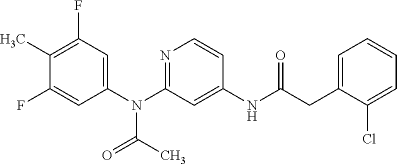 [SMILES: CC(=O)N(c1cc(F)c(C)c(F)c1)c1cc(NC(=O)Cc2ccccc2Cl)ccn1] |
| | N-{4-[2-(2-chlorophenyl)acetamido]pyridin-2-yl}-N-(3,5-difluoro-4- |
| | methylphenyl)acetamide |
| | LC-MS (Method 2): Rₜ = 1.21 min; MS (ESIneg): m/z = 428 [M − H]⁻ |
| | ¹H-NMR (400 MHz, DMSO-d6) δ [ppm]: 2.014 (16.00), 2.139 (7.39), |
| | 3.881 (7.97), 7.041 (2.73), 7.060 (2.83), 7.303 (2.92), 7.312 (2.45), |
| | 7.317 (2.59), 7.326 (4.22), 7.336 (0.70), 7.398 (1.66), 7.408 (1.13), |
| | 7.412 (1.24), 7.421 (1.12), 7.426 (0.50), 7.436 (1.85), 7.445 (1.53), |
| | 7.450 (1.19), 7.459 (1.38), 7.485 (1.69), 7.489 (1.77), 7.498 (1.77), |
| | 7.504 (1.82), 7.702 (2.01), 8.279 (2.25), 8.294 (2.20), 10.773 (2.63). |
| 93 | 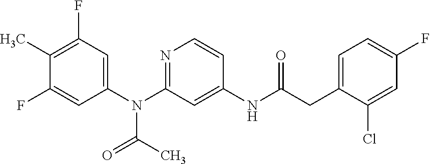 [SMILES: CC(=O)N(c1cc(F)c(C)c(F)c1)c1cc(NC(=O)Cc2ccc(F)cc2Cl)ccn1] |
| | N-{4-[2-(2-chloro-4-fluorophenyl)acetamido]pyridin-2-yl}-N-(3,5- |
| | difluoro-4-methylphenyl)acetamide |
| | LC-MS (Method 2): Rₜ = 1.24 min; MS (ESIneg): m/z = 446 [M − H]⁻ |
| | ¹H-NMR (400 MHz, DMSO-d6) δ [ppm]: 2.012 (16.00), 2.139 (6.03), |
| | 2.518 (0.82), 2.523 (0.55), 3.871 (5.72), 7.039 (2.29), 7.059 (2.30), |
| | 7.187 (0.68), 7.194 (0.74), 7.208 (1.37), 7.215 (1.60), 7.230 (0.80), |
| | 7.237 (0.85), 7.431 (1.48), 7.437 (1.49), 7.446 (1.32), 7.453 (1.57), |
| | 7.460 (2.04), 7.468 (1.23), 7.476 (1.65), 7.482 (2.09), 7.490 (1.54), |
| | 7.495 (1.65), 7.696 (1.58), 7.699 (1.56), 8.278 (1.98), 8.293 (1.87), |
| | 10.769 (2.20). |
| 94 | 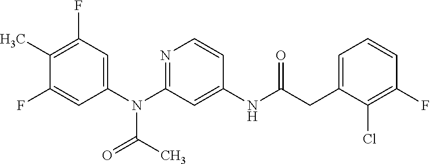 [SMILES: CC(=O)N(c1cc(F)c(C)c(F)c1)c1cc(NC(=O)Cc2cccc(F)c2Cl)ccn1] |
| | N-{4-[2-(2-chloro-3-fluorophenyl)acetamido]pyridin-2-yl}-N-(3,5- |
| | difluoro-4-methylphenyl)acetamide |
| | LC-MS (Method 2): Rₜ = 1.22 min; MS (ESIneg): m/z = 446 [M − H]⁻ |
| | ¹H-NMR (400 MHz, DMSO-d6) δ [ppm]: 2.013 (16.00), 2.139 (6.10), |
| | 2.518 (1.79), 2.523 (1.17), 3.943 (6.68), 7.042 (2.32), 7.062 (2.33), |
| | 7.263 (0.70), 7.270 (1.06), 7.276 (0.79), 7.280 (0.83), 7.286 (1.14), |
| | 7.324 (0.42), 7.339 (1.74), 7.343 (2.23), 7.360 (4.07), 7.367 (1.24), |
| | 7.375 (1.19), 7.477 (1.65), 7.482 (1.65), 7.491 (1.67), 7.496 (1.65), |
| | 7.698 (1.59), 7.701 (1.56), 8.281 (2.03), 8.295 (1.91), 10.809 (2.20). |
| 95 | 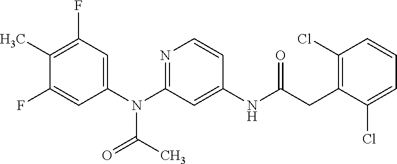 [SMILES: CC(=O)N(c1cc(F)c(C)c(F)c1)c1cc(NC(=O)Cc2c(Cl)cccc2Cl)ccn1] |
| | N-{4-[2-(2,6-dichlorophenyl)acetamido]pyridin-2-yl}-N-(3,5- |
| | difluoro-4-methylphenyl)acetamide |
| | LC-MS (Method 2): Rₜ = 1.26 min; MS (ESIneg): m/z = 462 [M − H]⁻ |
| | ¹H-NMR (400 MHz, DMSO-d6) δ [ppm]: 2.010 (16.00), 2.138 (6.70), |
| | 2.518 (5.23), 2.522 (3.78), 4.082 (7.30), 7.045 (2.48), 7.065 (2.60), |
| | 7.078 (0.44), 7.335 (1.37), 7.354 (1.96), 7.357 (2.07), 7.376 (2.46), |
| | 7.451 (1.57), 7.455 (1.72), 7.465 (1.70), 7.469 (1.79), 7.487 (6.67), |
| | 7.507 (4.12), 7.702 (1.73), 8.283 (2.11), 8.297 (2.05), 10.887 (2.41). |
| 96 | 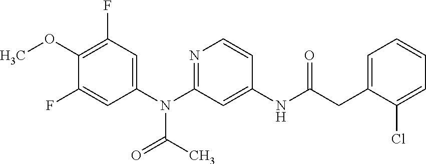 [SMILES: COc1c(F)cc(N(C(C)=O)c2cc(NC(=O)Cc3ccccc3Cl)ccn2)cc1F] |
| | N-{4-[2-(2-chlorophenyl)acetamido]pyridin-2-yl}-N-(3,5-difluoro-4- |
| | methoxyphenyl)acetamide |
| | LC-MS (Method 2): Rₜ = 1.15 min; MS (ESIpos): m/z = 446 [M + H]⁺ |
| | ¹H-NMR (400 MHz, DMSO-d6) δ [ppm]: 2.005 (16.00), 2.518 (1.69), |
| | 2.522 (1.09), 3.883 (7.23), 3.929 (11.28), 7.159 (2.56), 7.183 (2.61), |
| | 7.303 (3.01), 7.312 (1.82), 7.314 (1.91), 7.317 (2.04), 7.319 (2.05), |
| | 7.327 (4.04), 7.336 (0.41), 7.338 (0.48), 7.400 (1.53), 7.407 (0.75), |
| | 7.411 (0.93), 7.415 (1.00), 7.423 (0.98), 7.437 (1.79), 7.446 (1.44), |
| | 7.450 (0.97), 7.453 (0.69), 7.461 (1.22), 7.492 (1.63), 7.497 (1.56), |
| | 7.506 (1.59), 7.511 (1.67), 7.702 (1.59), 8.280 (2.09), 8.294 (1.97), |
| | 10.773 (2.26). |
| 97 | 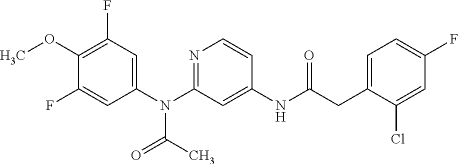 [SMILES: COc1c(F)cc(N(C(C)=O)c2cc(NC(=O)Cc3ccc(F)cc3Cl)ccn2)cc1F] |
| | N-{4-[2-(2-chloro-4-fluorophenyl)acetamido]pyridin-2-yl}-N-(3,5- |
| | difluoro-4-methoxyphenyl)acetamide |
| | LC-MS (Method 2): Rₜ = 1.18 min; MS (ESIpos): m/z = 464 [M + H]⁺ |
| | ¹H-NMR (400 MHz, DMSO-d6) δ [ppm]: 2.000 (16.00), 3.870 (8.19), |
| | 3.925 (14.27), 7.143 (0.54), 7.157 (3.38), 7.179 (3.66), 7.192 (1.59), |
| | 7.207 (1.78), 7.213 (1.96), 7.228 (0.96), 7.234 (1.06), 7.429 (1.62), |
| | 7.436 (1.80), 7.445 (1.83), 7.451 (2.10), 7.460 (2.60), 7.466 (1.94), |
| | 7.484 (3.13), 7.494 (1.99), 7.498 (2.19), 7.698 (2.62), 8.275 (2.59), |
| | 8.289 (2.54), 10.768 (3.36). |
| 98 | 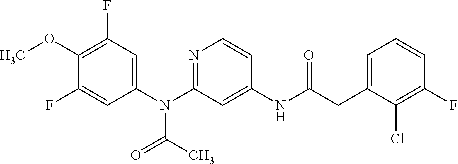 [SMILES: COc1c(F)cc(N(C(C)=O)c2cc(NC(=O)Cc3cccc(F)c3Cl)ccn2)cc1F] |
| | N-{4-[2-(2-chloro-3-fluorophenyl)acetamido]pyridin-2-yl}-N-(3,5- |
| | difluoro-4-methoxyphenyl)acetamide |
| | LC-MS (Method 2): Rₜ = 1.16 min; MS (ESIpos): m/z = 464 [M + H]⁺ |
| | ¹H-NMR (400 MHz, DMSO-d6) δ [ppm]: 2.005 (16.00), 2.518 (1.53), |
| | 2.523 (0.97), 3.930 (11.22), 3.946 (6.54), 7.162 (2.51), 7.185 (2.52), |
| | 7.265 (0.69), 7.272 (1.05), 7.279 (0.77), 7.283 (0.83), 7.287 (1.14), |
| | 7.340 (1.74), 7.345 (2.29), 7.361 (3.74), 7.369 (1.27), 7.377 (1.11), |
| | 7.484 (1.50), 7.489 (1.60), 7.499 (1.55), 7.503 (1.61), 7.702 (1.52), |
| | 8.283 (2.01), 8.297 (1.91), 10.810 (2.22). |
| 99 | 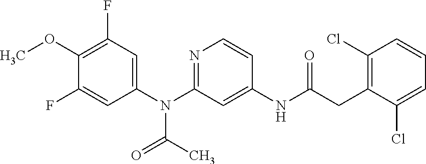 [SMILES: COc1c(F)cc(N(C(C)=O)c2cc(NC(=O)Cc3c(Cl)cccc3Cl)ccn2)cc1F] |
| | N-{4-[2-(2,6-dichlorophenyl)acetamido]pyridin-2-yl}-N-(3,5- |
| | difluoro-4-methoxyphenyl)acetamide |
| | LC-MS (Method 2): Rₜ = 1.20 min; MS (ESIneg): m/z = 478 [M − H]⁻ |
| | ¹H-NMR (400 MHz, DMSO-d6) δ [ppm]: 2.002 (16.00), 2.518 (1.89), |
| | 2.523 (1.30), 3.929 (11.02), 4.085 (6.96), 7.165 (2.46), 7.188 (2.55), |
| | 7.337 (1.39), 7.355 (1.89), 7.359 (2.00), 7.377 (2.32), 7.459 (1.55), |
| | 7.463 (1.49), 7.473 (1.54), 7.478 (1.63), 7.489 (6.28), 7.509 (3.82), |
| | 7.703 (1.48), 8.284 (2.01), 8.298 (1.92), 10.887 (2.23). |
| 100 | 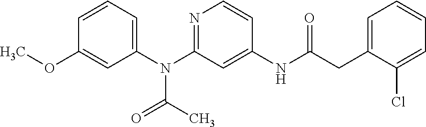 [SMILES: COc1cccc(N(C(C)=O)c2cc(NC(=O)Cc3ccccc3Cl)ccn2)c1] |
| | N-{4-[2-(2-chlorophenyl)acetamido]pyridin-2-yl}-N-(3- |
| | methoxyphenyl)acetamide |
| | LC-MS (Method 2): Rₜ = 1.06 min; MS (ESIneg): m/z = 408 [M − H]⁻ |
| | ¹H-NMR (400 MHz, DMSO-d6) δ [ppm]: 1.996 (16.00), 2.518 (0.51), |
| | 3.336 (5.17), 3.872 (7.46), 6.800 (1.05), 6.803 (1.11), 6.804 (1.06), |
| | 6.820 (1.07), 6.822 (1.24), 6.824 (1.13), 6.860 (1.24), 6.866 (2.48), |
| | 6.871 (1.75), 6.877 (1.41), 6.879 (1.46), 6.884 (0.70), 6.886 (0.65), |
| | 6.898 (1.30), 6.900 (1.25), 6.904 (0.99), 6.906 (0.87), 7.285 (1.67), |
| | 7.299 (3.19), 7.305 (3.33), 7.314 (2.40), 7.323 (4.40), 7.333 (0.55), |
| | 7.394 (1.61), 7.401 (0.80), 7.404 (0.98), 7.408 (1.14), 7.417 (1.01), |
| | 7.423 (0.40), 7.433 (1.86), 7.442 (1.53), 7.447 (1.07), 7.457 (1.33), |
| | 7.464 (1.71), 7.469 (1.72), 7.478 (1.58), 7.483 (1.72), 7.650 (1.94), |
| | 7.654 (1.87), 8.265 (2.44), 8.279 (2.31), 10.738 (2.37). |
| 101 | 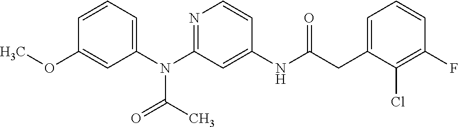 [SMILES: COc1cccc(N(C(C)=O)c2cc(NC(=O)Cc3cccc(F)c3Cl)ccn2)c1] |
| | N-{4-[2-(2-chloro-3-fluorophenyl)acetamido]pyridin-2-yl}-N-(3- |
| | methoxyphenyl)acetamide |
| | LC-MS (Method 2): Rₜ = 1.07 min; MS (ESIneg): m/z = 426 [M − H]⁻ |
| | ¹H-NMR (400 MHz, DMSO-d6) δ [ppm]: 1.995 (16.00), 2.518 (0.78), |
| | 2.522 (0.48), 3.333 (10.83), 3.934 (6.61), 6.801 (1.00), 6.803 (1.03), |
| | 6.805 (1.02), 6.818 (0.90), 6.820 (0.99), 6.823 (1.18), 6.825 (1.08), |
| | 6.860 (1.17), 6.866 (2.36), 6.871 (1.67), 6.879 (1.32), 6.881 (1.36), |
| | 6.885 (0.70), 6.887 (0.64), 6.899 (1.24), 6.901 (1.17), 6.905 (0.96), |
| | 6.908 (0.83), 7.258 (0.70), 7.265 (1.09), 7.272 (0.78), 7.276 (0.83), |
| | 7.282 (1.22), 7.286 (1.72), 7.306 (2.64), 7.322 (0.48), 7.326 (1.31), |
| | 7.336 (1.74), 7.341 (2.07), 7.357 (4.12), 7.365 (1.26), 7.372 (1.20), |
| | 7.455 (1.60), 7.460 (1.65), 7.470 (1.53), 7.474 (1.72), 7.648 (1.77), |
| | 7.652 (1.73), 8.267 (2.30), 8.281 (2.17), 10.775 (2.23). |
| 102 | 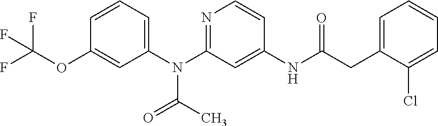 [SMILES: CC(=O)N(c1cccc(OC(F)(F)F)c1)c1cc(NC(=O)Cc2ccccc2Cl)ccn1] |
| | N-{4-[2-(2-chlorophenyl)acetamido]pyridin-2-yl}-N-[3- |
| | (trifluoromethoxy)phenyl]acetamide |
| | LC-MS (Method 2): Rₜ = 1.23 min; MS (ESIneg): m/z = 462 [M − H]⁻ |
| | ¹H-NMR (400 MHz, DMSO-d6) δ [ppm]: 2.005 (16.00), 2.518 (0.44), |
| | 3.336 (6.99), 7.244 (0.91), 7.247 (0.93), 7.264 (1.04), 7.266 (1.09), |
| | 7.300 (3.89), 7.309 (2.57), 7.315 (3.05), 7.323 (5.24), 7.333 (0.61), |
| | 7.366 (1.60), 7.395 (1.59), 7.403 (0.80), 7.406 (0.99), 7.410 (1.15), |
| | 7.419 (1.02), 7.424 (0.41), 7.434 (1.83), 7.443 (1.52), 7.447 (1.13), |
| | 7.457 (1.29), 7.501 (1.86), 7.509 (1.67), 7.514 (1.76), 7.521 (3.30), |
| | 7.523 (2.10), 7.528 (1.80), 7.541 (1.32), 7.675 (1.61), 8.305 (2.04), |
| | 8.319 (1.92), 10.782 (2.36). |
| 103 | 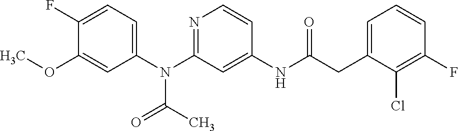 [SMILES: COc1cc(N(C(C)=O)c2cc(NC(=O)Cc3cccc(F)c3Cl)ccn2)ccc1F] |
| | N-{4-[2-(2-chloro-3-fluorophenyl)acetamido]pyridin-2-yl}-N-(4- |
| | fluoro-3-methoxyphenyl)acetamide |
| | LC-MS (Method 2): Rₜ = 1.10 min; MS (ESIpos): m/z = 446 [M + H]⁺ |
| | ¹H-NMR (400 MHz, DMSO-d6) δ [ppm]: 2.003 (13.52), 2.518 (0.96), |
| | 2.522 (0.58), 3.331 (16.00), 3.941 (5.80), 6.802 (0.48), 6.808 (0.57), |
| | 6.812 (0.56), 6.818 (0.53), 6.823 (0.60), 6.830 (0.65), 6.834 (0.61), |
| | 6.839 (0.56), 7.157 (1.02), 7.163 (1.04), 7.177 (1.05), 7.183 (0.99), |
| | 7.207 (1.35), 7.228 (1.28), 7.235 (1.36), 7.256 (1.23), 7.263 (0.66), |
| | 7.270 (0.95), 7.277 (0.68), 7.281 (0.72), 7.286 (1.02), 7.339 (1.55), |
| | 7.344 (2.01), 7.360 (3.36), 7.367 (1.16), 7.376 (1.05), 7.456 (1.42), |
| | 7.461 (1.44), 7.470 (1.37), 7.475 (1.51), 7.700 (1.40), 7.703 (1.38), |
| | 8.252 (2.00), 8.267 (1.88), 10.777 (1.96). |
| 104 | 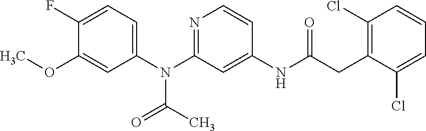 [SMILES: COc1cc(N(C(C)=O)c2cc(NC(=O)Cc3c(Cl)cccc3Cl)ccn2)ccc1F] |
| | N-{4-[2-(2,6-dichlorophenyl)acetamido]pyridin-2-yl}-N-(4-fluoro-3- |
| | methoxyphenyl)acetamide |
| | LC-MS (Method 2): Rₜ = 1.13 min; MS (ESIneg): m/z = 460 [M − H]⁻ |
| | ¹H-NMR (400 MHz, DMSO-d6) δ [ppm]: 2.000 (13.82), 2.522 (1.06), |
| | 3.804 (16.00), 4.080 (7.13), 6.806 (0.56), 6.813 (0.71), 6.816 (0.72), |
| | 6.823 (0.66), 6.828 (0.73), 6.834 (0.83), 6.837 (0.76), 6.844 (0.64), |
| | 7.158 (1.20), 7.164 (1.22), 7.177 (1.23), 7.184 (1.13), 7.206 (1.43), |
| | 7.228 (1.39), 7.234 (1.45), 7.256 (1.27), 7.336 (1.29), 7.355 (1.82), |
| | 7.357 (1.88), 7.376 (2.04), 7.429 (1.59), 7.434 (1.53), 7.443 (1.57), |
| | 7.448 (1.57), 7.488 (6.26), 7.508 (4.04), 7.701 (1.80), 8.253 (2.31), |
| | 8.267 (2.16), 10.852 (2.44). |
| 105 | 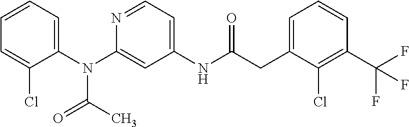 [SMILES: CC(=O)N(c1cc(NC(=O)Cc2cccc(C(F)(F)F)c2Cl)ccn1)c1ccccc1Cl] |
| | N-(2-chlorophenyl)-N-(4-{2-[2-chloro-3- |
| | (trifluoromethyl)phenyl]acetamido}pyridin-2-yl)acetamide |
| | LC-MS (Method 2): Rₜ = 1.23 min; MS (ESIneg): m/z = 480 [M − H]⁻ |
| | ¹H-NMR (400 MHz, DMSO-d6) δ [ppm]: 1.991 (16.00), 2.518 (1.43), |
| | 2.522 (0.91), 4.027 (14.23), 7.404 (0.78), 7.416 (5.07), 7.422 (3.84), |
| | 7.428 (4.48), 7.433 (4.39), 7.438 (9.76), 7.443 (5.03), 7.452 (4.91), |
| | 7.457 (4.40), 7.473 (2.98), 7.479 (1.62), 7.485 (1.89), 7.497 (1.28), |
| | 7.527 (1.66), 7.547 (3.75), 7.566 (2.20), 7.589 (0.66), 7.597 (4.08), |
| | 7.605 (2.23), 7.609 (3.51), 7.615 (2.22), 7.622 (3.12), 7.629 (0.44), |
| | 7.745 (3.08), 7.761 (2.39), 7.764 (2.42), 7.795 (3.30), 7.799 (2.97), |
| | 7.815 (2.95), 7.818 (2.59), 7.855 (1.27), 8.175 (3.48), 8.189 (3.32), |
| | 10.819 (5.42). |
| 106 | 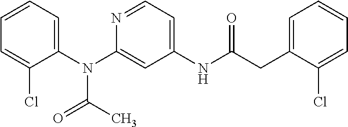 [SMILES: CC(=O)N(c1cc(NC(=O)Cc2ccccc2Cl)ccn1)c1ccccc1Cl] |
| | N-(2-chlorophenyl)-N-{4-[2-(2-chlorophenyl)acetamido]pyridin-2- |
| | yl}acetamide |
| | LC-MS (Method 2): Rₜ = 1.12 min; MS (ESIneg): m/z = 412 [M − H]⁻ |
| | ¹H-NMR (400 MHz, DMSO-d6) δ [ppm]: 1.995 (16.00), 2.518 (0.97), |
| | 2.522 (0.61), 3.337 (15.72), 7.292 (0.61), 7.303 (6.72), 7.311 (3.77), |
| | 7.313 (4.39), 7.317 (4.38), 7.319 (4.47), 7.326 (9.06), 7.335 (0.87), |
| | 7.337 (1.04), 7.392 (0.55), 7.401 (3.96), 7.407 (2.02), 7.414 (6.87), |
| | 7.420 (4.17), 7.425 (6.02), 7.431 (4.47), 7.437 (10.45), 7.446 (3.77), |
| | 7.451 (6.06), 7.456 (4.92), 7.461 (3.88), 7.466 (4.82), 7.470 (7.21), |
| | 7.476 (1.80), 7.482 (1.90), 7.494 (1.23), 7.589 (0.60), 7.597 (4.04), |
| | 7.604 (2.19), 7.608 (3.40), 7.614 (2.07), 7.620 (3.06), 7.628 (0.41), |
| | 7.846 (1.32), 8.174 (3.48), 8.188 (3.32), 10.739 (5.26). |
| 107 | 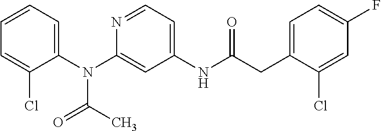 [SMILES: CC(=O)N(c1cc(NC(=O)Cc2ccc(F)cc2Cl)ccn1)c1ccccc1Cl] |
| | N-{4-[2-(2-chloro-4-fluorophenyl)acetamido]pyridin-2-yl}-N-(2- |
| | chlorophenyl)acetamide |
| | LC-MS (Method 2): Rₜ = 1.15 min; MS (ESIneg): m/z = 430 [M − H]⁻ |
| | ¹H-NMR (400 MHz, DMSO-d6) δ [ppm]: 1.993 (16.00), 2.518 (1.25), |
| | 2.523 (0.86), 3.345 (7.57), 7.188 (1.60), 7.195 (1.81), 7.210 (3.37), |
| | 7.216 (3.68), 7.231 (1.84), 7.238 (2.04), 7.403 (0.75), 7.414 (5.25), |
| | 7.421 (3.81), 7.427 (4.28), 7.432 (7.58), 7.439 (10.87), 7.446 (5.32), |
| | 7.450 (4.75), 7.456 (7.07), 7.461 (7.74), 7.465 (4.38), 7.470 (5.15), |
| | 7.475 (1.86), 7.482 (2.10), 7.487 (3.55), 7.493 (1.27), 7.589 (0.63), |
| | 7.597 (3.99), 7.606 (2.18), 7.608 (2.96), 7.615 (2.03), 7.621 (3.04), |
| | 7.629 (0.40), 7.841 (1.30), 8.172 (3.47), 8.186 (3.29), 10.736 (5.27). |
| 108 | 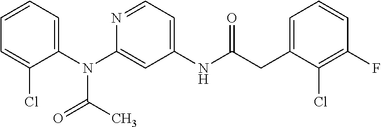 [SMILES: CC(=O)N(c1cc(NC(=O)Cc2cccc(F)c2Cl)ccn1)c1ccccc1Cl] |
| | N-{4-[2-(2-chloro-3-fluorophenyl)acetamido]pyridin-2-yl}-N-(2- |
| | chlorophenyl)acetamide |
| | LC-MS (Method 2): Rₜ = 1.14 min; MS (ESIneg): m/z = 430 [M − H]⁻ |
| | ¹H-NMR (400 MHz, DMSO-d6) δ [ppm]: 1.993 (16.00), 2.518 (1.04), |
| | 2.522 (0.65), 3.942 (15.18), 7.266 (1.64), 7.273 (2.42), 7.280 (1.80), |
| | 7.283 (1.97), 7.289 (2.68), 7.324 (0.96), 7.339 (4.05), 7.345 (5.95), |
| | 7.361 (8.37), 7.367 (2.93), 7.376 (2.69), 7.381 (0.83), 7.397 (0.71), |
| | 7.404 (0.76), 7.415 (5.18), 7.422 (3.69), 7.428 (4.31), 7.433 (4.41), |
| | 7.440 (6.92), 7.444 (4.71), 7.448 (4.37), 7.457 (4.41), 7.463 (4.38), |
| | 7.471 (3.00), 7.478 (1.59), 7.484 (1.85), 7.495 (1.20), 7.590 (0.60), |
| | 7.598 (4.07), 7.606 (2.30), 7.609 (3.28), 7.615 (2.09), 7.622 (3.18), |
| | 7.629 (0.42), 7.846 (1.27), 8.175 (3.46), 8.189 (3.28), 10.776 (5.17). |
| 109 | 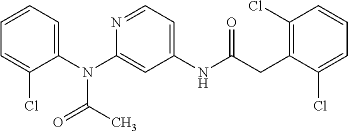 [SMILES: CC(=O)N(c1cc(NC(=O)Cc2c(Cl)cccc2Cl)ccn1)c1ccccc1Cl] |
| | N-(2-chlorophenyl)-N-{4-[2-(2,6-dichlorophenyl)acetamido]pyridin- |
| | 2-yl}acetamide |
| | LC-MS (Method 2): Rₜ = 1.18 min; MS (ESIneg): m/z = 446 [M − H]⁻ |
| | ¹H-NMR (400 MHz, DMSO-d6) δ [ppm]: 1.991 (15.63), 2.296 (0.47), |
| | 2.518 (0.95), 2.522 (0.62), 3.957 (0.44), 4.081 (15.81), 7.336 (3.32), |
| | 7.355 (4.21), 7.357 (4.66), 7.376 (5.34), 7.404 (0.80), 7.416 (5.20), |
| | 7.421 (6.28), 7.426 (6.60), 7.433 (5.86), 7.440 (10.22), 7.446 (1.02), |
| | 7.451 (1.40), 7.459 (0.51), 7.473 (2.95), 7.480 (1.85), 7.489 (16.00), |
| | 7.497 (1.53), 7.508 (9.06), 7.590 (0.60), 7.599 (3.93), 7.606 (2.24), |
| | 7.610 (3.32), 7.615 (2.18), 7.622 (3.10), 7.629 (0.42), 7.844 (1.21), |
| | 8.176 (3.37), 8.189 (3.19), 10.848 (5.18). |
| 110 | 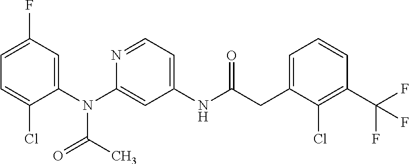 [SMILES: CC(=O)N(c1cc(NC(=O)Cc2cccc(C(F)(F)F)c2Cl)ccn1)c1cc(F)ccc1Cl] |
| | N-(2-chloro-5-fluorophenyl)-N-(4-{2-[2-chloro-3- |
| | (trifluoromethyl)phenyl]acetamido}pyridin-2-yl)acetamide |
| | LC-MS (Method 2): Rₜ = 1.27 min; MS (ESIneg): m/z = 498 [M − H]⁻ |
| | ¹H-NMR (400 MHz, DMSO-d6) δ [ppm]: 2.017 (16.00), 2.518 (1.52), |
| | 2.522 (0.99), 4.033 (14.28), 7.321 (1.52), 7.328 (1.84), 7.341 (2.00), |
| | 7.343 (2.17), 7.348 (2.31), 7.351 (2.58), 7.364 (1.73), 7.371 (2.02), |
| | 7.450 (3.78), 7.454 (3.76), 7.464 (3.80), 7.468 (3.77), 7.507 (1.64), |
| | 7.514 (1.69), 7.530 (3.41), 7.537 (1.76), 7.549 (4.07), 7.569 (2.31), |
| | 7.648 (3.69), 7.661 (3.83), 7.670 (3.68), 7.684 (3.41), 7.749 (3.24), |
| | 7.768 (2.53), 7.797 (3.44), 7.801 (3.12), 7.816 (3.00), 7.820 (2.64), |
| | 7.893 (1.07), 8.176 (3.29), 8.189 (3.09), 10.834 (5.60). |
| 111 | 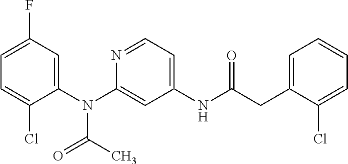 [SMILES: CC(=O)N(c1cc(NC(=O)Cc2ccccc2Cl)ccn1)c1cc(F)ccc1Cl] |
| | N-(2-chloro-5-fluorophenyl)-N-{4-[2-(2- |
| | chlorophenyl)acetamido]pyridin-2-yl}acetamide |
| | LC-MS (Method 1): Rₜ = 1.15 min; MS (ESIpos): m/z = 432 [M + H]⁺ |
| | ¹H-NMR (400 MHz, DMSO-d6) δ [ppm]: 2.023 (16.00), 2.518 (0.80), |
| | 2.522 (0.50), 3.337 (5.74), 7.294 (0.69), 7.297 (0.48), 7.305 (7.49), |
| | 7.312 (4.17), 7.315 (4.93), 7.318 (6.19), 7.321 (5.02), 7.328 (10.09), |
| | 7.340 (3.10), 7.347 (2.51), 7.348 (2.73), 7.361 (1.88), 7.369 (2.17), |
| | 7.396 (0.51), 7.405 (3.70), 7.411 (1.83), 7.416 (2.45), 7.420 (2.25), |
| | 7.428 (2.79), 7.439 (4.68), 7.447 (3.07), 7.452 (2.49), 7.455 (1.89), |
| | 7.464 (4.88), 7.469 (3.94), 7.478 (3.72), 7.483 (3.91), 7.502 (1.66), |
| | 7.510 (1.66), 7.525 (1.69), 7.532 (1.56), 7.646 (3.82), 7.660 (3.94), |
| | 7.668 (3.79), 7.682 (3.51), 7.883 (1.10), 8.174 (3.26), 8.188 (3.12), |
| | 10.754 (5.38). |
| 112 | 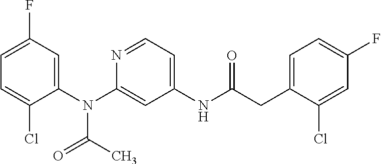 [SMILES: CC(=O)N(c1cc(NC(=O)Cc2ccc(F)cc2Cl)ccn1)c1cc(F)ccc1Cl] |
| | N-(2-chloro-5-fluorophenyl)-N-{4-[2-(2-chloro-4- |
| | fluorophenyl)acetamido]pyridin-2-yl}acetamide |
| | LC-MS (Method 1): Rₜ = 1.18 min; MS (ESIpos): m/z = 450 [M + H]⁺ |
| | ¹H-NMR (400 MHz, DMSO-d6) δ [ppm]: 2.021 (16.00), 2.518 (0.70), |
| | 2.522 (0.46), 3.342 (3.91), 7.190 (1.70), 7.197 (1.87), 7.211 (3.46), |
| | 7.218 (3.95), 7.232 (1.93), 7.239 (2.12), 7.319 (1.58), 7.326 (1.94), |
| | 7.339 (2.05), 7.341 (2.20), 7.347 (2.40), 7.349 (2.57), 7.361 (1.80), |
| | 7.369 (2.03), 7.433 (3.65), 7.439 (3.85), 7.455 (8.68), 7.461 (6.90), |
| | 7.469 (7.07), 7.475 (6.68), 7.491 (2.82), 7.502 (1.67), 7.509 (1.67), |
| | 7.525 (1.68), 7.532 (1.55), 7.646 (3.73), 7.660 (3.76), 7.668 (3.62), |
| | 7.682 (3.46), 7.880 (1.12), 8.173 (3.32), 8.187 (3.14), 10.751 (5.63). |
| 113 | 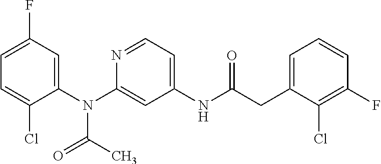 [SMILES: CC(=O)N(c1cc(NC(=O)Cc2cccc(F)c2Cl)ccn1)c1cc(F)ccc1Cl] |
| | N-(2-chloro-5-fluorophenyl)-N-{4-[2-(2-chloro-3- |
| | fluorophenyl)acetamido]pyridin-2-yl}acetamide |
| | LC-MS (Method 1): Rₜ = 1.17 min; MS (ESIneg): m/z = 448 [M − H]⁻ |
| | ¹H-NMR (400 MHz, DMSO-d6) δ [ppm]: 2.021 (16.00), 2.518 (1.23), |
| | 2.522 (0.78), 3.948 (15.79), 7.270 (1.74), 7.276 (2.55), 7.283 (1.90), |
| | 7.287 (2.09), 7.293 (2.88), 7.321 (1.90), 7.328 (2.23), 7.341 (6.24), |
| | 7.347 (7.54), 7.363 (9.87), 7.369 (3.98), 7.378 (2.86), 7.384 (0.92), |
| | 7.399 (0.51), 7.456 (3.88), 7.460 (4.11), 7.469 (4.06), 7.474 (4.23), |
| | 7.506 (1.63), 7.513 (1.63), 7.528 (1.67), 7.535 (1.54), 7.648 (3.79), |
| | 7.661 (3.82), 7.670 (3.61), 7.684 (3.48), 7.884 (1.08), 8.176 (3.32), |
| | 8.189 (3.14), 10.790 (5.55). |
| 114 | 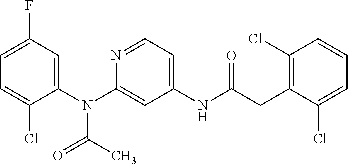 [SMILES: CC(=O)N(c1cc(NC(=O)Cc2c(Cl)cccc2Cl)ccn1)c1cc(F)ccc1Cl] |
| | N-(2-chloro-5-fluorophenyl)-N-{4-[2-(2,6- |
| | dichlorophenyl)acetamido]pyridin-2-yl}acetamide |
| | LC-MS (Method 1): Rₜ = 1.21 min; MS (ESIpos): m/z = 466 [M + H]⁺ |
| | ¹H-NMR (400 MHz, DMSO-d6) δ [ppm]: 2.018 (16.00), 2.073 (2.49), |
| | 2.518 (0.72), 2.522 (0.43), 4.088 (15.85), 7.319 (1.55), 7.328 (1.90), |
| | 7.336 (3.87), 7.340 (2.41), 7.342 (2.37), 7.347 (2.48), 7.350 (2.81), |
| | 7.355 (4.91), 7.358 (5.14), 7.362 (2.20), 7.370 (2.20), 7.377 (5.79), |
| | 7.435 (3.38), 7.439 (3.49), 7.449 (3.50), 7.453 (3.56), 7.489 (15.61), |
| | 7.509 (11.06), 7.530 (1.69), 7.537 (1.56), 7.647 (3.64), 7.661 (3.66), |
| | 7.670 (3.60), 7.684 (3.41), 7.884 (1.03), 8.135 (0.56), 8.176 (3.20), |
| | 8.190 (3.03), 10.863 (5.37). |
| 115 | 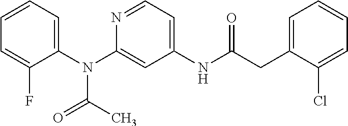 [SMILES: CC(=O)N(c1cc(NC(=O)Cc2ccccc2Cl)ccn1)c1ccccc1F] |
| | N-{4-[2-(2-chlorophenyl)acetamido]pyridin-2-yl}-N-(2- |
| | fluorophenyl)acetamide |
| | LC-MS (Method 2): Rₜ = 1.09 min; MS (ESIneg): m/z = 396 [M − H]⁻ |
| | ¹H-NMR (400 MHz, DMSO-d6) δ [ppm]: 2.031 (16.00), 2.518 (0.64), |
| | 2.522 (0.42), 3.335 (9.06), 7.229 (0.85), 7.232 (0.94), 7.248 (1.79), |
| | 7.251 (2.00), 7.266 (1.35), 7.270 (1.46), 7.292 (0.41), 7.302 (4.11), |
| | 7.312 (2.92), 7.316 (2.97), 7.318 (3.17), 7.321 (1.49), 7.326 (6.45), |
| | 7.336 (0.85), 7.342 (1.58), 7.346 (1.84), 7.350 (1.05), 7.357 (0.93), |
| | 7.361 (1.12), 7.367 (1.64), 7.371 (1.60), 7.376 (1.55), 7.380 (1.68), |
| | 7.390 (0.60), 7.399 (3.57), 7.409 (2.08), 7.413 (2.67), 7.422 (1.79), |
| | 7.426 (1.40), 7.433 (1.26), 7.437 (2.93), 7.445 (2.39), 7.452 (3.19), |
| | 7.457 (2.53), 7.459 (2.55), 7.467 (2.18), 7.471 (2.24), 7.757 (1.53), |
| | 8.215 (2.61), 8.229 (2.49), 10.753 (3.22). |
| 116 | 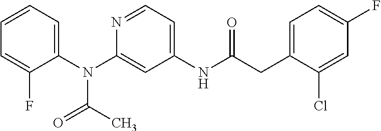 [SMILES: CC(=O)N(c1cc(NC(=O)Cc2ccc(F)cc2Cl)ccn1)c1ccccc1F] |
| | N-{4-[2-(2-chloro-4-fluorophenyl)acetamido]pyridin-2-yl}-N-(2- |
| | fluorophenyl)acetamide |
| | LC-MS (Method 2): Rₜ = 1.11 min; MS (ESIneg): m/z = 414 [M − H]⁻ |
| | ¹H-NMR (400 MHz, DMSO-d6) δ [ppm]: 2.030 (16.00), 2.518 (0.49), |
| | 3.338 (7.76), 7.187 (1.02), 7.194 (1.10), 7.209 (2.05), 7.215 (2.38), |
| | 7.230 (1.89), 7.232 (1.19), 7.237 (1.38), 7.249 (1.76), 7.252 (1.91), |
| | 7.267 (1.31), 7.270 (1.39), 7.322 (0.78), 7.325 (0.85), 7.343 (1.49), |
| | 7.347 (1.76), 7.351 (1.03), 7.357 (0.93), 7.361 (1.10), 7.368 (1.63), |
| | 7.372 (1.60), 7.376 (1.52), 7.380 (1.64), 7.395 (1.45), 7.400 (1.38), |
| | 7.409 (0.98), 7.413 (1.33), 7.416 (0.96), 7.420 (0.74), 7.426 (0.99), |
| | 7.431 (2.97), 7.438 (2.71), 7.445 (2.96), 7.449 (4.00), 7.453 (2.86), |
| | 7.459 (4.09), 7.464 (4.40), 7.469 (1.86), 7.485 (1.61), 7.753 (1.51), |
| | 8.214 (2.63), 8.228 (2.49), 10.751 (3.27). |
| 117 | 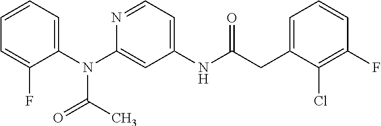 [SMILES: CC(=O)N(c1cc(NC(=O)Cc2cccc(F)c2Cl)ccn1)c1ccccc1F] |
| | N-{4-[2-(2-chloro-3-fluorophenyl)acetamido]pyridin-2-yl}-N-(2- |
| | fluorophenyl)acetamide |
| | LC-MS (Method 2): Rₜ = 1.10 min; MS (ESIneg): m/z = 414 [M − H]⁻ |
| | ¹H-NMR (400 MHz, DMSO-d6) δ [ppm]: 2.031 (16.00), 2.518 (0.65), |
| | 2.523 (0.41), 3.941 (9.77), 7.230 (0.88), 7.233 (0.96), 7.250 (1.93), |
| | 7.252 (2.08), 7.271 (3.03), 7.278 (1.25), 7.282 (1.35), 7.287 (1.85), |
| | 7.323 (1.28), 7.326 (1.10), 7.338 (2.56), 7.343 (5.08), 7.352 (1.44), |
| | 7.359 (6.46), 7.367 (2.81), 7.374 (2.52), 7.381 (1.98), 7.397 (1.47), |
| | 7.401 (1.36), 7.409 (1.06), 7.414 (1.35), 7.428 (0.90), 7.430 (0.92), |
| | 7.435 (0.93), 7.440 (0.57), 7.446 (2.21), 7.450 (2.28), 7.460 (2.09), |
| | 7.464 (2.12), 7.758 (1.60), 8.217 (2.68), 8.231 (2.55), 10.791 (3.36). |
| 118 | 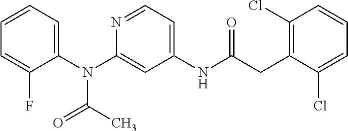 [SMILES: CC(=O)N(c1cc(NC(=O)Cc2c(Cl)cccc2Cl)ccn1)c1ccccc1F] |
| | N-{4-[2-(2,6-dichlorophenyl)acetamido]pyridin-2-yl}-N-(2- |
| | fluorophenyl)acetamide |
| | LC-MS (Method 2): Rₜ = 1.14 min; MS (ESIneg): m/z = 430 [M − H]⁻ |
| | ¹H-NMR (400 MHz, DMSO-d6) δ [ppm]: 2.028 (16.00), 2.518 (0.64), |
| | 2.522 (0.41), 4.080 (9.85), 7.230 (0.87), 7.233 (0.93), 7.250 (1.77), |
| | 7.252 (1.96), 7.268 (1.32), 7.271 (1.43), 7.322 (0.80), 7.326 (0.89), |
| | 7.335 (2.08), 7.343 (1.58), 7.347 (1.86), 7.354 (3.00), 7.357 (3.10), |
| | 7.361 (1.10), 7.369 (1.87), 7.376 (3.81), 7.381 (1.57), 7.385 (1.67), |
| | 7.396 (1.03), 7.400 (1.36), 7.405 (0.93), 7.409 (1.10), 7.414 (1.36), |
| | 7.417 (1.12), 7.422 (2.59), 7.427 (2.85), 7.436 (2.54), 7.441 (2.34), |
| | 7.448 (0.52), 7.487 (9.21), 7.507 (5.63), 7.752 (1.44), 8.217 (2.58), |
| | 8.231 (2.44), 10.865 (3.23). |
| 119 | 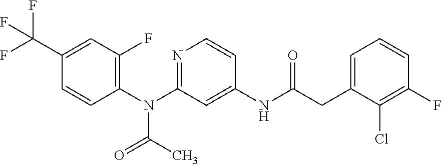 [SMILES: CC(=O)N(c1cc(NC(=O)Cc2cccc(F)c2Cl)ccn1)c1ccc(C(F)(F)F)cc1F] |
| | N-{4-[2-(2-chloro-3-fluorophenyl)acetamido]pyridin-2-yl}-N-[2- |
| | fluoro-4-(trifluoromethyl)phenyl]acetamide |
| | LC-MS (Method 2): Rₜ = 1.26 min; MS (ESIneg): m/z = 482 [M − H]⁻ |
| | ¹H-NMR (400 MHz, DMSO-d6) δ [ppm]: 2.080 (16.00), 2.523 (1.96), |
| | 3.948 (10.66), 7.267 (1.15), 7.274 (1.71), 7.280 (1.32), 7.284 (1.43), |
| | 7.290 (2.00), 7.327 (0.62), 7.341 (2.78), 7.346 (3.64), 7.362 (6.16), |
| | 7.369 (2.32), 7.377 (1.97), 7.383 (0.72), 7.473 (2.42), 7.478 (2.62), |
| | 7.488 (2.60), 7.492 (2.77), 7.611 (0.48), 7.632 (1.35), 7.650 (4.15), |
| | 7.654 (3.48), 7.671 (0.78), 7.676 (0.83), 7.776 (1.32), 7.870 (1.60), |
| | 7.873 (1.63), 7.896 (1.83), 8.239 (2.28), 8.253 (2.24), 10.827 (3.81). |
| 120 | 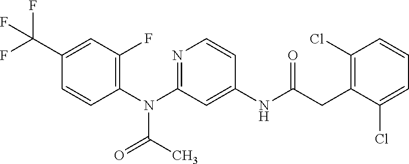 [SMILES: CC(=O)N(c1cc(NC(=O)Cc2c(Cl)cccc2Cl)ccn1)c1ccc(C(F)(F)F)cc1F] |
| | N-{4-[2-(2,6-dichlorophenyl)acetamido]pyridin-2-yl}-N-[2-fluoro-4- |
| | (trifluoromethyl)phenyl]acetamide |
| | LC-MS (Method 2): Rₜ = 1.30 min; MS (ESIneg): m/z = 498 [M − H]⁻ |
| | ¹H-NMR (400 MHz, DMSO-d6) δ [ppm]: 2.077 (16.00), 2.522 (3.26), |
| | 4.087 (11.22), 7.338 (2.23), 7.357 (2.98), 7.360 (3.36), 7.378 (3.65), |
| | 7.454 (2.35), 7.459 (2.55), 7.469 (2.54), 7.473 (2.72), 7.490 (10.30), |
| | 7.509 (6.32), 7.613 (0.46), 7.634 (1.39), 7.650 (4.73), 7.653 (3.96), |
| | 7.671 (0.78), 7.675 (0.83), 7.769 (1.25), 7.869 (1.63), 7.872 (1.67), |
| | 7.895 (1.91), 8.239 (2.24), 8.253 (2.23), 10.902 (3.89). |
| 121 | 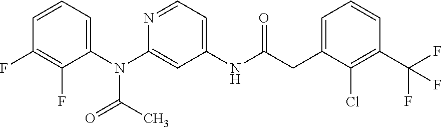 [SMILES: CC(=O)N(c1cc(NC(=O)Cc2cccc(C(F)(F)F)c2Cl)ccn1)c1cccc(F)c1F] |
| | N-(4-{2-[2-chloro-3-(trifluoromethyl)phenyl]acetamido}pyridin-2- |
| | yl)-N-(2,3-difluorophenyl)acetamide |
| | LC-MS (Method 2): Rₜ = 1.23 min; MS (ESIpos): m/z = 484 [M + H]⁺ |
| | ¹H-NMR (400 MHz, DMSO-d6) δ [ppm]: 2.065 (16.00), 2.518 (0.86), |
| | 2.522 (0.53), 4.032 (8.64), 7.195 (0.44), 7.215 (1.03), 7.230 (0.97), |
| | 7.237 (1.11), 7.249 (0.70), 7.253 (0.70), 7.257 (0.95), 7.260 (0.95), |
| | 7.270 (0.95), 7.274 (0.91), 7.445 (0.43), 7.450 (0.48), 7.459 (2.35), |
| | 7.464 (2.85), 7.474 (2.73), 7.478 (2.62), 7.490 (0.93), 7.494 (0.79), |
| | 7.511 (0.41), 7.515 (0.41), 7.528 (0.99), 7.547 (2.24), 7.566 (1.31), |
| | 7.746 (1.89), 7.765 (1.61), 7.784 (1.35), 7.796 (2.25), 7.799 (2.00), |
| | 7.816 (1.73), 7.820 (1.52), 8.240 (2.30), 8.254 (2.18), 10.860 (3.23). |
| 122 | 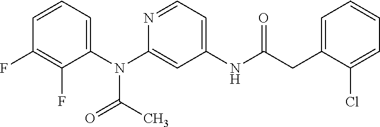 [SMILES: CC(=O)N(c1cc(NC(=O)Cc2ccccc2Cl)ccn1)c1cccc(F)c1F] |
| | N-{4-[2-(2-chlorophenyl)acetamido]pyridin-2-yl}-N-(2,3- |
| | difluorophenyl)acetamide |
| | LC-MS (Method 1): Rₜ = 1.12 min; MS (ESIpos): m/z = 416 [M + H]⁺ |
| | ¹H-NMR (400 MHz, DMSO-d6) 8 [ppm]: 2.069 (16.00), 3.339 (3.05), |
| | 7.192 (0.44), 7.212 (1.03), 7.227 (0.95), 7.236 (0.97), 7.248 (0.70), |
| | 7.252 (0.71), 7.256 (0.95), 7.259 (0.92), 7.268 (0.94), 7.273 (0.90), |
| | 7.279 (0.41), 7.289 (0.42), 7.293 (0.69), 7.304 (4.24), 7.312 (2.48), |
| | 7.314 (2.76), 7.317 (2.81), 7.319 (2.80), 7.328 (5.36), 7.336 (0.55), |
| | 7.338 (0.62), 7.402 (2.09), 7.409 (1.01), 7.412 (1.31), 7.417 (1.39), |
| | 7.425 (1.40), 7.438 (2.55), 7.447 (2.25), 7.450 (1.55), 7.453 (1.13), |
| | 7.461 (2.15), 7.469 (1.35), 7.474 (2.86), 7.478 (2.56), 7.488 (3.19), |
| | 7.492 (3.07), 7.509 (0.42), 7.775 (1.35), 8.239 (2.28), 8.253 (2.18), |
| | 10.778 (3.08). |
| 123 | 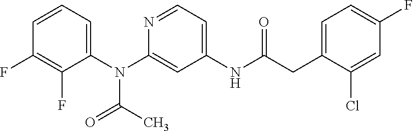 [SMILES: CC(=O)N(c1cc(NC(=O)Cc2ccc(F)cc2Cl)ccn1)c1cccc(F)c1F] |
| | N-{4-[2-(2-chloro-4-fluorophenyl)acetamido]pyridin-2-yl}-N-(2,3- |
| | difluorophenyl)acetamide |
| | LC-MS (Method 1): Rₜ = 1.14 min; MS (ESIpos): m/z = 434 [M + H]⁺ |
| | ¹H-NMR (400 MHz, DMSO-d6) δ [ppm]: 2.068 (16.00), 2.073 (3.48), |
| | 3.340 (1.70), 7.188 (1.25), 7.195 (1.47), 7.209 (2.88), 7.216 (3.04), |
| | 7.230 (1.90), 7.237 (2.13), 7.248 (0.72), 7.252 (0.70), 7.256 (0.96), |
| | 7.259 (0.94), 7.268 (0.95), 7.272 (0.90), 7.279 (0.40), 7.431 (2.18), |
| | 7.438 (2.20), 7.444 (0.63), 7.453 (2.98), 7.460 (2.56), 7.466 (4.61), |
| | 7.471 (3.94), 7.480 (2.63), 7.485 (2.97), 7.488 (2.76), 7.509 (0.41), |
| | 7.772 (1.34), 8.134 (0.40), 8.238 (2.27), 8.252 (2.17), 10.776 (3.11). |
| 124 | 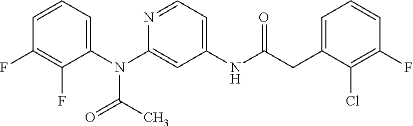 [SMILES: CC(=O)N(c1cc(NC(=O)Cc2cccc(F)c2Cl)ccn1)c1cccc(F)c1F] |
| | N-{4-[2-(2-chloro-3-fluorophenyl)acetamido]pyridin-2-yl}-N-(2,3- |
| | difluorophenyl)acetamide |
| | LC-MS (Method 1): Rₜ = 1.13 min; MS (ESIpos): m/z = 434 [M + H]⁺ |
| | ¹H-NMR (400 MHz, DMSO-d6) δ [ppm]: 2.069 (16.00), 2.073 (4.84), |
| | 3.949 (9.25), 7.195 (0.45), 7.215 (1.04), 7.229 (0.99), 7.237 (1.15), |
| | 7.249 (0.76), 7.252 (0.76), 7.257 (1.01), 7.259 (1.02), 7.269 (1.57), |
| | 7.273 (2.34), 7.281 (1.44), 7.285 (1.32), 7.290 (2.01), 7.323 (0.56), |
| | 7.338 (2.33), 7.344 (3.64), 7.360 (4.70), 7.366 (1.84), 7.375 (1.58), |
| | 7.381 (0.50), 7.444 (0.43), 7.449 (0.45), 7.468 (3.10), 7.473 (2.88), |
| | 7.482 (2.74), 7.487 (2.97), 7.509 (0.42), 7.777 (1.35), 8.241 (2.31), |
| | 8.255 (2.20), 10.817 (3.16). |
| 125 | 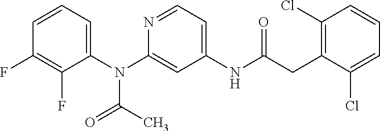 [SMILES: CC(=O)N(c1cc(NC(=O)Cc2c(Cl)cccc2Cl)ccn1)c1cccc(F)c1F] |
| | N-{4-[2-(2,6-dichlorophenyl)acetamido]pyridin-2-yl}-N-(2,3- |
| | difluorophenyl)acetamide |
| | LC-MS (Method 1): Rₜ = 1.17 min; MS (ESIpos): m/z = 450 [M + H]⁺ |
| | ¹H-NMR (400 MHz, DMSO-d6) δ [ppm]: 2.066 (16.00), 2.073 (1.96), |
| | 2.518 (0.41), 3.341 (0.54), 4.087 (9.39), 7.197 (0.44), 7.217 (1.04), |
| | 7.231 (1.03), 7.237 (1.44), 7.249 (0.75), 7.252 (0.75), 7.256 (0.97), |
| | 7.259 (0.96), 7.270 (0.94), 7.273 (0.96), 7.293 (0.47), 7.336 (2.03), |
| | 7.354 (2.64), 7.357 (2.91), 7.376 (3.23), 7.446 (2.23), 7.451 (2.16), |
| | 7.461 (2.27), 7.465 (2.59), 7.473 (0.74), 7.488 (9.93), 7.507 (5.75), |
| | 7.770 (1.25), 8.241 (2.21), 8.255 (2.09), 10.895 (3.02). |
| 126 | 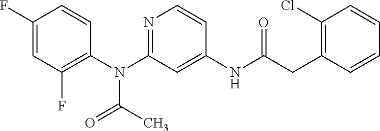 [SMILES: CC(=O)N(c1cc(NC(=O)Cc2ccccc2Cl)ccn1)c1ccc(F)cc1F] |
| | N-{4-[2-(2-chlorophenyl)acetamido]pyridin-2-yl}-N-(2,4- |
| | difluorophenyl)acetamide |
| | LC-MS (Method 1): Rₜ = 1.14 min; MS (ESIpos): m/z = 416 [M + H]⁺ |
| | ¹H-NMR (400 MHz, DMSO-d6) δ [ppm]: 2.032 (16.00), 2.074 (0.57), |
| | 2.332 (0.87), 2.518 (3.87), 2.522 (2.48), 2.673 (0.88), 3.880 (10.71), |
| | 7.128 (0.54), 7.132 (0.64), 7.135 (0.61), 7.148 (1.08), 7.154 (1.14), |
| | 7.171 (0.63), 7.174 (0.67), 7.178 (0.64), 7.295 (0.40), 7.305 (4.71), |
| | 7.315 (2.98), 7.319 (2.93), 7.321 (3.02), 7.329 (5.97), 7.339 (0.68), |
| | 7.401 (2.78), 7.410 (1.83), 7.416 (1.62), 7.425 (2.42), 7.428 (1.74), |
| | 7.432 (1.46), 7.435 (1.54), 7.438 (3.08), 7.447 (2.83), 7.450 (4.14), |
| | 7.455 (4.09), 7.464 (4.07), 7.469 (3.10), 7.480 (1.23), 7.487 (0.68), |
| | 7.502 (0.56), 7.782 (1.42), 8.209 (2.61), 8.223 (2.44), 10.759 (3.35). |
| 127 | 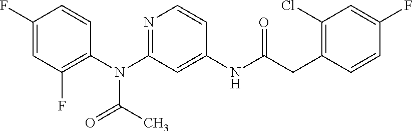 [SMILES: CC(=O)N(c1cc(NC(=O)Cc2ccc(F)cc2Cl)ccn1)c1ccc(F)cc1F] |
| | N-{4-[2-(2-chloro-4-fluorophenyl)acetamido]pyridin-2-yl}-N-(2,4- |
| | difluorophenyl)acetamide |
| | LC-MS (Method 1): Rₜ = 1.17 min; MS (ESIpos): m/z = 434 [M + H]⁺ |
| | ¹H-NMR (400 MHz, DMSO-d6) δ [ppm]: 2.031 (16.00), 2.074 (0.90), |
| | 2.332 (0.62), 2.518 (2.96), 2.522 (1.87), 2.673 (0.63), 3.872 (8.66), |
| | 7.129 (0.54), 7.133 (0.63), 7.136 (0.60), 7.149 (1.08), 7.155 (1.15), |
| | 7.169 (0.62), 7.171 (0.64), 7.176 (0.68), 7.179 (0.66), 7.190 (1.04), |
| | 7.197 (1.14), 7.211 (2.15), 7.218 (2.47), 7.233 (1.20), 7.240 (1.25), |
| | 7.404 (0.92), 7.411 (0.99), 7.426 (1.23), 7.429 (1.34), 7.434 (3.32), |
| | 7.442 (5.11), 7.447 (3.32), 7.450 (3.01), 7.456 (5.71), 7.461 (4.26), |
| | 7.463 (4.34), 7.471 (2.10), 7.480 (1.33), 7.487 (2.23), 7.502 (0.58), |
| | 7.779 (1.41), 8.208 (2.58), 8.222 (2.47), 10.758 (3.39). |
| 128 | 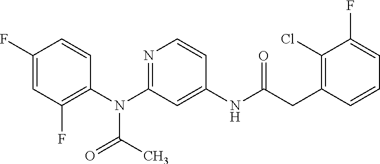 [SMILES: CC(=O)N(c1cc(NC(=O)Cc2cccc(F)c2Cl)ccn1)c1ccc(F)cc1F] |
| | N-{4-[2-(2-chloro-3-fluorophenyl)acetamido]pyridin-2-yl}-N-(2,4- |
| | difluorophenyl)acetamide |
| | LC-MS (Method 1): Rₜ = 1.15 min; MS (ESIpos): m/z = 434 [M + H]⁺ |
| | ¹H-NMR (400 MHz, DMSO-d6) δ [ppm]: 2.031 (16.00), 2.074 (0.56), |
| | 2.518 (2.20), 2.522 (1.42), 2.673 (0.45), 3.944 (10.16), 7.129 (0.56), |
| | 7.133 (0.63), 7.137 (0.61), 7.150 (1.09), 7.155 (1.16), 7.169 (0.62), |
| | 7.173 (0.63), 7.176 (0.70), 7.180 (0.66), 7.266 (1.07), 7.273 (1.61), |
| | 7.280 (1.15), 7.284 (1.27), 7.290 (1.74), 7.327 (0.59), 7.341 (2.59), |
| | 7.347 (3.50), 7.362 (5.86), 7.369 (1.96), 7.378 (1.80), 7.383 (0.54), |
| | 7.404 (0.92), 7.411 (0.96), 7.426 (1.17), 7.430 (1.21), 7.433 (1.27), |
| | 7.437 (1.25), 7.444 (3.27), 7.448 (2.90), 7.452 (1.38), 7.457 (3.41), |
| | 7.463 (3.31), 7.466 (1.42), 7.482 (1.23), 7.489 (0.68), 7.504 (0.55), |
| | 7.784 (1.40), 8.212 (2.64), 8.226 (2.51), 10.798 (3.39). |
| 129 | 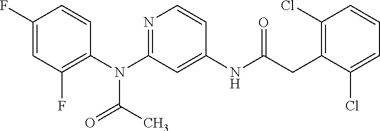 [SMILES: CC(=O)N(c1cc(NC(=O)Cc2c(Cl)cccc2Cl)ccn1)c1ccc(F)cc1F] |
| | N-{4-[2-(2,6-dichlorophenyl)acetamido]pyridin-2-yl}-N-(2,4- |
| | difluorophenyl)acetamide |
| | LC-MS (Method 1): Rₜ = 1.20 min; MS (ESIpos): m/z = 450 [M + H]⁺ |
| | ¹H-NMR (400 MHz, DMSO-d6) δ [ppm]: 1.867 (0.44), 2.029 (16.00), |
| | 2.074 (0.80), 2.332 (0.56), 2.518 (2.76), 2.522 (1.74), 2.673 (0.58), |
| | 4.083 (10.68), 7.126 (0.53), 7.129 (0.56), 7.133 (0.63), 7.136 (0.62), |
| | 7.150 (1.11), 7.155 (1.16), 7.169 (0.63), 7.172 (0.64), 7.176 (0.70), |
| | 7.179 (0.64), 7.338 (2.23), 7.357 (2.82), 7.360 (3.14), 7.379 (3.43), |
| | 7.403 (0.92), 7.411 (1.00), 7.421 (2.74), 7.426 (3.61), 7.435 (3.40), |
| | 7.440 (2.88), 7.447 (0.83), 7.452 (1.16), 7.459 (1.21), 7.463 (0.87), |
| | 7.469 (1.26), 7.490 (10.42), 7.510 (6.18), 7.779 (1.33), 8.212 (2.60), |
| | 8.226 (2.44), 10.872 (3.40). |
| 130 | 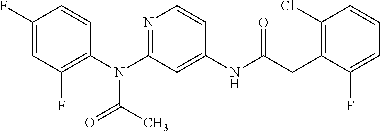 [SMILES: CC(=O)N(c1cc(NC(=O)Cc2c(F)cccc2Cl)ccn1)c1ccc(F)cc1F] |
| | N-{4-[2-(2-chloro-6-fluorophenyl)acetamido]pyridin-2-yl}-N-(2,4- |
| | difluorophenyl)acetamide |
| | LC-MS (Method 1): Rₜ = 1.15 min; MS (ESIpos): m/z = 434 [M + H]⁺ |
| | ¹H-NMR (400 MHz, DMSO-d6) δ [ppm]: 2.030 (16.00), 2.518 (2.62), |
| | 2.522 (1.68), 3.927 (6.11), 3.930 (6.01), 7.125 (0.52), 7.128 (0.55), |
| | 7.132 (0.66), 7.135 (0.61), 7.148 (1.09), 7.154 (1.18), 7.168 (0.63), |
| | 7.171 (0.64), 7.175 (0.69), 7.178 (0.65), 7.230 (0.90), 7.235 (0.90), |
| | 7.249 (1.29), 7.254 (2.09), 7.257 (1.16), 7.273 (1.32), 7.277 (1.25), |
| | 7.341 (0.94), 7.345 (1.21), 7.361 (4.99), 7.366 (4.60), 7.380 (1.87), |
| | 7.385 (1.73), 7.400 (2.24), 7.405 (0.88), 7.409 (1.05), 7.420 (0.95), |
| | 7.425 (3.78), 7.430 (3.32), 7.435 (1.46), 7.439 (2.83), 7.444 (3.24), |
| | 7.450 (1.19), 7.458 (1.28), 7.468 (1.23), 7.483 (1.20), 7.489 (0.67), |
| | 7.505 (0.54), 7.780 (1.38), 8.213 (2.61), 8.227 (2.47), 10.860 (3.34). |
| 131 | 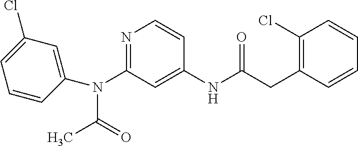 [SMILES: CC(=O)N(c1cccc(Cl)c1)c1cc(NC(=O)Cc2ccccc2Cl)ccn1] |
| | N-(3-chlorophenyl)-N-{4-[2-(2-chlorophenyl)acetamido]pyridin-2- |
| | yl}acetamide |
| | LC-MS (Method 2): Rₜ = 1.12 min; MS (ESIpos): m/z = 414 [M + H]⁺ |
| | ¹H-NMR (400 MHz, DMSO-d6) δ [ppm]: 2.005 (16.00), 2.518 (4.97), |
| | 2.522 (3.57), 3.877 (7.48), 7.197 (0.69), 7.201 (0.99), 7.205 (0.85), |
| | 7.217 (0.88), 7.221 (1.26), 7.224 (1.00), 7.301 (3.10), 7.310 (2.38), |
| | 7.316 (2.58), 7.324 (4.42), 7.334 (0.73), 7.363 (0.53), 7.366 (0.81), |
| | 7.371 (0.82), 7.383 (1.44), 7.388 (2.20), 7.391 (1.73), 7.395 (1.90), |
| | 7.403 (1.05), 7.408 (3.22), 7.417 (2.32), 7.422 (3.14), 7.427 (4.17), |
| | 7.434 (2.24), 7.443 (1.86), 7.448 (1.95), 7.458 (1.46), 7.490 (1.66), |
| | 7.495 (1.74), 7.504 (1.67), 7.509 (1.88), 7.667 (1.66), 8.290 (2.11), |
| | 8.304 (2.07), 10.771 (2.33). |
| 132 | 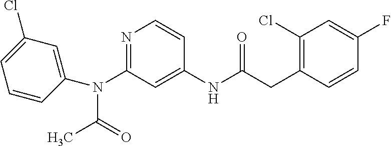 [SMILES: CC(=O)N(c1cccc(Cl)c1)c1cc(NC(=O)Cc2ccc(F)cc2Cl)ccn1] |
| | N-{4-[2-(2-chloro-4-fluorophenyl)acetamido]pyridin-2-yl}-N-(3- |
| | chlorophenyl)acetamide |
| | LC-MS (Method 2): Rₜ = 1.15 min; MS (ESIpos): m/z = 432 [M + H]⁺ |
| | ¹H-NMR (400 MHz, DMSO-d6) δ [ppm]: 2.003 (16.00), 2.518 (3.83), |
| | 2.522 (2.43), 3.867 (6.87), 7.186 (0.75), 7.193 (0.96), 7.197 (0.88), |
| | 7.201 (1.22), 7.207 (2.04), 7.214 (2.14), 7.220 (1.54), 7.224 (1.17), |
| | 7.228 (1.10), 7.235 (1.04), 7.363 (0.51), 7.368 (0.83), 7.372 (0.78), |
| | 7.384 (1.40), 7.388 (2.15), 7.392 (1.62), 7.408 (2.58), 7.416 (1.85), |
| | 7.421 (2.98), 7.427 (3.76), 7.437 (1.90), 7.445 (1.74), 7.447 (1.60), |
| | 7.453 (1.86), 7.459 (3.01), 7.466 (1.55), 7.481 (2.91), 7.486 (1.87), |
| | 7.495 (1.71), 7.500 (1.87), 7.663 (1.85), 8.289 (2.32), 8.303 (2.22), |
| | 10.770 (2.65). |
| 133 | 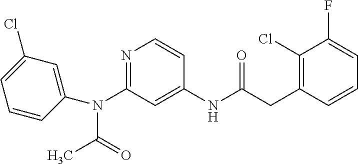 [SMILES: CC(=O)N(c1cccc(Cl)c1)c1cc(NC(=O)Cc2cccc(F)c2Cl)ccn1] |
| | N-{4-[2-(2-chloro-3-fluorophenyl)acetamido]pyridin-2-yl}-N-(3- |
| | chlorophenyl)acetamide |
| | LC-MS (Method 2): Rₜ = 1.13 min; MS (ESIpos): m/z = 432 [M + H]⁺ |
| | ¹H-NMR (400 MHz, DMSO-d6) δ [ppm]: 2.004 (16.00), 2.522 (6.38), |
| | 3.939 (7.00), 7.199 (0.68), 7.202 (1.00), 7.207 (0.82), 7.222 (1.26), |
| | 7.226 (0.96), 7.260 (0.75), 7.267 (1.16), 7.274 (0.85), 7.277 (0.91), |
| | 7.284 (1.31), 7.324 (0.41), 7.338 (1.71), 7.342 (2.12), 7.358 (4.39), |
| | 7.367 (1.86), 7.373 (1.92), 7.379 (0.63), 7.385 (1.44), 7.389 (2.13), |
| | 7.393 (1.69), 7.409 (2.45), 7.418 (1.67), 7.423 (2.81), 7.428 (3.86), |
| | 7.448 (1.00), 7.483 (1.61), 7.488 (1.66), 7.497 (1.64), 7.502 (1.82), |
| | 7.666 (1.64), 8.292 (2.09), 8.306 (2.05), 10.810 (2.33). |
| 134 | 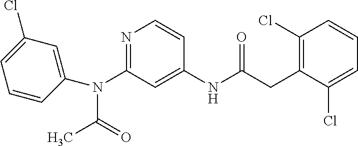 [SMILES: CC(=O)N(c1cccc(Cl)c1)c1cc(NC(=O)Cc2c(Cl)cccc2Cl)ccn1] |
| | N-(3-chlorophenyl)-N-{4-[2-(2,6-dichlorophenyl)acetamido]pyridin- |
| | 2-yl}acetamide |
| | LC-MS (Method 2): Rₜ = 1.17 min; MS (ESIneg): m/z = 446 [M − H]⁻ |
| | ¹H-NMR (400 MHz, DMSO-d6) δ [ppm]: 1.868 (0.85), 1.952 (0.68), |
| | 2.002 (16.00), 2.518 (1.24), 2.523 (0.90), 4.079 (7.43), 7.202 (0.72), |
| | 7.206 (0.99), 7.211 (0.81), 7.226 (1.19), 7.230 (0.87), 7.335 (1.57), |
| | 7.353 (2.04), 7.356 (2.27), 7.364 (0.58), 7.367 (0.85), 7.374 (2.63), |
| | 7.384 (1.48), 7.386 (1.29), 7.388 (2.23), 7.392 (1.47), 7.409 (2.43), |
| | 7.421 (1.67), 7.427 (5.32), 7.431 (1.50), 7.447 (0.97), 7.459 (1.62), |
| | 7.464 (1.71), 7.473 (1.66), 7.478 (1.84), 7.485 (6.72), 7.505 (4.06), |
| | 7.663 (1.52), 8.293 (2.11), 8.307 (2.01), 10.886 (2.30). |
| 135 | 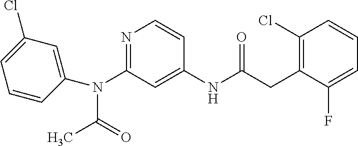 [SMILES: CC(=O)N(c1cccc(Cl)c1)c1cc(NC(=O)Cc2c(F)cccc2Cl)ccn1] |
| | N-{4-[2-(2-chloro-6-fluorophenyl)acetamido]pyridin-2-yl}-N-(3- |
| | chlorophenyl)acetamide |
| | LC-MS (Method 2): Rₜ = 1.13 min; MS (ESIpos): m/z = 432 [M + H]⁺ |
| | ¹H-NMR (400 MHz, DMSO-d6) δ [ppm]: 2.003 (16.00), 2.323 (0.68), |
| | 2.327 (0.80), 2.665 (0.72), 2.669 (0.81), 3.922 (7.00), 7.203 (1.55), |
| | 7.223 (2.30), 7.245 (1.62), 7.249 (1.91), 7.268 (1.34), 7.337 (1.00), |
| | 7.341 (0.97), 7.357 (4.50), 7.362 (3.98), 7.372 (1.61), 7.377 (2.21), |
| | 7.383 (3.20), 7.387 (2.92), 7.391 (2.35), 7.396 (1.85), 7.403 (1.74), |
| | 7.407 (2.72), 7.425 (5.20), 7.447 (1.12), 7.463 (2.25), 7.468 (1.80), |
| | 7.478 (2.36), 7.482 (1.82), 7.664 (2.71), 8.293 (2.50), 8.307 (2.43), |
| | 10.874 (3.31). |
| 136 | 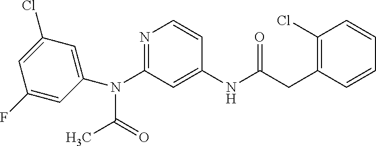 [SMILES: CC(=O)N(c1cc(F)cc(Cl)c1)c1cc(NC(=O)Cc2ccccc2Cl)ccn1] |
| | N-(3-chloro-5-fluorophenyl)-N-{4-[2-(2- |
| | chlorophenyl)acetamido]pyridin-2-yl}acetamide |
| | LC-MS (Method 1): Rₜ = 1.23 min; MS (ESIpos): m/z = 432 [M + H]⁺ |
| | ¹H-NMR (400 MHz, DMSO-d6) δ [ppm]: 2.018 (16.00), 2.332 (0.67), |
| | 2.518 (3.33), 2.522 (2.15), 2.673 (0.68), 3.884 (6.78), 7.211 (0.48), |
| | 7.216 (0.84), 7.221 (0.64), 7.240 (0.88), 7.245 (0.81), 7.256 (1.75), |
| | 7.303 (2.78), 7.312 (1.96), 7.318 (2.02), 7.326 (3.91), 7.337 (0.49), |
| | 7.399 (1.80), 7.407 (1.38), 7.414 (1.11), 7.419 (0.97), 7.423 (1.88), |
| | 7.430 (0.89), 7.437 (1.74), 7.445 (1.45), 7.450 (0.99), 7.460 (1.20), |
| | 7.510 (1.41), 7.515 (1.55), 7.525 (1.48), 7.529 (1.58), 7.697 (1.45), |
| | 8.304 (1.83), 8.317 (1.75), 10.796 (2.08). |
| 137 | 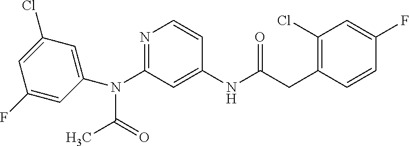 [SMILES: CC(=O)N(c1cc(F)cc(Cl)c1)c1cc(NC(=O)Cc2ccc(F)cc2Cl)ccn1] |
| | N-(3-chloro-5-fluorophenyl)-N-{4-[2-(2-chloro-4- |
| | fluorophenyl)acetamido]pyridin-2-yl}acetamide |
| | LC-MS (Method 2): Rₜ = 1.20 min; MS (ESIpos): m/z = 450 [M + H]⁺ |
| | ¹H-NMR (400 MHz, DMSO-d6) δ [ppm]: 1.161 (0.60), 1.179 (1.29), |
| | 1.197 (0.62), 2.017 (16.00), 2.518 (6.09), 2.523 (4.28), 3.792 (0.84), |
| | 3.875 (6.20), 4.084 (0.51), 4.102 (0.51), 7.189 (0.72), 7.195 (0.82), |
| | 7.210 (1.90), 7.217 (2.66), 7.227 (0.48), 7.231 (1.11), 7.238 (1.62), |
| | 7.246 (1.03), 7.256 (2.03), 7.399 (0.80), 7.405 (1.28), 7.410 (0.88), |
| | 7.421 (0.88), 7.426 (1.36), 7.433 (1.90), 7.440 (1.73), 7.448 (1.66), |
| | 7.455 (1.71), 7.462 (2.08), 7.470 (1.62), 7.486 (1.28), 7.502 (1.60), |
| | 7.507 (1.74), 7.516 (1.67), 7.521 (1.76), 7.697 (1.61), 8.303 (1.97), |
| | 8.317 (1.93), 10.795 (2.35). |
| 138 | 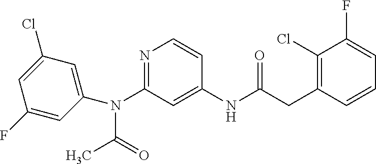 [SMILES: CC(=O)N(c1cc(F)cc(Cl)c1)c1cc(NC(=O)Cc2cccc(F)c2Cl)ccn1] |
| | N-(3-chloro-5-fluorophenyl)-N-{4-[2-(2-chloro-3- |
| | fluorophenyl)acetamido]pyridin-2-yl}acetamide |
| | LC-MS (Method 1): Rₜ = 1.23 min; MS (ESIpos): m/z = 450 [M + H]⁺ |
| | ¹H-NMR (400 MHz, DMSO-d6) δ [ppm]: 2.017 (16.00), 2.332 (0.44), |
| | 2.518 (2.54), 2.522 (1.61), 2.673 (0.44), 3.947 (6.22), 7.212 (0.48), |
| | 7.218 (0.83), 7.223 (0.64), 7.242 (0.87), 7.247 (0.83), 7.257 (1.76), |
| | 7.263 (1.44), 7.271 (1.07), 7.278 (0.75), 7.282 (0.78), 7.288 (1.08), |
| | 7.339 (1.58), 7.344 (2.07), 7.361 (3.72), 7.367 (1.20), 7.376 (1.10), |
| | 7.399 (0.75), 7.404 (1.15), 7.409 (0.80), 7.421 (0.76), 7.426 (1.15), |
| | 7.431 (0.73), 7.503 (1.38), 7.508 (1.51), 7.517 (1.46), 7.521 (1.52), |
| | 7.697 (1.39), 8.306 (1.79), 8.320 (1.71), 10.834 (2.05). |
| 139 | 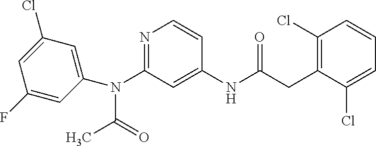 [SMILES: CC(=O)N(c1cc(F)cc(Cl)c1)c1cc(NC(=O)Cc2c(Cl)cccc2Cl)ccn1] |
| | N-(3-chloro-5-fluorophenyl)-N-{4-[2-(2,6- |
| | dichlorophenyl)acetamido]pyridin-2-yl}acetamide |
| | LC-MS (Method 1): Rₜ = 1.28 min; MS (ESIneg): m/z = 466 [M − H]⁻ |
| | ¹H-NMR (400 MHz, DMSO-d6) δ [ppm]: 1.147 (0.52), 1.162 (0.50), |
| | 2.016 (16.00), 2.332 (0.70), 2.518 (3.80), 2.522 (2.48), 2.673 (0.72), |
| | 4.086 (6.88), 7.221 (0.87), 7.226 (0.66), 7.245 (0.89), 7.250 (0.77), |
| | 7.263 (1.78), 7.336 (1.46), 7.355 (1.81), 7.358 (2.07), 7.377 (2.24), |
| | 7.397 (0.79), 7.403 (1.23), 7.408 (0.84), 7.419 (0.80), 7.425 (1.23), |
| | 7.430 (0.98), 7.480 (1.62), 7.485 (2.06), 7.488 (6.68), 7.494 (1.67), |
| | 7.499 (1.70), 7.508 (3.98), 7.695 (1.42), 8.306 (1.85), 8.321 (1.76), |
| | 10.913 (2.10). |
| 140 | 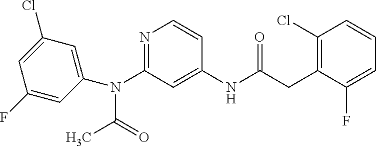 [SMILES: CC(=O)N(c1cc(F)cc(Cl)c1)c1cc(NC(=O)Cc2c(F)cccc2Cl)ccn1] |
| | N-(3-chloro-5-fluorophenyl)-N-{4-[2-(2-chloro-6- |
| | fluorophenyl)acetamido]pyridin-2-yl}acetamide |
| | LC-MS (Method 1): Rₜ = 1.25 min; MS (ESIpos): m/z = 444 [M + H]⁺ |
| | ¹H-NMR (400 MHz, DMSO-d6) δ [ppm]: 0.805 (6.68), 0.823 (16.00), |
| | 2.518 (10.40), 2.523 (7.20), 3.930 (4.08), 3.934 (4.13), 7.219 (0.91), |
| | 7.224 (0.76), 7.228 (0.81), 7.233 (0.82), 7.243 (1.09), 7.247 (1.56), |
| | 7.252 (2.00), 7.255 (2.05), 7.260 (2.15), 7.271 (1.13), 7.275 (1.06), |
| | 7.339 (0.63), 7.343 (0.80), 7.360 (3.23), 7.365 (2.97), 7.379 (1.32), |
| | 7.384 (1.29), 7.398 (1.85), 7.403 (1.69), 7.408 (0.97), 7.419 (1.25), |
| | 7.425 (1.31), 7.430 (0.87), 7.484 (1.48), 7.488 (1.60), 7.498 (1.57), |
| | 7.502 (1.64), 7.700 (1.52), 8.308 (1.89), 8.321 (1.85), 10.900 (2.20). |
| 141 | 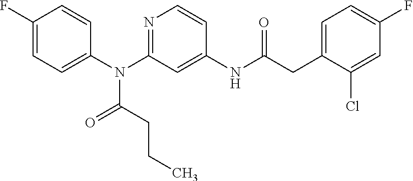 [SMILES: CCCC(=O)N(c1ccc(F)cc1)c1cc(NC(=O)Cc2ccc(F)cc2Cl)ccn1] |
| | N-{4-[2-(2-chloro-4-fluorophenyl)acetamido]pyridin-2-yl}-N-(4- |
| | fluorophenyl)butanamide |
| | LC-MS (Method 2): Rₜ = 1.18 min; MS (ESIpos): m/z = 450 [M + H]⁺ |
| | ¹H-NMR (400 MHz, DMSO-d6) δ [ppm]: 1.182 (0.74), 2.016 (16.00), |
| | 0.842 (7.54), 1.136 (1.19), 1.153 (1.22), 1.485 (0.50), 1.503 (2.27), |
| | 1.522 (4.31), 1.540 (4.42), 1.558 (2.27), 1.577 (0.46), 2.189 (3.54), |
| | 2.208 (6.36), 2.226 (3.22), 2.518 (2.12), 2.522 (1.30), 3.865 (10.61), |
| | 7.186 (1.20), 7.193 (1.37), 7.208 (2.88), 7.211 (3.35), 7.214 (3.41), |
| | 7.229 (2.66), 7.234 (6.97), 7.249 (1.32), 7.255 (4.68), 7.263 (0.65), |
| | 7.286 (0.53), 7.294 (3.96), 7.300 (1.68), 7.307 (4.30), 7.317 (2.71), |
| | 7.324 (1.09), 7.330 (2.31), 7.430 (2.68), 7.437 (3.04), 7.441 (3.48), |
| | 7.445 (5.07), 7.453 (3.58), 7.455 (3.82), 7.460 (6.96), 7.466 (2.33), |
| | 7.482 (1.95), 7.685 (3.23), 7.688 (3.14), 8.255 (3.88), 8.270 (3.70), |
| | 10.745 (4.18). |
| 142 | 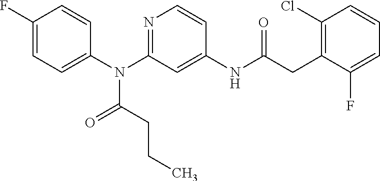 [SMILES: CCCC(=O)N(c1ccc(F)cc1)c1cc(NC(=O)Cc2c(F)cccc2Cl)ccn1] |
| | N-{4-[2-(2-chloro-6-fluorophenyl)acetamido]pyridin-2-yl}-N-(4- |
| | fluorophenyl)butanamide |
| | LC-MS (Method 1): Rₜ = 1.24 min; MS (ESIpos): m/z = 444 [M + H]⁺ |
| | ¹H-NMR (400 MHz, DMSO-d6) δ [ppm]: 0.804 (7.10), 0.823 (16.00), |
| | 0.842 (7.69), 1.147 (0.70), 1.163 (0.77), 1.484 (0.52), 1.503 (2.41), |
| | 1.521 (4.63), 1.539 (4.71), 1.558 (2.42), 1.576 (0.50), 2.188 (3.80), |
| | 2.206 (6.82), 2.225 (3.43), 2.518 (3.07), 2.522 (1.96), 3.920 (7.94), |
| | 3.924 (7.57), 7.210 (2.89), 7.216 (1.13), 7.227 (2.46), 7.233 (7.57), |
| | 7.238 (1.61), 7.245 (2.05), 7.250 (3.73), 7.255 (5.64), 7.262 (0.77), |
| | 7.269 (1.63), 7.273 (1.61), 7.298 (4.28), 7.303 (1.98), 7.310 (4.49), |
| | 7.320 (2.96), 7.327 (1.20), 7.333 (2.57), 7.338 (1.46), 7.341 (1.72), |
| | 7.358 (5.94), 7.363 (5.24), 7.377 (2.29), 7.382 (2.07), 7.397 (2.29), |
| | 7.402 (0.75), 7.420 (3.11), 7.424 (2.88), 7.434 (2.97), 7.439 (3.01), |
| | 7.690 (3.37), 7.694 (3.25), 8.260 (4.13), 8.274 (3.94), 10.849 (4.35). |
| 143 | 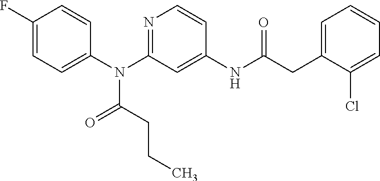 [SMILES: CCCC(=O)N(c1ccc(F)cc1)c1cc(NC(=O)Cc2ccccc2Cl)ccn1] |
| | N-{4-[2-(2-chlorophenyl)acetamido]pyridin-2-yl}-N-(4- |
| | fluorophenyl)butanamide |
| | LC-MS (Method 1): Rₜ = 1.23 min; MS (ESIpos): m/z = 426 [M + H]⁺ |
| | ¹H-NMR (400 MHz, DMSO-d6) δ [ppm]: 0.806 (6.70), 0.824 (16.00), |
| | 0.842 (7.55), 1.130 (1.28), 1.146 (1.29), 1.486 (0.50), 1.505 (2.23), |
| | 1.522 (4.28), 1.541 (4.36), 1.560 (2.24), 1.578 (0.46), 2.191 (3.61), |
| | 2.209 (6.45), 2.228 (3.19), 2.518 (2.03), 2.522 (1.25), 3.711 (0.42), |
| | 3.875 (12.49), 7.210 (2.65), 7.217 (1.01), 7.228 (1.50), 7.233 (6.60), |
| | 7.238 (1.58), 7.241 (0.88), 7.249 (1.56), 7.255 (4.84), 7.263 (0.85), |
| | 7.267 (0.49), 7.287 (0.65), 7.295 (4.17), 7.301 (6.87), 7.309 (6.33), |
| | 7.317 (6.20), 7.325 (8.11), 7.331 (2.71), 7.388 (0.55), 7.396 (2.73), |
| | 7.403 (1.43), 7.407 (1.83), 7.411 (2.00), 7.420 (1.73), 7.425 (0.70), |
| | 7.435 (3.09), 7.444 (2.72), 7.450 (4.11), 7.455 (3.27), 7.458 (2.65), |
| | 7.464 (2.98), 7.469 (3.07), 7.689 (3.21), 7.693 (3.11), 8.256 (3.86), |
| | 8.271 (3.66), 10.749 (4.02). |
| 144 | 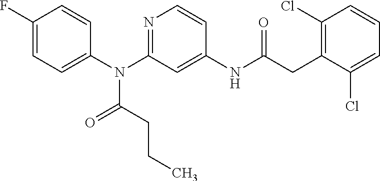 [SMILES: CCCC(=O)N(c1ccc(F)cc1)c1cc(NC(=O)Cc2c(Cl)cccc2Cl)ccn1] |
| | N-{4-[2-(2,6-dichlorophenyl)acetamido]pyridin-2-yl}-N-(4- |
| | fluorophenyl)butanamide |
| | LC-MS (Method 1): Rₜ = 1.28 min; MS (ESIneg): m/z = 458 [M − H]⁻ |
| | ¹H-NMR (400 MHz, DMSO-d6) δ [ppm]: 0.804 (7.05), 0.823 (16.00), |
| | 0.842 (7.57), 1.485 (0.51), 1.503 (2.28), 1.521 (4.35), 1.539 (4.42), |
| | 1.558 (2.30), 1.577 (0.48), 2.187 (3.66), 2.206 (6.46), 2.223 (3.27), |
| | 2.331 (0.61), 2.518 (3.57), 2.522 (2.22), 2.673 (0.65), 4.077 (12.51), |
| | 7.211 (2.76), 7.217 (1.07), 7.228 (1.44), 7.234 (6.77), 7.238 (1.41), |
| | 7.242 (0.82), 7.250 (1.30), 7.255 (4.83), 7.264 (0.62), 7.293 (0.56), |
| | 7.301 (3.94), 7.306 (1.66), 7.313 (4.25), 7.317 (1.88), 7.323 (2.87), |
| | 7.331 (1.16), 7.335 (4.52), 7.354 (3.40), 7.357 (3.49), 7.376 (4.21), |
| | 7.414 (2.84), 7.418 (2.90), 7.427 (3.07), 7.432 (2.96), 7.487 (11.31), |
| | 7.507 (6.94), 7.691 (3.06), 7.695 (3.01), 8.259 (3.94), 8.273 (3.76), |
| | 10.861 (4.10). |
| 145 | 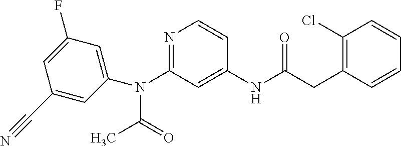 [SMILES: CC(=O)N(c1cc(F)cc(C#N)c1)c1cc(NC(=O)Cc2ccccc2Cl)ccn1] |
| | N-{4-[2-(2-Chlorphenyl)acetamido]pyridin-2-yl}-N-(3-cyan-5- |
| | fluorphenyl)acetamide |
| | LC-MS (Method 1): Rₜ = 1.14 min; MS (ESIpos): m/z = 423 [M + H]⁺ |
| | ¹H-NMR (400 MHz, DMSO-d6) δ [ppm]: 2.026 (16.00), 2.074 (0.73), |
| | 2.518 (5.84), 2.523 (4.08), 3.888 (7.83), 7.305 (3.20), 7.314 (2.26), |
| | 7.319 (2.33), 7.328 (4.28), 7.338 (0.53), 7.402 (1.68), 7.408 (0.88), |
| | 7.412 (1.07), 7.416 (1.17), 7.425 (1.12), 7.438 (1.93), 7.447 (1.55), |
| | 7.451 (1.08), 7.461 (1.38), 7.514 (1.66), 7.518 (1.70), 7.528 (1.69), |
| | 7.532 (1.68), 7.636 (0.98), 7.660 (1.03), 7.690 (2.09), 7.736 (1.57), |
| | 7.814 (0.87), 7.817 (0.97), 7.819 (1.06), 7.823 (0.88), 7.835 (0.91), |
| | 7.838 (1.00), 7.840 (1.00), 7.844 (0.85), 8.296 (1.81), 8.309 (1.74), |
| | 10.808 (2.54). |
| 146 | 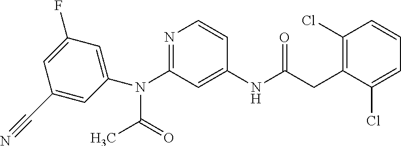 [SMILES: CC(=O)N(c1cc(F)cc(C#N)c1)c1cc(NC(=O)Cc2c(Cl)cccc2Cl)ccn1] |
| | N-(3-Cyan-5-fluorphenyl)-N-{4-[2-(2,6- |
| | dichlorphenyl)acetamido]pyridin-2-yl}acetamide |
| | LC-MS (Method 1): Rₜ = 1.18 min; MS (ESIpos): m/z = 457 [M + H]⁺ |
| | ¹H-NMR (400 MHz, DMSO-d6) δ [ppm]: 2.024 (16.00), 2.074 (6.32), |
| | 2.084 (13.82), 2.518 (4.15), 2.523 (2.91), 4.089 (7.58), 7.338 (1.46), |
| | 7.356 (1.95), 7.359 (2.14), 7.377 (2.32), 7.484 (1.86), 7.490 (7.78), |
| | 7.498 (1.85), 7.502 (1.90), 7.509 (4.37), 7.640 (0.95), 7.645 (0.71), |
| | 7.664 (0.96), 7.669 (0.73), 7.694 (1.98), 7.735 (1.42), 7.813 (0.85), |
| | 7.816 (0.93), 7.819 (1.01), 7.823 (0.86), 7.834 (0.88), 7.837 (0.98), |
| | 7.840 (0.97), 7.844 (0.81), 8.299 (1.77), 8.313 (1.69), 10.929 (2.30). |
| 147 | 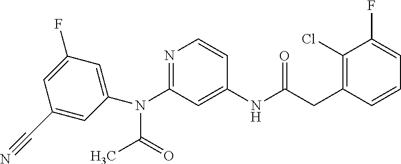 [SMILES: CC(=O)N(c1cc(F)cc(C#N)c1)c1cc(NC(=O)Cc2cccc(F)c2Cl)ccn1] |
| | N-{4-[2-(2-Chlor-3-fluorphenyl)acetamido]pyridin-2-yl}-N-(3-cyan- |
| | 5-fluorphenyl)acetamide |
| | LC-MS (Method 1): Rₜ = 1.16 min; MS (ESIpos): m/z = 441 [M + H]⁺ |
| | ¹H-NMR (400 MHz, DMSO-d6) δ [ppm]: 2.026 (16.00), 2.518 (5.41), |
| | 2.523 (3.77), 3.951 (7.44), 7.266 (0.81), 7.273 (1.20), 7.280 (0.89), |
| | 7.284 (0.98), 7.289 (1.34), 7.327 (0.42), 7.341 (1.87), 7.346 (2.41), |
| | 7.362 (4.27), 7.369 (1.42), 7.377 (1.29), 7.383 (0.41), 7.506 (1.66), |
| | 7.511 (1.67), 7.520 (1.68), 7.525 (1.72), 7.638 (0.99), 7.643 (0.74), |
| | 7.662 (0.98), 7.691 (2.06), 7.736 (1.54), 7.816 (0.89), 7.819 (0.96), |
| | 7.821 (1.04), 7.825 (0.88), 7.837 (0.92), 7.840 (1.00), 7.842 (1.02), |
| | 7.846 (0.83), 8.297 (1.80), 8.311 (1.72), 10.846 (2.57). |
| 148 | 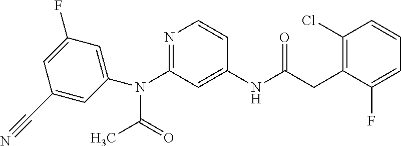 [SMILES: CC(=O)N(c1cc(F)cc(C#N)c1)c1cc(NC(=O)Cc2c(F)cccc2Cl)ccn1] |
| | N-{4-[2-(2-Chlor-6-fluorphenyl)acetamido]pyridin-2-yl}-N-(3-cyan- |
| | 5-fluorphenyl)acetamide |
| | LC-MS (Method 1): Rₜ = 1.15 min; MS (ESIpos): m/z = 441 [M + H]⁺ |
| | ¹H-NMR (400 MHz, DMSO-d6) δ [ppm]: 2.023 (16.00), 2.074 (2.62), |
| | 2.518 (7.91), 2.523 (5.50), 3.933 (4.65), 3.936 (4.52), 7.229 (0.65), |
| | 7.234 (0.66), 7.247 (0.95), 7.252 (1.56), 7.272 (0.96), 7.275 (0.96), |
| | 7.340 (0.69), 7.344 (0.87), 7.360 (3.64), 7.365 (3.30), 7.379 (1.37), |
| | 7.384 (1.26), 7.399 (1.32), 7.405 (0.45), 7.420 (0.42), 7.486 (1.56), |
| | 7.491 (1.57), 7.500 (1.57), 7.505 (1.69), 7.636 (0.95), 7.661 (0.97), |
| | 7.691 (2.05), 7.736 (1.50), 7.818 (1.06), 7.822 (0.87), 7.833 (0.90), |
| | 7.837 (0.99), 7.839 (1.00), 8.299 (1.77), 8.314 (1.69), 10.911 (2.44). |
| 149 | 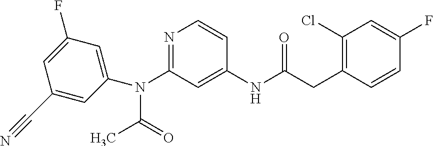 [SMILES: CC(=O)N(c1cc(F)cc(C#N)c1)c1cc(NC(=O)Cc2ccc(F)cc2Cl)ccn1] |
| | N-{4-[2-(2-Chlor-4-fluorphenyl)acetamido]pyridin-2-yl}-N-(3-cyan- |
| | 5-fluorphenyl)acetamide |
| | LC-MS (Method 1): Rₜ = 1.17 min; MS (ESIpos): m/z = 441 [M + H]⁺ |
| | ¹H-NMR (400 MHz, DMSO-d6) δ [ppm]: 2.025 (16.00), 2.074 (0.63), |
| | 2.518 (4.38), 2.523 (3.04), 3.878 (6.52), 7.190 (0.74), 7.196 (0.78), |
| | 7.211 (1.51), 7.218 (1.73), 7.233 (0.87), 7.240 (0.92), 7.434 (1.64), |
| | 7.440 (1.64), 7.450 (1.53), 7.456 (1.77), 7.463 (1.98), 7.471 (1.39), |
| | 7.487 (1.24), 7.504 (1.62), 7.509 (1.60), 7.519 (1.61), 7.523 (1.67), |
| | 7.634 (0.98), 7.659 (0.98), 7.664 (0.76), 7.688 (2.04), 7.733 (1.53), |
| | 7.814 (0.86), 7.818 (0.96), 7.820 (1.05), 7.823 (0.86), 7.835 (0.90), |
| | 7.838 (0.98), 7.841 (1.01), 7.844 (0.83), 8.294 (1.76), 8.309 (1.69), |
| | 10.806 (2.57). |
| 150 | 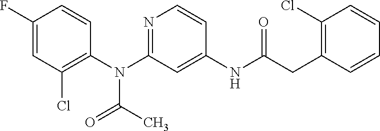 [SMILES: CC(=O)N(c1cc(NC(=O)Cc2ccccc2Cl)ccn1)c1ccc(F)cc1Cl] |
| | N-(2-Chlor-4-fluorphenyl)-N-{4-[2-(2- |
| | chlorphenyl)acetamido]pyridin-2-yl}acetamide |
| | LC-MS (Method 1): Rₜ = 1.19 min; MS (ESIpos): m/z = 432 [M + H]⁺ |
| | ¹H-NMR (400 MHz, DMSO-d6) δ [ppm]: 2.000 (14.96), 2.518 (3.08), |
| | 2.522 (1.90), 3.881 (16.00), 7.295 (0.80), 7.298 (1.77), 7.305 (8.89), |
| | 7.313 (4.06), 7.316 (4.95), 7.319 (6.55), 7.321 (5.90), 7.329 (10.25), |
| | 7.338 (1.24), 7.340 (2.74), 7.348 (2.13), 7.394 (0.48), 7.402 (3.49), |
| | 7.409 (1.75), 7.414 (2.18), 7.418 (2.19), 7.426 (2.26), 7.439 (4.22), |
| | 7.449 (5.99), 7.454 (5.25), 7.463 (6.29), 7.468 (4.35), 7.546 (1.80), |
| | 7.560 (1.94), 7.568 (1.73), 7.582 (1.55), 7.622 (3.50), 7.629 (3.79), |
| | 7.643 (3.49), 7.650 (3.64), 7.867 (1.17), 8.170 (3.30), 8.184 (3.13), |
| | 10.745 (5.05). |
| 151 | 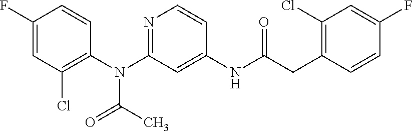 [SMILES: CC(=O)N(c1cc(NC(=O)Cc2ccc(F)cc2Cl)ccn1)c1ccc(F)cc1Cl] |
| | N-(2-Chlor-4-fluorphenyl)-N-{4-[2-(2-chlor-4- |
| | fluorphenyl)acetamido]pyridin-2-yl}acetamide |
| | LC-MS (Method 1): Rₜ = 1.21 min; MS (ESIpos): m/z = 450 [M + H]⁺ |
| | ¹H-NMR (400 MHz, DMSO-d6) δ [ppm]: 1.998 (16.00), 2.332 (0.89), |
| | 2.518 (4.36), 2.523 (2.70), 2.673 (0.89), 3.944 (15.91), 7.268 (1.66), |
| | 7.275 (2.55), 7.282 (1.84), 7.286 (2.01), 7.290 (2.78), 7.300 (1.66), |
| | 7.307 (2.01), 7.322 (2.72), 7.328 (3.38), 7.342 (5.51), 7.347 (6.80), |
| | 7.363 (8.66), 7.370 (3.24), 7.379 (2.78), 7.385 (0.92), 7.399 (0.49), |
| | 7.442 (4.01), 7.447 (4.24), 7.456 (4.16), 7.461 (4.39), 7.548 (1.95), |
| | 7.563 (2.09), 7.570 (1.86), 7.585 (1.66), 7.623 (3.73), 7.630 (3.90), |
| | 7.644 (3.81), 7.651 (3.67), 7.868 (1.23), 8.172 (3.50), 8.186 (3.33), |
| | 10.783 (5.36). |
| 152 | 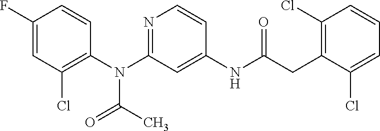 [SMILES: CC(=O)N(c1cc(NC(=O)Cc2c(Cl)cccc2Cl)ccn1)c1ccc(F)cc1Cl] |
| | N-(2-Chlor-4-fluorphenyl)-N-{4-[2-(2,6- |
| | dichlorphenyl)acetamido]pyridin-2-yl}acetamide |
| | LC-MS (Method 1): Rₜ = 1.24 min; MS (ESIpos): m/z = 466 [M + H]⁺ |
| | ¹H-NMR (400 MHz, DMSO-d6) δ [ppm]: 1.996 (15.23), 2.332 (1.23), |
| | 2.336 (0.54), 2.518 (5.82), 2.523 (3.62), 2.673 (1.27), 2.678 (0.58), |
| | 4.083 (16.00), 7.300 (1.62), 7.307 (1.85), 7.322 (2.43), 7.329 (2.66), |
| | 7.339 (3.78), 7.342 (2.12), 7.350 (2.16), 7.357 (4.51), 7.361 (4.93), |
| | 7.379 (5.44), 7.419 (3.82), 7.424 (4.01), 7.433 (3.97), 7.438 (4.05), |
| | 7.491 (15.11), 7.511 (9.37), 7.551 (1.85), 7.565 (2.00), 7.572 (1.77), |
| | 7.587 (1.58), 7.623 (3.62), 7.630 (3.70), 7.644 (3.66), 7.651 (3.59), |
| | 7.865 (1.12), 8.172 (3.28), 8.186 (3.12), 10.854 (5.17). |
| 153 | 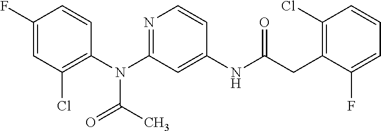 [SMILES: CC(=O)N(c1cc(NC(=O)Cc2c(F)cccc2Cl)ccn1)c1ccc(F)cc1Cl] |
| | N-(2-Chlor-4-fluorphenyl)-N-{4-[2-(2-chlor-6- |
| | fluorphenyl)acetamido]pyridin-2-yl}acetamide |
| | LC-MS (Method 1): Rₜ = 1.20 min; MS (ESIpos): m/z = 450 [M + H]⁺ |
| | ¹H-NMR (400 MHz, DMSO-d6) δ [ppm]: 1.997 (16.00), 2.332 (0.54), |
| | 2.518 (2.42), 2.523 (1.51), 2.673 (0.54), 3.927 (9.72), 3.931 (9.42), |
| | 7.231 (1.40), 7.236 (1.47), 7.249 (2.07), 7.254 (3.40), 7.257 (1.80), |
| | 7.274 (2.14), 7.278 (2.03), 7.299 (1.61), 7.307 (1.86), 7.321 (2.47), |
| | 7.328 (2.80), 7.341 (3.40), 7.345 (2.35), 7.349 (2.29), 7.362 (8.19), |
| | 7.367 (7.47), 7.380 (3.03), 7.385 (2.82), 7.400 (3.03), 7.405 (0.95), |
| | 7.423 (4.06), 7.428 (4.20), 7.437 (4.11), 7.442 (4.24), 7.550 (1.94), |
| | 7.565 (2.07), 7.572 (1.84), 7.586 (1.65), 7.622 (3.68), 7.629 (3.96), |
| | 7.643 (3.66), 7.650 (3.78), 7.868 (1.21), 8.173 (3.45), 8.187 (3.26), |
| | 10.844 (5.23). |
| 154 | 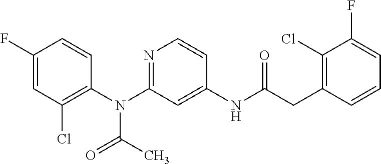 [SMILES: CC(=O)N(c1cc(NC(=O)Cc2cccc(F)c2Cl)ccn1)c1ccc(F)cc1Cl] |
| | N-(2-Chlor-4-fluorphenyl)-N-{4-[2-(2-chlor-3- |
| | fluorphenyl)acetamido]pyridin-2-yl}acetamide |
| | LC-MS (Method 1): Rₜ = 1.20 min; MS (ESIpos): m/z = 450 [M + H]⁺ |
| | ¹H-NMR (400 MHz, DMSO-d6) δ [ppm]: 1.998 (16.00), 2.518 (2.66), |
| | 2.523 (1.67), 3.872 (14.07), 7.191 (1.70), 7.197 (1.93), 7.212 (3.63), |
| | 7.219 (4.12), 7.233 (1.99), 7.240 (2.05), 7.299 (1.60), 7.306 (1.93), |
| | 7.321 (2.51), 7.328 (2.82), 7.341 (1.94), 7.348 (2.15), 7.435 (3.94), |
| | 7.441 (7.75), 7.445 (4.66), 7.452 (3.96), 7.455 (6.01), 7.459 (5.30), |
| | 7.463 (4.39), 7.467 (3.54), 7.473 (2.91), 7.489 (2.63), 7.546 (1.96), |
| | 7.560 (2.11), 7.568 (1.87), 7.582 (1.67), 7.622 (3.78), 7.629 (3.96), |
| | 7.644 (3.81), 7.651 (3.84), 7.863 (1.27), 8.169 (3.54), 8.183 (3.35), |
| | 10.742 (5.54). |
| 155 | 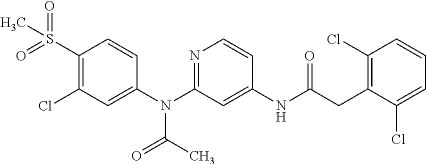 [SMILES: CC(=O)N(c1ccc(S(C)(=O)=O)c(Cl)c1)c1cc(NC(=O)Cc2c(Cl)cccc2Cl)ccn1] |
| | N-[3-Chlor-4-(methylsulfonyl)phenyl]-N-{4-[2-(2,6- |
| | dichlorphenyl)acetamido]pyridin-2-yl}acetamide |
| | LC-MS (Method 2): Rₜ = 1.14 min; MS (ESIpos): m/z = 528 [M + H]⁺ |
| | ¹H-NMR (400 MHz, DMSO-d6) δ [ppm]: 2.518 (4.33), 2.523 (2.98), |
| | 3.858 (16.00), 6.804 (2.33), 6.824 (2.66), 7.150 (2.76), 7.170 (5.43), |
| | 7.189 (3.12), 7.210 (1.64), 7.228 (4.85), 7.231 (4.29), 7.245 (11.58), |
| | 7.256 (14.44), 7.268 (5.43), 7.272 (5.75), 7.281 (2.11), 7.288 (3.36), |
| | 7.299 (0.98), 7.305 (1.51), 7.310 (1.71), 7.325 (7.15), 7.332 (7.38), |
| | 7.348 (9.45), 7.353 (4.83), 7.364 (3.13), 7.370 (1.22), 7.384 (0.69), |
| | 7.483 (3.44), 7.487 (5.70), 7.492 (3.28), 10.282 (5.35). |
| 156 | 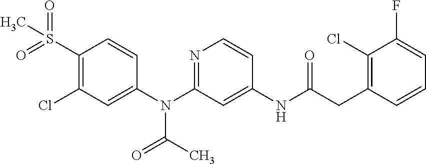 [SMILES: CC(=O)N(c1ccc(S(C)(=O)=O)c(Cl)c1)c1cc(NC(=O)Cc2cccc(F)c2Cl)ccn1] |
| | N-{4-[2-(2-Chlor-3-fluorphenyl)acetamido]pyridin-2-yl}-N-[3- |
| | chlor-4-(methylsulfonyl)phenyl]acetamide |
| | LC-MS (Method 2): Rₜ = 1.11 min; MS (ESIpos): m/z = 510 [M + H]⁺ |
| | ¹H-NMR (400 MHz, DMSO-d6) δ [ppm]: 2.037 (14.26), 2.518 (2.64), |
| | 2.523 (1.85), 3.366 (0.50), 3.382 (16.00), 3.948 (6.37), 7.261 (0.71), |
| | 7.268 (1.04), 7.275 (0.76), 7.279 (0.83), 7.284 (1.17), 7.338 (1.69), |
| | 7.343 (1.98), 7.358 (4.13), 7.367 (2.23), 7.374 (1.29), 7.383 (1.14), |
| | 7.388 (1.14), 7.542 (1.33), 7.547 (1.42), 7.556 (1.38), 7.561 (1.47), |
| | 7.676 (1.71), 7.733 (2.68), 7.738 (2.57), 8.014 (3.26), 8.035 (3.04), |
| | 8.364 (1.80), 8.378 (1.69), 10.870 (2.35). |
| 157 | 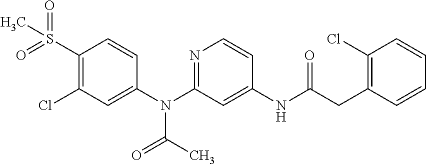 [SMILES: CC(=O)N(c1ccc(S(C)(=O)=O)c(Cl)c1)c1cc(NC(=O)Cc2ccccc2Cl)ccn1] |
| | N-[3-Chlor-4-(methylsulfonyl)phenyl]-N-{4-[2-(2- |
| | chlorphenyl)acetamido]pyridin-2-yl}acetamide |
| | LC-MS (Method 2): Rₜ = 1.09 min; MS (ESIpos): m/z = 492 [M + H]⁺ |
| | ¹H-NMR (400 MHz, DMSO-d6) δ [ppm]: 2.038 (13.97), 2.518 (1.46), |
| | 2.522 (0.97), 3.381 (16.00), 3.885 (6.52), 7.301 (2.77), 7.310 (2.28), |
| | 7.315 (2.39), 7.324 (3.85), 7.334 (0.63), 7.360 (1.07), 7.365 (1.06), |
| | 7.381 (1.15), 7.386 (1.30), 7.396 (1.57), 7.407 (0.98), 7.411 (1.10), |
| | 7.420 (1.00), 7.434 (1.72), 7.443 (1.40), 7.448 (1.07), 7.457 (1.27), |
| | 7.549 (1.31), 7.554 (1.33), 7.563 (1.36), 7.568 (1.35), 7.678 (1.81), |
| | 7.733 (2.74), 7.738 (2.58), 8.013 (3.22), 8.034 (3.05), 8.362 (1.80), |
| | 8.376 (1.72), 10.833 (2.31). |
| 158 | 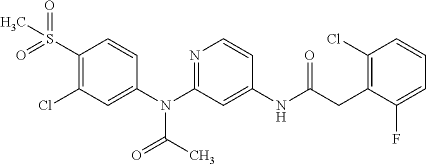 [SMILES: CC(=O)N(c1ccc(S(C)(=O)=O)c(Cl)c1)c1cc(NC(=O)Cc2c(F)cccc2Cl)ccn1] |
| | N-{4-[2-(2-Chlor-6-fluorphenyl)acetamido]pyridin-2-yl}-N-[3- |
| | chlor-4-(methylsulfonyl)phenyl]acetamide |
| | LC-MS (Method 2): Rₜ = 1.10 min; MS (ESIpos): m/z = 510 [M + H]⁺ |
| | ¹H-NMR (400 MHz, DMSO-d6) δ [ppm]: 2.035 (14.96), 2.074 (0.67), |
| | 2.518 (1.91), 2.523 (1.37), 3.381 (16.00), 3.932 (4.06), 3.935 (3.95), |
| | 7.225 (0.59), 7.230 (0.60), 7.244 (0.88), 7.249 (1.38), 7.268 (0.87), |
| | 7.272 (0.83), 7.337 (0.65), 7.341 (0.80), 7.357 (3.47), 7.362 (3.78), |
| | 7.376 (1.44), 7.382 (1.91), 7.388 (1.23), 7.396 (1.38), 7.402 (0.46), |
| | 7.417 (0.44), 7.524 (1.35), 7.529 (1.36), 7.538 (1.35), 7.543 (1.44), |
| | 7.678 (1.69), 7.736 (2.74), 7.741 (2.67), 8.012 (3.54), 8.033 (3.24), |
| | 8.366 (1.80), 8.380 (1.73), 10.936 (2.33). |
| 159 | 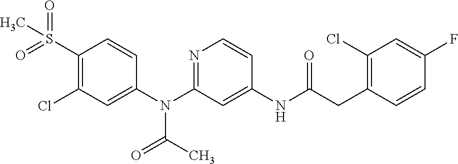 [SMILES: CC(=O)N(c1ccc(S(C)(=O)=O)c(Cl)c1)c1cc(NC(=O)Cc2ccc(F)cc2Cl)ccn1] |
| | N-{4-[2-(2-Chlor-4-fluorphenyl)acetamido]pyridin-2-yl}-N-[3- |
| | chlor-4-(methylsulfonyl)phenyl]acetamide |
| | LC-MS (Method 2): Rₜ = 1.12 min; MS (ESIpos): m/z = 510 [M + H]⁺ |
| | ¹H-NMR (400 MHz, DMSO-d6) δ [ppm]: 2.036 (14.66), 2.074 (0.76), |
| | 2.518 (1.80), 2.523 (1.27), 3.382 (16.00), 3.876 (5.64), 7.186 (0.65), |
| | 7.193 (0.71), 7.207 (1.38), 7.214 (1.55), 7.229 (0.79), 7.236 (0.82), |
| | 7.360 (1.04), 7.365 (1.05), 7.381 (1.09), 7.386 (1.10), 7.431 (1.51), |
| | 7.437 (1.54), 7.445 (1.34), 7.453 (1.66), 7.460 (2.50), 7.466 (1.25), |
| | 7.482 (1.08), 7.540 (1.32), 7.545 (1.35), 7.554 (1.33), 7.559 (1.40), |
| | 7.674 (1.78), 7.731 (2.74), 7.736 (2.65), 8.013 (3.36), 8.035 (3.15), |
| | 8.361 (1.81), 8.375 (1.71), 10.830 (2.34). |
| 160 | 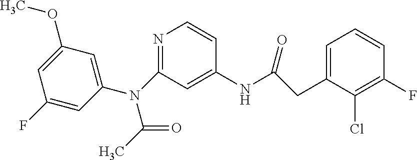 [SMILES: COc1cc(F)cc(N(C(C)=O)c2cc(NC(=O)Cc3cccc(F)c3Cl)ccn2)c1] |
| | N-{4-[2-(2-Chlor-3-fluorphenyl)acetamido]pyridin-2-yl}-N-(3-fluor- |
| | 5-methoxyphenyl)acetamide |
| | LC-MS (Method 2): Rₜ = 1.17 min; MS (ESIpos): m/z = 446 [M + H]⁺ |
| | ¹H-NMR (400 MHz, DMSO-d6) δ [ppm]: 1.877 (0.56), 1.951 (0.47), |
| | 2.011 (14.54), 2.518 (1.38), 2.522 (0.90), 2.669 (0.42), 3.746 (16.00), |
| | 3.941 (6.17), 6.694 (1.68), 6.732 (0.62), 6.738 (1.04), 6.743 (0.66), |
| | 6.756 (0.63), 6.762 (0.98), 6.767 (0.64), 6.796 (0.76), 6.802 (1.32), |
| | 6.808 (0.66), 6.824 (0.77), 6.829 (1.30), 6.835 (0.63), 7.262 (0.66), |
| | 7.269 (1.01), 7.275 (0.72), 7.280 (0.78), 7.285 (1.06), 7.338 (1.66), |
| | 7.343 (1.98), 7.359 (3.78), 7.366 (1.21), 7.374 (1.09), 7.478 (1.52), |
| | 7.483 (1.49), 7.492 (1.44), 7.497 (1.56), 7.670 (1.62), 7.674 (1.55), |
| | 8.285 (2.02), 8.299 (1.92), 10.798 (2.07). |
| 161 | 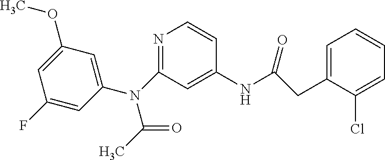 [SMILES: COc1cc(F)cc(N(C(C)=O)c2cc(NC(=O)Cc3ccccc3Cl)ccn2)c1] |
| | N-{4-[2-(2-Chlorphenyl)acetamido]pyridin-2-yl}-N-(3-fluor-5- |
| | methoxyphenyl)acetamide |
| | LC-MS (Method 2): Rₜ = 1.16 min; MS (ESIpos): m/z = 428 [M + H]⁺ |
| | ¹H-NMR (400 MHz, DMSO-d6) δ [ppm]: 1.868 (0.41), 2.012 (13.33), |
| | 2.518 (1.56), 2.522 (1.03), 3.746 (16.00), 3.878 (6.16), 6.692 (1.55), |
| | 6.731 (0.56), 6.736 (0.97), 6.741 (0.62), 6.755 (0.57), 6.760 (0.92), |
| | 6.765 (0.60), 6.795 (0.72), 6.800 (1.26), 6.806 (0.63), 6.822 (0.71), |
| | 6.827 (1.20), 6.833 (0.58), 7.302 (2.82), 7.311 (1.81), 7.317 (1.93), |
| | 7.325 (3.60), 7.335 (0.45), 7.397 (1.32), 7.404 (0.64), 7.407 (0.81), |
| | 7.411 (0.90), 7.420 (0.84), 7.435 (1.53), 7.444 (1.31), 7.449 (0.92), |
| | 7.450 (0.65), 7.458 (1.11), 7.486 (1.41), 7.490 (1.39), 7.500 (1.41), |
| | 7.505 (1.45), 7.671 (1.50), 7.674 (1.45), 8.282 (1.90), 8.297 (1.79), |
| | 10.760 (1.89). |
| 162 | 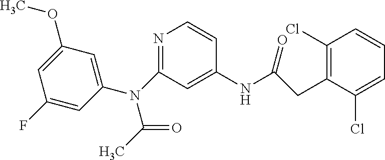 [SMILES: COc1cc(F)cc(N(C(C)=O)c2cc(NC(=O)Cc3c(Cl)cccc3Cl)ccn2)c1] |
| | N-{4-[2-(2,6-Dichlorphenyl)acetamido]pyridin-2-yl}-N-(3-fluor-5- |
| | methoxyphenyl)acetamide |
| | LC-MS (Method 2): Rₜ = 1.22 min; MS (ESIpos): m/z = 462 [M + H]⁺ |
| | ¹H-NMR (400 MHz, DMSO-d6) δ [ppm]: 2.009 (14.08), 2.518 (2.01), |
| | 2.522 (1.28), 3.746 (16.00), 4.080 (6.08), 6.696 (1.58), 6.736 (0.58), |
| | 6.741 (0.98), 6.746 (0.64), 6.760 (0.59), 6.765 (0.92), 6.770 (0.62), |
| | 6.795 (0.74), 6.801 (1.27), 6.807 (0.63), 6.822 (0.73), 6.828 (1.24), |
| | 6.834 (0.59), 7.335 (1.24), 7.354 (1.71), 7.357 (1.79), 7.375 (2.08), |
| | 7.453 (1.39), 7.458 (1.39), 7.467 (1.33), 7.472 (1.47), 7.487 (5.63), |
| | 7.506 (3.44), 7.669 (1.46), 7.672 (1.42), 8.286 (1.90), 8.300 (1.81), |
| | 10.873 (1.98). |
| 163 | 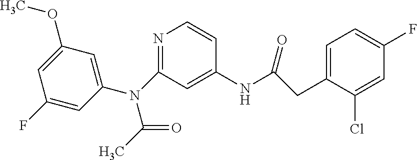 [SMILES: COc1cc(F)cc(N(C(C)=O)c2cc(NC(=O)Cc3ccc(F)cc3Cl)ccn2)c1] |
| | N-{4-[2-(2-Chlor-4-fluorphenyl)acetamido]pyridin-2-yl}-N-(3-fluor- |
| | 5-methoxyphenyl)acetamide |
| | LC-MS (Method 2): Rₜ = 1.19 min; MS (ESIpos): m/z = 446 [M + H]⁺ |
| | ¹H-NMR (400 MHz, DMSO-d6) δ [ppm]: 2.011 (16.00), 2.518 (0.74), |
| | 2.522 (0.49), 3.337 (2.35), 3.868 (5.81), 6.692 (1.82), 6.731 (0.67), |
| | 6.736 (1.13), 6.741 (0.72), 6.755 (0.67), 6.760 (1.07), 6.765 (0.70), |
| | 6.795 (0.80), 6.801 (1.41), 6.807 (0.71), 6.823 (0.84), 6.829 (1.44), |
| | 6.834 (0.70), 7.186 (0.69), 7.193 (0.81), 7.207 (1.52), 7.214 (1.65), |
| | 7.229 (0.79), 7.235 (0.88), 7.430 (1.55), 7.437 (1.53), 7.445 (1.35), |
| | 7.452 (1.65), 7.459 (2.00), 7.467 (1.24), 7.478 (1.69), 7.483 (2.69), |
| | 7.492 (1.52), 7.497 (1.67), 7.668 (1.76), 7.672 (1.70), 8.282 (2.21), |
| | 8.296 (2.09), 10.758 (2.27). |
| 164 | 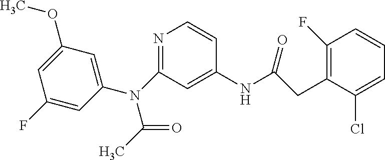 [SMILES: COc1cc(F)cc(N(C(C)=O)c2cc(NC(=O)Cc3c(F)cccc3Cl)ccn2)c1] |
| | N-{4-[2-(2-Chlor-6-fluorphenyl)acetamido]pyridin-2-yl}-N-(3-fluor- |
| | 5-methoxyphenyl)acetamide |
| | LC-MS (Method 2): Rₜ = 1.17 min; MS (ESIpos): m/z = 446 [M + H]⁺ |
| | ¹H-NMR (400 MHz, DMSO-d6) δ [ppm]: 1.874 (0.59), 1.951 (0.51), |
| | 2.010 (16.00), 2.518 (0.72), 2.522 (0.47), 3.333 (4.52), 3.924 (4.02), |
| | 3.928 (3.79), 6.694 (1.75), 6.734 (0.63), 6.739 (1.08), 6.744 (0.68), |
| | 6.758 (0.64), 6.763 (1.03), 6.769 (0.66), 6.794 (0.78), 6.800 (1.35), |
| | 6.806 (0.68), 6.822 (0.81), 6.827 (1.39), 6.833 (0.67), 7.225 (0.57), |
| | 7.230 (0.58), 7.244 (0.85), 7.249 (1.34), 7.252 (0.73), 7.269 (0.84), |
| | 7.273 (0.81), 7.337 (0.60), 7.341 (0.77), 7.357 (3.19), 7.362 (2.81), |
| | 7.376 (1.22), 7.381 (1.14), 7.396 (1.22), 7.416 (0.41), 7.459 (1.60), |
| | 7.464 (1.52), 7.473 (1.55), 7.478 (1.63), 7.671 (1.65), 7.675 (1.60), |
| | 8.286 (2.14), 8.300 (2.02), 10.861 (2.16). |
| 165 | 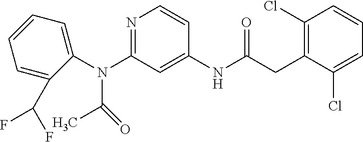 [SMILES: CC(=O)N(c1cc(NC(=O)Cc2c(Cl)cccc2Cl)ccn1)c1ccccc1C(F)F] |
| | N-{4-[2-(2,6-Dichlorphenyl)acetamido]pyridin-2-yl}-N-[2- |
| | (difluormethyl)phenyl]acetamide |
| | LC-MS (Method 2): Rₜ = 1.24 min; MS (ESIneg): m/z = 461 [M − H]⁻ |
| | ¹H-NMR (400 MHz, DMSO-d6) δ [ppm]: 1.970 (16.00), 2.332 (0.69), |
| | 2.518 (3.81), 2.522 (2.40), 2.673 (0.69), 4.082 (14.14), 6.910 (1.98), |
| | 7.047 (3.70), 7.184 (1.73), 7.337 (2.83), 7.356 (3.87), 7.359 (3.96), |
| | 7.378 (4.90), 7.430 (4.37), 7.435 (4.95), 7.444 (3.53), 7.449 (4.49), |
| | 7.489 (13.31), 7.509 (8.00), 7.537 (0.98), 7.540 (0.99), 7.556 (2.61), |
| | 7.558 (2.51), 7.575 (2.04), 7.612 (1.66), 7.631 (2.14), 7.649 (0.80), |
| | 7.703 (2.58), 7.720 (2.02), 7.796 (1.89), 8.221 (3.33), 8.235 (3.18), |
| | 10.875 (4.56). |
| 166 | 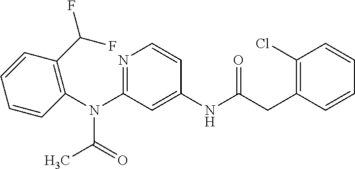 [SMILES: CC(=O)N(c1cc(NC(=O)Cc2ccccc2Cl)ccn1)c1ccccc1C(F)F] |
| | N-{4-[2-(2-Chlorphenyl)acetamido]pyridin-2-yl}-N-[2- |
| | (difluormethyl)phenyl]acetamide |
| | LC-MS (Method 2): Rₜ = 1.13 min; MS (ESIpos): m/z = 430 [M + H]⁺ |
| | ¹H-NMR (400 MHz, DMSO-d6) δ [ppm]: 1.088 (0.42), 1.105 (0.44), |
| | 1.141 (0.43), 1.157 (0.41), 1.974 (16.00), 2.332 (0.56), 2.518 (2.76), |
| | 2.522 (1.74), 2.669 (0.77), 2.673 (0.55), 3.880 (13.98), 6.906 (2.00), |
| | 7.043 (3.74), 7.180 (1.71), 7.293 (0.60), 7.303 (6.02), 7.313 (3.97), |
| | 7.317 (4.28), 7.319 (4.26), 7.327 (8.31), 7.336 (0.90), 7.338 (1.04), |
| | 7.390 (0.48), 7.399 (3.12), 7.405 (1.63), 7.409 (1.94), 7.413 (2.18), |
| | 7.422 (2.76), 7.427 (2.63), 7.437 (4.07), 7.446 (5.32), 7.450 (3.42), |
| | 7.462 (4.02), 7.467 (3.43), 7.476 (3.25), 7.481 (3.25), 7.539 (0.97), |
| | 7.554 (2.59), 7.557 (2.47), 7.573 (2.04), 7.611 (1.64), 7.630 (2.11), |
| | 7.648 (0.79), 7.701 (2.53), 7.720 (1.98), 7.796 (1.98), 8.219 (3.36), |
| | 8.233 (3.18), 10.765 (4.42). |
| 167 | 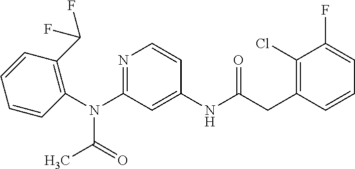 [SMILES: CC(=O)N(c1cc(NC(=O)Cc2cccc(F)c2Cl)ccn1)c1ccccc1C(F)F] |
| | N-{4-[2-(2-Chlor-3-fluorphenyl)acetamido]pyridin-2-yl}-N-[2- |
| | (difluormethyl)phenyl]acetamide |
| | LC-MS (Method 2): Rₜ = 1.14 min; MS (ESIpos): m/z = 448 [M + H]⁺ |
| | ¹H-NMR (400 MHz, DMSO-d6) δ [ppm]: 1.092 (0.43), 1.109 (0.44), |
| | 1.164 (0.43), 1.180 (0.42), 1.972 (16.00), 2.518 (4.71), 2.522 (3.00), |
| | 3.943 (13.63), 6.904 (2.01), 7.041 (3.76), 7.178 (1.75), 7.263 (1.46), |
| | 7.270 (2.19), 7.277 (1.59), 7.281 (1.71), 7.285 (2.40), 7.326 (0.85), |
| | 7.340 (3.50), 7.345 (4.52), 7.361 (8.19), 7.368 (2.64), 7.376 (2.52), |
| | 7.382 (0.76), 7.397 (0.44), 7.428 (1.92), 7.448 (2.45), 7.454 (4.13), |
| | 7.459 (3.61), 7.468 (3.39), 7.473 (3.56), 7.539 (0.97), 7.556 (2.61), |
| | 7.558 (2.50), 7.575 (2.03), 7.612 (1.64), 7.631 (2.12), 7.649 (0.78), |
| | 7.702 (2.54), 7.720 (1.98), 7.795 (1.96), 8.221 (3.39), 8.234 (3.25), |
| | 10.803 (4.61). |
| 168 | 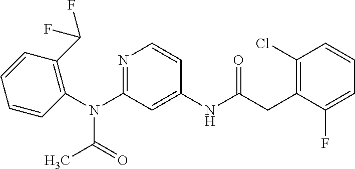 [SMILES: CC(=O)N(c1cc(NC(=O)Cc2c(F)cccc2Cl)ccn1)c1ccccc1C(F)F] |
| | N-{4-[2-(2-Chlor-6-fluorphenyl)acetamido]pyridin-2-yl}-N-[2- |
| | (difluormethyl)phenyl]acetamide |
| | LC-MS (Method 2): Rₜ = 1.13 min; MS (ESIpos): m/z = 448 [M + H]⁺ |
| | ¹H-NMR (400 MHz, DMSO-d6) δ [ppm]: 1.088 (0.42), 1.906 (0.56), |
| | 1.971 (16.00), 2.518 (13.92), 2.522 (9.48), 3.307 (0.84), 3.926 (8.60), |
| | 3.929 (8.39), 6.906 (1.96), 7.044 (3.69), 7.181 (1.73), 7.229 (1.17), |
| | 7.234 (1.18), 7.248 (1.74), 7.253 (2.84), 7.272 (1.77), 7.275 (1.71), |
| | 7.340 (1.37), 7.344 (1.62), 7.360 (6.48), 7.365 (5.76), 7.379 (2.57), |
| | 7.384 (2.28), 7.400 (2.59), 7.405 (0.81), 7.420 (0.95), 7.433 (4.95), |
| | 7.438 (3.96), 7.447 (4.73), 7.452 (5.52), 7.537 (0.98), 7.556 (2.67), |
| | 7.575 (2.13), 7.612 (1.73), 7.630 (2.21), 7.648 (0.86), 7.702 (2.64), |
| | 7.719 (2.13), 7.798 (2.03), 8.221 (3.34), 8.236 (3.23), 10.865 (4.67). |
| 169 | 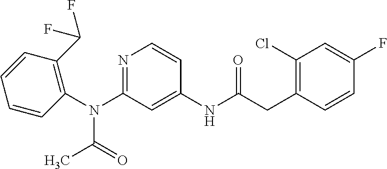 [SMILES: CC(=O)N(c1cc(NC(=O)Cc2ccc(F)cc2Cl)ccn1)c1ccccc1C(F)F] |
| | N-{4-[2-(2-Chlor-4-fluorphenyl)acetamido]pyridin-2-yl}-N-[2- |
| | (difluormethyl)phenyl]acetamide |
| | LC-MS (Method 2): Rₜ = 1.15 min; MS (ESIpos): m/z = 448 [M + H]⁺ |
| | ¹H-NMR (400 MHz, DMSO-d6) δ [ppm]: 1.085 (0.56), 1.102 (0.55), |
| | 1.156 (0.52), 1.173 (0.53), 1.907 (0.45), 1.973 (16.00), 2.208 (0.58), |
| | 2.518 (6.69), 2.522 (4.57), 3.309 (0.43), 3.871 (12.17), 6.904 (1.93), |
| | 7.041 (3.65), 7.178 (1.72), 7.189 (1.42), 7.196 (1.57), 7.210 (2.91), |
| | 7.217 (3.21), 7.232 (1.58), 7.238 (1.72), 7.427 (2.12), 7.433 (3.64), |
| | 7.440 (3.82), 7.447 (5.28), 7.452 (4.81), 7.456 (5.57), 7.462 (5.90), |
| | 7.466 (4.83), 7.471 (4.64), 7.485 (2.35), 7.539 (0.98), 7.555 (2.68), |
| | 7.575 (2.09), 7.611 (1.75), 7.630 (2.26), 7.648 (0.88), 7.701 (2.65), |
| | 7.719 (2.12), 7.792 (2.10), 8.217 (3.37), 8.231 (3.20), 10.764 (4.73). |
| 170 | 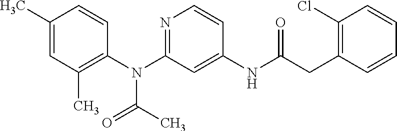 [SMILES: CC(=O)N(c1cc(NC(=O)Cc2ccccc2Cl)ccn1)c1ccc(C)cc1C] |
| | N-{4-[2-(2-Chlorphenyl)acetamido]pyridin-2-yl}-N-(2,4- |
| | dimethylphenyl)acetamide |
| | LC-MS (Method 1): Rₜ = 1.22 min; MS (ESIpos): m/z = 408 [M + H]⁺ |
| | ¹H-NMR (500 MHz, DMSO-d6) δ [ppm]: 1.957 (10.76), 2.099 (16.00), |
| | 2.285 (14.83), 2.514 (1.85), 2.518 (1.82), 2.522 (1.48), 3.869 (10.35), |
| | 7.039 (0.83), 7.052 (1.80), 7.054 (1.98), 7.075 (3.62), 7.091 (1.52), |
| | 7.119 (2.65), 7.305 (4.31), 7.311 (2.43), 7.313 (2.65), 7.316 (2.58), |
| | 7.317 (2.73), 7.323 (5.76), 7.332 (0.61), 7.397 (2.11), 7.402 (1.00), |
| | 7.405 (1.31), 7.409 (1.43), 7.416 (1.59), 7.418 (2.61), 7.422 (2.53), |
| | 7.429 (2.73), 7.433 (2.59), 7.438 (2.47), 7.445 (1.85), 7.448 (1.32), |
| | 7.450 (0.95), 7.456 (1.79), 7.721 (1.02), 8.179 (2.50), 8.190 (2.43), |
| | 10.697 (3.21). |
| 171 | 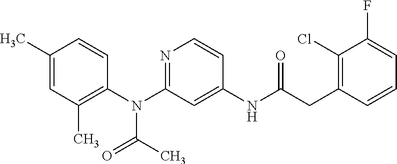 [SMILES: CC(=O)N(c1cc(NC(=O)Cc2cccc(F)c2Cl)ccn1)c1ccc(C)cc1C] |
| | N-{4-[2-(2-Chlor-3-fluorphenyl)acetamido]pyridin-2-yl}-N-(2,4- |
| | dimethylphenyl)acetamide |
| | LC-MS (Method 1): Rₜ = 1.23 min; MS (ESIpos): m/z = 426 [M + H]⁺ |
| | ¹H-NMR (500 MHz, DMSO-d6) δ [ppm]: 1.955 (10.87), 2.097 (16.00), |
| | 2.286 (14.66), 2.514 (1.10), 2.518 (1.09), 2.522 (0.88), 3.932 (9.85), |
| | 7.040 (0.84), 7.052 (1.81), 7.056 (1.97), 7.077 (3.63), 7.092 (1.52), |
| | 7.119 (2.65), 7.263 (1.06), 7.267 (1.45), 7.274 (1.09), 7.277 (1.21), |
| | 7.281 (1.54), 7.329 (0.57), 7.340 (2.28), 7.346 (3.34), 7.359 (3.61), |
| | 7.363 (2.04), 7.371 (1.51), 7.376 (0.54), 7.412 (2.41), 7.416 (2.36), |
| | 7.423 (2.42), 7.427 (2.49), 7.723 (1.01), 8.181 (2.58), 8.193 (2.46), |
| | 10.736 (3.25). |
| 172 | 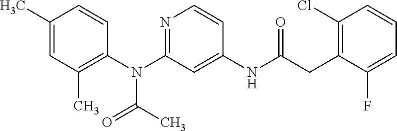 [SMILES: CC(=O)N(c1cc(NC(=O)Cc2c(F)cccc2Cl)ccn1)c1ccc(C)cc1C] |
| | N-{4-[2-(2-Chlor-6-fluorphenyl)acetamido]pyridin-2-yl}-N-(2,4- |
| | dimethylphenyl)acetamide |
| | LC-MS (Method 1): Rₜ = 1.22 min; MS (ESIpos): m/z = 426 [M + H]⁺ |
| | ¹H-NMR (500 MHz, DMSO-d6) δ [ppm]: 1.954 (11.00), 2.098 (16.00), |
| | 2.286 (15.07), 2.514 (2.15), 2.518 (2.02), 2.522 (1.65), 3.915 (5.94), |
| | 3.917 (5.82), 7.039 (0.87), 7.056 (1.99), 7.078 (3.57), 7.094 (1.58), |
| | 7.119 (2.70), 7.231 (0.83), 7.235 (0.87), 7.247 (1.20), 7.250 (1.94), |
| | 7.267 (1.08), 7.269 (1.11), 7.342 (1.04), 7.344 (1.12), 7.358 (3.64), |
| | 7.361 (2.33), 7.367 (1.69), 7.378 (1.54), 7.383 (1.68), 7.391 (2.49), |
| | 7.395 (4.10), 7.399 (0.80), 7.402 (2.32), 7.406 (2.35), 7.411 (0.65), |
| | 7.720 (1.02), 8.182 (2.60), 8.193 (2.48), 10.797 (3.27). |
| 173 | 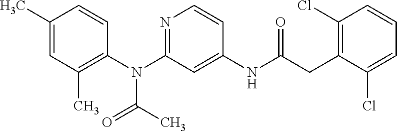 [SMILES: CC(=O)N(c1cc(NC(=O)Cc2c(Cl)cccc2Cl)ccn1)c1ccc(C)cc1C] |
| | N-{4-[2-(2,6-Dichlorphenyl)acetamido]pyridin-2-yl}-N-(2,4- |
| | dimethylphenyl)acetamide |
| | LC-MS (Method 1): Rₜ = 1.27 min; MS (ESIpos): m/z = 442 [M + H]⁺ |
| | ¹H-NMR (500 MHz, DMSO-d6) δ [ppm]: 1.953 (10.84), 2.100 (16.00), |
| | 2.286 (14.91), 2.514 (1.61), 2.518 (1.55), 2.522 (1.25), 4.071 (10.40), |
| | 7.040 (0.89), 7.053 (1.82), 7.056 (1.97), 7.079 (3.62), 7.095 (1.61), |
| | 7.120 (2.68), 7.340 (2.01), 7.356 (2.63), 7.357 (2.94), 7.373 (2.94), |
| | 7.388 (2.25), 7.392 (2.25), 7.399 (2.19), 7.403 (2.24), 7.489 (8.81), |
| | 7.505 (6.54), 7.719 (1.00), 8.182 (2.55), 8.193 (2.46), 10.808 (3.30). |
| 174 | 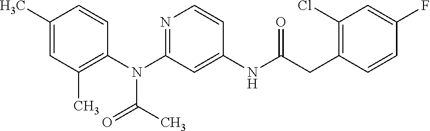 [SMILES: CC(=O)N(c1cc(NC(=O)Cc2ccc(F)cc2Cl)ccn1)c1ccc(C)cc1C] |
| | N-{4-[2-(2-Chlor-4-fluorphenyl)acetamido]pyridin-2-yl}-N-(2,4- |
| | dimethylphenyl)acetamide |
| | LC-MS (Method 1): Rₜ = 1.24 min; MS (ESIpos): m/z = 426 [M + H]⁺ |
| | ¹H-NMR (500 MHz, DMSO-d6) δ [ppm]: 1.955 (10.72), 2.097 (16.00), |
| | 2.285 (14.84), 2.514 (1.21), 2.518 (1.21), 2.522 (0.97), 3.859 (8.72), |
| | 7.039 (0.82), 7.051 (1.83), 7.055 (1.99), 7.075 (3.61), 7.091 (1.52), |
| | 7.119 (2.64), 7.192 (1.01), 7.197 (1.07), 7.209 (2.10), 7.214 (2.21), |
| | 7.226 (1.11), 7.231 (1.16), 7.410 (2.30), 7.414 (2.30), 7.421 (2.31), |
| | 7.425 (2.38), 7.434 (2.05), 7.439 (2.11), 7.447 (1.83), 7.451 (2.21), |
| | 7.457 (2.20), 7.460 (1.92), 7.465 (1.68), 7.477 (1.56), 7.719 (1.00), |
| | 8.178 (2.51), 8.189 (2.43), 10.695 (3.31). |
| 175 | 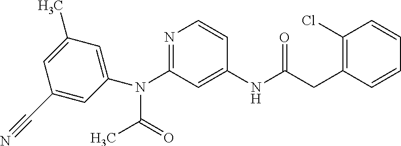 [SMILES: CC(=O)N(c1cc(C)cc(C#N)c1)c1cc(NC(=O)Cc2ccccc2Cl)ccn1] |
| | N-{4-[2-(2-Chlorphenyl)acetamido]pyridin-2-yl}-N-(3-cyan-5- |
| | methylphenyl)acetamide |
| | LC-MS (Method 1): Rₜ = 1.14 min; MS (ESIpos): m/z = 419 [M + H]⁺ |
| | ¹H-NMR (400 MHz, DMSO-d6) δ [ppm]: 2.005 (16.00), 2.335 (11.69), |
| | 2.518 (2.26), 2.523 (1.61), 2.669 (0.43), 3.880 (8.22), 7.303 (3.43), |
| | 7.313 (2.43), 7.319 (2.52), 7.327 (4.54), 7.337 (0.59), 7.398 (1.78), |
| | 7.406 (0.92), 7.409 (1.16), 7.414 (1.26), 7.422 (1.20), 7.426 (0.52), |
| | 7.437 (3.31), 7.445 (3.25), 7.450 (1.66), 7.460 (1.49), 7.495 (1.81), |
| | 7.500 (1.78), 7.509 (1.80), 7.514 (1.87), 7.616 (2.59), 7.618 (2.67), |
| | 7.633 (2.24), 7.685 (1.65), 8.277 (2.11), 8.292 (2.03), 10.777 (2.74). |
| 176 | 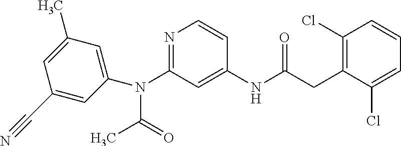 [SMILES: CC(=O)N(c1cc(C)cc(C#N)c1)c1cc(NC(=O)Cc2c(Cl)cccc2Cl)ccn1] |
| | N-(3-Cyan-5-methylphenyl)-N-{4-[2-(2,6- |
| | dichlorphenyl)acetamido]pyridin-2-yl}acetamide |
| | LC-MS (Method 2): Rₜ = 1.18 min; MS (ESIpos): m/z = 453 [M + H]⁺ |
| | ¹H-NMR (400 MHz, DMSO-d6) δ [ppm]: 2.002 (16.00), 2.074 (1.20), |
| | 2.084 (2.71), 2.335 (11.67), 2.518 (2.71), 2.523 (1.94), 2.669 (0.47), |
| | 4.082 (8.02), 7.336 (1.56), 7.355 (2.19), 7.358 (2.30), 7.376 (2.64), |
| | 7.447 (2.23), 7.464 (1.73), 7.469 (1.73), 7.478 (1.78), 7.483 (2.16), |
| | 7.488 (7.48), 7.508 (4.49), 7.616 (2.56), 7.618 (2.62), 7.637 (2.20), |
| | 7.685 (1.54), 8.281 (2.07), 8.295 (1.99), 10.892 (2.71). |
| 177 | 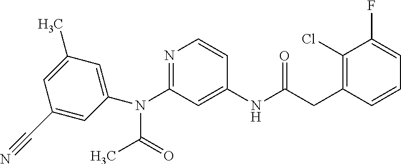 [SMILES: CC(=O)N(c1cc(C)cc(C#N)c1)c1cc(NC(=O)Cc2cccc(F)c2Cl)ccn1] |
| | N-{4-[2-(2-Chlor-3-fluorphenyl)acetamido]pyridin-2-yl}-N-(3-cyan-5- |
| | methylphenyl)acetamide |
| | LC-MS (Method 1): Rₜ = 1.16 min; MS (ESIpos): m/z = 437 [M + H]⁺ |
| | ¹H-NMR (400 MHz, DMSO-d6) δ [ppm]: 0.897 (0.43), 0.914 (1.09), |
| | 0.932 (0.81), 1.164 (0.52), 1.465 (0.76), 2.004 (16.00), 2.335 (12.34), |
| | 2.518 (4.49), 2.523 (3.09), 2.539 (7.30), 2.665 (0.56), 2.669 (0.78), |
| | 2.673 (0.56), 3.944 (5.44), 7.264 (0.85), 7.270 (1.16), 7.286 (1.36), |
| | 7.325 (0.48), 7.340 (1.98), 7.345 (2.54), 7.361 (4.39), 7.368 (1.58), |
| | 7.376 (1.29), 7.382 (0.43), 7.443 (2.37), 7.493 (1.15), 7.504 (1.18), |
| | 7.619 (2.92), 7.633 (2.39), 7.686 (1.71), 8.280 (1.86), 8.294 (1.78), |
| | 10.821 (2.00). |
| 178 | 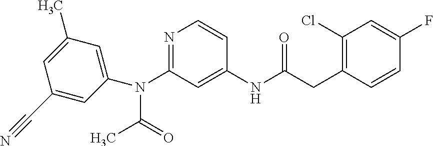 [SMILES: CC(=O)N(c1cc(C)cc(C#N)c1)c1cc(NC(=O)Cc2ccc(F)cc2Cl)ccn1] |
| | N-{4-[2-(2-Chlor-4-fluorphenyl)acetamido]pyridin-2-yl}-N-(3-cyan-5- |
| | methylphenyl)acetamide |
| | LC-MS (Method 1): Rₜ = 1.17 min; MS (ESIneg): m/z = 437 [M + H]⁺ |
| | ¹H-NMR (400 MHz, DMSO-d6) δ [ppm]: 2.003 (16.00), 2.074 (0.63), |
| | 2.334 (11.55), 2.518 (2.36), 2.523 (1.63), 2.540 (1.50), 2.669 (0.40), |
| | 3.871 (6.80), 7.188 (0.76), 7.195 (0.87), 7.210 (1.65), 7.217 (1.78), |
| | 7.231 (0.89), 7.238 (0.98), 7.433 (2.05), 7.439 (3.55), 7.447 (2.78), |
| | 7.455 (1.95), 7.462 (2.72), 7.469 (1.51), 7.486 (2.21), 7.491 (1.74), |
| | 7.501 (1.66), 7.505 (1.75), 7.617 (2.62), 7.619 (2.76), 7.631 (2.27), |
| | 7.682 (1.64), 8.276 (2.09), 8.290 (1.99), 10.776 (2.69). |
| 179 | 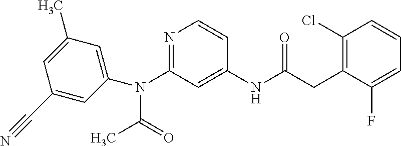 [SMILES: CC(=O)N(c1cc(C)cc(C#N)c1)c1cc(NC(=O)Cc2c(F)cccc2Cl)ccn1] |
| | N-{4-[2-(2-Chlor-6-fluorphenyl)acetamido]pyridin-2-yl}-N-(3-cyan-5- |
| | methylphenyl)acetamide |
| | LC-MS (Method 1): Rₜ = 1.15 min; MS (ESIpos): m/z = 437 [M + H]⁺ |
| | ¹H-NMR (400 MHz, DMSO-d6) δ [ppm]: 2.002 (16.00), 2.334 (12.21), |
| | 2.518 (6.68), 2.523 (4.72), 2.665 (0.83), 2.669 (1.14), 2.673 (0.81), |
| | 3.926 (5.10), 3.929 (4.99), 7.228 (0.69), 7.233 (0.73), 7.247 (1.00), |
| | 7.251 (1.65), 7.271 (1.04), 7.274 (1.00), 7.339 (0.73), 7.343 (0.94), |
| | 7.359 (3.76), 7.364 (3.48), 7.378 (1.48), 7.383 (1.35), 7.398 (1.48), |
| | 7.403 (0.47), 7.418 (0.48), 7.442 (2.31), 7.467 (1.70), 7.472 (1.74), |
| | 7.481 (1.70), 7.486 (1.68), 7.617 (2.80), 7.635 (2.31), 7.686 (1.67), |
| | 8.281 (2.11), 8.296 (2.04), 10.881 (2.66). |
| 180 | 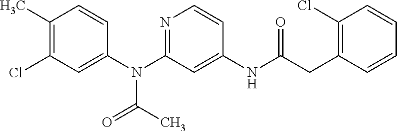 [SMILES: CC(=O)N(c1ccc(C)c(Cl)c1)c1cc(NC(=O)Cc2ccccc2Cl)ccn1] |
| | N-(3-Chlor-4-methylphenyl)-N-{4-[2-(2- |
| | chlorphenyl)acetamido]pyridin-2-yl}acetamide |
| | LC-MS (Method 2): Rₜ = 1.25 min; MS (ESIpos): m/z = 428 [M + H]⁺ |
| | ¹H-NMR (400 MHz, DMSO-d6) δ [ppm]: 1.130 (0.92), 1.146 (0.94), |
| | 1.997 (16.00), 2.319 (11.24), 2.518 (1.45), 2.522 (0.96), 3.873 (7.48), |
| | 7.112 (1.11), 7.118 (1.11), 7.132 (1.28), 7.138 (1.28), 7.300 (3.36), |
| | 7.309 (2.46), 7.315 (2.49), 7.324 (4.38), 7.334 (0.61), 7.359 (2.02), |
| | 7.381 (1.98), 7.388 (2.84), 7.393 (3.93), 7.401 (1.02), 7.404 (1.13), |
| | 7.408 (1.28), 7.417 (1.03), 7.424 (0.44), 7.433 (1.83), 7.442 (1.52), |
| | 7.447 (1.15), 7.457 (1.32), 7.474 (1.70), 7.478 (1.70), 7.488 (1.72), |
| | 7.493 (1.73), 7.657 (1.73), 8.268 (2.22), 8.282 (2.10), 10.754 (2.38). |
| 181 | 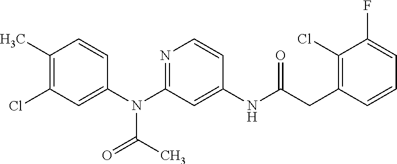 [SMILES: CC(=O)N(c1ccc(C)c(Cl)c1)c1cc(NC(=O)Cc2cccc(F)c2Cl)ccn1] |
| | N-{4-[2-(2-Chlor-3-fluorphenyl)acetamido]pyridin-2-yl}-N-(3-chlor- |
| | 4-methylphenyl)acetamide |
| | LC-MS (Method 2): Rₜ = 1.26 min; MS (ESIpos): m/z = 446 [M + H]⁺ |
| | ¹H-NMR (400 MHz, DMSO-d6) δ [ppm]: 1.139 (0.87), 1.154 (0.89), |
| | 1.997 (16.00), 2.321 (11.17), 2.518 (1.22), 2.523 (0.82), 3.937 (6.95), |
| | 7.115 (1.09), 7.120 (1.14), 7.135 (1.26), 7.140 (1.29), 7.259 (0.83), |
| | 7.266 (1.15), 7.272 (0.84), 7.277 (0.96), 7.282 (1.28), 7.322 (0.42), |
| | 7.337 (1.80), 7.341 (2.23), 7.358 (4.81), 7.365 (1.80), 7.372 (1.35), |
| | 7.382 (1.92), 7.389 (2.61), 7.395 (2.56), 7.467 (1.57), 7.472 (1.69), |
| | 7.481 (1.65), 7.486 (1.70), 7.657 (1.68), 8.271 (2.20), 8.285 (2.10), |
| | 10.792 (2.36). |
| 182 | 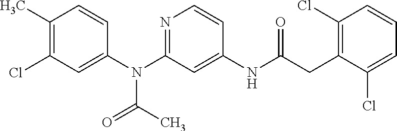 [SMILES: CC(=O)N(c1ccc(C)c(Cl)c1)c1cc(NC(=O)Cc2c(Cl)cccc2Cl)ccn1] |
| | N-(3-Chlor-4-methylphenyl)-N-{4-[2-(2,6- |
| | dichlorphenyl)acetamido]pyridin-2-yl}acetamide |
| | LC-MS (Method 2): Rₜ = 1.30 min; MS (ESIneg): m/z = 460 [M − H]⁻ |
| | ¹H-NMR (400 MHz, DMSO-d6) δ [ppm]: 1.995 (16.00), 2.320 (11.25), |
| | 2.518 (1.66), 2.523 (1.19), 4.076 (7.36), 7.117 (1.10), 7.122 (1.12), |
| | 7.138 (1.27), 7.143 (1.32), 7.334 (1.53), 7.353 (1.98), 7.355 (2.43), |
| | 7.360 (2.17), 7.374 (2.63), 7.381 (1.83), 7.392 (2.59), 7.398 (2.48), |
| | 7.440 (1.69), 7.445 (1.65), 7.454 (1.68), 7.459 (1.77), 7.485 (6.72), |
| | 7.505 (4.06), 7.658 (1.64), 8.271 (2.20), 8.286 (2.10), 10.869 (2.40). |
| 183 | 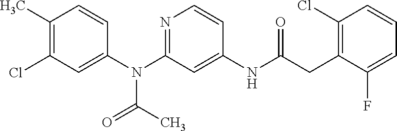 [SMILES: CC(=O)N(c1ccc(C)c(Cl)c1)c1cc(NC(=O)Cc2c(F)cccc2Cl)ccn1] |
| | N-{4-[2-(2-Chlor-6-fluorphenyl)acetamido]pyridin-2-yl}-N-(3-chlor- |
| | 4-methylphenyl)acetamide |
| | LC-MS (Method 2): Rₜ = 1.26 min; MS (ESIpos): m/z = 446 [M + H]⁺ |
| | ¹H-NMR (400 MHz, DMSO-d6) δ [ppm]: 1.995 (16.00), 2.320 (10.77), |
| | 2.518 (1.42), 2.523 (1.05), 3.920 (4.14), 3.923 (4.02), 7.115 (1.07), |
| | 7.120 (1.06), 7.135 (1.22), 7.140 (1.25), 7.224 (0.59), 7.229 (0.61), |
| | 7.243 (0.88), 7.248 (1.40), 7.268 (0.85), 7.272 (0.84), 7.336 (0.69), |
| | 7.340 (0.80), 7.356 (3.76), 7.361 (4.23), 7.376 (1.61), 7.381 (2.88), |
| | 7.390 (2.62), 7.396 (3.55), 7.416 (0.46), 7.445 (1.55), 7.450 (1.68), |
| | 7.460 (1.63), 7.464 (1.69), 7.659 (1.60), 8.272 (2.09), 8.286 (2.02), |
| | 10.856 (2.25) |
| 184 | 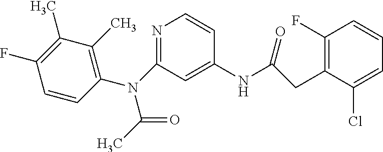 [SMILES: CC(=O)N(c1cc(NC(=O)Cc2c(F)cccc2Cl)ccn1)c1ccc(F)c(C)c1C] |
| | N-{4-[2-(2-Chlor-6-fluorphenyl)acetamido]pyridin-2-yl}-N-(4-fluor- |
| | 2,3-dimethylphenyl)acetamide |
| | LC-MS (Method 2): Rₜ = 1.23 min; MS (ESIpos): m/z = 444 [M + H]⁺ |
| | ¹H-NMR (400 MHz, DMSO-d6) δ [ppm]: 1.958 (11.35), 2.094 (16.00), |
| | 2.169 (8.22), 2.174 (8.15), 2.332 (0.63), 2.518 (2.83), 2.522 (1.85), |
| | 2.673 (0.60), 3.919 (5.76), 3.923 (5.54), 7.034 (0.95), 7.057 (2.42), |
| | 7.079 (1.69), 7.110 (1.45), 7.124 (1.57), 7.131 (0.93), 7.145 (0.81), |
| | 7.228 (0.86), 7.233 (0.85), 7.247 (1.28), 7.252 (2.03), 7.256 (1.09), |
| | 7.272 (1.29), 7.276 (1.22), 7.340 (0.95), 7.343 (1.18), 7.360 (5.06), |
| | 7.365 (4.51), 7.378 (1.84), 7.384 (1.68), 7.399 (2.38), 7.401 (2.80), |
| | 7.405 (2.69), 7.415 (2.57), 7.419 (2.92), 7.746 (1.19), 8.193 (2.72), |
| | 8.207 (2.58), 10.813 (3.15). |

### Example 185 (Examples with Pyridazine Core)

### N-{5-[2-(2,6-dichlorophenyl)acetamido]pyridazin-3-yl}-N-phenylacetamide

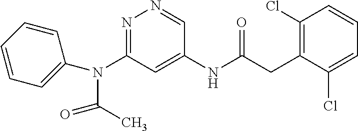 [SMILES: CC(=O)N(c1ccccc1)c1cc(NC(=O)Cc2c(Cl)cccc2Cl)cnn1]

According to GP J, N-(6-anilinopyridazin-4-yl)-2-(2,6-dichlorophenyl)acetamide (Int. 123, 56 mg, 0.15 mmol) was dissolved in dichloromethane (2 mL) and acetyl chloride (18 mg, 0.22 mmol, 1.5 eq) and triethylamine (27 mg, 0.27, 1.8 eq) were added. The mixture was stirred at rt for 18 h, then concentrated in vacuo and purified via preparative HPLC to yield 45 mg (73% of theory) of the title compound.

¹H-NMR (400 MHz, DMSO-d6) δ[ppm]: −0.009 (0.55), 0.008 (0.44), 1.109 (11.45), 2.016 (10.62), 2.324 (0.40), 2.329 (0.55), 2.334 (0.40), 2.520 (1.83), 2.525 (1.23), 2.542 (16.00), 2.666 (0.40), 2.671 (0.57), 2.676 (0.40), 4.130 (4.81), 4.196 (0.95), 7.346 (0.51), 7.349 (1.32), 7.360 (1.56), 7.363 (2.20), 7.368 (1.82), 7.371 (1.94), 7.379 (2.97), 7.382 (2.46), 7.390 (1.61), 7.431 (1.96), 7.446 (1.58), 7.449 (1.56), 7.455 (0.42), 7.468 (0.84), 7.500 (4.26), 7.519 (2.61), 8.102 (1.30), 8.107 (1.28), 9.124 (2.46), 9.129 (2.40), 11.166 (1.38).

LC-MS (Method 1): Rₜ=1.10 min; MS (ESIpos): m/z=415 [M+H]⁺

TABLE 9
Examples 186-208: According to GP J, the following examples were
prepared

| | |
|---|---|
| 186 | 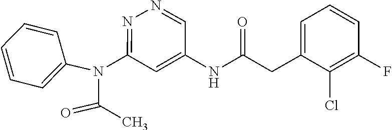 |
| | N-{5-[2-(2-chloro-3-fluorophenyl)acetamido]pyridazin-3-yl}-N- |
| | phenylacetamide |
| | LC-MS (Method 1): Rₜ = 1.05 min; MS (ESIneg): m/z = 397 [M − H]⁻ |
| | ¹H-NMR (400 MHz, DMSO-d6) δ [ppm]: −0.008 (1.19), 0.008 (1.10), |
| | 1.343 (0.45), 2.019 (16.00), 2.070 (0.67), 2.171 (0.41), 2.206 (1.22), |
| | 2.521 (2.84), 2.525 (1.93), 2.585 (0.42), 3.995 (6.91), 4.004 (0.81), |
| | 4.029 (0.49), 7.277 (0.73), 7.285 (1.15), 7.290 (0.81), 7.294 (0.88), |
| | 7.301 (1.23), 7.339 (0.50), 7.344 (0.45), 7.347 (0.94), 7.355 (3.75), |
| | 7.360 (2.71), 7.364 (2.39), 7.372 (5.23), 7.375 (4.21), 7.377 (3.93), |
| | 7.382 (4.08), 7.387 (1.87), 7.392 (0.46), 7.426 (0.59), 7.432 (2.69), |
| | 7.445 (1.03), 7.448 (1.93), 7.450 (2.57), 7.453 (1.64), 7.456 (0.67), |
| | 7.465 (0.59), 7.469 (1.27), 7.472 (0.70), 8.104 (2.03), 8.110 (2.01), |
| | 9.147 (3.89), 9.153 (3.83), 11.075 (1.19). |
| 187 | 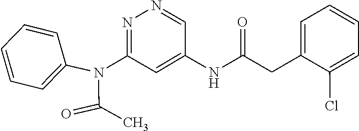 [SMILES: CC(=O)N(c1ccccc1)c1cc(NC(=O)Cc2ccccc2Cl)cnn1] |
| | N-{5-[2-(2-chlorophenyl)acetamido]pyridazin-3-yl}-N- |
| | phenylacetamide |
| | LC-MS (Method 2): Rₜ = 1.00 min; MS (ESIpos): m/z = 381 [M + H]⁺ |
| | ¹H-NMR (400 MHz, DMSO-d6) δ [ppm]: −0.008 (0.69), 0.008 (0.72), |
| | 2.020 (16.00), 2.334 (0.50), 2.520 (2.25), 2.525 (1.39), 2.676 (0.44), |
| | 3.932 (7.34), 7.304 (0.43), 7.315 (2.71), 7.323 (2.73), 7.328 (3.00), |
| | 7.337 (3.85), 7.347 (1.19), 7.353 (1.92), 7.356 (2.26), 7.363 (2.17), |
| | 7.368 (1.67), 7.376 (3.67), 7.380 (2.69), 7.411 (1.56), 7.421 (1.30), |
| | 7.426 (1.69), 7.431 (3.03), 7.448 (3.65), 7.455 (1.61), 7.462 (1.67), |
| | 7.469 (1.52), 7.470 (1.89), 8.104 (2.05), 8.109 (2.05), 9.153 (3.41), |
| | 9.159 (3.33), 11.038 (2.24). |
| 188 | 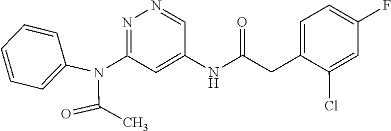 [SMILES: CC(=O)N(c1ccccc1)c1cc(NC(=O)Cc2ccc(F)cc2Cl)cnn1] |
| | N-{5-[2-(2-chloro-4-fluorophenyl)acetamido]pyridazin-3-yl}-N- |
| | phenylacetamide |
| | LC-MS (Method 2): Rₜ = 1.06 min; MS (ESIpos): m/z = 399 [M + H]⁺ |
| | ¹H-NMR (400 MHz, DMSO-d6) δ [ppm]: −0.008 (1.19), 0.008 (1.12), |
| | 2.019 (16.00), 2.334 (0.71), 2.520 (3.36), 2.525 (2.18), 2.676 (0.71), |
| | 3.311 (0.44), 3.923 (6.92), 7.199 (0.71), 7.206 (0.81), 7.220 (1.53), |
| | 7.227 (1.68), 7.242 (0.84), 7.248 (0.89), 7.346 (0.92), 7.356 (2.37), |
| | 7.364 (2.20), 7.368 (1.83), 7.373 (3.68), 7.376 (3.73), 7.381 (2.37), |
| | 7.431 (2.97), 7.447 (3.64), 7.450 (3.27), 7.453 (3.02), 7.462 (1.78), |
| | 7.469 (2.92), 7.476 (2.57), 7.483 (1.36), 7.499 (1.21), 8.100 (2.23), |
| | 8.105 (2.22), 9.145 (3.88), 9.151 (3.83), 11.035 (2.12). |
| 189 | 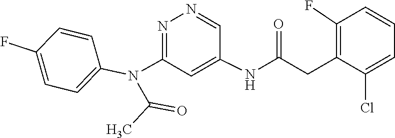 [SMILES: CC(=O)N(c1ccc(F)cc1)c1cc(NC(=O)Cc2c(F)cccc2Cl)cnn1] |
| | N-{5-[2-(2-chloro-6-fluorophenyl)acetamido]pyridazin-3-yl}-N-(4- |
| | fluorophenyl)acetamide |
| | LC-MS (Method 1): Rₜ = 1.03 min; MS (ESIpos): m/z = 417 [M + H]⁺ |
| | ¹H-NMR (400 MHz, DMSO-d6) δ [ppm]: 2.011 (16.00), 2.465 (0.42), |
| | 2.470 (0.56), 2.520 (3.11), 2.525 (2.13), 3.298 (0.50), 3.304 (0.44), |
| | 3.312 (0.81), 3.320 (1.24), 3.350 (1.16), 3.355 (0.47), 3.978 (4.00), |
| | 3.982 (3.84), 7.241 (0.63), 7.246 (0.66), 7.258 (2.28), 7.265 (1.92), |
| | 7.268 (0.91), 7.275 (0.85), 7.281 (4.02), 7.285 (1.50), 7.288 (1.12), |
| | 7.296 (0.72), 7.302 (2.47), 7.352 (0.69), 7.356 (0.78), 7.372 (3.07), |
| | 7.377 (1.89), 7.379 (1.79), 7.393 (1.23), 7.398 (1.15), 7.413 (1.33), |
| | 7.418 (0.63), 7.427 (2.13), 7.433 (1.18), 7.440 (2.27), 7.444 (1.00), |
| | 7.450 (1.87), 7.457 (0.65), 7.463 (1.65), 8.137 (1.77), 8.142 (1.74), |
| | 9.130 (3.66), 9.135 (3.64), 11.146 (0.63). |
| 190 | 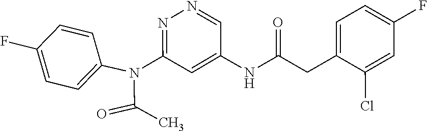 [SMILES: CC(=O)N(c1ccc(F)cc1)c1cc(NC(=O)Cc2ccc(F)cc2Cl)cnn1] |
| | N-{5-[2-(2-chloro-4-fluorophenyl)acetamido]pyridazin-3-yl}-N-(4- |
| | fluorophenyl)acetamide |
| | LC-MS (Method 1): Rₜ = 1.10 min; MS (ESIpos): m/z = 417 [M + H]⁺ |
| | ¹H-NMR (400 MHz, DMSO-d6) δ [ppm]: 0.970 (0.41), 1.112 (0.56), |
| | 2.014 (16.00), 2.334 (0.63), 2.521 (2.91), 2.525 (1.85), 2.676 (0.63), |
| | 3.927 (6.54), 7.202 (0.73), 7.208 (0.85), 7.223 (1.61), 7.230 (1.79), |
| | 7.244 (0.94), 7.251 (1.04), 7.261 (2.12), 7.266 (0.78), 7.278 (0.88), |
| | 7.283 (4.44), 7.288 (0.86), 7.292 (0.42), 7.299 (0.77), 7.305 (2.68), |
| | 7.427 (2.33), 7.432 (0.91), 7.439 (2.49), 7.444 (1.26), 7.449 (3.67), |
| | 7.455 (2.27), 7.462 (2.14), 7.465 (1.73), 7.471 (1.89), 7.477 (1.88), |
| | 7.481 (1.63), 7.487 (1.36), 7.503 (1.21), 8.138 (1.97), 8.144 (1.99), |
| | 9.144 (4.00), 9.150 (3.94), 11.048 (1.27). |
| 191 | 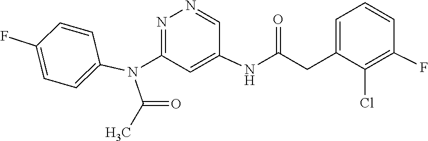 [SMILES: CC(=O)N(c1ccc(F)cc1)c1cc(NC(=O)Cc2cccc(F)c2Cl)cnn1] |
| | N-{5-[2-(2-chloro-3-fluorophenyl)acetamido]pyridazin-3-yl}-N-(4- |
| | fluorophenyl)acetamide |
| | LC-MS (Method 1): Rₜ = 1.03 min; MS (ESIpos): m/z = 417 [M + H]⁺ |
| | ¹H-NMR (400 MHz, DMSO-d6) δ [ppm]: 0.967 (0.88), 1.107 (11.11) |
| | 1.109 (2.26), 1.144 (0.57), 2.011 (16.00), 2.332 (0.89), 2.447 (0.48), |
| | 2.518 (3.94), 2.523 (2.56), 2.673 (0.89), 3.995 (6.64), 4.188 (0.91), |
| | 7.258 (2.05), 7.264 (0.79), 7.280 (4.25), 7.285 (2.00), 7.290 (1.12), |
| | 7.296 (1.27), 7.302 (3.41), 7.311 (0.45), 7.338 (0.42), 7.352 (2.07), |
| | 7.355 (2.12), 7.359 (1.42), 7.371 (4.24), 7.380 (1.33), 7.386 (1.32), |
| | 7.425 (2.12), 7.431 (0.82), 7.437 (2.25), 7.443 (1.03), 7.447 (1.94), |
| | 7.454 (0.69), 7.460 (1.73), 8.139 (1.79), 8.145 (1.74), 9.141 (3.72), |
| | 9.147 (3.68), 11.080 (1.21). |
| 192 | 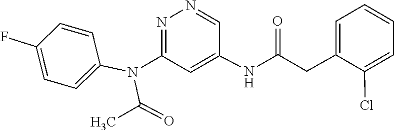 [SMILES: CC(=O)N(c1ccc(F)cc1)c1cc(NC(=O)Cc2ccccc2Cl)cnn1] |
| | N-{5-[2-(2-chlorophenyl)acetamido]pyridazin-3-yl}-N-(4- |
| | fluorophenyl)acetamide |
| | LC-MS (Method 1): Rₜ = 1.02 min; MS (ESIpos): m/z = 399 [M + H]⁺ |
| | ¹H-NMR (400 MHz, DMSO-d6) δ [ppm]: 0.967 (0.93), 1.107 (8.33), |
| | 1.109 (2.29), 1.144 (0.56), 2.012 (16.00), 2.332 (0.68), 2.518 (2.98), |
| | 2.523 (2.00), 2.673 (0.69), 3.932 (7.28), 4.189 (0.66), 7.257 (2.11), |
| | 7.263 (0.77), 7.274 (0.96), 7.280 (4.07), 7.284 (0.87), 7.289 (0.43), |
| | 7.295 (0.85), 7.301 (2.81), 7.314 (3.38), 7.322 (3.28), 7.328 (3.57), |
| | 7.337 (4.31), 7.347 (0.79), 7.412 (1.68), 7.424 (2.70), 7.426 (2.02), |
| | 7.436 (3.40), 7.442 (1.24), 7.447 (3.62), 7.456 (1.79), 7.459 (2.12), |
| | 7.461 (1.83), 7.470 (1.29), 8.138 (1.87), 8.143 (1.79), 9.148 (4.11), |
| | 9.154 (3.98), 11.044 (1.34). |
| 193 | 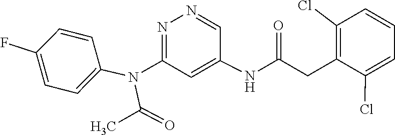 [SMILES: CC(=O)N(c1ccc(F)cc1)c1cc(NC(=O)Cc2c(Cl)cccc2Cl)cnn1] |
| | N-{5-[2-(2,6-dichlorophenyl)acetamido]pyridazin-3-yl}-N-(4- |
| | fluorophenyl)acetamide |
| | LC-MS (Method 1): Rₜ = 1.11 min; MS (ESIpos): m/z = 433 [M + H]⁺ |
| | ¹H-NMR (400 MHz, DMSO-d6) δ [ppm]: 1.107 (16.00), 2.007 (6.63), |
| | 2.518 (0.78), 2.522 (0.48), 4.130 (3.05), 4.191 (1.37), 7.256 (0.82), |
| | 7.278 (1.72), 7.299 (1.03), 7.348 (0.59), 7.367 (0.79), 7.369 (0.83), |
| | 7.388 (0.99), 7.427 (0.89), 7.440 (0.95), 7.445 (0.45), 7.450 (0.80), |
| | 7.462 (0.73), 7.498 (2.77), 7.518 (1.69), 8.138 (0.73), 8.143 (0.76), |
| | 9.117 (1.48), 9.123 (1.48). |
| 194 | 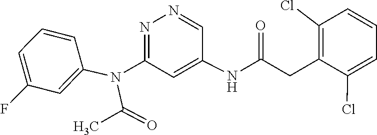 [SMILES: CC(=O)N(c1cccc(F)c1)c1cc(NC(=O)Cc2c(Cl)cccc2Cl)cnn1] |
| | N-{5-[2-(2,6-dichlorophenyl)acetamido]pyridazin-3-yl}-N-(3- |
| | fluorophenyl)acetamide |
| | LC-MS (Method 1): Rₜ = 1.07 min; MS (ESIpos): m/z = 433 [M + H]⁺ |
| 195 | 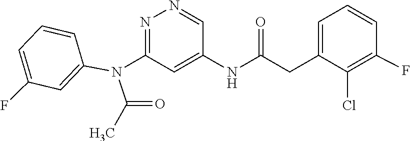 [SMILES: CC(=O)N(c1cccc(F)c1)c1cc(NC(=O)Cc2cccc(F)c2Cl)cnn1] |
| | N-{5-[2-(2-chloro-3-fluorophenyl)acetamido]pyridazin-3-yl}-N-(3- |
| | fluorophenyl)acetamide |
| | LC-MS (Method 1): Rₜ = 1.04 min; MS (ESIpos): m/z = 417 [M + H]⁺ |
| 196 | 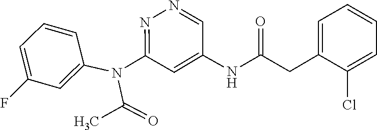 [SMILES: CC(=O)N(c1cccc(F)c1)c1cc(NC(=O)Cc2ccccc2Cl)cnn1] |
| | N-{5-[2-(2-chlorophenyl)acetamido]pyridazin-3-yl}-N-(3- |
| | fluorophenyl)acetamide |
| | LC-MS (Method 1): Rₜ = 1.04 min; MS (ESIneg): m/z = 397 [M − H]⁻ |
| | ¹H-NMR (400 MHz, DMSO-d6) δ [ppm]: 1.107 (16.00), 2.038 (8.05), |
| | 2.518 (0.52), 3.936 (3.60), 4.191 (1.35), 7.182 (0.49), 7.184 (0.47), |
| | 7.186 (0.45), 7.202 (0.71), 7.205 (0.74), 7.224 (0.45), 7.226 (0.43), |
| | 7.231 (0.54), 7.233 (0.47), 7.313 (1.45), 7.322 (1.39), 7.328 (1.58), |
| | 7.337 (2.31), 7.344 (0.44), 7.346 (0.45), 7.363 (0.52), 7.413 (0.76), |
| | 7.422 (0.55), 7.427 (0.59), 7.436 (0.63), 7.447 (0.98), 7.456 (0.57), |
| | 7.461 (0.73), 7.466 (0.55), 7.470 (1.24), 7.486 (0.63), 8.132 (0.88), |
| | 8.137 (0.89), 9.170 (1.82), 9.176 (1.78), 11.062 (0.82). |
| 197 | 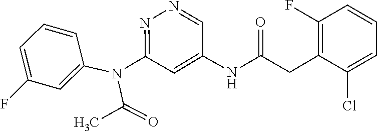 [SMILES: CC(=O)N(c1cccc(F)c1)c1cc(NC(=O)Cc2c(F)cccc2Cl)cnn1] |
| | N-{5-[2-(2-chloro-6-fluorophenyl)acetamido]pyridazin-3-yl}-N-(3- |
| | fluorophenyl)acetamide |
| | LC-MS (Method 1): Rₜ = 1.03 min; MS (ESIpos): m/z = 417 [M + H]⁺ |
| 198 | 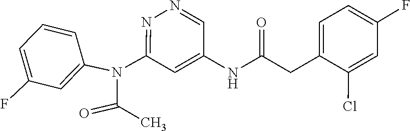 [SMILES: CC(=O)N(c1cccc(F)c1)c1cc(NC(=O)Cc2ccc(F)cc2Cl)cnn1] |
| | N-{5-[2-(2-chloro-4-fluorophenyl)acetamido]pyridazin-3-yl}-N-(3- |
| | fluorophenyl)acetamide |
| | LC-MS (Method 1): Rₜ = 1.07 min; MS (ESIneg): m/z = 415 [M − H]⁻ |
| | ¹H-NMR (400 MHz, DMSO-d6) δ [ppm]: 1.109 (1.18), 2.039 (3.88), |
| | 2.520 (1.43), 2.525 (0.95), 2.542 (16.00), 2.671 (0.44), 3.929 (1.46), |
| | 7.201 (0.40), 7.208 (0.53), 7.229 (0.62), 7.455 (0.44), 7.470 (0.59), |
| | 7.477 (0.44), 8.130 (0.43), 8.135 (0.43), 9.164 (0.91), 9.170 (0.90). |
| 199 | 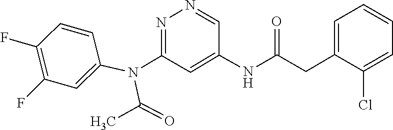 [SMILES: CC(=O)N(c1ccc(F)c(F)c1)c1cc(NC(=O)Cc2ccccc2Cl)cnn1] |
| | N-{5-[2-(2-chlorophenyl)acetamido]pyridazin-3-yl}-N-(3,4- |
| | difluorophenyl)acetamide |
| | LC-MS (Method 1): Rₜ = 1.05 min; MS (ESIpos): m/z = 417 [M + H]⁺ |
| 200 | 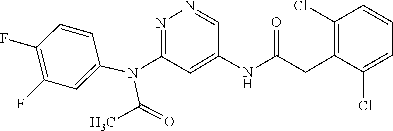 [SMILES: CC(=O)N(c1ccc(F)c(F)c1)c1cc(NC(=O)Cc2c(Cl)cccc2Cl)cnn1] |
| | N-{5-[2-(2,6-dichlorophenyl)acetamido]pyridazin-3-yl}-N-(3,4- |
| | difluorophenyl)acetamide |
| | LC-MS (Method 1): Rₜ = 1.13 min; MS (ESIneg): m/z = 449 [M − H]⁻ |
| | ¹H-NMR (400 MHz, DMSO-d6) δ [ppm]: 0.967 (0.56), 1.107 (16.00), |
| | 2.027 (10.25), 2.332 (0.40), 2.518 (2.07), 2.522 (1.27), 2.669 (0.56), |
| | 2.673 (0.41), 4.136 (4.80), 4.189 (1.55), 7.255 (0.42), 7.277 (0.49), |
| | 7.350 (1.09), 7.369 (1.27), 7.372 (1.49), 7.391 (1.62), 7.482 (0.41), |
| | 7.501 (4.55), 7.521 (2.69), 7.530 (0.84), 7.658 (0.43), 7.661 (0.43), |
| | 8.176 (1.08), 8.181 (1.10), 9.132 (2.02), 9.138 (2.03), 11.179 (1.42). |
| 201 | 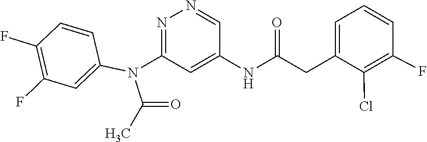 [SMILES: CC(=O)N(c1ccc(F)c(F)c1)c1cc(NC(=O)Cc2cccc(F)c2Cl)cnn1] |
| | N-{5-[2-(2-chloro-3-fluorophenyl)acetamido]pyridazin-3-yl}-N-(3,4- |
| | difluorophenyl)acetamide |
| | LC-MS (Method 1): Rₜ = 1.06 min; MS (ESIpos): m/z = 435 [M + H]⁺ |
| | ¹H-NMR (400 MHz, DMSO-d6) δ [ppm]: 0.967 (1.23), 1.107 (6.07), |
| | 1.109 (2.38), 1.144 (0.75), 1.232 (0.42), 2.030 (16.00), 2.332 (1.17), |
| | 2.336 (0.49), 2.446 (0.51), 2.452 (0.53), 2.518 (5.28), 2.523 (3.48), |
| | 2.673 (1.17), 2.678 (0.51), 3.998 (6.02), 4.189 (0.47), 7.249 (0.60), |
| | 7.271 (0.76), 7.278 (1.04), 7.287 (1.12), 7.293 (0.82), 7.297 (0.85), |
| | 7.303 (1.14), 7.352 (1.88), 7.355 (1.98), 7.359 (1.32), 7.373 (4.11), |
| | 7.381 (1.27), 7.387 (1.22), 7.482 (0.56), 7.505 (1.11), 7.509 (0.74), |
| | 7.527 (0.69), 7.531 (1.09), 7.554 (0.51), 7.630 (0.48), 7.636 (0.48), |
| | 7.648 (0.53), 7.655 (0.59), 7.665 (0.51), 7.677 (0.51), 7.682 (0.47), |
| | 8.172 (1.48), 8.177 (1.45), 9.151 (2.86), 9.157 (2.77), 11.103 (0.56). |
| 202 | 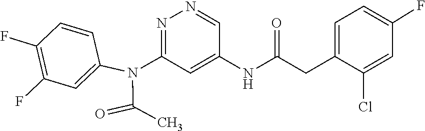 [SMILES: CC(=O)N(c1ccc(F)c(F)c1)c1cc(NC(=O)Cc2ccc(F)cc2Cl)cnn1] |
| | N-{5-[2-(2-chloro-4-fluorophenyl)acetamido]pyridazin-3-yl}-N-(3,4- |
| | difluorophenyl)acetamide |
| | LC-MS (Method 1): Rₜ = 1.13 min; MS (ESIpos): m/z = 435 [M + H]⁺ |
| | ¹H-NMR (400 MHz, DMSO-d6) δ [ppm]: 0.970 (0.44), 1.110 (1.23), |
| | 1.112 (0.74), 2.032 (16.00), 2.334 (0.65), 2.521 (2.99), 2.525 (1.94), |
| | 2.672 (0.91), 2.676 (0.66), 3.931 (5.84), 7.204 (0.70), 7.211 (0.75), |
| | 7.225 (1.42), 7.232 (1.66), 7.246 (1.20), 7.253 (1.36), 7.263 (0.54), |
| | 7.270 (0.55), 7.274 (0.71), 7.450 (1.52), 7.456 (1.50), 7.469 (1.42), |
| | 7.472 (1.70), 7.479 (1.60), 7.484 (1.77), 7.489 (1.32), 7.506 (1.55), |
| | 7.512 (0.78), 7.531 (0.66), 7.534 (1.17), 7.557 (0.54), 7.632 (0.50), |
| | 7.639 (0.51), 7.651 (0.56), 7.657 (0.61), 7.661 (0.63), 7.667 (0.55), |
| | 7.679 (0.53), 7.686 (0.51), 8.173 (1.58), 8.179 (1.60), 9.155 (3.45), |
| | 9.161 (3.40), 11.063 (0.61). |
| 203 | 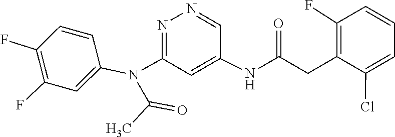 [SMILES: CC(=O)N(c1ccc(F)c(F)c1)c1cc(NC(=O)Cc2c(F)cccc2Cl)cnn1] |
| | N-{5-[2-(2-chloro-6-fluorophenyl)acetamido]pyridazin-3-yl}-N-(3,4- |
| | difluorophenyl)acetamide |
| | LC-MS (Method 1): Rₜ = 1.06 min; MS (ESIpos): m/z = 435 [M + H]⁺ |
| | ¹H-NMR (400 MHz, DMSO-d6) δ [ppm]: −0.002 (1.32), 2.026 (16.00), |
| | 2.331 (0.91), 2.336 (0.41), 2.518 (3.89), 2.523 (2.55), 2.673 (0.91), |
| | 3.977 (3.88), 3.980 (3.72), 7.240 (0.87), 7.245 (1.01), 7.248 (0.65), |
| | 7.258 (1.24), 7.263 (1.82), 7.266 (1.26), 7.270 (0.76), 7.282 (1.01), |
| | 7.286 (0.89), 7.350 (0.66), 7.354 (0.79), 7.370 (3.28), 7.376 (2.34), |
| | 7.390 (1.18), 7.396 (1.10), 7.410 (1.27), 7.431 (0.41), 7.480 (0.58), |
| | 7.502 (1.16), 7.506 (0.73), 7.524 (0.66), 7.529 (1.15), 7.551 (0.52), |
| | 7.627 (0.47), 7.634 (0.48), 7.646 (0.52), 7.652 (0.58), 7.656 (0.58), |
| | 7.662 (0.53), 7.674 (0.50), 7.681 (0.47), 8.165 (1.44), 8.170 (1.44), |
| | 9.133 (2.59), 9.139 (2.55), 11.174 (0.57). |
| 204 | 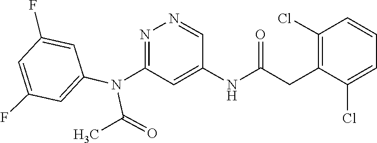 [SMILES: CC(=O)N(c1cc(F)cc(F)c1)c1cc(NC(=O)Cc2c(Cl)cccc2Cl)cnn1] |
| | N-{5-[2-(2,6-dichlorophenyl)acetamido]pyridazin-3-yl}-N-(3,5- |
| | difluorophenyl)acetamide |
| | LC-MS (Method 1): Rₜ = 1.11 min; MS (ESIneg): m/z = 449 [M − H]⁻ |
| 205 | 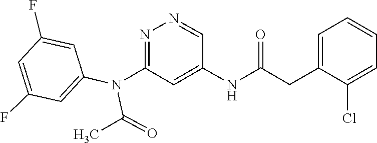 [SMILES: CC(=O)N(c1cc(F)cc(F)c1)c1cc(NC(=O)Cc2ccccc2Cl)cnn1] |
| | N-{5-[2-(2-chlorophenyl)acetamido]pyridazin-3-yl}-N-(3,5- |
| | difluorophenyl)acetamide |
| | LC-MS (Method 1): Rₜ = 1.06 min; MS (ESIpos): m/z = 417 [M + H]⁺ |
| 206 | 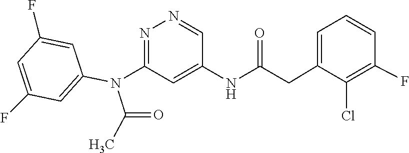 [SMILES: CC(=O)N(c1cc(F)cc(F)c1)c1cc(NC(=O)Cc2cccc(F)c2Cl)cnn1] |
| | N-{5-[2-(2-chloro-3-fluorophenyl)acetamido]pyridazin-3-yl}-N-(3,5- |
| | difluorophenyl)acetamide |
| | LC-MS (Method 1): Rₜ = 1.08 min; MS (ESIpos): m/z = 435 [M + H]⁺ |
| 207 | 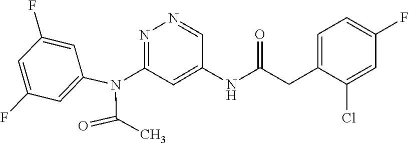 [SMILES: CC(=O)N(c1cc(F)cc(F)c1)c1cc(NC(=O)Cc2ccc(F)cc2Cl)cnn1] |
| | N-{5-[2-(2-chloro-4-fluorophenyl)acetamido]pyridazin-3-yl}-N-(3,5- |
| | difluorophenyl)acetamide |
| | LC-MS (Method 1): Rₜ = 1.14 min; MS (ESIpos): m/z = 435 [M + H]⁺ |
| | 1H-NMR (400 MHz, DMSO-d6) delta [ppm]: −0.008 (1.12), 0.008 |
| | (1.17), 0.970 (0.53), 1.109 (4.03), 2.058 (16.00), 2.334 |
| | (0.86), 2.520 (3.85), 2.525 (2.53), 2.676 (0.88), 3.933 |
| | (5.83), 7.203 (0.77), 7.210 (0.97), 7.224 (2.62), 7.231 (2.75), |
| | 7.246 (2.00), 7.253 (1.28), 7.283 (0.62), 7.301 (0.79), 7.306 |
| | (1.25), 7.313 (0.64), 7.330 (0.62), 7.450 (1.52), 7.456 (1.54), |
| | 7.469 (1.47), 7.472 (1.80), 7.479 (1.56), 7.484 (1.43), |
| | 7.490 (1.23), 7.506 (1.14), 8.160 (1.56), 8.165 (1.58), 9.174 (3.15), |
| | 9.180 (3.08), 11.079 (0.57). |
| 208 | 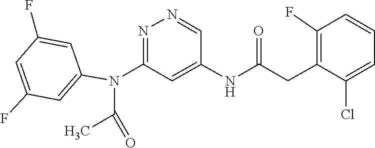 [SMILES: CC(=O)N(c1cc(F)cc(F)c1)c1cc(NC(=O)Cc2c(F)cccc2Cl)cnn1] |
| | N-{5-[2-(2-chloro-6-fluorophenyl)acetamido]pyridazin-3-yl}-N-(3,5- |
| | difluorophenyl)acetamide |
| | LC-MS (Method 1): Rₜ = 1.07 min; MS (ESIpos): m/z = 435 [M + H]⁺ |
| | ¹H-NMR (400 MHz, DMSO-d6) δ [ppm]: −0.002 (0.68), 2.052 (16.00), |
| | 2.331 (1.04), 2.336 (0.48), 2.518 (4.74), 2.523 (3.10), 2.673 (1.08), |
| | 2.678 (0.47), 3.982 (3.65), 3.985 (3.54), 7.221 (1.22), 7.226 (1.57), |
| | 7.240 (1.85), 7.245 (1.89), 7.258 (1.02), 7.263 (1.47), 7.272 (0.45), |
| | 7.278 (0.67), 7.284 (1.07), 7.287 (0.90), 7.295 (0.72), 7.301 (1.17), |
| | 7.307 (0.62), 7.325 (0.58), 7.351 (0.62), 7.355 (0.73), 7.372 (2.91), |
| | 7.377 (2.24), 7.391 (1.16), 7.396 (1.07), 7.412 (1.17), 8.154 (1.37), |
| | 8.160 (1.37), 9.157 (2.44), 9.163 (2.39), 11.190 (0.58). |

### Experimental Section—Biological Assays

Examples were tested in selected biological assays one or more times. When tested more than once, data are reported as either average values or as median values, wherein
the average value, also referred to as the arithmetic mean value, represents the sum of the values obtained divided by the number of times tested, and
the median value represents the middle number of the group of values when ranked in ascending or descending order. If the number of values in the data set is odd, the median is the middle value. If the number of values in the data set is even, the median is the arithmetic mean of the two middle values.

Examples were synthesized one or more times. When synthesized more than once, data from biological assays represent average values or median values calculated utilizing data sets obtained from testing of one or more synthetic batch.

In Vitro Studies

The in vitro activity of the compounds of the present invention can be demonstrated in the following assays:

Human P2X4 HEK Cell FLIPR Assay

Compounds were tested on a HEK293 cell line stably expressing human P2X4. Cells were cultured on poly-D-lysine-coated 384-well plates at a density of 15,000 cells/well and incubated overnight at 37° C., 5% CO₂. P2X4 function was assessed by measuring intracellular calcium fluxes caused by Benzoylbenzoyl-ATP (Bz-ATP) using the calcium-chelating dye Fluo8-AM (Molecular Devices) with the Fluorescent Imaging Plate Reader Tetra (FLIPR^{Tetra}; Molecular Devices CA). On the day of the assay, the medium was removed and the cells were incubated for 30 min at 37° C. and 5% CO₂ in 30 μl of dye buffer (Hank's balanced salt solution, 10 mM HEPES, 1.8 mM CaCl₂, 1 mM MgCl₂, 2 mM probenecid, 5 mM D-glucose monohydrate, 5 μM Fluo8-AM, pH=7.4). Compounds diluted in probenecid buffer (Hank's balanced salt solution, 10 mM HEPES, 1.8 mM CaCl₂, 1 mM MgCl₂, 2 mM probenecid, 5 mM D-glucose monohydrate, pH=7.4) at ten concentrations ranging from 25 μM to 1 nM (final concentration) were dispensed and incubated for 30 min at room temperature. The agonist, Bz-ATP (Tocris Bio-Techne GmbH, DE), was added at a final concentration of 3 μM, representing the EC80 routinely determined. The final assay volume was 50 μl and final DMSO concentration was 0.5%.

The fluorescence intensity reflecting intarcellular calcium changes was recorded before and after Bz-ATP addition, at an excitation and emission wavelengths of 470-495 nm and 515-575 nm respectively.

The compounds were tested in triplicates and fluorescence intensity raw data normalized to the agonist control and fitted to the four-parameter logistic equation:

Y=Bottom+(Top−Bottom)/(1+10\hat{ }((Log IC50−X)*HillSlope))

The efficacy of saturating concentrations of the agonist BzATP (3 μM) was set as maximal response (100% Emax) and the bottom defined by the signal achieved with 0.5% DMSO.

Assay plate acceptance was based on the signal window (S/B)≥1.8, Z′≥0.5 and the reference compound pIC50 within ±3σ the mean of historic pIC50 of the compound. Failure to meet two of the three criteria determined exclusion of the plate's results.

FLIPR Method for Rat P2X4 1321N1 Astrocytoma Cells

Compounds were tested on a 1321N1 cell line stably expressing rat P2X4. Cells were cultured on collagen-I-coated 384-well plates at a density of 10,000 cells/well and incubated overnight at 37° C., 5% CO₂. P2X4 function was assessed by measuring intracellular calcium fluxes caused by Magnesium-ATP (MgATP) using the calcium-chelating dye Fluo8-AM (Molecular Devices) with the Fluorescent Imaging Plate Reader Tetra (FLIPR^{Tetra}; Molecular Devices CA). On the day of the assay, the medium was removed and the cells were incubated for 30 minutes at 37° C. and 5% CO₂ in 30 μl of dye buffer (Hank's balanced salt solution, 10 mM HEPES, 1.8 mM CaCl₂, 1 mM MgCl₂, 2 mM probenecid, 5 mM D-glucose monohydrate, 5 μM Fluo8-AM, pH=7.4).

Compounds diluted in probenecid buffer (Hank's balanced salt solution, 10 mM HEPES, 1.8 mM CaCl₂, 1 mM MgCl₂, 2 mM probenecid, 5 mM D-glucose monohydrate, pH=7.4) at ten concentrations ranging from 25 μM to 1 nM (final concentration) were dispensed and incubated for 30 minutes at room temperature. The agonist, MgATP (Sigma-Aldrich Chemie GmbH, DE), was added at a final concentration of 5 μM, representing the EC₈₀ routinely determined. The final assay volume was 50 μl and final DMSO concentration was 0.5%.

The fluorescence intensity reflecting intracellular calcium changes was recorded before and after MgATP addition, at an excitation and emission wavelengths of 470-495 nm and 515-575 nm respectively.

The compounds were tested in triplicates and fluorescence intensity raw data normalized to the agonist control and fitted to the four-parameter logistic equation:

Y=Bottom+(Top−Bottom)/(1+10\hat{ }((Log IC50−X)*HillSlope))

The efficacy of saturating concentrations of the agonist MgATP (5 μM) was set as maximal response (100% Emax) and the bottom defined by the signal achieved with 0.5% DMSO.

Assay plate acceptance was based on the signal window (S/B)≥1.5, Z′≥0.5 and the reference compound pIC50 within ±3a the mean of historic pIC50 of the compound. Failure to meet two of the three criteria determined exclusion of the plate's results.

In Table 10 below the results for the assays are reported

TABLE 10

| | Human | Human P2X4 | Rat P2X4 | Rat P2X4 |
|---|---|---|---|---|
| | P2X4 | HEK | 1321N1 | 1321N1 |
| | HEK Cells | Cells (FLIPR | Astrocytoma | Astrocytoma |
| | (FLIPR | Assay) | Cells | Cells |
| | Assay) | avg | (FLIPR Assay) | (FLIPR Assay) |
| Ex. N. | avg IC₅₀ [nM] | Efficacy [%] | avg IC₅₀ [nM] | avg Efficacy [%] |
| 1 | 224 | 89 | 96.7 | 82 |
| 2 | 212 | 97 | 358 | 95 |
| 3 | 657 | 98 | | |
| 4 | 397 | 100 | 855 | 100 |
| 5 | 386 | 102 | 440 | 100 |
| 6 | 689 | 97 | | |
| 7 | 846 | 97 | | |
| 8 | 681 | 97 | | |
| 9 | 749 | 99 | | |
| 10 | 875 | 94 | | |
| 11 | 977 | 100 | | |
| 12 | 973 | 101 | | |
| 13 | 289 | 96 | 790 | 93 |
| 14 | 243 | 100 | 201 | 98 |
| 15 | 408 | 94 | 441 | 88 |
| 16 | 388 | 95 | | |
| 17 | 638 | 99 | | |
| 18 | 437 | 99 | | |
| 19 | 542 | 103 | | |
| 20 | 748 | 99 | | |
| 21 | 343 | 91 | 374 | 90 |
| 22 | 482 | 98 | 217 | 95 |
| 23 | 356 | 99 | 483 | 96 |
| 24 | 346 | 100 | 174 | 99 |
| 25 | 524 | 100 | 851 | 100 |
| 26 | 660 | 102 | 649 | 101 |
| 27 | 242 | 97 | 652 | 99 |
| 28 | 777 | 91 | | |
| 29 | 391 | 91 | 92.5 | 77 |
| 30 | 384 | 98 | 727 | 96 |
| 31 | 909 | 100 | | |
| 32 | 355 | 101 | 801 | 100 |
| 33 | 723 | 93 | 1540 | 98 |
| 34 | 199 | 97 | 170 | 94 |
| 35 | 938 | 99 | | |
| 36 | 206 | 91 | 290 | 90 |
| 37 | 894 | 95 | 242 | 96 |
| 38 | 232 | 96 | 136 | 98 |
| 39 | 335 | 100 | 152 | 98 |
| 40 | 308 | 90 | 194 | 75 |
| 41 | 589 | 94 | 115 | 91 |
| 42 | 350 | 98 | 1360 | 96 |
| 43 | 406 | 100 | | |
| 44 | 607 | 99 | | |
| 45 | 485 | 102 | 1180 | 100 |
| 46 | 551 | 103 | | |
| 47 | 519 | 98 | | |
| 48 | 579 | 99 | | |
| 49 | 520 | 100 | | |
| 50 | 351 | 95 | 213 | 96 |
| 51 | 493 | 98 | 177 | 96 |
| 52 | 355 | 98 | 358 | 99 |
| 53 | 455 | 100 | 143 | 100 |
| 54 | 269 | 97 | 447 | 97 |
| 55 | 611 | 102 | 289 | 99 |
| 56 | 260 | 97 | 190 | 98 |
| 57 | 377 | 99 | 122 | 97 |
| 58 | 351 | 95 | 350 | 86 |
| 59 | 532 | 97 | 240 | 81 |
| 60 | 454 | 96 | 434 | 94 |
| 61 | 604 | 98 | | |
| 62 | 251 | 91 | 368 | 91 |
| 63 | 424 | 97 | 372 | 97 |
| 64 | 829 | 96 | 497 | 100 |
| 65 | 505 | 94 | 122 | 93 |
| 66 | 303 | 90 | 64.2 | 73 |
| 67 | 604 | 94 | 159 | 97 |
| 68 | 411 | 95 | 233 | 89 |
| 69 | 816 | 94 | 228 | 100 |
| 70 | 714 | 88 | 714 | 100 |
| 71 | 222 | 96 | 165 | 90 |
| 72 | 355 | 97 | 58.5 | 89 |
| 73 | 428 | 97 | 67.1 | 93 |
| 74 | 585 | 89 | 99.1 | 97 |
| 75 | 919 | 96 | | |
| 76 | 397 | 97 | 667 | 99 |
| 77 | 354 | 94 | 993 | 84 |
| 78 | 573 | 94 | | |
| 79 | 399 | 97 | 1110 | 96 |
| 80 | 451 | 99 | 338 | 97 |
| 81 | 895 | 97 | | |
| 82 | 504 | 97 | | |
| 83 | 350 | 97 | 143 | 90 |
| 84 | 314 | 100 | 1030 | 100 |
| 85 | 251 | 98 | 229 | 97 |
| 86 | 196 | 98 | 393 | 98 |
| 87 | 313 | 98 | 983 | 94 |
| 88 | 198 | 97 | 451 | 100 |
| 89 | 169 | 99 | 103 | 98 |
| 90 | 272 | 98 | 110 | 97 |
| 91 | 345 | 98 | 1630 | 100 |
| 92 | 568 | 97 | | |
| 93 | 705 | 98 | | |
| 94 | 710 | 97 | | |
| 95 | 694 | 100 | | |
| 96 | 362 | 89 | | |
| 97 | 461 | 89 | | |
| 98 | 296 | 92 | | |
| 99 | 449 | 95 | | |
| 100 | 408 | 98 | 965 | 98 |
| 101 | 406 | 97 | 971 | 100 |
| 102 | 937 | 99 | | |
| 103 | 767 | 97 | | |
| 104 | 846 | 96 | | |
| 105 | 770 | 100 | | |
| 106 | 765 | 95 | | |
| 107 | 550 | 98 | | |
| 108 | 769 | 97 | | |
| 109 | 643 | 100 | | |
| 110 | 745 | 95 | | |
| 111 | 204 | 96 | 320 | 100 |
| 112 | 403 | 96 | 408 | 97 |
| 113 | 286 | 97 | 567 | 100 |
| 114 | 256 | 97 | 181 | 99 |
| 115 | 391 | 96 | | |
| 116 | 520 | 97 | | |
| 117 | 789 | 95 | | |
| 118 | 545 | 94 | | |
| 119 | 941 | 101 | | |
| 120 | 871 | 102 | | |
| 121 | 843 | 97 | | |
| 122 | 357 | 95 | | |
| 123 | 435 | 97 | 633 | 89 |
| 124 | 289 | 94 | | |
| 125 | 577 | 95 | | |
| 126 | 176 | 96 | 377 | 83 |
| 127 | 283 | 97 | 261 | 72 |
| 128 | 184 | 101 | 454 | 87 |
| 129 | 197 | 104 | 212 | 96 |
| 130 | 259 | 97 | 1520 | 80 |
| 131 | 206 | 98 | 262 | 98 |
| 132 | 299 | 98 | 228 | 97 |
| 133 | 193 | 100 | 325 | 100 |
| 134 | 504 | 99 | | |
| 135 | 281 | 99 | 264 | 96 |
| 136 | 310 | 94 | | |
| 137 | 290 | 104 | | |
| 138 | 316 | 96 | | |
| 139 | 356 | 100 | | |
| 140 | 271 | 105 | | |
| 141 | 891 | 99 | 726 | 100 |
| 142 | 796 | 86 | 767 | 98 |
| 143 | 723 | 94 | 934 | 101 |
| 144 | 648 | 90 | 557 | 100 |
| 145 | 234 | 98 | | |
| 146 | 337 | 103 | | |
| 147 | 384 | 97 | | |
| 148 | 436 | 96 | | |
| 149 | 450 | 100 | | |
| 150 | 297 | 102 | | |
| 151 | 355 | 104 | | |
| 152 | 441 | 104 | | |
| 153 | 476 | 102 | | |
| 154 | 511 | 101 | | |
| 155 | 339 | 100 | | |
| 156 | 512 | 96 | | |
| 157 | 644 | 97 | | |
| 158 | 649 | 97 | | |
| 159 | 949 | 99 | | |
| 160 | 383 | 101 | | |
| 161 | 479 | 101 | | |
| 162 | 577 | 104 | | |
| 163 | 665 | 100 | | |
| 164 | 683 | 100 | | |
| 165 | 402 | 99 | | |
| 166 | 663 | 97 | | |
| 167 | 677 | 100 | | |
| 168 | 761 | 99 | | |
| 169 | 813 | 101 | | |
| 170 | 468 | 104 | | |
| 171 | 511 | 103 | | |
| 172 | 520 | 104 | | |
| 173 | 526 | 104 | | |
| 174 | 528 | 103 | | |
| 175 | 516 | 97 | | |
| 176 | 567 | 101 | | |
| 177 | 578 | 96 | | |
| 178 | 621 | 99 | | |
| 179 | 673 | 98 | | |
| 180 | 569 | 101 | | |
| 181 | 574 | 101 | | |
| 182 | 593 | 102 | | |
| 183 | 599 | 101 | | |
| 184 | 893 | 104 | | |
| 185 | 438 | 101 | | |
| 186 | 655 | 99 | | |
| 187 | 700 | 100 | | |
| 188 | 829 | 98 | | |
| 189 | 654 | 93 | | |
| 190 | 716 | 95 | | |
| 191 | 739 | 92 | | |
| 192 | 766 | 90 | | |
| 193 | 868 | 93 | | |
| 194 | 407 | 96 | | |
| 195 | 486 | 95 | | |
| 196 | 546 | 94 | | |
| 197 | 674 | 93 | | |
| 198 | 943 | 96 | | |
| 199 | 447 | 86 | | |
| 200 | 465 | 91 | | |
| 201 | 510 | 89 | | |
| 202 | 557 | 94 | | |
| 203 | 737 | 86 | | |
| 204 | 383 | 95 | | |
| 205 | 433 | 93 | | |
| 206 | 449 | 93 | | |
| 207 | 477 | 96 | | |
| 208 | 669 | 91 | | |

Expression of P24 Receptor in Hug Pan Corneal Tissue

Human cornea was acquired through corneal bank SightLife (Seattle, Wash.), and fixed in 10% natural buffered formalin (Millipore, Germany) for 4 hours at 4° C. before embedded in OCT. Cornea tissue were blotted with primary antibody: 1:100 P2X4 (Abcam ab134559) for 18 hours at 4° C., and secondary antibody 1:500 Alexa Fluror555 (Molecular Probe A21432) for 2 hours at room temperature.

Images were acquired by LSM700 fluorescent microscope (Zeiss).

Corneal tissue expression of P2X4 receptor was examined. Corneal tissue from three donors was processed and stained for P2X4 receptor localization. In all three samples P2X4 receptor expression was seen in central and peripheral regions of the cornea as well as the limbal boundary region connecting the conjunctival and corneal tissue layers. No labeling was seen in tissue stained with secondary antibody alone.

In Vivo Studies

Lacrimal Gland Removal Model with Pain Behavior Read Out

Material and Methods:

Animal Model:

Sprague Dawley rats were anesthetized by intraperitoneal injection of 75 mg/kg ketamine +7.5 mg/kg xylazine hydrochloride. After removal of the facial hair, a 2 mm incision was carefully made between temporal lid margin and zygomatic arch. Bluntly separating the underlying tissue, exposing the fascia covering the intra-orbital lacrimal gland, avoiding the nerves and blood vessels. A small incision was made on the fascia, and the intra-orbital gland was gently pulled out and excised. The incision was sutured with a 8-0 polysorb suture and the skin was closed with a 5-0 polysorb suture (Ethilon; Ethicon Inc, Somerville, N.J.). A 3 mm incision was made just below the earlobe, and the extra-orbital lacrimal gland was pulled out and excised along its stem. This incision was closed in a manner similar as described above.

Wipe Test:

Acclimate rat in a quiet, clean, clear cage for 15 minutes. Gently hold head of rat, add one drop of 5M NaCl in the rat right eye, and start count wipes (front limb wipe across the face) for 30 seconds. Repeat wipe test in left eye. Wash each eye with 10 ml NS. Eye wiping frequency is used as a behavioral endpoint to assess pain level in animals to assess the level of pain amelioration after treatment with compound as compared to vehicle alone. The number of eye wipes over a period of thirty seconds was determined for each animal and averaged at various timepoints. A baseline wipe number was established prior to lacrimal gland removal. Two weeks post lacrimal gland removal a second counting of wiping behavior was again conducted prior to the start of treatment. Additional wiping behavior counts were conducted after two and four weeks of treatment. An increase of wipe rate was observed in all animals during the two weeks after lacrimal gland removal and prior to the start of treatment. While an increase in wipe count was observed in animals treated with vehicle alone no apparent further increase in wipe count was observed in animals treated with compound.

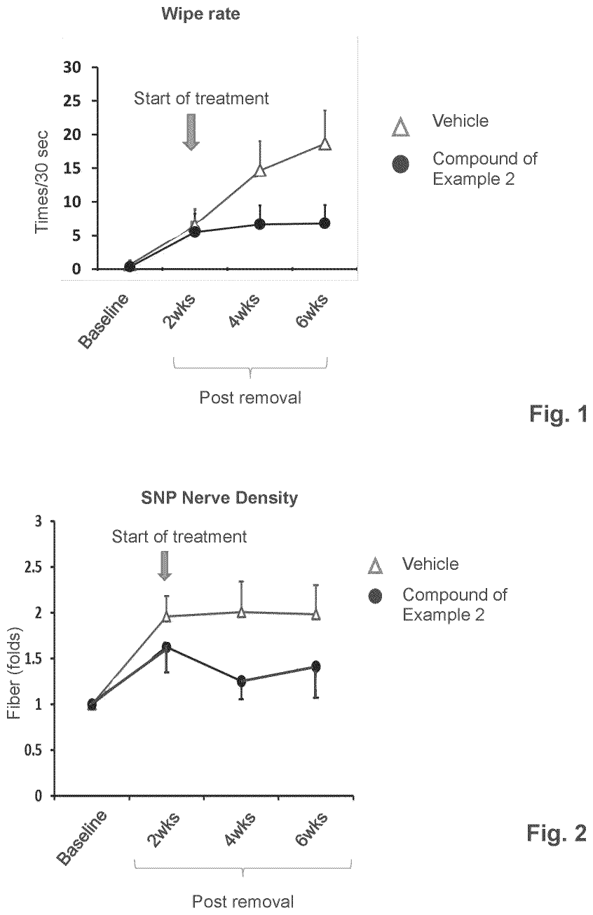

(FIG. 1—treatment with a P2X4 antagonist, Example 2).

Nerve Density Measurement:

SNP Measurement:

For every post-surgery 4 wks rat, 10 random SNP areas were recorded by IVCM (in vivo confocal microscope), and nerve density, nerve length, nerve reflectiveness were analyzed by ImageJ (Java-based image processing program developed at the National Institutes of Health and the Laboratory for Optical and Computational Instrumentation (LOCI, University of Wisconsin. Schneider C A, Rasband W S, Eliceiri K W (2012). “NIH Image to ImageJ: 25 years of image analysis”. Nat Methods. 9 (7): 671-6751, and nerve tortuosity were analyzed by CCMetrics (CCMetrics is an image analysis software which allows manual tracing of the nerves and automatic quantification of nerve fibre measures from corneal confocal microscopy (CCM) images: Dabbah M A et al. Automatic Analysis of Diabetic Peripheral Neuropathy using Multi-scale Quantitative Morphology of Nerve Fibres in Corneal Confocal Microscopy Imaging. Journal of Medical Image Analysis. 2011; 15(5), 738-747; Petropoulos I N et al. Rapid automated diagnosis of diabetic peripheral neuropathy with in vivo corneal confocal microscopy. Investigative ophthalmology & visual science 2014; 55:2071-2078; Tavakoli M et al. Normative values for corneal nerve morphology assessed using corneal confocal microscopy: a multinational normative data set. Diabetes Care 2015; 38:838-843).
Longitudinal corneal monitoring through individual stromal nerve tracing: For every pre-surgery rat, 3 individual stromal nerves at the depth of 50-80 um were mapped and recorded manually in 3 out of 4 quadrans. Volume acquisition from whole thickness of cornea was made by IVCM. In the following weeks of just before treatment (2 wks post-surgery), 2 wks post-treatment (4 wks post-surgery), and 4 wks post-treatment (6 wks post-surgery), the same stroma nerve will be found and volume acquisition were made. SNP nerve density were measured by ImageJ, and nerve fiber numbers were normalized by pre-surgery baseline.

Corneal nerve density tracings were performed at baseline (prior to lacrimal gland removal), two, four and six weeks post lacrimal gland removal with treatment starting after the two week measurement was performed. Nerve density was plotted as a fold difference from baseline with an increase seen in all animals after two weeks post gland removal prior to the start of treatment. While no appreciable change was seen in animals treated with vehicle alone a decline in nerve density could be observed in animals treated with compound.

(FIG. 2—treatment with a P2X4 antagonist Example 2 and effect on corneal nerve density)

Evaluation of Pharmacokinetics

To evaluate the pharmacokinetics of test substances in vivo, these test substances are dissolved in appropriate formulation vehicles (e.g. castor oil). The test substances are then administered to rats topically as eye drops over four to seven days. Administered doses range usually between 0.1 to 5 mg/mL. Blood samples and ocular tissue samples are taken at the time of expected trough concentration, i.e. just before the next dosing would have occurred. Blood samples are retrieved via exsanguination in vials containing appropriate anticoagulants, such as lithium heparinate or potassium EDTA. Plasma is generated from the blood via centrifugation. Eyes are enucleated and briefly washed in PBS to remove possible remains of the topical formulation. Eyes are dissected and tissues of interest, e.g. cornea, lacrimal gland and duct, lens and humor and back of the eye tissue, are collected separately. Ocular tissues are pooled per individual animal, diluted in appropriate amounts of 0.9% NaCl in water and homogenized using a Tissuelyzer or equivalent system. The quantitative measurement of the test substances in the samples is performed using calibration curves in the respective matrices. The protein content of the samples is precipitated using acetonitrile or methanol. Thereafter, the samples are separated using HPLC in combination with reversed phase chromatography columns. The HPLC system is coupled to a triple quadrupole mass spectrometer via an electrospray interface. Particularly, HPLC was performed using a C18 column (e.g. Phenomenex Kinetex C18 5 mm 5 μm 2.1×50) and a mixture of 10 mM ammonium acetate pH6.8 and acetonitrile in a gradient with an increasing fraction of organic solvent. The retention time for the compound of example 2 was for example ca. 2.6 min.

Data: Mean concentration of test compound (μg/L of plasma or μg/kg of wet tissue weight), samples with concentrations below the lower limit of quantification (LOQ) are denoted as “<LOQ”, n corresponds to the number of the treated animals

| | | | |
|---|---|---|---|
| Example 2 | 1 mg/mL | Plasma | 0.4 |
| N-{4-[2-(2-chloro-3- | in | Cornea | 44.9 |
| fluorophenyl)acetamido]pyridin-2- | castor oil | Lens and humor | 1.8 |
| yl}-N-(4-fluorophenyl)acetamide | | Lacrimal gland | 17.4 |
| (n = 2) | | and duct | |
| | | Back of the eye | 13.8 |
| 2-(2-Chlorophenyl)-N-[4-(4-cyano- | 1 mg/mL | Plasma | 2.37 |
| 1H-pyrazol-1-yl)-3- | in | Cornea | 13.4 |
| sulfamoylphenyl]acetamide | castor oil | Lens and humor | <4.0 |
| (n = 2) | | Lacrimal gland | 33.4 |
| | | and duct | |
| | | Back of the eye | 14.5 |

1: A method of treatment or prophylaxis of dry eye syndrome, dry eye, ocular neuropathic pain, ocular trauma, or post-operative ocular pain, comprising administering a P2X4 inhibitor, or a pharmaceutically acceptable salt thereof, to a mammal in need thereof.

2: The method of claim 1, wherein the P2X4 inhibitor is a compound of formula (I): 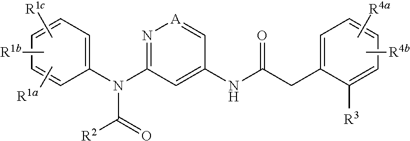
wherein
A is CH or N;
R^{1a}, R^{1b}, and R^{1c} are each independently a hydrogen atom, a halogen atom, cyano, (C₁-C₃)-alkyl, (C₁-C₃)-haloalkyl, or (C₁-C₃)-alkoxy,
R² is (C₁-C₃)-alkyl;
R³ is a halogen atom, cyano, (C₁-C₃)-alkyl, (C₁-C₃)-haloalkyl, or (C₁-C₃)-alkoxy; and
R^{4a} and R^{4b} are each independently a hydrogen atom, a halogen atom, cyano, (C₁-C₃)-alkyl, (C₁-C₃)-haloalkyl, or (C₁-C₃)-alkoxy,
or a stereoisomer, a tautomer, an N-oxide, a hydrate, a solvate, or a pharmaceutically acceptable salt thereof, or a mixture of any of the foregoing.

3: The method of claim 2, wherein:
R^{1a} and R^{1b} are each independently a halogen atom, cyano, (C₁-C₃)-alkyl, (C₁-C₃)-haloalkyl, or (C₁-C₃)-alkoxy; and
R^{1c} is a hydrogen atom.

4: The method of claim 2, wherein:
R^{1a} is in position 4 of the phenyl ring and is a halogen atom, cyano, (C₁-C₃)-alkyl, (C₁-C₃)-haloalkyl, or (C₁-C₃)-alkoxy;
R^{1b} is a hydrogen atom a halogen atom, cyano, (C₁-C₃)-alkyl, (C₁-C₃)-haloalkyl, or (C₁-C₃)-alkoxy; and
R^{1c} is a hydrogen atom.

5: The method of claim 2, wherein:
R^{1a} is in position 4 of the phenyl ring and is a halogen atom, cyano, (C₁-C₃)-alkyl, (C₁-C₃)-haloalkyl, or (C₁-C₃)-alkoxy;
R^{1b} is in position 3 of the phenyl ring and is a hydrogen atom, a halogen atom, cyano, (C₁-C₃)-alkyl, (C₁-C₃)-haloalkyl, or (C₁-C₃)-alkoxy; and
R^{1c} is a hydrogen atom.

6: The method of claim 2, wherein:
R^{1a} is a halogen atom, cyano, (C₁-C₃)-alkyl, (C₁-C₃)-haloalkyl, or (C₁-C₃)-alkoxy; and
R^{1b} and R^{1c} are each a hydrogen atom.

7: The method of claim 2, wherein:
R² is methyl.

8: The method of claim 2, wherein:
R³ is chlorine, fluorine, cyano, or a hydrogen atom.

9: The method of claim 2, wherein:
R^{4a} is a halogen atom, cyano, (C₁-C₃)-alkyl, (C₁-C₃)-haloalkyl, or (C₁-C₃)-alkoxy, and
R^{4b} is a hydrogen atom; or
R^{4a} is a halogen atom, cyano, (C₁-C₃)-alkyl, (C₁-C₃)-haloalkyl, or (C₁-C₃)-alkoxy, and
R^{4b} is a halogen atom, cyano, (C₁-C₃)-alkyl, (C₁-C₃)-haloalkyl, or (C₁-C₃)-alkoxy.

10: The method of claim 2, wherein:
R³ is chlorine, fluorine, or cyano,
R^{4a} is a halogen atom, cyano, (C₁-C₃)-alkyl, (C₁-C₃)-haloalkyl, or (C₁-C₃)-alkoxy in position 3 or 6 of the phenyl group, and
R^{4b} is a hydrogen atom; or
R³ is chlorine, fluorine, or cyano,
R^{4a} is a halogen atom, cyano, (C₁-C₃)-alkyl, (C₁-C₃)-haloalkyl, or (C₁-C₃)-alkoxy in position 6 of the phenyl group, and
R^{4b} is a halogen atom, cyano, (C₁-C₃)-alkyl, (C₁-C₃)-haloalkyl, or (C₁-C₃)-alkoxy in position 4 of the phenyl group.

11: The method of claim 1, wherein the P2X4 inhibitor is selected from the group consisting of
N-{4-[2-(2-chlorophenyl)acetamido]pyridin-2-yl}-N-(3,4-difluorophenyl)acetamide;
N-{4-[2-(2-chloro-3-fluorophenyl)acetamido]pyridin-2-yl}-N-(4-fluorophenyl)acetamide;
N-{4-[2-(2-chloro-4-fluorophenyl)acetamido]pyridin-2-yl}-N-[3-(difluoromethyl)phenyl]acetamide;
N-{4-[2-(2-chloro-3-fluorophenyl)acetamido]pyridin-2-yl}-N-[3-(difluoromethyl)phenyl]acetamide;
N-{4-[2-(2,6-dichlorophenyl)acetamido]pyridin-2-yl}-N-[3-(difluoromethyl)phenyl]acetamide;
N-{4-[2-(2-chlorophenyl)acetamido]pyridin-2-yl}-N-[3-(trifluoromethyl)phenyl]acetamide;
N-{4-[2-(2-chloro-4-fluorophenyl)acetamido]pyridin-2-yl}-N-[3-(trifluoromethyl)phenyl]acetamide;
N-{4-[2-(2-chloro-3-fluorophenyl)acetamido]pyridin-2-yl}-N-[3-(trifluoromethyl)phenyl]acetamide;
N-{4-[2-(2,6-dichlorophenyl)acetamido]pyridin-2-yl}-N-[3-(trifluoromethyl)phenyl]acetamide;
N-{4-[2-(2-chlorophenyl)acetamido]pyridin-2-yl}-N-[4-cyano-3-(trifluoromethyl)phenyl]acetamide;
N-{4-[2-(2-chloro-3-fluorophenyl)acetamido]pyridin-2-yl}-N-[4-cyano-3-(trifluoromethyl)phenyl]acetamide;
N-[4-cyano-3-(trifluoromethyl)phenyl]-N-{4-[2-(2,6-dichlorophenyl)acetamido]pyridin-2-yl}acetamide;
N-{4-[2-(2-chlorophenyl)acetamido]pyridin-2-yl}-N-(3-cyanophenyl)acetamide;
N-(3-cyanophenyl)-N-{4-[2-(2,6-dichlorophenyl)acetamido]pyridin-2-yl}acetamide;
N-{4-[2-(2-chloro-6-fluorophenyl)acetamido]pyridin-2-yl}-N-(3-cyanophenyl)acetamide;
N-(4-chloro-3-cyanophenyl)-N-{4-[2-(2-chlorophenyl)acetamido]pyridin-2-yl}acetamide;
N-(4-chloro-3-cyanophenyl)-N-{4-[2-(2-chloro-4-fluorophenyl)acetamido]pyridin-2-yl}acetamide;
N-(4-chloro-3-cyanophenyl)-N-{4-[2-(2-chloro-3-fluorophenyl)acetamido]pyridin-2-yl}acetamide;
N-(4-chloro-3-cyanophenyl)-N-{4-[2-(2,6-dichlorophenyl)acetamido]pyridin-2-yl}acetamide;
N-(4-{2-[2-chloro-3-(trifluoromethyl)phenyl]acetamido}pyridin-2-yl)-N-(3-cyano-4-fluorophenyl)acetamide;
N-{4-[2-(2-chlorophenyl)acetamido]pyridin-2-yl}-N-(3-cyano-4-fluorophenyl)acetamide;
N-{4-[2-(2-chloro-4-fluorophenyl)acetamido]pyridin-2-yl}-N-(3-cyano-4-fluorophenyl)acetamide;
N-{4-[2-(2-chloro-3-fluorophenyl)acetamido]pyridin-2-yl}-N-(3-cyano-4-fluorophenyl)acetamide;
N-(3-cyano-4-fluorophenyl)-N-{4-[2-(2,6-dichlorophenyl)acetamido]pyridin-2-yl}acetamide;
N-{4-[2-(2-chlorophenyl)acetamido]pyridin-2-yl}-N-phenylacetamide;
N-{4-[2-(2-chloro-4-fluorophenyl)acetamido]pyridin-2-yl}-N-phenylacetamide;
N-{4-[2-(2,6-dichlorophenyl)acetamido]pyridin-2-yl}-N-phenylacetamide;
N-{4-[2-(2-chloro-6-fluorophenyl)acetamido]pyridin-2-yl}-N-phenylacetamide;
N-{4-[2-(2,3-dimethylphenyl)acetamido]pyridin-2-yl}-N-(4-fluorophenyl)acetamide;
N-(4-fluorophenyl)-N-(4-{2-[2-(trifluoromethyl)phenyl]acetamido}pyridin-2-yl)acetamide;
N-(4-{2-[4-chloro-2-(trifluoromethyl)phenyl]acetamido}pyridin-2-yl)-N-(4-fluorophenyl)acetamide;
N-(4-fluorophenyl)-N-(4-{2-[3-fluoro-2-(trifluoromethyl)phenyl]acetamido}pyridin-2-yl)acetamide;
N-{4-[2-(2-chloro-6-cyanophenyl)acetamido]pyridin-2-yl}-N-(4-fluorophenyl)acetamide;
N-{4-[2-(2,6-dimethylphenyl)acetamido]pyridin-2-yl}-N-(4-fluorophenyl)acetamide;
N-(4-{2-[2-chloro-3-(trifluoromethyl)phenyl]acetamido}pyridin-2-yl)-N-(4-fluorophenyl)acetamide;
N-{4-[2-(2-chlorophenyl)acetamido]pyridin-2-yl}-N-(4-fluorophenyl)acetamide;
N-{4-[2-(2-chloro-4-fluorophenyl)acetamido]pyridin-2-yl}-N-(4-fluorophenyl)acetamide;
N-{4-[2-(2,6-dichlorophenyl)acetamido]pyridin-2-yl}-N-(4-fluorophenyl)acetamide;
N-{4-[2-(2,6-dichloro-4-fluorophenyl)acetamido]pyridin-2-yl}-N-(4-fluorophenyl)acetamide;
N-{4-[2-(2-chloro-6-fluorophenyl)acetamido]pyridin-2-yl}-N-(4-fluorophenyl)acetamide;
N-{4-[2-(2-chloro-4,6-difluorophenyl)acetamido]pyridin-2-yl}-N-(4-fluorophenyl)acetamide;
N-(4-fluorophenyl)-N-(4-{2-[2-(trifluoromethoxy)phenyl]acetamido}pyridin-2-yl)acetamide;
N-{4-[2-(2-chlorophenyl)acetamido]pyridin-2-yl}-N-(4-methylphenyl)acetamide;
N-{4-[2-(2-chloro-4-fluorophenyl)acetamido]pyridin-2-yl}-N-(4-methylphenyl)acetamide;
N-{4-[2-(2-chloro-3-fluorophenyl)acetamido]pyridin-2-yl}-N-(4-methylphenyl)acetamide;
N-{4-[2-(2,6-dichlorophenyl)acetamido]pyridin-2-yl}-N-(4-methylphenyl)acetamide;
N-{4-[2-(2-chlorophenyl)acetamido]pyridin-2-yl}-N-[4-(difluoromethoxy)phenyl]acetamide;
N-{4-[2-(2-chloro-3-fluorophenyl)acetamido]pyridin-2-yl}-N-[4-(difluoromethoxy)phenyl]acetamide;
N-{4-[2-(2,6-dichlorophenyl)acetamido]pyridin-2-yl}-N-[4-(difluoromethoxy)phenyl]acetamide;
N-(3-chloro-4-fluorophenyl)-N-{4-[2-(2-chlorophenyl)acetamido]pyridin-2-yl}acetamide;
N-(3-chloro-4-fluorophenyl)-N-{4-[2-(2-chloro-4-fluorophenyl)acetamido]pyridin-2-yl}acetamide;
N-(3-chloro-4-fluorophenyl)-N-{4-[2-(2-chloro-3-fluorophenyl)acetamido]pyridin-2-yl}acetamide;
N-(3-chloro-4-fluorophenyl)-N-{4-[2-(2,6-dichlorophenyl)acetamido]pyridin-2-yl}acetamide;
N-{4-[2-(2-chlorophenyl)acetamido]pyridin-2-yl}-N-(3-fluorophenyl)acetamide;
N-{4-[2-(2-chloro-4-fluorophenyl)acetamido]pyridin-2-yl}-N-(3-fluorophenyl)acetamide;
N-{4-[2-(2,6-dichlorophenyl)acetamido]pyridin-2-yl}-N-(3-fluorophenyl)acetamide;
N-{4-[2-(2,6-dichloro-4-fluorophenyl)acetamido]pyridin-2-yl}-N-(3-fluorophenyl)acetamide;
N-(4-chloro-3-fluorophenyl)-N-{4-[2-(2-chlorophenyl)acetamido]pyridin-2-yl}acetamide;
N-(4-chloro-3-fluorophenyl)-N-{4-[2-(2-chloro-4-fluorophenyl)acetamido]pyridin-2-yl}acetamide;
N-(4-chloro-3-fluorophenyl)-N-{4-[2-(2-chloro-3-fluorophenyl)acetamido]pyridin-2-yl}acetamide;
N-(4-chloro-3-fluorophenyl)-N-{4-[2-(2,6-dichlorophenyl)acetamido]pyridin-2-yl}acetamide;
N-(3,4-difluorophenyl)-N-{4-[2-(2,3-dimethylphenyl)acetamido]pyridin-2-yl}acetamide;
N-(3,4-difluorophenyl)-N-(4-{2-[2-(trifluoromethyl)phenyl]acetamido}pyridin-2-yl)acetamide;
N-{4-[2-(2,4-dichloro-6-methylphenyl)acetamido]pyridin-2-yl}-N-(3,4-difluorophenyl)acetamide;
N-{4-[2-(2-chloro-4,6-dimethylphenyl)acetamido]pyridin-2-yl}-N-(3,4-difluorophenyl)acetamide;
N-(3,4-difluorophenyl)-N-{4-[2-(2,6-dimethylphenyl)acetamido]pyridin-2-yl}acetamide;
N-{4-[2-(2,4-dichlorophenyl)acetamido]pyridin-2-yl}-N-(3,4-difluorophenyl)acetamide;
N-{4-[2-(2-chloro-4-fluorophenyl)acetamido]pyridin-2-yl}-N-(3,4-difluorophenyl)acetamide;
N-{4-[2-(2-chloro-4-nitrophenyl)acetamido]pyridin-2-yl}-N-(3,4-difluorophenyl)acetamide;
N-{4-[2-(2-chloro-4-methoxyphenyl)acetamido]pyridin-2-yl}-N-(3,4-difluorophenyl)acetamide;
N-{4-[2-(2-chloro-3-fluorophenyl)acetamido]pyridin-2-yl}-N-(3,4-difluorophenyl)acetamide;
N-{4-[2-(2,6-dichlorophenyl)acetamido]pyridin-2-yl}-N-(3,4-difluorophenyl)acetamide;
N-{4-[2-(2,6-dichloro-4-fluorophenyl)acetamido]pyridin-2-yl}-N-(3,4-difluorophenyl)acetamide;
N-{4-[2-(2,6-dichloro-4-methylphenyl)acetamido]pyridin-2-yl}-N-(3,4-difluorophenyl)acetamide;
N-{4-[2-(2,6-dichloro-4-ethylphenyl)acetamido]pyridin-2-yl}-N-(3,4-difluorophenyl)acetamide;
N-(3,4-difluorophenyl)-N-(4-{2-[2-(trifluoromethoxy)phenyl]acetamido}pyridin-2-yl)acetamide;
N-{4-[2-(2-chlorophenyl)acetamido]pyridin-2-yl}-N-(3-fluoro-4-methoxyphenyl)acetamide;
N-{4-[2-(2-chloro-4-fluorophenyl)acetamido]pyridin-2-yl}-N-(3-fluoro-4-methoxyphenyl)acetamide;
N-{4-[2-(2-chloro-3-fluorophenyl)acetamido]pyridin-2-yl}-N-(3-fluoro-4-methoxyphenyl)acetamide;
N-{4-[2-(2,6-dichlorophenyl)acetamido]pyridin-2-yl}-N-(3-fluoro-4-methoxyphenyl)acetamide;
N-{4-[2-(2-chloro-3-fluorophenyl)acetamido]pyridin-2-yl}-N-[3-fluoro-4-(methanesulfonyl)phenyl]acetamide;
N-{4-[2-(2,6-dichlorophenyl)acetamido]pyridin-2-yl}-N-[3-fluoro-4-(methanesulfonyl)phenyl]acetamide;
N-(3,5-difluorophenyl)-N-{4-[2-(2,3-dimethylphenyl)acetamido]pyridin-2-yl}acetamide;
N-(3,5-difluorophenyl)-N-(4-{2-[2-(trifluoromethyl)phenyl]acetamido}pyridin-2-yl)acetamide;
N-(3,5-difluorophenyl)-N-{4-[2-(2,6-dimethylphenyl)acetamido]pyridin-2-yl}acetamide;
N-{4-[2-(2-chlorophenyl)acetamido]pyridin-2-yl}-N-(3,5-difluorophenyl)acetamide;
N-{4-[2-(2-chloro-4-fluorophenyl)acetamido]pyridin-2-yl}-N-(3,5-difluorophenyl)acetamide;
N-{4-[2-(2-chloro-3-fluorophenyl)acetamido]pyridin-2-yl}-N-(3,5-difluorophenyl)acetamide;
N-{4-[2-(2,6-dichlorophenyl)acetamido]pyridin-2-yl}-N-(3,5-difluorophenyl)acetamide;
N-{4-[2-(2,6-dichloro-4-fluorophenyl)acetamido]pyridin-2-yl}-N-(3,5-difluorophenyl)acetamide;
N-(3,5-difluorophenyl)-N-(4-{2-[2-(trifluoromethoxy)phenyl]acetamido}pyridin-2-yl)acetamide;
N-{4-[2-(2-chlorophenyl)acetamido]pyridin-2-yl}-N-(3,5-difluoro-4-methylphenyl)acetamide;
N-{4-[2-(2-chloro-4-fluorophenyl)acetamido]pyridin-2-yl}-N-(3,5-difluoro-4-methylphenyl)acetamide;
N-{4-[2-(2-chloro-3-fluorophenyl)acetamido]pyridin-2-yl}-N-(3,5-difluoro-4-methylphenyl)acetamide;
N-{4-[2-(2,6-dichlorophenyl)acetamido]pyridin-2-yl}-N-(3,5-difluoro-4-methylphenyl)acetamide;
N-{4-[2-(2-chlorophenyl)acetamido]pyridin-2-yl}-N-(3,5-difluoro-4-methoxyphenyl)acetamide;
N-{4-[2-(2-chloro-4-fluorophenyl)acetamido]pyridin-2-yl}-N-(3,5-difluoro-4-methoxyphenyl)acetamide;
N-{4-[2-(2-chloro-3-fluorophenyl)acetamido]pyridin-2-yl}-N-(3,5-difluoro-4-methoxyphenyl)acetamide;
N-{4-[2-(2,6-dichlorophenyl)acetamido]pyridin-2-yl}-N-(3,5-difluoro-4-methoxyphenyl)acetamide;
N-{4-[2-(2-chlorophenyl)acetamido]pyridin-2-yl}-N-(3-methoxyphenyl)acetamide;
N-{4-[2-(2-chloro-3-fluorophenyl)acetamido]pyridin-2-yl}-N-(3-methoxyphenyl)acetamide;
N-{4-[2-(2-chlorophenyl)acetamido]pyridin-2-yl}-N-[3-(trifluoromethoxy)phenyl]acetamide;
N-{4-[2-(2-chloro-3-fluorophenyl)acetamido]pyridin-2-yl}-N-(4-fluoro-3-methoxyphenyl)acetamide;
N-{4-[2-(2,6-dichlorophenyl)acetamido]pyridin-2-yl}-N-(4-fluoro-3-methoxyphenyl)acetamide;
N-(2-chlorophenyl)-N-(4-{2-[2-chloro-3-(trifluoromethyl)phenyl]acetamido}pyridin-2-yl)acetamide;
N-(2-chlorophenyl)-N-{4-[2-(2-chlorophenyl)acetamido]pyridin-2-yl}acetamide;
N-{4-[2-(2-chloro-4-fluorophenyl)acetamido]pyridin-2-yl}-N-(2-chlorophenyl)acetamide;
N-{4-[2-(2-chloro-3-fluorophenyl)acetamido]pyridin-2-yl}-N-(2-chlorophenyl)acetamide;
N-(2-chlorophenyl)-N-{4-[2-(2,6-dichlorophenyl)acetamido]pyridin-2-yl}acetamide;
N-(2-chloro-5-fluorophenyl)-N-(4-{2-[2-chloro-3-(trifluoromethyl)phenyl]acetamido}pyridin-2-yl)acetamide;
N-(2-chloro-5-fluorophenyl)-N-{4-[2-(2-chlorophenyl)acetamido]pyridin-2-yl}acetamide;
N-(2-chloro-5-fluorophenyl)-N-{4-[2-(2-chloro-4-fluorophenyl)acetamido]pyridin-2-yl}acetamide;
N-(2-chloro-5-fluorophenyl)-N-{4-[2-(2-chloro-3-fluorophenyl)acetamido]pyridin-2-yl}acetamide;
N-(2-chloro-5-fluorophenyl)-N-{4-[2-(2,6-dichlorophenyl)acetamido]pyridin-2-yl}acetamide;
N-{4-[2-(2-chlorophenyl)acetamido]pyridin-2-yl}-N-(2-fluorophenyl)acetamide;
N-{4-[2-(2-chloro-4-fluorophenyl)acetamido]pyridin-2-yl}-N-(2-fluorophenyl)acetamide;
N-{4-[2-(2-chloro-3-fluorophenyl)acetamido]pyridin-2-yl}-N-(2-fluorophenyl)acetamide;
N-{4-[2-(2,6-dichlorophenyl)acetamido]pyridin-2-yl}-N-(2-fluorophenyl)acetamide;
N-{4-[2-(2-chloro-3-fluorophenyl)acetamido]pyridin-2-yl}-N-[2-fluoro-4-(trifluoromethyl)phenyl]acetamide;
N-{4-[2-(2,6-dichlorophenyl)acetamido]pyridin-2-yl}-N-[2-fluoro-4-(trifluoromethyl)phenyl]acetamide;
N-(4-{2-[2-chloro-3-(trifluoromethyl)phenyl]acetamido}pyridin-2-yl)-N-(2,3-difluorophenyl)acetamide;
N-{4-[2-(2-chlorophenyl)acetamido]pyridin-2-yl}-N-(2,3-difluorophenyl)acetamide;
N-{4-[2-(2-chloro-4-fluorophenyl)acetamido]pyridin-2-yl}-N-(2,3-difluorophenyl)acetamide;
N-{4-[2-(2-chloro-3-fluorophenyl)acetamido]pyridin-2-yl}-N-(2,3-difluorophenyl)acetamide;
N-{4-[2-(2,6-dichlorophenyl)acetamido]pyridin-2-yl}-N-(2,3-difluorophenyl)acetamide;
N-{4-[2-(2-chlorophenyl)acetamido]pyridin-2-yl}-N-(2,4-difluorophenyl)acetamide;
N-{4-[2-(2-chloro-4-fluorophenyl)acetamido]pyridin-2-yl}-N-(2,4-difluorophenyl)acetamide;
N-{4-[2-(2-chloro-3-fluorophenyl)acetamido]pyridin-2-yl}-N-(2,4-difluorophenyl)acetamide;
N-{4-[2-(2,6-dichlorophenyl)acetamido]pyridin-2-yl}-N-(2,4-difluorophenyl)acetamide;
N-{4-[2-(2-chloro-6-fluorophenyl)acetamido]pyridin-2-yl}-N-(2,4-difluorophenyl)acetamide;
N-(3-chlorophenyl)-N-{4-[2-(2-chlorophenyl)acetamido]pyridin-2-yl}acetamide;
N-{4-[2-(2-chloro-4-fluorophenyl)acetamido]pyridin-2-yl}-N-(3-chlorophenyl)acetamide;
N-{4-[2-(2-chloro-3-fluorophenyl)acetamido]pyridin-2-yl}-N-(3-chlorophenyl)acetamide;
N-(3-chlorophenyl)-N-{4-[2-(2,6-dichlorophenyl)acetamido]pyridin-2-yl}acetamide;
N-{4-[2-(2-chloro-6-fluorophenyl)acetamido]pyridin-2-yl}-N-(3-chlorophenyl)acetamide;
N-(3-chloro-5-fluorophenyl)-N-{4-[2-(2-chlorophenyl)acetamido]pyridin-2-yl}acetamide;
N-(3-chloro-5-fluorophenyl)-N-{4-[2-(2-chloro-4-fluorophenyl)acetamido]pyridin-2-yl}acetamide;
N-(3-chloro-5-fluorophenyl)-N-{4-[2-(2-chloro-3-fluorophenyl)acetamido]pyridin-2-yl}acetamide;
N-(3-chloro-5-fluorophenyl)-N-{4-[2-(2,6-dichlorophenyl)acetamido]pyridin-2-yl}acetamide;
N-(3-chloro-5-fluorophenyl)-N-{4-[2-(2-chloro-6-fluorophenyl)acetamido]pyridin-2-yl}acetamide;
N-{4-[2-(2-chloro-4-fluorophenyl)acetamido]pyridin-2-yl}-N-(4-fluorophenyl)butanamide;
N-{4-[2-(2-chloro-6-fluorophenyl)acetamido]pyridin-2-yl}-N-(4-fluorophenyl)butanamide;
N-{4-[2-(2-chlorophenyl)acetamido]pyridin-2-yl}-N-(4-fluorophenyl)butanamide;
N-{4-[2-(2,6-dichlorophenyl)acetamido]pyridin-2-yl}-N-(4-fluorophenyl)butanamide;
N-{4-[2-(2-Chlorphenyl)acetamido]pyridin-2-yl}-N-(3-cyan-5-fluorphenyl)acetamide;
N-(3-Cyan-5-fluorphenyl)-N-{4-[2-(2,6-dichlorphenyl)acetamido]pyridin-2-yl}acetamide;
N-{4-[2-(2-Chlor-3-fluorphenyl)acetamido]pyridin-2-yl}-N-(3-cyan-5-fluorphenyl)acetamide;
N-{4-[2-(2-Chlor-6-fluorphenyl)acetamido]pyridin-2-yl}-N-(3-cyan-5-fluorphenyl)acetamide;
N-{4-[2-(2-Chlor-4-fluorphenyl)acetamido]pyridin-2-yl}-N-(3-cyan-5-fluorphenyl)acetamide;
N-(2-Chlor-4-fluorphenyl)-N-{4-[2-(2-chlorphenyl)acetamido]pyridin-2-yl}acetamide;
N-(2-Chlor-4-fluorphenyl)-N-{4-[2-(2-chlor-4-fluorphenyl)acetamido]pyridin-2-yl}acetamide;
N-(2-Chlor-4-fluorphenyl)-N-{4-[2-(2,6-dichlorphenyl)acetamido]pyridin-2-yl}acetamide;
N-(2-Chlor-4-fluorphenyl)-N-{4-[2-(2-chlor-6-fluorphenyl)acetamido]pyridin-2-yl}acetamide;
N-(2-Chlor-4-fluorphenyl)-N-{4-[2-(2-chlor-3-fluorphenyl)acetamido]pyridin-2-yl}acetamide;
N-[3-Chlor-4-(methylsulfonyl)phenyl]-N-{4-[2-(2,6-dichlorphenyl)acetamido]pyridin-2-yl}acetamide;
N-{4-[2-(2-Chlor-3-fluorphenyl)acetamido]pyridin-2-yl}-N-[3-chlor-4-(methylsulfonyl)phenyl]acetamide;
N-[3-Chlor-4-(methylsulfonyl)phenyl]-N-{4-[2-(2-chlorphenyl)acetamido]pyridin-2-yl}acetamide;
N-{4-[2-(2-Chlor-6-fluorphenyl)acetamido]pyridin-2-yl}-N-[3-chlor-4-(methylsulfonyl)phenyl]acetamide;
N-{4-[2-(2-Chlor-4-fluorphenyl)acetamido]pyridin-2-yl}-N-[3-chlor-4-(methylsulfonyl)phenyl]acetamide;
N-{4-[2-(2-Chlor-3-fluorphenyl)acetamido]pyridin-2-yl}-N-(3-fluor-5-methoxyphenyl)acetamide;
N-{4-[2-(2-Chlorphenyl)acetamido]pyridin-2-yl}-N-(3-fluor-5-methoxyphenyl)acetamide;
N-{4-[2-(2,6-Dichlorphenyl)acetamido]pyridin-2-yl}-N-(3-fluor-5-methoxyphenyl)acetamide;
N-{4-[2-(2-Chlor-4-fluorphenyl)acetamido]pyridin-2-yl}-N-(3-fluor-5-methoxyphenyl)acetamide;
N-{4-[2-(2-Chlor-6-fluorphenyl)acetamido]pyridin-2-yl}-N-(3-fluor-5-methoxyphenyl)acetamide;
N-{4-[2-(2,6-Dichlorphenyl)acetamido]pyridin-2-yl}-N-[2-(difluormethyl)phenyl]acetamide;
N-{4-[2-(2-Chlorphenyl)acetamido]pyridin-2-yl}-N-[2-(difluormethyl)phenyl]acetamide;
N-{4-[2-(2-Chlor-3-fluorphenyl)acetamido]pyridin-2-yl}-N-[2-(difluormethyl)phenyl]acetamide;
N-{4-[2-(2-Chlor-6-fluorphenyl)acetamido]pyridin-2-yl}-N-[2-(difluormethyl)phenyl]acetamide;
N-{4-[2-(2-Chlor-4-fluorphenyl)acetamido]pyridin-2-yl}-N-[2-(difluormethyl)phenyl]acetamide;
N-{4-[2-(2-Chlorphenyl)acetamido]pyridin-2-yl}-N-(2,4-dimethylphenyl)acetamide;
N-{4-[2-(2-Chlor-3-fluorphenyl)acetamido]pyridin-2-yl}-N-(2,4-dimethylphenyl)acetamide;
N-{4-[2-(2-Chlor-6-fluorphenyl)acetamido]pyridin-2-yl}-N-(2,4-dimethylphenyl)acetamide;
N-{4-[2-(2,6-Dichlorphenyl)acetamido]pyridin-2-yl}-N-(2,4-dimethylphenyl)acetamide;
N-{4-[2-(2-Chlor-4-fluorphenyl)acetamido]pyridin-2-yl}-N-(2,4-dimethylphenyl)acetamide;
N-{4-[2-(2-Chlorphenyl)acetamido]pyridin-2-yl}-N-(3-cyan-5-methylphenyl)acetamide;
N-(3-Cyan-5-methylphenyl)-N-{4-[2-(2,6-dichlorphenyl)acetamido]pyridin-2-yl}acetamide;
N-{4-[2-(2-Chlor-3-fluorphenyl)acetamido]pyridin-2-yl}-N-(3-cyan-5-methylphenyl)acetamide;
N-{4-[2-(2-Chlor-4-fluorphenyl)acetamido]pyridin-2-yl}-N-(3-cyan-5-methylphenyl)acetamide;
N-{4-[2-(2-Chlor-6-fluorphenyl)acetamido]pyridin-2-yl}-N-(3-cyan-5-methylphenyl)acetamide;
N-(3-Chlor-4-methylphenyl)-N-{4-[2-(2-chlorphenyl)acetamido]pyridin-2-yl}acetamide;
N-{4-[2-(2-Chlor-3-fluorphenyl)acetamido]pyridin-2-yl}-N-(3-chlor-4-methylphenyl)acetamide;
N-(3-Chlor-4-methylphenyl)-N-{4-[2-(2,6-dichlorphenyl)acetamido]pyridin-2-yl}acetamide;
N-{4-[2-(2-Chlor-6-fluorphenyl)acetamido]pyridin-2-yl}-N-(3-chlor-4-methylphenyl)acetamide;
N-{4-[2-(2-Chlor-6-fluorphenyl)acetamido]pyridin-2-yl}-N-(4-fluor-2,3-dimethylphenyl)acetamide;
N-{5-[2-(2,6-dichlorophenyl)acetamido]pyridazin-3-yl}-N-phenylacetamide;
N-{5-[2-(2-chloro-3-fluorophenyl)acetamido]pyridazin-3-yl}-N-phenylacetamide;
N-{5-[2-(2-chlorophenyl)acetamido]pyridazin-3-yl}-N-phenylacetamide;
N-{5-[2-(2-chloro-4-fluorophenyl)acetamido]pyridazin-3-yl}-N-phenylacetamide;
N-{5-[2-(2-chloro-6-fluorophenyl)acetamido]pyridazin-3-yl}-N-(4-fluorophenyl)acetamide;
N-{5-[2-(2-chloro-4-fluorophenyl)acetamido]pyridazin-3-yl}-N-(4-fluorophenyl)acetamide;
N-{5-[2-(2-chloro-3-fluorophenyl)acetamido]pyridazin-3-yl}-N-(4-fluorophenyl)acetamide;
N-{5-[2-(2-chlorophenyl)acetamido]pyridazin-3-yl}-N-(4-fluorophenyl)acetamide;
N-{5-[2-(2,6-dichlorophenyl)acetamido]pyridazin-3-yl}-N-(4-fluorophenyl)acetamide;
N-{5-[2-(2,6-dichlorophenyl)acetamido]pyridazin-3-yl}-N-(3-fluorophenyl)acetamide;
N-{5-[2-(2-chloro-3-fluorophenyl)acetamido]pyridazin-3-yl}-N-(3-fluorophenyl)acetamide;
N-{5-[2-(2-chlorophenyl)acetamido]pyridazin-3-yl}-N-(3-fluorophenyl)acetamide;
N-{5-[2-(2-chloro-6-fluorophenyl)acetamido]pyridazin-3-yl}-N-(3-fluorophenyl)acetamide;
N-{5-[2-(2-chloro-4-fluorophenyl)acetamido]pyridazin-3-yl}-N-(3-fluorophenyl)acetamide;
N-{5-[2-(2-chlorophenyl)acetamido]pyridazin-3-yl}-N-(3,4-difluorophenyl)acetamide;
N-{5-[2-(2,6-dichlorophenyl)acetamido]pyridazin-3-yl}-N-(3,4-difluorophenyl)acetamide;
N-{5-[2-(2-chloro-3-fluorophenyl)acetamido]pyridazin-3-yl}-N-(3,4-difluorophenyl)acetamide;
N-{5-[2-(2-chloro-4-fluorophenyl)acetamido]pyridazin-3-yl}-N-(3,4-difluorophenyl)acetamide;
N-{5-[2-(2-chloro-6-fluorophenyl)acetamido]pyridazin-3-yl}-N-(3,4-difluorophenyl)acetamide;
N-{5-[2-(2,6-dichlorophenyl)acetamido]pyridazin-3-yl}-N-(3,5-difluorophenyl)acetamide;
N-{5-[2-(2-chlorophenyl)acetamido]pyridazin-3-yl}-N-(3,5-difluorophenyl)acetamide;
N-{5-[2-(2-chloro-3-fluorophenyl)acetamido]pyridazin-3-yl}-N-(3,5-difluorophenyl)acetamide;
N-{5-[2-(2-chloro-4-fluorophenyl)acetamido]pyridazin-3-yl}-N-(3,5-difluorophenyl)acetamide; and
N-{5-[2-(2-chloro-6-fluorophenyl)acetamido]pyridazin-3-yl}-N-(3,5-difluorophenyl)acetamide;
or a stereoisomer, a tautomer, an N-oxide, a hydrate, a solvate, or a pharmaceutically acceptable salt thereof, or a mixture of any of the foregoing.

12: A method of treatment or prophylaxis of dry eye syndrome, dry eye, ocular neuropathic pain, ocular trauma, and post-operative ocular pain, comprising administering a compound selected from the group consisting of
2-(Dimethylamino)-N-[4-(2-oxo-2,3-dihydro-1H-naphtho[1,2-e][1,4]diazepin-5-yl)phenyl]nicotinamide;
5-[4-(2-Iodobenzoylamino)phenyl]-1H-naphtho[1,2-b][1,4]diazepine-2,4(3H,5H)-dione,
5-[4-[(2-(Trifluoromethyl)benzoyl]aminophenyl]-1H-naphtho[1,2-b][1,4]diazepine-2,4(3H,5H)-dione,
5-[3-(1H-Tetrazol-5-yl)phenyl]-1H-naphtho[1,2-b][1,4]diazepine-2,4(3H,5H)-dione,
2-(2-chlorophenyl)-N-[2-(difluoromethyl)-4-sulfamoyl-2H-indazol-6-yl]acetamide;
2-(2-chlorophenyl)-N-(4-sulfamoyl-2H-indazol-6-yl)acetamide;
2-(2-chlorophenyl)-N-(2-methyl-4-sulfamoyl-2H-indazol-6-yl)acetamide; and
2-(2-chlorophenyl)-N-(1-methyl-4-sulfamoyl-1H-indazol-6-yl)acetamide;
or a stereoisomer, a tautomer, an N-oxide, a hydrate, a solvate, or a pharmaceutically acceptable pharmaceutically acceptable salt thereof, or a mixture of any of the foregoing, to a mammal in need thereof.

13: A method of treatment or prophylaxis of dry eye syndrome, dry eye, ocular neuropathic pain, ocular trauma, and post-operative trauma, comprising administering a compound N-{4-[2-(2-chloro-3-fluorophenyl)acetamido]pyridin-2-yl}-N-(4-fluorophenyl)acetamide or a stereoisomer, a tautomer, an N-oxide, a hydrate, a solvate, or a pharmaceutically acceptable salt thereof, or a mixture of any of the foregoing, to a mammal in need thereof.

14: The method of claim 2, wherein the compound is administered in the form of a pharmaceutical composition comprising the compound and one or more pharmaceutically acceptable excipients.

15-16. (canceled)

17: The method of claim 11, wherein the compound is administered in the form of a pharmaceutical composition comprising the compound and one or more pharmaceutically acceptable excipients.

18: The method of claim 12, wherein the compound is administered in the form of a pharmaceutical composition comprising the compound and one or more pharmaceutically acceptable excipients.

19: The method of claim 1, wherein the mammal is a human.

20: The method of claim 13, where in the mammal is a human.
